SEQUENCE LISTING (89 sequences)

SEQ ID NO: 1 - DNA, 20 bp, synthetic construct
  source 1..20: mol_type = other DNA
gctactctctctttctggcc

SEQ ID NO: 2 - DNA, 20 bp, synthetic construct
  source 1..20: mol_type = other DNA
ggccgagatgtctcgctccg

SEQ ID NO: 3 - DNA, 20 bp, synthetic construct
  source 1..20: mol_type = other DNA
cgcgagcacagctaaggcca

SEQ ID NO: 4 - DNA, 20 bp, synthetic construct
  source 1..20: mol_type = other DNA
tataagtggaggcgtcgcgc

SEQ ID NO: 5 - DNA, 20 bp, synthetic construct
  source 1..20: mol_type = other DNA
gagtagcgcgagcacagcta

SEQ ID NO: 6 - DNA, 20 bp, synthetic construct
  source 1..20: mol_type = other DNA
actggacgcgtcgcgctggc

SEQ ID NO: 7 - DNA, 20 bp, synthetic construct
  source 1..20: mol_type = other DNA
aagtggaggcgtcgcgctgg

SEQ ID NO: 8 - DNA, 20 bp, synthetic construct
  source 1..20: mol_type = other DNA
ggccacggagcgagacatct

SEQ ID NO: 9 - DNA, 20 bp, synthetic construct
  source 1..20: mol_type = other DNA
gcccgaatgctgtcagcttc

SEQ ID NO: 10 - DNA, 20 bp, synthetic construct
  source 1..20: mol_type = other DNA
ctcgcgctactctctctttc

SEQ ID NO: 11 - DNA, 20 bp, synthetic construct
  source 1..20: mol_type = other DNA
tcctgaagctgacagcattc

SEQ ID NO: 12 - DNA, 20 bp, synthetic construct
  source 1..20: mol_type = other DNA
ttcctgaagctgacagcatt

SEQ ID NO: 13 - DNA, 20 bp, synthetic construct
  source 1..20: mol_type = other DNA
actctctctttctggcctgg

SEQ ID NO: 14 - DNA, 130 bp, synthetic construct
  source 1..130: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 15 - DNA, 800 bp, synthetic construct
  source 1..800: mol_type = other DNA
gttctagggtggaaactaagagaatgatgtacctagagggcgctggaagctctaaagccctagcagttactgcttttactattagtggtcgtttttttctcccccccgccccccgacaaatcaacagaacaaagaaaattacctaaacagcaaggacatagggaggaacttcttggcacagaactttccaaacactttttcctgaagggatacaagaagcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagct

SEQ ID NO: 16 - DNA, 1667 bp, synthetic construct
  source 1..1667: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacag

SEQ ID NO: 17 - DNA, 546 bp, synthetic construct
  source 1..546: mol_type = other DNA
atgaggaggatgtgggccacgcaggggctggcggtggcgctggctctgagcgtgctgccgggcagccgggcgctgcggccgggcgactgcgaagtttgtatttcttatctgggaagattttaccaggacctcaaagacagagatgtcacattctcaccagccactattgaaaacgaacttataaagttctgccgggaagcaagaggcaaagagaatcggttgtgctactatatcggggccacagatgatgcagccaccaaaatcatcaatgaggtatcaaagcctctggcccaccacatccctgtggagaagatctgtgagaagcttaagaagaaggacagccagatatgtgagcttaagtatgacaagcagatcgacctgagcacagtggacctgaagaagctccgagttaaagagctgaagaagattctggatgactggggggagacatgcaaaggctgtgcagaaaagtctgactacatccggaagataaatgaactgatgcctaaatatgcccccaaggcagccagtgcacggaccgatttg

SEQ ID NO: 18 - DNA, 57 bp, synthetic construct
  source 1..57: mol_type = other DNA
gctactaacttcagcctgctgaagcaggctggagacgtggaggagaaccctggacct

SEQ ID NO: 19 - DNA, 2370 bp, synthetic construct
  source 1..2370: mol_type = other DNA
atggctgaacaagtccttcctcaggctttgtatttgagcaatatgcggaaagctgtgaagatacgggagagaactccagaagacatttttaaacctactaatgggatcattcatcattttaaaaccatgcaccgatacacactggaaatgttcagaacttgccagttttgtcctcagtttcgggagatcatccacaaagccctcatcgacagaaacatccaggccaccctggaaagccagaagaaactcaactggtgtcgagaagtccggaagcttgtggcgctgaaaacgaacggtgacggcaattgcctcatgcatgccacttctcagtacatgtggggcgttcaggacacagacttggtactgaggaaggcgctgttcagcacgctcaaggaaacagacacacgcaactttaaattccgctggcaactggagtctctcaaatctcaggaatttgttgaaacggggctttgctatgatactcggaactggaatgatgaatgggacaatcttatcaaaatggcttccacagacacacccatggcccgaagtggacttcagtacaactcactggaagaaatacacatatttgtcctttgcaacatcctcagaaggccaatcattgtcatttcagacaaaatgctaagaagtttggaatcaggttccaatttcgcccctttgaaagtgggtggaatttacttgcctctccactggcctgcccaggaatgctacagataccccattgttctcggctatgacagccatcattttgtacccttggtgaccctgaaggacagtgggcctgaaatccgagctgttccacttgttaacagagaccggggaagatttgaagacttaaaagttcactttttgacagatcctgaaaatgagatgaaggagaagctcttaaaagagtacttaatggtgatagaaatccccgtccaaggctgggaccatggcacaactcatctcatcaatgccgcaaagttggatgaagctaacttaccaaaagaaatcaatctggtagatgattactttgaacttgttcagcatgagtacaagaaatggcaggaaaacagcgagcaggggaggagagaggggcacgcccagaatcccatggaaccttccgtgccccagctttctctcatggatgtaaaatgtgaaacgcccaactgccccttcttcatgtctgtgaacacccagcctttatgccatgagtgctcagagaggcggcaaaagaatcaaaacaaactcccaaagctgaactccaagccgggccctgaggggctccctggcatggcgctcggggcctctcggggagaagcctatgagcccttggcgtggaaccctgaggagtccactggggggcctcattcggccccaccgacagcacccagcccttttctgttcagtgagaccactgccatgaagtgcaggagccccggctgccccttcacactgaatgtgcagcacaacggattttgtgaacgttgccacaacgcccggcaacttcacgccagccacgccccagaccacacaaggcacttggatcccgggaagtgccaagcctgcctccaggatgttaccaggacatttaatgggatctgcagtacttgcttcaaaaggactacagcagaggcctcctccagcctcagcaccagcctccctccttcctgtcaccagcgttccaagtcagatccctcgcggctcgtccggagcccctccccgcattcttgccacagagctggaaacgacgcccctgctggctgcctgtctcaagctgcacggactcctggggacaggacggggacgagcaagtgcagaaaagccggctgcgtgtattttgggactccagaaaacaagggcttttgcacactgtgtttcatcgagtacagagaaaacaaacattttgctgctgcctcagggaaagtcagtcccacagcgtccaggttccagaacaccattccgtgcctggggagggaatgcggcacccttggaagcaccatgtttgaaggatactgccagaagtgtttcattgaagctcagaatcagagatttcatgaggccaaaaggacagaagagcaactgagatcgagccagcgcagagatgtgcctcgaaccacacaaagcacctcaaggcccaagtgcgcccgggcctcctgcaagaacatcctggcctgccgcagcgaggagctctgcatggagtgtcagcatcccaaccagaggatgggccctggggcccaccggggtgagcctgcccccgaagacccccccaagcagcgttgccgggcccccgcctgtgatcattttggcaatgccaagtgcaacggctactgcaacgaatgctttcagttcaagcagatgtatggc

SEQ ID NO: 20 - DNA, 873 bp, synthetic construct
  source 1..873: mol_type = other DNA
atgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaaacttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaa

SEQ ID NO: 21 - DNA, 225 bp, synthetic construct
  source 1..225: mol_type = other DNA
gtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggt

SEQ ID NO: 22 - DNA, 800 bp, synthetic construct
  source 1..800: mol_type = other DNA
ccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttct

SEQ ID NO: 23 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 24 - DNA, 10181 bp, synthetic construct
  source 1..10181: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgttctagggtggaaactaagagaatgatgtacctagagggcgctggaagctctaaagccctagcagttactgcttttactattagtggtcgtttttttctcccccccgccccccgacaaatcaacagaacaaagaaaattacctaaacagcaaggacatagggaggaacttcttggcacagaactttccaaacactttttcctgaagggatacaagaagcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctaagcttgtggacgatatcgaattcgcacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggggggatccgtttatctgcagaattcgcccttgacgtcgccaccatgaggaggatgtgggccacgcaggggctggcggtggcgctggctctgagcgtgctgccgggcagccgggcgctgcggccgggcgactgcgaagtttgtatttcttatctgggaagattttaccaggacctcaaagacagagatgtcacattctcaccagccactattgaaaacgaacttataaagttctgccgggaagcaagaggcaaagagaatcggttgtgctactatatcggggccacagatgatgcagccaccaaaatcatcaatgaggtatcaaagcctctggcccaccacatccctgtggagaagatctgtgagaagcttaagaagaaggacagccagatatgtgagcttaagtatgacaagcagatcgacctgagcacagtggacctgaagaagctccgagttaaagagctgaagaagattctggatgactggggggagacatgcaaaggctgtgcagaaaagtctgactacatccggaagataaatgaactgatgcctaaatatgcccccaaggcagccagtgcacggaccgatttgggaagcggagctactaacttcagcctgctgaagcaggctggagacgtggaggagaaccctggacctatggctgaacaagtccttcctcaggctttgtatttgagcaatatgcggaaagctgtgaagatacgggagagaactccagaagacatttttaaacctactaatgggatcattcatcattttaaaaccatgcaccgatacacactggaaatgttcagaacttgccagttttgtcctcagtttcgggagatcatccacaaagccctcatcgacagaaacatccaggccaccctggaaagccagaagaaactcaactggtgtcgagaagtccggaagcttgtggcgctgaaaacgaacggtgacggcaattgcctcatgcatgccacttctcagtacatgtggggcgttcaggacacagacttggtactgaggaaggcgctgttcagcacgctcaaggaaacagacacacgcaactttaaattccgctggcaactggagtctctcaaatctcaggaatttgttgaaacggggctttgctatgatactcggaactggaatgatgaatgggacaatcttatcaaaatggcttccacagacacacccatggcccgaagtggacttcagtacaactcactggaagaaatacacatatttgtcctttgcaacatcctcagaaggccaatcattgtcatttcagacaaaatgctaagaagtttggaatcaggttccaatttcgcccctttgaaagtgggtggaatttacttgcctctccactggcctgcccaggaatgctacagataccccattgttctcggctatgacagccatcattttgtacccttggtgaccctgaaggacagtgggcctgaaatccgagctgttccacttgttaacagagaccggggaagatttgaagacttaaaagttcactttttgacagatcctgaaaatgagatgaaggagaagctcttaaaagagtacttaatggtgatagaaatccccgtccaaggctgggaccatggcacaactcatctcatcaatgccgcaaagttggatgaagctaacttaccaaaagaaatcaatctggtagatgattactttgaacttgttcagcatgagtacaagaaatggcaggaaaacagcgagcaggggaggagagaggggcacgcccagaatcccatggaaccttccgtgccccagctttctctcatggatgtaaaatgtgaaacgcccaactgccccttcttcatgtctgtgaacacccagcctttatgccatgagtgctcagagaggcggcaaaagaatcaaaacaaactcccaaagctgaactccaagccgggccctgaggggctccctggcatggcgctcggggcctctcggggagaagcctatgagcccttggcgtggaaccctgaggagtccactggggggcctcattcggccccaccgacagcacccagcccttttctgttcagtgagaccactgccatgaagtgcaggagccccggctgccccttcacactgaatgtgcagcacaacggattttgtgaacgttgccacaacgcccggcaacttcacgccagccacgccccagaccacacaaggcacttggatcccgggaagtgccaagcctgcctccaggatgttaccaggacatttaatgggatctgcagtacttgcttcaaaaggactacagcagaggcctcctccagcctcagcaccagcctccctccttcctgtcaccagcgttccaagtcagatccctcgcggctcgtccggagcccctccccgcattcttgccacagagctggaaacgacgcccctgctggctgcctgtctcaagctgcacggactcctggggacaggacggggacgagcaagtgcagaaaagccggctgcgtgtattttgggactccagaaaacaagggcttttgcacactgtgtttcatcgagtacagagaaaacaaacattttgctgctgcctcagggaaagtcagtcccacagcgtccaggttccagaacaccattccgtgcctggggagggaatgcggcacccttggaagcaccatgtttgaaggatactgccagaagtgtttcattgaagctcagaatcagagatttcatgaggccaaaaggacagaagagcaactgagatcgagccagcgcagagatgtgcctcgaaccacacaaagcacctcaaggcccaagtgcgcccgggcctcctgcaagaacatcctggcctgccgcagcgaggagctctgcatggagtgtcagcatcccaaccagaggatgggccctggggcccaccggggtgagcctgcccccgaagacccccccaagcagcgttgccgggcccccgcctgtgatcattttggcaatgccaagtgcaacggctactgcaacgaatgctttcagttcaagcagatgtatggcggaagcggagctactaacttcagcctgctgaagcaggctggagacgtggaggagaaccctggacctatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaaacttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtcgacccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttctggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 25 - DNA, 20 bp, synthetic construct
  source 1..20: mol_type = other DNA
ggtccatctggtcatagaag

SEQ ID NO: 26 - DNA, 800 bp, synthetic construct
  source 1..800: mol_type = other DNA
catatttatggggtatatgtgaatatttattacatgcatagaaggtataatgatcatgtcaggatatttgaggtatccacatttgggattgtttaaagattaaatgaaatagtgttaaaagtatttaatatgcccttcaacaaatgatgaggaaatcttagaatctgctcagactccttcagtttacatattaggaaactgaggcacagaaaggagcagagacttgctcaagtccacccaaagcagtagagcattgtggttaaatgcaggacttcagtcagactgtctgggttcaaatcctggttccacttggacatgggtttccttacataaatcacttcacctctctgagcctcagttttctcatatgcaaagtgaggataataataataccttccttacatggttactgatatgagtattaaatgtgccagctcatgtgcctggcgtataggaggtgctttataaaccttagctgttaccactcatggcattgccaaatgtgggacgggtctcctgactctctggtgtgagattgatggaatccacactttccagttcccttttctacctcctgggtatcttctcatatggttgtaagttccttggaggaagggaatgtggcttgctctctccaccacgctgagcatataagaggtgctgaatgagcgcttttattcactcctctcatccccagccctcaccagctgggagttgttgtaggtgtcaattttctgcctctttccaacaccctgtgaggtgactgagcattgtcttccctcccaggcagctcacagtgt

SEQ ID NO: 27 - DNA, 1530 bp, synthetic construct
  source 1..1530: mol_type = other DNA
atggaagatacaaaggagtctaacgtgaagacattttgctccaagaatatcctagccatccttggcttctcctctatcatagctgtgatagctttgcttgctgtggggttgacccagaacaaagcattgccagaaaacgttaagtatgggattgtgctggatgcgggttcttctcacacaagtttatacatctataagtggccagcagaaaaggagaatgacacaggcgtggtgcatcaagtagaagaatgcagggttaaaggtcctggaatctcaaaatttgttcagaaagtaaatgaaataggcatttacctgactgattgcatggaaagagctagggaagtgattccaaggtcccagcaccaagagacacccgtttacctgggagccacggcaggcatgcggttgctcaggatggaaagtgaagagttggcagacagggttctggatgtggtggagaggagcctcagcaactacccctttgacttccagggtgccaggatcattactggccaagaggaaggtgcctatggctggattactatcaactatctgctgggcaaattcagtcagaaaacaaggtggttcagcatagtcccatatgaaaccaataatcaggaaacctttggagctttggaccttgggggagcctctacacaagtcacttttgtaccccaaaaccagactatcgagtccccagataatgctctgcaatttcgcctctatggcaaggactacaatgtctacacacatagcttcttgtgctatgggaaggatcaggcactctggcagaaactggccaaggacattcaggttgcaagtaatgaaattctcagggacccatgctttcatcctggatataagaaggtagtgaacgtaagtgacctttacaagaccccctgcaccaagagatttgagatgactcttccattccagcagtttgaaatccagggtattggaaactatcaacaatgccatcaaagcatcctggagctcttcaacaccagttactgcccttactcccagtgtgccttcaatgggattttcttgccaccactccagggggattttggggcattttcagctttttactttgtgatgaagtttttaaacttgacatcagagaaagtctctcaggaaaaggtgactgagatgatgaaaaagttctgtgctcagccttgggaggagataaaaacatcttacgctggagtaaaggagaagtacctgagtgaatactgcttttctggtacctacattctctccctccttctgcaaggctatcatttcacagctgattcctgggagcacatccatttcattggcaagatccagggcagcgacgccggctggactttgggctacatgctgaacctgaccaacatgatcccagctgagcaaccattgtccacacctctctcccactccacctatgtcttcctcatggttctattctccctggtccttttcacagtggccatcataggcttgcttatctttcacaagccttcatatttctggaaagatatggta

SEQ ID NO: 28 - DNA, 800 bp, synthetic construct
  source 1..800: mol_type = other DNA
tgaccagatggacctggctggagaagaagagattgagctctactcaggtgggccctcctccctctggtctcttccggtatcccccacccctcagcttgctgtagagacggcaatcaggggaaattctggtccctgccctcccgtcagcaccacggacagctcccacgtctgtgggacgctctctgcagatggggatgatctcccagccctgccccgcctctccctcgttccccaccagccctctttccagaaatttccttcttcatccaagggacttttcctcccagaacccgacacagacaccatcaactgcgaccagttcagcaggctgttgtgtgacatggaaggtgatgaagagaccagggaggcttatgccaatatcggtgaggaagcacctgagcccagaaaaggacaatcaagggcaagagttctttgctgccacttgtcaatatcacccattcatcatgagccacgtcagtcccctcccacagaaatcattgcaagggggatgcggagcaatggctggaggaacggagactccagggaagagaggggagatggaggccagtgggggaaataggccccttcactaatgaccaccaagaaaacaaaatctcatgtttacatcctccacctccatttctatacgcatttctgcttcttgctcttctgtccatcctttctacaaagcccataccatacacccctttcccttttcctcccagctccttagccaagctactctagtatttgtaataactagcatttactggatactcatagtatgctcattgctgtcc

SEQ ID NO: 29 - DNA, 7799 bp, synthetic construct
  source 1..7799: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtcatatttatggggtatatgtgaatatttattacatgcatagaaggtataatgatcatgtcaggatatttgaggtatccacatttgggattgtttaaagattaaatgaaatagtgttaaaagtatttaatatgcccttcaacaaatgatgaggaaatcttagaatctgctcagactccttcagtttacatattaggaaactgaggcacagaaaggagcagagacttgctcaagtccacccaaagcagtagagcattgtggttaaatgcaggacttcagtcagactgtctgggttcaaatcctggttccacttggacatgggtttccttacataaatcacttcacctctctgagcctcagttttctcatatgcaaagtgaggataataataataccttccttacatggttactgatatgagtattaaatgtgccagctcatgtgcctggcgtataggaggtgctttataaaccttagctgttaccactcatggcattgccaaatgtgggacgggtctcctgactctctggtgtgagattgatggaatccacactttccagttcccttttctacctcctgggtatcttctcatatggttgtaagttccttggaggaagggaatgtggcttgctctctccaccacgctgagcatataagaggtgctgaatgagcgcttttattcactcctctcatccccagccctcaccagctgggagttgttgtaggtgtcaattttctgcctctttccaacaccctgtgaggtgactgagcattgtcttccctcccaggcagctcacagtgtaagcttgtggacgatatcgaattcgcacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggggggatccgtttatctgcagaattcgcccttgacgtcgccaccatggaagatacaaaggagtctaacgtgaagacattttgctccaagaatatcctagccatccttggcttctcctctatcatagctgtgatagctttgcttgctgtggggttgacccagaacaaagcattgccagaaaacgttaagtatgggattgtgctggatgcgggttcttctcacacaagtttatacatctataagtggccagcagaaaaggagaatgacacaggcgtggtgcatcaagtagaagaatgcagggttaaaggtcctggaatctcaaaatttgttcagaaagtaaatgaaataggcatttacctgactgattgcatggaaagagctagggaagtgattccaaggtcccagcaccaagagacacccgtttacctgggagccacggcaggcatgcggttgctcaggatggaaagtgaagagttggcagacagggttctggatgtggtggagaggagcctcagcaactacccctttgacttccagggtgccaggatcattactggccaagaggaaggtgcctatggctggattactatcaactatctgctgggcaaattcagtcagaaaacaaggtggttcagcatagtcccatatgaaaccaataatcaggaaacctttggagctttggaccttgggggagcctctacacaagtcacttttgtaccccaaaaccagactatcgagtccccagataatgctctgcaatttcgcctctatggcaaggactacaatgtctacacacatagcttcttgtgctatgggaaggatcaggcactctggcagaaactggccaaggacattcaggttgcaagtaatgaaattctcagggacccatgctttcatcctggatataagaaggtagtgaacgtaagtgacctttacaagaccccctgcaccaagagatttgagatgactcttccattccagcagtttgaaatccagggtattggaaactatcaacaatgccatcaaagcatcctggagctcttcaacaccagttactgcccttactcccagtgtgccttcaatgggattttcttgccaccactccagggggattttggggcattttcagctttttactttgtgatgaagtttttaaacttgacatcagagaaagtctctcaggaaaaggtgactgagatgatgaaaaagttctgtgctcagccttgggaggagataaaaacatcttacgctggagtaaaggagaagtacctgagtgaatactgcttttctggtacctacattctctccctccttctgcaaggctatcatttcacagctgattcctgggagcacatccatttcattggcaagatccagggcagcgacgccggctggactttgggctacatgctgaacctgaccaacatgatcccagctgagcaaccattgtccacacctctctcccactccacctatgtcttcctcatggttctattctccctggtccttttcacagtggccatcataggcttgcttatctttcacaagccttcatatttctggaaagatatggtataatgatagccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtcgactgaccagatggacctggctggagaagaagagattgagctctactcaggtgggccctcctccctctggtctcttccggtatcccccacccctcagcttgctgtagagacggcaatcaggggaaattctggtccctgccctcccgtcagcaccacggacagctcccacgtctgtgggacgctctctgcagatggggatgatctcccagccctgccccgcctctccctcgttccccaccagccctctttccagaaatttccttcttcatccaagggacttttcctcccagaacccgacacagacaccatcaactgcgaccagttcagcaggctgttgtgtgacatggaaggtgatgaagagaccagggaggcttatgccaatatcggtgaggaagcacctgagcccagaaaaggacaatcaagggcaagagttctttgctgccacttgtcaatatcacccattcatcatgagccacgtcagtcccctcccacagaaatcattgcaagggggatgcggagcaatggctggaggaacggagactccagggaagagaggggagatggaggccagtgggggaaataggccccttcactaatgaccaccaagaaaacaaaatctcatgtttacatcctccacctccatttctatacgcatttctgcttcttgctcttctgtccatcctttctacaaagcccataccatacacccctttcccttttcctcccagctccttagccaagctactctagtatttgtaataactagcatttactggatactcatagtatgctcattgctgtccggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 30 - DNA, 8729 bp, synthetic construct
  source 1..8729: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgttctagggtggaaactaagagaatgatgtacctagagggcgctggaagctctaaagccctagcagttactgcttttactattagtggtcgtttttttctcccccccgccccccgacaaatcaacagaacaaagaaaattacctaaacagcaaggacatagggaggaacttcttggcacagaactttccaaacactttttcctgaagggatacaagaagcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctaagcttgtggacgatatcgaattcgcacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggggggatccgtttatctgcagaattcgcccttgacgtcgccaccatggaagatacaaaggagtctaacgtgaagacattttgctccaagaatatcctagccatccttggcttctcctctatcatagctgtgatagctttgcttgctgtggggttgacccagaacaaagcattgccagaaaacgttaagtatgggattgtgctggatgcgggttcttctcacacaagtttatacatctataagtggccagcagaaaaggagaatgacacaggcgtggtgcatcaagtagaagaatgcagggttaaaggtcctggaatctcaaaatttgttcagaaagtaaatgaaataggcatttacctgactgattgcatggaaagagctagggaagtgattccaaggtcccagcaccaagagacacccgtttacctgggagccacggcaggcatgcggttgctcaggatggaaagtgaagagttggcagacagggttctggatgtggtggagaggagcctcagcaactacccctttgacttccagggtgccaggatcattactggccaagaggaaggtgcctatggctggattactatcaactatctgctgggcaaattcagtcagaaaacaaggtggttcagcatagtcccatatgaaaccaataatcaggaaacctttggagctttggaccttgggggagcctctacacaagtcacttttgtaccccaaaaccagactatcgagtccccagataatgctctgcaatttcgcctctatggcaaggactacaatgtctacacacatagcttcttgtgctatgggaaggatcaggcactctggcagaaactggccaaggacattcaggttgcaagtaatgaaattctcagggacccatgctttcatcctggatataagaaggtagtgaacgtaagtgacctttacaagaccccctgcaccaagagatttgagatgactcttccattccagcagtttgaaatccagggtattggaaactatcaacaatgccatcaaagcatcctggagctcttcaacaccagttactgcccttactcccagtgtgccttcaatgggattttcttgccaccactccagggggattttggggcattttcagctttttactttgtgatgaagtttttaaacttgacatcagagaaagtctctcaggaaaaggtgactgagatgatgaaaaagttctgtgctcagccttgggaggagataaaaacatcttacgctggagtaaaggagaagtacctgagtgaatactgcttttctggtacctacattctctccctccttctgcaaggctatcatttcacagctgattcctgggagcacatccatttcattggcaagatccagggcagcgacgccggctggactttgggctacatgctgaacctgaccaacatgatcccagctgagcaaccattgtccacacctctctcccactccacctatgtcttcctcatggttctattctccctggtccttttcacagtggccatcataggcttgcttatctttcacaagccttcatatttctggaaagatatggtaggaagcggagctactaacttcagcctgctgaagcaggctggagacgtggaggagaaccctggacctatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaaacttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtcgacccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttctggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 31 - DNA, 9569 bp, synthetic construct
  source 1..9569: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgttctagggtggaaactaagagaatgatgtacctagagggcgctggaagctctaaagccctagcagttactgcttttactattagtggtcgtttttttctcccccccgccccccgacaaatcaacagaacaaagaaaattacctaaacagcaaggacatagggaggaacttcttggcacagaactttccaaacactttttcctgaagggatacaagaagcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctaagcttgtggacgatatcgaattcgcacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggggggatccgtttatctgcagaattcgcccttgacgtcgccaccatggctgaacaagtccttcctcaggctttgtatttgagcaatatgcggaaagctgtgaagatacgggagagaactccagaagacatttttaaacctactaatgggatcattcatcattttaaaaccatgcaccgatacacactggaaatgttcagaacttgccagttttgtcctcagtttcgggagatcatccacaaagccctcatcgacagaaacatccaggccaccctggaaagccagaagaaactcaactggtgtcgagaagtccggaagcttgtggcgctgaaaacgaacggtgacggcaattgcctcatgcatgccacttctcagtacatgtggggcgttcaggacacagacttggtactgaggaaggcgctgttcagcacgctcaaggaaacagacacacgcaactttaaattccgctggcaactggagtctctcaaatctcaggaatttgttgaaacggggctttgctatgatactcggaactggaatgatgaatgggacaatcttatcaaaatggcttccacagacacacccatggcccgaagtggacttcagtacaactcactggaagaaatacacatatttgtcctttgcaacatcctcagaaggccaatcattgtcatttcagacaaaatgctaagaagtttggaatcaggttccaatttcgcccctttgaaagtgggtggaatttacttgcctctccactggcctgcccaggaatgctacagataccccattgttctcggctatgacagccatcattttgtacccttggtgaccctgaaggacagtgggcctgaaatccgagctgttccacttgttaacagagaccggggaagatttgaagacttaaaagttcactttttgacagatcctgaaaatgagatgaaggagaagctcttaaaagagtacttaatggtgatagaaatccccgtccaaggctgggaccatggcacaactcatctcatcaatgccgcaaagttggatgaagctaacttaccaaaagaaatcaatctggtagatgattactttgaacttgttcagcatgagtacaagaaatggcaggaaaacagcgagcaggggaggagagaggggcacgcccagaatcccatggaaccttccgtgccccagctttctctcatggatgtaaaatgtgaaacgcccaactgccccttcttcatgtctgtgaacacccagcctttatgccatgagtgctcagagaggcggcaaaagaatcaaaacaaactcccaaagctgaactccaagccgggccctgaggggctccctggcatggcgctcggggcctctcggggagaagcctatgagcccttggcgtggaaccctgaggagtccactggggggcctcattcggccccaccgacagcacccagcccttttctgttcagtgagaccactgccatgaagtgcaggagccccggctgccccttcacactgaatgtgcagcacaacggattttgtgaacgttgccacaacgcccggcaacttcacgccagccacgccccagaccacacaaggcacttggatcccgggaagtgccaagcctgcctccaggatgttaccaggacatttaatgggatctgcagtacttgcttcaaaaggactacagcagaggcctcctccagcctcagcaccagcctccctccttcctgtcaccagcgttccaagtcagatccctcgcggctcgtccggagcccctccccgcattcttgccacagagctggaaacgacgcccctgctggctgcctgtctcaagctgcacggactcctggggacaggacggggacgagcaagtgcagaaaagccggctgcgtgtattttgggactccagaaaacaagggcttttgcacactgtgtttcatcgagtacagagaaaacaaacattttgctgctgcctcagggaaagtcagtcccacagcgtccaggttccagaacaccattccgtgcctggggagggaatgcggcacccttggaagcaccatgtttgaaggatactgccagaagtgtttcattgaagctcagaatcagagatttcatgaggccaaaaggacagaagagcaactgagatcgagccagcgcagagatgtgcctcgaaccacacaaagcacctcaaggcccaagtgcgcccgggcctcctgcaagaacatcctggcctgccgcagcgaggagctctgcatggagtgtcagcatcccaaccagaggatgggccctggggcccaccggggtgagcctgcccccgaagacccccccaagcagcgttgccgggcccccgcctgtgatcattttggcaatgccaagtgcaacggctactgcaacgaatgctttcagttcaagcagatgtatggcggaagcggagctactaacttcagcctgctgaagcaggctggagacgtggaggagaaccctggacctatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaacttacatgtcaggctgagggctaccccaaggccgaagtcatctggacaagcagtgaccatcaagtcctgagtggtaagaccaccaccaccaattccaagagagaggagaaacttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtcgacccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttctggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 32 - DNA, 20 bp, synthetic construct
  source 1..20: mol_type = other DNA
gaagcgtgtcttcatagcgc

SEQ ID NO: 33 - DNA, 20 bp, synthetic construct
  source 1..20: mol_type = other DNA
ttactcgtgtcaaagccgtt

SEQ ID NO: 34 - DNA, 20 bp, synthetic construct
  source 1..20: mol_type = other DNA
tgtcaaagccgttaggatcc

SEQ ID NO: 35 - DNA, 20 bp, synthetic construct
  source 1..20: mol_type = other DNA
gccgttaggatcctggcttg

SEQ ID NO: 36 - DNA, 20 bp, synthetic construct
  source 1..20: mol_type = other DNA
gcggagtggctaaagtgctt

SEQ ID NO: 37 - DNA, 20 bp, synthetic construct
  source 1..20: mol_type = other DNA
tccgcaagccaggatcctaa

SEQ ID NO: 38 - DNA, 20 bp, synthetic construct
  source 1..20: mol_type = other DNA
gttcggctttgagcttcctc

SEQ ID NO: 39 - DNA, 20 bp, synthetic construct
  source 1..20: mol_type = other DNA
gagatggtgatcatgagacc

SEQ ID NO: 40 - DNA, 20 bp, synthetic construct
  source 1..20: mol_type = other DNA
ttgtactcatatttgtttcc

SEQ ID NO: 41 - DNA, 20 bp, synthetic construct
  source 1..20: mol_type = other DNA
aacaaatatgagtacaagtt

SEQ ID NO: 42 - DNA, 800 bp, synthetic construct
  source 1..800: mol_type = other DNA
accgctctcagaccagaaacgtccacacccgccctccgatggcctgtcgccctggctaggttttagggtcagtgggatcctccttccactggacccgggagaagacgctcaacagccccctccttcccctccttcctctccttcctctccttcccccctccctgcgccgctccagagcgcaacaaccattttcccagccaggagcacaccgtgtccacgcgccacagcgatctcactgattggtcgggctcctggtaaacaaggaccgggcagccaatgggagggatgtgcacgagggcagcacgagcctccgggccagcgctcgcgtggctcttctggcccgggctactatatagagacgtttccgcctcctgcttgaaactaacccctctttttctccaaaggagtgcttgtggagatcggatcttttctccagcaattgggggaaagaaggctttttctctgaattagcttagtgtaaccagcggcgtatattttttaggcgccttttcgaaaacctagtagttaatattcatttgtttaaatcttattttatttttaagctcaaactgcttaagaataccttaattccttaaagtgaaataattttttgcaaaggggtttcctcgatttggagctttttttttcttccaccgtcatttctaactcttaaaaccaactcagttccatcatggtgatgttcaagaagatcaagtcttttgaggtggtctttaacgaccctgaaaaggtgtacggcagtggcgagaaggtggctggccgggtgatagtggaggtgtgtg

SEQ ID NO: 43 - DNA, 1500 bp, synthetic construct
  source 1..1500: mol_type = other DNA
atgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttg

SEQ ID NO: 44 - DNA, 800 bp, synthetic construct
  source 1..800: mol_type = other DNA
cagggatcccagcagtgcaaacagacttcggagtacctgcgctatgaagacacgcttcttctggaagaccagccaacaggtaagcggcccaattcattgttggagggtgaaagctgattagagaagagaattgaatacacaaaacctgtacgaaatgttttaagttgctcagtttgagtggtttgaattacgtgttgttgcttccttttttctgttttaatttgcagacattctcctccccccccaaaaaaaagggtgatttgtacaattttttatggtgctgtgtcctaaaggggatcctgaggggcgttgcctcgggtagttaaagtcttatgtgtgcataagttgcttattctttgtctacttcctatttgagatgttagtagagaactgtcctgggtgaatctttcagtattgcagggcttggcaacttgctgcccgacaaaatacatcagaatttctctttaagaacaatatgggatggattaaaaaatatatatatgggatgaaattgggggtacttcaataccttgcatgccacccaagcattccttatcacacagatgcattttaagtgtaacagcaagcctaatggctactcgattttctttcccttcaggtgagaatgagatggtgatcatgagacctggaaacaaatatgagtacaagttcggctttgagcttcctcaggggtaaatatcagctaaatgcatctttgaacttttctgtctaaaatatcttgccctcctttgatcacttactgttcttggagagcgttttaaaattttcattttcttgac

SEQ ID NO: 45 - DNA, 8381 bp, synthetic construct
  source 1..8381: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtaccgctctcagaccagaaacgtccacacccgccctccgatggcctgtcgccctggctaggttttagggtcagtgggatcctccttccactggacccgggagaagacgctcaacagccccctccttcccctccttcctctccttcctctccttcccccctccctgcgccgctccagagcgcaacaaccattttcccagccaggagcacaccgtgtccacgcgccacagcgatctcactgattggtcgggctcctggtaaacaaggaccgggcagccaatgggagggatgtgcacgagggcagcacgagcctccgggccagcgctcgcgtggctcttctggcccgggctactatatagagacgtttccgcctcctgcttgaaactaacccctctttttctccaaaggagtgcttgtggagatcggatcttttctccagcaattgggggaaagaaggctttttctctgaattagcttagtgtaaccagcggcgtatattttttaggcgccttttcgaaaacctagtagttaatattcatttgtttaaatcttattttatttttaagctcaaactgcttaagaataccttaattccttaaagtgaaataattttttgcaaaggggtttcctcgatttggagctttttttttcttccaccgtcatttctaactcttaaaaccaactcagttccatcatggtgatgttcaagaagatcaagtcttttgaggtggtctttaacgaccctgaaaaggtgtacggcagtggcgagaaggtggctggccgggtgatagtggaggtgtgtgaagcttgtggacgatatcgaattcgcacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggggggatccgtttatctgcagaattcgcccttgacgtcgccaccatgaggaggatgtgggccacgcaggggctggcggtggcgctggctctgagcgtgctgccgggcagccgggcgctgcggccgggcgactgcgaagtttgtatttcttatctgggaagattttaccaggacctcaaagacagagatgtcacattctcaccagccactattgaaaacgaacttataaagttctgccgggaagcaagaggcaaagagaatcggttgtgctactatatcggggccacagatgatgcagccaccaaaatcatcaatgaggtatcaaagcctctggcccaccacatccctgtggagaagatctgtgagaagcttaagaagaaggacagccagatatgtgagcttaagtatgacaagcagatcgacctgagcacagtggacctgaagaagctccgagttaaagagctgaagaagattctggatgactggggggagacatgcaaaggctgtgcagaaaagtctgactacatccggaagataaatgaactgatgcctaaatatgcccccaaggcagccagtgcacggaccgatttgggaagcggagctactaacttcagcctgctgaagcaggctggagacgtggaggagaaccctggacctatgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatgatagccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtcgaccagggatcccagcagtgcaaacagacttcggagtacctgcgctatgaagacacgcttcttctggaagaccagccaacaggtaagcggcccaattcattgttggagggtgaaagctgattagagaagagaattgaatacacaaaacctgtacgaaatgttttaagttgctcagtttgagtggtttgaattacgtgttgttgcttccttttttctgttttaatttgcagacattctcctccccccccaaaaaaaagggtgatttgtacaattttttatggtgctgtgtcctaaaggggatcctgaggggcgttgcctcgggtagttaaagtcttatgtgtgcataagttgcttattctttgtctacttcctatttgagatgttagtagagaactgtcctgggtgaatctttcagtattgcagggcttggcaacttgctgcccgacaaaatacatcagaatttctctttaagaacaatatgggatggattaaaaaatatatatatgggatgaaattgggggtacttcaataccttgcatgccacccaagcattccttatcacacagatgcattttaagtgtaacagcaagcctaatggctactcgattttctttcccttcaggtgagaatgagatggtgatcatgagacctggaaacaaatatgagtacaagttcggctttgagcttcctcaggggtaaatatcagctaaatgcatctttgaacttttctgtctaaaatatcttgccctcctttgatcacttactgttcttggagagcgttttaaaattttcattttcttgacggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 46 - DNA, 1722 bp, synthetic construct
  source 1..1722: mol_type = other DNA
atgtgtccccgagccgcgcgggcgcccgcgacgctactcctcgccctgggcgcggtgctgtggcctgcggctggcgcctgggagcttacgattttgcacaccaacgacgtgcacagccggctggagcagaccagcgaggactccagcaagtgcgtcaacgccagccgctgcatgggtggcgtggctcggctcttcaccaaggttcagcagatccgccgcgccgaacccaacgtgctgctgctggacgccggcgaccagtaccagggcactatctggttcaccgtgtacaagggcgccgaggtggcgcacttcatgaacgccctgcgctacgatgccatggcactgggaaatcatgaatttgataatggtgtggaaggactgatcgagccactcctcaaagaggccaaatttccaattctgagtgcaaacattaaagcaaaggggccactagcatctcaaatatcaggactttatttgccatataaagttcttcctgttggtgatgaagttgtgggaatcgttggatacacttccaaagaaaccccttttctctcaaatccagggacaaatttagtgtttgaagatgaaatcactgcattacaacctgaagtagataagttaaaaactctaaatgtgaacaaaattattgcactgggacattcgggttttgaaatggataaactcatcgctcagaaagtgaggggtgtggacgtcgtggtgggaggacactccaacacatttctttacacaggcaatccaccttccaaagaggtgcctgctgggaagtacccattcatagtcacttctgatgatgggcggaaggttcctgtagtccaggcctatgcttttggcaaatacctaggctatctgaagatcgagtttgatgaaagaggaaacgtcatctcttcccatggaaatcccattcttctaaacagcagcattcctgaagatccaagcataaaagcagacattaacaaatggaggataaaattggataattattctacccaggaattagggaaaacaattgtctatctggatggctcctctcaatcatgccgctttagagaatgcaacatgggcaacctgatttgtgatgcaatgattaacaacaacctgagacacacggatgaaatgttctggaaccacgtatccatgtgcattttaaatggaggtggtatccggtcgcccattgatgaacgcaacaatggcacaattacctgggagaacctggctgctgtattgccctttggaggcacatttgacctagtccagttaaaaggttccaccctgaagaaggcctttgagcatagcgtgcaccgctacggccagtccactggagagttcctgcaggtgggcggaatccatgtggtgtatgatctttcccgaaaacctggagacagagtagtcaaattagatgttctttgcaccaagtgtcgagtgcccagttatgaccctctcaaaatggacgaggtatataaggtgatcctcccaaacttcctggccaatggtggagatgggttccagatgataaaagatgaattattaagacatgactctggtgaccaagatatcaacgtggtttctacatatatctccaaaatgaaagtaatttatccagcagttgaaggtcggatcaagttttccacaggaagtcactgccatggaagcttttctttaatatttctttcactttgggcagtgatctttgttttataccaa

SEQ ID NO: 47 - DNA, 10517 bp, synthetic construct
  source 1..10517: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgttctagggtggaaactaagagaatgatgtacctagagggcgctggaagctctaaagccctagcagttactgcttttactattagtggtcgtttttttctcccccccgccccccgacaaatcaacagaacaaagaaaattacctaaacagcaaggacatagggaggaacttcttggcacagaactttccaaacactttttcctgaagggatacaagaagcaagaaaggtactctttcactaggaccttctctgagctgtcctcaggatgcttttgggactatttttcttacccagagaatggagaaaccctgcagggaattcccaagctgtagttataaacagaagttctccttctgctaggtagcattcaaagatcttaatcttctgggtttccgttttctcgaatgaaaaatgcaggtccgagcagttaactggctggggcaccattagcaagtcacttagcatctctggggccagtctgcaaagcgagggggcagccttaatgtgcctccagcctgaagtcctagaatgagcgcccggtgtcccaagctggggcgcgcaccccagatcggagggcgccgatgtacagacagcaaactcacccagtctagtgcatgccttcttaaacatcacgagactctaagaaaaggaaactgaaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctaagcttgtggacgatatcgaattcgcacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacaggggggatccgtttatctgcagaattcgcccttgacgtcgccaccatggaagatacaaaggagtctaacgtgaagacattttgctccaagaatatcctagccatccttggcttctcctctatcatagctgtgatagctttgcttgctgtggggttgacccagaacaaagcattgccagaaaacgttaagtatgggattgtgctggatgcgggttcttctcacacaagtttatacatctataagtggccagcagaaaaggagaatgacacaggcgtggtgcatcaagtagaagaatgcagggttaaaggtcctggaatctcaaaatttgttcagaaagtaaatgaaataggcatttacctgactgattgcatggaaagagctagggaagtgattccaaggtcccagcaccaagagacacccgtttacctgggagccacggcaggcatgcggttgctcaggatggaaagtgaagagttggcagacagggttctggatgtggtggagaggagcctcagcaactacccctttgacttccagggtgccaggatcattactggccaagaggaaggtgcctatggctggattactatcaactatctgctgggcaaattcagtcagaaaacaaggtggttcagcatagtcccatatgaaaccaataatcaggaaacctttggagctttggaccttgggggagcctctacacaagtcacttttgtaccccaaaaccagactatcgagtccccagataatgctctgcaatttcgcctctatggcaaggactacaatgtctacacacatagcttcttgtgctatgggaaggatcaggcactctggcagaaactggccaaggacattcaggttgcaagtaatgaaattctcagggacccatgctttcatcctggatataagaaggtagtgaacgtaagtgacctttacaagaccccctgcaccaagagatttgagatgactcttccattccagcagtttgaaatccagggtattggaaactatcaacaatgccatcaaagcatcctggagctcttcaacaccagttactgcccttactcccagtgtgccttcaatgggattttcttgccaccactccagggggattttggggcattttcagctttttactttgtgatgaagtttttaaacttgacatcagagaaagtctctcaggaaaaggtgactgagatgatgaaaaagttctgtgctcagccttgggaggagataaaaacatcttacgctggagtaaaggagaagtacctgagtgaatactgcttttctggtacctacattctctccctccttctgcaaggctatcatttcacagctgattcctgggagcacatccatttcattggcaagatccagggcagcgacgccggctggactttgggctacatgctgaacctgaccaacatgatcccagctgagcaaccattgtccacacctctctcccactccacctatgtcttcctcatggttctattctccctggtccttttcacagtggccatcataggcttgcttatctttcacaagccttcatatttctggaaagatatggtaggaagcggagctactaacttcagcctgctgaagcaggctggagacgtggaggagaaccctggacctatgtgtccccgagccgcgcgggcgcccgcgacgctactcctcgccctgggcgcggtgctgtggcctgcggctggcgcctgggagcttacgattttgcacaccaacgacgtgcacagccggctggagcagaccagcgaggactccagcaagtgcgtcaacgccagccgctgcatgggtggcgtggctcggctcttcaccaaggttcagcagatccgccgcgccgaacccaacgtgctgctgctggacgccggcgaccagtaccagggcactatctggttcaccgtgtacaagggcgccgaggtggcgcacttcatgaacgccctgcgctacgatgccatggcactgggaaatcatgaatttgataatggtgtggaaggactgatcgagccactcctcaaagaggccaaatttccaattctgagtgcaaacattaaagcaaaggggccactagcatctcaaatatcaggactttatttgccatataaagttcttcctgttggtgatgaagttgtgggaatcgttggatacacttccaaagaaaccccttttctctcaaatccagggacaaatttagtgtttgaagatgaaatcactgcattacaacctgaagtagataagttaaaaactctaaatgtgaacaaaattattgcactgggacattcgggttttgaaatggataaactcatcgctcagaaagtgaggggtgtggacgtcgtggtgggaggacactccaacacatttctttacacaggcaatccaccttccaaagaggtgcctgctgggaagtacccattcatagtcacttctgatgatgggcggaaggttcctgtagtccaggcctatgcttttggcaaatacctaggctatctgaagatcgagtttgatgaaagaggaaacgtcatctcttcccatggaaatcccattcttctaaacagcagcattcctgaagatccaagcataaaagcagacattaacaaatggaggataaaattggataattattctacccaggaattagggaaaacaattgtctatctggatggctcctctcaatcatgccgctttagagaatgcaacatgggcaacctgatttgtgatgcaatgattaacaacaacctgagacacacggatgaaatgttctggaaccacgtatccatgtgcattttaaatggaggtggtatccggtcgcccattgatgaacgcaacaatggcacaattacctgggagaacctggctgctgtattgccctttggaggcacatttgacctagtccagttaaaaggttccaccctgaagaaggcctttgagcatagcgtgcaccgctacggccagtccactggagagttcctgcaggtgggcggaatccatgtggtgtatgatctttcccgaaaacctggagacagagtagtcaaattagatgttctttgcaccaagtgtcgagtgcccagttatgaccctctcaaaatggacgaggtatataaggtgatcctcccaaacttcctggccaatggtggagatgggttccagatgataaaagatgaattattaagacatgactctggtgaccaagatatcaacgtggtttctacatatatctccaaaatgaaagtaatttatccagcagttgaaggtcggatcaagttttccacaggaagtcactgccatggaagcttttctttaatatttctttcactttgggcagtgatctttgttttataccaaggaagcggagctactaacttcagcctgctgaagcaggctggagacgtggaggagaaccctggacctatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaaacttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtcgacccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttctggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 48 - DNA, 20 bp, synthetic construct
  source 1..20: mol_type = other DNA
gctccaggtagccaccttct

SEQ ID NO: 49 - DNA, 20 bp, synthetic construct
  source 1..20: mol_type = other DNA
taggggccccaactccatgg

SEQ ID NO: 50 - DNA, 20 bp, synthetic construct
  source 1..20: mol_type = other DNA
ggcttatgccaatatcggtg

SEQ ID NO: 51 - DNA, 20 bp, synthetic construct
  source 1..20: mol_type = other DNA
aggtgatgaagagaccaggg

SEQ ID NO: 52 - DNA, 3921 bp, synthetic construct
  source 1..3921: mol_type = other DNA
atgaggaggatgtgggccacgcaggggctggcggtggcgctggctctgagcgtgctgccgggcagccgggcgctgcggccgggcgactgcgaagtttgtatttcttatctgggaagattttaccaggacctcaaagacagagatgtcacattctcaccagccactattgaaaacgaacttataaagttctgccgggaagcaagaggcaaagagaatcggttgtgctactatatcggggccacagatgatgcagccaccaaaatcatcaatgaggtatcaaagcctctggcccaccacatccctgtggagaagatctgtgagaagcttaagaagaaggacagccagatatgtgagcttaagtatgacaagcagatcgacctgagcacagtggacctgaagaagctccgagttaaagagctgaagaagattctggatgactggggggagacatgcaaaggctgtgcagaaaagtctgactacatccggaagataaatgaactgatgcctaaatatgcccccaaggcagccagtgcacggaccgatttgggaagcggagctactaacttcagcctgctgaagcaggctggagacgtggaggagaaccctggacctatggctgaacaagtccttcctcaggctttgtatttgagcaatatgcggaaagctgtgaagatacgggagagaactccagaagacatttttaaacctactaatgggatcattcatcattttaaaaccatgcaccgatacacactggaaatgttcagaacttgccagttttgtcctcagtttcgggagatcatccacaaagccctcatcgacagaaacatccaggccaccctggaaagccagaagaaactcaactggtgtcgagaagtccggaagcttgtggcgctgaaaacgaacggtgacggcaattgcctcatgcatgccacttctcagtacatgtggggcgttcaggacacagacttggtactgaggaaggcgctgttcagcacgctcaaggaaacagacacacgcaactttaaattccgctggcaactggagtctctcaaatctcaggaatttgttgaaacggggctttgctatgatactcggaactggaatgatgaatgggacaatcttatcaaaatggcttccacagacacacccatggcccgaagtggacttcagtacaactcactggaagaaatacacatatttgtcctttgcaacatcctcagaaggccaatcattgtcatttcagacaaaatgctaagaagtttggaatcaggttccaatttcgcccctttgaaagtgggtggaatttacttgcctctccactggcctgcccaggaatgctacagataccccattgttctcggctatgacagccatcattttgtacccttggtgaccctgaaggacagtgggcctgaaatccgagctgttccacttgttaacagagaccggggaagatttgaagacttaaaagttcactttttgacagatcctgaaaatgagatgaaggagaagctcttaaaagagtacttaatggtgatagaaatccccgtccaaggctgggaccatggcacaactcatctcatcaatgccgcaaagttggatgaagctaacttaccaaaagaaatcaatctggtagatgattactttgaacttgttcagcatgagtacaagaaatggcaggaaaacagcgagcaggggaggagagaggggcacgcccagaatcccatggaaccttccgtgccccagctttctctcatggatgtaaaatgtgaaacgcccaactgccccttcttcatgtctgtgaacacccagcctttatgccatgagtgctcagagaggcggcaaaagaatcaaaacaaactcccaaagctgaactccaagccgggccctgaggggctccctggcatggcgctcggggcctctcggggagaagcctatgagcccttggcgtggaaccctgaggagtccactggggggcctcattcggccccaccgacagcacccagcccttttctgttcagtgagaccactgccatgaagtgcaggagccccggctgccccttcacactgaatgtgcagcacaacggattttgtgaacgttgccacaacgcccggcaacttcacgccagccacgccccagaccacacaaggcacttggatcccgggaagtgccaagcctgcctccaggatgttaccaggacatttaatgggatctgcagtacttgcttcaaaaggactacagcagaggcctcctccagcctcagcaccagcctccctccttcctgtcaccagcgttccaagtcagatccctcgcggctcgtccggagcccctccccgcattcttgccacagagctggaaacgacgcccctgctggctgcctgtctcaagctgcacggactcctggggacaggacggggacgagcaagtgcagaaaagccggctgcgtgtattttgggactccagaaaacaagggcttttgcacactgtgtttcatcgagtacagagaaaacaaacattttgctgctgcctcagggaaagtcagtcccacagcgtccaggttccagaacaccattccgtgcctggggagggaatgcggcacccttggaagcaccatgtttgaaggatactgccagaagtgtttcattgaagctcagaatcagagatttcatgaggccaaaaggacagaagagcaactgagatcgagccagcgcagagatgtgcctcgaaccacacaaagcacctcaaggcccaagtgcgcccgggcctcctgcaagaacatcctggcctgccgcagcgaggagctctgcatggagtgtcagcatcccaaccagaggatgggccctggggcccaccggggtgagcctgcccccgaagacccccccaagcagcgttgccgggcccccgcctgtgatcattttggcaatgccaagtgcaacggctactgcaacgaatgctttcagttcaagcagatgtatggcggaagcggagctactaacttcagcctgctgaagcaggctggagacgtggaggagaaccctggacctatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaaacttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaa

SEQ ID NO: 53 - DNA, 2469 bp, synthetic construct
  source 1..2469: mol_type = other DNA
atggaagatacaaaggagtctaacgtgaagacattttgctccaagaatatcctagccatccttggcttctcctctatcatagctgtgatagctttgcttgctgtggggttgacccagaacaaagcattgccagaaaacgttaagtatgggattgtgctggatgcgggttcttctcacacaagtttatacatctataagtggccagcagaaaaggagaatgacacaggcgtggtgcatcaagtagaagaatgcagggttaaaggtcctggaatctcaaaatttgttcagaaagtaaatgaaataggcatttacctgactgattgcatggaaagagctagggaagtgattccaaggtcccagcaccaagagacacccgtttacctgggagccacggcaggcatgcggttgctcaggatggaaagtgaagagttggcagacagggttctggatgtggtggagaggagcctcagcaactacccctttgacttccagggtgccaggatcattactggccaagaggaaggtgcctatggctggattactatcaactatctgctgggcaaattcagtcagaaaacaaggtggttcagcatagtcccatatgaaaccaataatcaggaaacctttggagctttggaccttgggggagcctctacacaagtcacttttgtaccccaaaaccagactatcgagtccccagataatgctctgcaatttcgcctctatggcaaggactacaatgtctacacacatagcttcttgtgctatgggaaggatcaggcactctggcagaaactggccaaggacattcaggttgcaagtaatgaaattctcagggacccatgctttcatcctggatataagaaggtagtgaacgtaagtgacctttacaagaccccctgcaccaagagatttgagatgactcttccattccagcagtttgaaatccagggtattggaaactatcaacaatgccatcaaagcatcctggagctcttcaacaccagttactgcccttactcccagtgtgccttcaatgggattttcttgccaccactccagggggattttggggcattttcagctttttactttgtgatgaagtttttaaacttgacatcagagaaagtctctcaggaaaaggtgactgagatgatgaaaaagttctgtgctcagccttgggaggagataaaaacatcttacgctggagtaaaggagaagtacctgagtgaatactgcttttctggtacctacattctctccctccttctgcaaggctatcatttcacagctgattcctgggagcacatccatttcattggcaagatccagggcagcgacgccggctggactttgggctacatgctgaacctgaccaacatgatcccagctgagcaaccattgtccacacctctctcccactccacctatgtcttcctcatggttctattctccctggtccttttcacagtggccatcataggcttgcttatctttcacaagccttcatatttctggaaagatatggtaggaagcggagctactaacttcagcctgctgaagcaggctggagacgtggaggagaaccctggacctatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaaacttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaa

SEQ ID NO: 54 - DNA, 3309 bp, synthetic construct
  source 1..3309: mol_type = other DNA
atggctgaacaagtccttcctcaggctttgtatttgagcaatatgcggaaagctgtgaagatacgggagagaactccagaagacatttttaaacctactaatgggatcattcatcattttaaaaccatgcaccgatacacactggaaatgttcagaacttgccagttttgtcctcagtttcgggagatcatccacaaagccctcatcgacagaaacatccaggccaccctggaaagccagaagaaactcaactggtgtcgagaagtccggaagcttgtggcgctgaaaacgaacggtgacggcaattgcctcatgcatgccacttctcagtacatgtggggcgttcaggacacagacttggtactgaggaaggcgctgttcagcacgctcaaggaaacagacacacgcaactttaaattccgctggcaactggagtctctcaaatctcaggaatttgttgaaacggggctttgctatgatactcggaactggaatgatgaatgggacaatcttatcaaaatggcttccacagacacacccatggcccgaagtggacttcagtacaactcactggaagaaatacacatatttgtcctttgcaacatcctcagaaggccaatcattgtcatttcagacaaaatgctaagaagtttggaatcaggttccaatttcgcccctttgaaagtgggtggaatttacttgcctctccactggcctgcccaggaatgctacagataccccattgttctcggctatgacagccatcattttgtacccttggtgaccctgaaggacagtgggcctgaaatccgagctgttccacttgttaacagagaccggggaagatttgaagacttaaaagttcactttttgacagatcctgaaaatgagatgaaggagaagctcttaaaagagtacttaatggtgatagaaatccccgtccaaggctgggaccatggcacaactcatctcatcaatgccgcaaagttggatgaagctaacttaccaaaagaaatcaatctggtagatgattactttgaacttgttcagcatgagtacaagaaatggcaggaaaacagcgagcaggggaggagagaggggcacgcccagaatcccatggaaccttccgtgccccagctttctctcatggatgtaaaatgtgaaacgcccaactgccccttcttcatgtctgtgaacacccagcctttatgccatgagtgctcagagaggcggcaaaagaatcaaaacaaactcccaaagctgaactccaagccgggccctgaggggctccctggcatggcgctcggggcctctcggggagaagcctatgagcccttggcgtggaaccctgaggagtccactggggggcctcattcggccccaccgacagcacccagcccttttctgttcagtgagaccactgccatgaagtgcaggagccccggctgccccttcacactgaatgtgcagcacaacggattttgtgaacgttgccacaacgcccggcaacttcacgccagccacgccccagaccacacaaggcacttggatcccgggaagtgccaagcctgcctccaggatgttaccaggacatttaatgggatctgcagtacttgcttcaaaaggactacagcagaggcctcctccagcctcagcaccagcctccctccttcctgtcaccagcgttccaagtcagatccctcgcggctcgtccggagcccctccccgcattcttgccacagagctggaaacgacgcccctgctggctgcctgtctcaagctgcacggactcctggggacaggacggggacgagcaagtgcagaaaagccggctgcgtgtattttgggactccagaaaacaagggcttttgcacactgtgtttcatcgagtacagagaaaacaaacattttgctgctgcctcagggaaagtcagtcccacagcgtccaggttccagaacaccattccgtgcctggggagggaatgcggcacccttggaagcaccatgtttgaaggatactgccagaagtgtttcattgaagctcagaatcagagatttcatgaggccaaaaggacagaagagcaactgagatcgagccagcgcagagatgtgcctcgaaccacacaaagcacctcaaggcccaagtgcgcccgggcctcctgcaagaacatcctggcctgccgcagcgaggagctctgcatggagtgtcagcatcccaaccagaggatgggccctggggcccaccggggtgagcctgcccccgaagacccccccaagcagcgttgccgggcccccgcctgtgatcattttggcaatgccaagtgcaacggctactgcaacgaatgctttcagttcaagcagatgtatggcggaagcggagctactaacttcagcctgctgaagcaggctggagacgtggaggagaaccctggacctatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaacttacatgtcaggctgagggctaccccaaggccgaagtcatctggacaagcagtgaccatcaagtcctgagtggtaagaccaccaccaccaattccaagagagaggagaaacttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaa

SEQ ID NO: 55 - DNA, 2112 bp, synthetic construct
  source 1..2112: mol_type = other DNA
atgaggaggatgtgggccacgcaggggctggcggtggcgctggctctgagcgtgctgccgggcagccgggcgctgcggccgggcgactgcgaagtttgtatttcttatctgggaagattttaccaggacctcaaagacagagatgtcacattctcaccagccactattgaaaacgaacttataaagttctgccgggaagcaagaggcaaagagaatcggttgtgctactatatcggggccacagatgatgcagccaccaaaatcatcaatgaggtatcaaagcctctggcccaccacatccctgtggagaagatctgtgagaagcttaagaagaaggacagccagatatgtgagcttaagtatgacaagcagatcgacctgagcacagtggacctgaagaagctccgagttaaagagctgaagaagattctggatgactggggggagacatgcaaaggctgtgcagaaaagtctgactacatccggaagataaatgaactgatgcctaaatatgcccccaaggcagccagtgcacggaccgatttgggaagcggagctactaacttcagcctgctgaagcaggctggagacgtggaggagaaccctggacctatgtctcgctccgttgccttagctgtgctcgcgctactctctctttctggattagaggctgtcatggcgccccgaaccctcttcctgggtggaggcggttcaggcggaggtggctctggcggtggcggatcgatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggtggtggttctggtggtggtggttctggcggcggcggctccggtggtggtggatccggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttg

SEQ ID NO: 56 - DNA, 4257 bp, synthetic construct
  source 1..4257: mol_type = other DNA
atggaagatacaaaggagtctaacgtgaagacattttgctccaagaatatcctagccatccttggcttctcctctatcatagctgtgatagctttgcttgctgtggggttgacccagaacaaagcattgccagaaaacgttaagtatgggattgtgctggatgcgggttcttctcacacaagtttatacatctataagtggccagcagaaaaggagaatgacacaggcgtggtgcatcaagtagaagaatgcagggttaaaggtcctggaatctcaaaatttgttcagaaagtaaatgaaataggcatttacctgactgattgcatggaaagagctagggaagtgattccaaggtcccagcaccaagagacacccgtttacctgggagccacggcaggcatgcggttgctcaggatggaaagtgaagagttggcagacagggttctggatgtggtggagaggagcctcagcaactacccctttgacttccagggtgccaggatcattactggccaagaggaaggtgcctatggctggattactatcaactatctgctgggcaaattcagtcagaaaacaaggtggttcagcatagtcccatatgaaaccaataatcaggaaacctttggagctttggaccttgggggagcctctacacaagtcacttttgtaccccaaaaccagactatcgagtccccagataatgctctgcaatttcgcctctatggcaaggactacaatgtctacacacatagcttcttgtgctatgggaaggatcaggcactctggcagaaactggccaaggacattcaggttgcaagtaatgaaattctcagggacccatgctttcatcctggatataagaaggtagtgaacgtaagtgacctttacaagaccccctgcaccaagagatttgagatgactcttccattccagcagtttgaaatccagggtattggaaactatcaacaatgccatcaaagcatcctggagctcttcaacaccagttactgcccttactcccagtgtgccttcaatgggattttcttgccaccactccagggggattttggggcattttcagctttttactttgtgatgaagtttttaaacttgacatcagagaaagtctctcaggaaaaggtgactgagatgatgaaaaagttctgtgctcagccttgggaggagataaaaacatcttacgctggagtaaaggagaagtacctgagtgaatactgcttttctggtacctacattctctccctccttctgcaaggctatcatttcacagctgattcctgggagcacatccatttcattggcaagatccagggcagcgacgccggctggactttgggctacatgctgaacctgaccaacatgatcccagctgagcaaccattgtccacacctctctcccactccacctatgtcttcctcatggttctattctccctggtccttttcacagtggccatcataggcttgcttatctttcacaagccttcatatttctggaaagatatggtaggaagcggagctactaacttcagcctgctgaagcaggctggagacgtggaggagaaccctggacctatgtgtccccgagccgcgcgggcgcccgcgacgctactcctcgccctgggcgcggtgctgtggcctgcggctggcgcctgggagcttacgattttgcacaccaacgacgtgcacagccggctggagcagaccagcgaggactccagcaagtgcgtcaacgccagccgctgcatgggtggcgtggctcggctcttcaccaaggttcagcagatccgccgcgccgaacccaacgtgctgctgctggacgccggcgaccagtaccagggcactatctggttcaccgtgtacaagggcgccgaggtggcgcacttcatgaacgccctgcgctacgatgccatggcactgggaaatcatgaatttgataatggtgtggaaggactgatcgagccactcctcaaagaggccaaatttccaattctgagtgcaaacattaaagcaaaggggccactagcatctcaaatatcaggactttatttgccatataaagttcttcctgttggtgatgaagttgtgggaatcgttggatacacttccaaagaaaccccttttctctcaaatccagggacaaatttagtgtttgaagatgaaatcactgcattacaacctgaagtagataagttaaaaactctaaatgtgaacaaaattattgcactgggacattcgggttttgaaatggataaactcatcgctcagaaagtgaggggtgtggacgtcgtggtgggaggacactccaacacatttctttacacaggcaatccaccttccaaagaggtgcctgctgggaagtacccattcatagtcacttctgatgatgggcggaaggttcctgtagtccaggcctatgcttttggcaaatacctaggctatctgaagatcgagtttgatgaaagaggaaacgtcatctcttcccatggaaatcccattcttctaaacagcagcattcctgaagatccaagcataaaagcagacattaacaaatggaggataaaattggataattattctacccaggaattagggaaaacaattgtctatctggatggctcctctcaatcatgccgctttagagaatgcaacatgggcaacctgatttgtgatgcaatgattaacaacaacctgagacacacggatgaaatgttctggaaccacgtatccatgtgcattttaaatggaggtggtatccggtcgcccattgatgaacgcaacaatggcacaattacctgggagaacctggctgctgtattgccctttggaggcacatttgacctagtccagttaaaaggttccaccctgaagaaggcctttgagcatagcgtgcaccgctacggccagtccactggagagttcctgcaggtgggcggaatccatgtggtgtatgatctttcccgaaaacctggagacagagtagtcaaattagatgttctttgcaccaagtgtcgagtgcccagttatgaccctctcaaaatggacgaggtatataaggtgatcctcccaaacttcctggccaatggtggagatgggttccagatgataaaagatgaattattaagacatgactctggtgaccaagatatcaacgtggtttctacatatatctccaaaatgaaagtaatttatccagcagttgaaggtcggatcaagttttccacaggaagtcactgccatggaagcttttctttaatatttctttcactttgggcagtgatctttgttttataccaaggaagcggagctactaacttcagcctgctgaagcaggctggagacgtggaggagaaccctggacctatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaaacttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaa

SEQ ID NO: 57 - DNA, 20 bp, synthetic construct
  source 1..20: mol_type = other DNA
gttcatgcgcaagaggatcg

SEQ ID NO: 58 - DNA, 3318 bp, synthetic construct
  source 1..3318: mol_type = other DNA
atggaagatacaaaggagtctaacgtgaagacattttgctccaagaatatcctagccatccttggcttctcctctatcatagctgtgatagctttgcttgctgtggggttgacccagaacaaagcattgccagaaaacgttaagtatgggattgtgctggatgcgggttcttctcacacaagtttatacatctataagtggccagcagaaaaggagaatgacacaggcgtggtgcatcaagtagaagaatgcagggttaaaggtcctggaatctcaaaatttgttcagaaagtaaatgaaataggcatttacctgactgattgcatggaaagagctagggaagtgattccaaggtcccagcaccaagagacacccgtttacctgggagccacggcaggcatgcggttgctcaggatggaaagtgaagagttggcagacagggttctggatgtggtggagaggagcctcagcaactacccctttgacttccagggtgccaggatcattactggccaagaggaaggtgcctatggctggattactatcaactatctgctgggcaaattcagtcagaaaacaaggtggttcagcatagtcccatatgaaaccaataatcaggaaacctttggagctttggaccttgggggagcctctacacaagtcacttttgtaccccaaaaccagactatcgagtccccagataatgctctgcaatttcgcctctatggcaaggactacaatgtctacacacatagcttcttgtgctatgggaaggatcaggcactctggcagaaactggccaaggacattcaggttgcaagtaatgaaattctcagggacccatgctttcatcctggatataagaaggtagtgaacgtaagtgacctttacaagaccccctgcaccaagagatttgagatgactcttccattccagcagtttgaaatccagggtattggaaactatcaacaatgccatcaaagcatcctggagctcttcaacaccagttactgcccttactcccagtgtgccttcaatgggattttcttgccaccactccagggggattttggggcattttcagctttttactttgtgatgaagtttttaaacttgacatcagagaaagtctctcaggaaaaggtgactgagatgatgaaaaagttctgtgctcagccttgggaggagataaaaacatcttacgctggagtaaaggagaagtacctgagtgaatactgcttttctggtacctacattctctccctccttctgcaaggctatcatttcacagctgattcctgggagcacatccatttcattggcaagatccagggcagcgacgccggctggactttgggctacatgctgaacctgaccaacatgatcccagctgagcaaccattgtccacacctctctcccactccacctatgtcttcctcatggttctattctccctggtccttttcacagtggccatcataggcttgcttatctttcacaagccttcatatttctggaaagatatggtaggaagcggagctactaacttcagcctgctgaagcaggctggagacgtggaggagaaccctggacctatgtgtccccgagccgcgcgggcgcccgcgacgctactcctcgccctgggcgcggtgctgtggcctgcggctggcgcctgggagcttacgattttgcacaccaacgacgtgcacagccggctggagcagaccagcgaggactccagcaagtgcgtcaacgccagccgctgcatgggtggcgtggctcggctcttcaccaaggttcagcagatccgccgcgccgaacccaacgtgctgctgctggacgccggcgaccagtaccagggcactatctggttcaccgtgtacaagggcgccgaggtggcgcacttcatgaacgccctgcgctacgatgccatggcactgggaaatcatgaatttgataatggtgtggaaggactgatcgagccactcctcaaagaggccaaatttccaattctgagtgcaaacattaaagcaaaggggccactagcatctcaaatatcaggactttatttgccatataaagttcttcctgttggtgatgaagttgtgggaatcgttggatacacttccaaagaaaccccttttctctcaaatccagggacaaatttagtgtttgaagatgaaatcactgcattacaacctgaagtagataagttaaaaactctaaatgtgaacaaaattattgcactgggacattcgggttttgaaatggataaactcatcgctcagaaagtgaggggtgtggacgtcgtggtgggaggacactccaacacatttctttacacaggcaatccaccttccaaagaggtgcctgctgggaagtacccattcatagtcacttctgatgatgggcggaaggttcctgtagtccaggcctatgcttttggcaaatacctaggctatctgaagatcgagtttgatgaaagaggaaacgtcatctcttcccatggaaatcccattcttctaaacagcagcattcctgaagatccaagcataaaagcagacattaacaaatggaggataaaattggataattattctacccaggaattagggaaaacaattgtctatctggatggctcctctcaatcatgccgctttagagaatgcaacatgggcaacctgatttgtgatgcaatgattaacaacaacctgagacacacggatgaaatgttctggaaccacgtatccatgtgcattttaaatggaggtggtatccggtcgcccattgatgaacgcaacaatggcacaattacctgggagaacctggctgctgtattgccctttggaggcacatttgacctagtccagttaaaaggttccaccctgaagaaggcctttgagcatagcgtgcaccgctacggccagtccactggagagttcctgcaggtgggcggaatccatgtggtgtatgatctttcccgaaaacctggagacagagtagtcaaattagatgttctttgcaccaagtgtcgagtgcccagttatgaccctctcaaaatggacgaggtatataaggtgatcctcccaaacttcctggccaatggtggagatgggttccagatgataaaagatgaattattaagacatgactctggtgaccaagatatcaacgtggtttctacatatatctccaaaatgaaagtaatttatccagcagttgaaggtcggatcaagttttccacaggaagtcactgccatggaagcttttctttaatatttctttcactttgggcagtgatctttgttttataccaa

SEQ ID NO: 59 - DNA, 22 bp, synthetic construct
  source 1..22: mol_type = other DNA
aggatacgtttttctgttgggc

SEQ ID NO: 60 - DNA, 20 bp, synthetic construct
  source 1..20: mol_type = other DNA
ggagaacgggaaaagagcga

SEQ ID NO: 61 - RNA, 20 nt, synthetic construct
  source 1..20: mol_type = other RNA
gctactctctctttctggcc

SEQ ID NO: 62 - RNA, 20 nt, synthetic construct
  source 1..20: mol_type = other RNA
ggccgagatgtctcgctccg

SEQ ID NO: 63 - RNA, 20 nt, synthetic construct
  source 1..20: mol_type = other RNA
cgcgagcacagctaaggcca

SEQ ID NO: 64 - RNA, 20 nt, synthetic construct
  source 1..20: mol_type = other RNA
tataagtggaggcgtcgcgc

SEQ ID NO: 65 - RNA, 20 nt, synthetic construct
  source 1..20: mol_type = other RNA
gagtagcgcgagcacagcta

SEQ ID NO: 66 - RNA, 20 nt, synthetic construct
  source 1..20: mol_type = other RNA
actggacgcgtcgcgctggc

SEQ ID NO: 67 - RNA, 20 nt, synthetic construct
  source 1..20: mol_type = other RNA
aagtggaggcgtcgcgctgg

SEQ ID NO: 68 - RNA, 20 nt, synthetic construct
  source 1..20: mol_type = other RNA
ggccacggagcgagacatct

SEQ ID NO: 69 - RNA, 20 nt, synthetic construct
  source 1..20: mol_type = other RNA
gcccgaatgctgtcagcttc

SEQ ID NO: 70 - RNA, 20 nt, synthetic construct
  source 1..20: mol_type = other RNA
ctcgcgctactctctctttc

SEQ ID NO: 71 - RNA, 20 nt, synthetic construct
  source 1..20: mol_type = other RNA
tcctgaagctgacagcattc

SEQ ID NO: 72 - RNA, 20 nt, synthetic construct
  source 1..20: mol_type = other RNA
ttcctgaagctgacagcatt

SEQ ID NO: 73 - RNA, 20 nt, synthetic construct
  source 1..20: mol_type = other RNA
actctctctttctggcctgg

SEQ ID NO: 74 - RNA, 20 nt, synthetic construct
  source 1..20: mol_type = other RNA
ggtccatctggtcatagaag

SEQ ID NO: 75 - RNA, 20 nt, synthetic construct
  source 1..20: mol_type = other RNA
gaagcgtgtcttcatagcgc

SEQ ID NO: 76 - RNA, 20 nt, synthetic construct
  source 1..20: mol_type = other RNA
ttactcgtgtcaaagccgtt

SEQ ID NO: 77 - RNA, 20 nt, synthetic construct
  source 1..20: mol_type = other RNA
tgtcaaagccgttaggatcc

SEQ ID NO: 78 - RNA, 20 nt, synthetic construct
  source 1..20: mol_type = other RNA
gccgttaggatcctggcttg

SEQ ID NO: 79 - RNA, 20 nt, synthetic construct
  source 1..20: mol_type = other RNA
gcggagtggctaaagtgctt

SEQ ID NO: 80 - RNA, 20 nt, synthetic construct
  source 1..20: mol_type = other RNA
tccgcaagccaggatcctaa

SEQ ID NO: 81 - RNA, 20 nt, synthetic construct
  source 1..20: mol_type = other RNA
gttcggctttgagcttcctc

SEQ ID NO: 82 - RNA, 20 nt, synthetic construct
  source 1..20: mol_type = other RNA
gagatggtgatcatgagacc

SEQ ID NO: 83 - RNA, 20 nt, synthetic construct
  source 1..20: mol_type = other RNA
ttgtactcatatttgtttcc

SEQ ID NO: 84 - RNA, 20 nt, synthetic construct
  source 1..20: mol_type = other RNA
aacaaatatgagtacaagtt

SEQ ID NO: 85 - RNA, 20 nt, synthetic construct
  source 1..20: mol_type = other RNA
gctccaggtagccaccttct

SEQ ID NO: 86 - RNA, 20 nt, synthetic construct
  source 1..20: mol_type = other RNA
taggggccccaactccatgg

SEQ ID NO: 87 - RNA, 20 nt, synthetic construct
  source 1..20: mol_type = other RNA
ggcttatgccaatatcggtg

SEQ ID NO: 88 - RNA, 20 nt, synthetic construct
  source 1..20: mol_type = other RNA
aggtgatgaagagaccaggg

SEQ ID NO: 89 - RNA, 20 nt, synthetic construct
  source 1..20: mol_type = other RNA
gttcatgcgcaagaggatcg

(19) United States
(12) Patent Application Publication
(10) Pub. No.: US 2025/0090596 A1
(43) Pub. Date: Mar. 20, 2025

(54) MODULATING INFLAMMATION ASSOCIATED WITH FOREIGN BODY RESPONSE

(71) Applicants: CRISPR Therapeutics AG, Zug (CH); VIACYTE, INC., San Diego, CA (US)

(72) Inventors: Sandeep Soni, South Boston, MA (US); Evert Kroon, San Diego, CA (US)

(21) Appl. No.: 18/885,643

(22) Filed: Sep. 14, 2024

Related U.S. Application Data
(60) Provisional application No. 63/582,727, filed on Sep. 14, 2023.

(51) Int. Cl. A61K 35/39; A61K 9/00; A61K 31/436; A61K 31/519; A61K 38/13; A61K 38/17; A61K 38/20; A61K 38/28; A61P 3/10

(52) U.S. Cl. CPC A61K 35/39; A61K 9/0024; A61K 31/436; A61K 31/519; A61K 38/13; A61K 38/1793; A61K 38/2006; A61K 38/28; A61P 3/10

(57) ABSTRACT

Disclosed herein include methods, compositions, and kits suitable for use in producing insulin in a mammalian subject. In some embodiments, the method comprises implanting a perforated cell delivery device comprising a genetically modified cell or population thereof into the subject. Also provided are combination products comprising the perforated cell delivery device comprising a genetically modified cell or population thereof.

### CROSS-REFERENCE TO RELATED APPLICATION

The present application claims the benefit under 35 U.S.C. β119 (e) of U.S. Provisional Patent Application No. 63/582,727, filed Sep. 14, 2023, the content of which is incorporated by references herein in its entirety.

### REFERENCE TO SEQUENCE LISTING

The present application is being filed along with a Sequence Listing in electronic format. The Sequence Listing is provided as a file entitled 80EM-341774-US_SequenceListing, created Aug. 3, 2023, which is 165 kilobytes in size. The information in the electronic format of the Sequence Listing is incorporated herein by reference in its entirety.

### BACKGROUND

### Field

The present disclosure relates to methods of producing insulin and/or treating a pancreatic disorder or disease in a subject. In some embodiments, the methods provided herein comprise use of gene edited allogenic cells and their delivery thereof.

### Description of the Related Art

Diabetes mellitus, a life-threatening disease growing at an alarming rate, has nearly quadrupled in prevalence between 1980 and 2014. Of the people diagnosed with diabetes, approximately 5% to 10% have type 1 diabetes mellitus (T1D) and require exogenous insulin injections every day to survive. The hallmark of diabetes is elevated blood glucose levels. Over time, this chronic hyperglycemia frequently results in both microvascular (e.g., retinopathy, neuropathy, nephropathy) and macrovascular (e.g., atherosclerosis, cardiac disease) complications, which severely increase morbidity and mortality, and diminish the patient's quality of life.

Diabetes patients who require insulin treatment include all patients with T1D, which is caused by loss of insulin-producing pancreatic beta cell mass, and 20-30% of patients with type 2 diabetes mellitus (T2D), who require insulin as adjunctive therapy. There is no known way to prevent or cure T1D. Treatment involves frequent, painful, and cumbersome blood glucose monitoring followed by insulin injection. This standard of care can be woefully inadequate. Insulin therapy also leads to periods of hypoglycemia. Hypoglycemia, like hyperglycemia, should be avoided if possible.

Additional approaches to treatment of T1D being pursued include immunologic intervention in disease mechanisms, beta-cell regenerative and/or replacement therapies, and engineering solutions to enhance glucose monitoring and insulin delivery. One such treatment with extensive development is the pancreatic islet transplant which may employ cells of either porcine or human origin. Although allogeneic islet cell transplantation has achieved insulin independence in some patients, the success rate from center to center has varied widely, and the duration of effect has been limited. Furthermore, islet transplant has two significant disadvantages. First, pancreatic islet transplantation requires chronic immunosuppression for the lifetime of the graft, which adds substantial risk including severe infection and potential progression of occult cancers. Second, its availability is limited by an insufficient supply of acceptable human pancreases as source material.

A foreign body response (FBR) is an immune-mediated reaction to implanted materials where a cascade of inflammatory events and wound-healing processes can result in fibrosis, or the cellular and collagenous deposition that encapsulates implants (Anderson et al., 2008; Wick et al., 2013; Wynn and Ramalingam, 2012). While the FBR is necessary to enable subsequent vascularization, these events can compromise the performance and durability of implantable devices that require use over extended periods. Implant isolation by fibrosis often interferes with function, as a thick fibrotic layer can cut off the nourishment for cell-based implants, and ultimately lead to impaired graft survival and function.

Therefore, there is a need to deliver allogeneic cells using an implantable device without triggering a foreign body response in the host cell. As described herein, a combination product is provided that has the potential to overcome the disadvantages represented by pancreatic islet transplant and provide homeostatic control of blood glucose levels that is difficult to achieve with continuous glucose monitoring and insulin pumps alone.

### SUMMARY

Disclosed herein include methods of producing insulin in a mammalian subject. In some embodiments, the method comprises: a) administering to a mammalian subject at least one immune attenuating drug; b) implanting a perforated cell delivery device comprising a genetically modified cell, or population thereof, into the mammalian subject; and c) maturing the genetically modified cell, or population thereof, in the perforated cell delivery device in the mammalian subject such that the mature cell, or population thereof, produces insulin secreting cells, thereby producing insulin in the mammalian subject. In some embodiments, inflammation associated with the foreign body response of the perforated cell delivery device loaded with the genetically modified cell, or population thereof in the mammalian subject is reduced compared to administering a comparative perforated cell delivery device loaded with the genetically modified cell, or population thereof, without the at least one immune attenuating drug. In some embodiments, reducing the foreign body response comprises reducing cell trafficking and/or inflammation external to the device in a host tissue, facilitating integration of the device into the host tissue, and/or improving or accelerating healing of the host tissue around the device.

Disclosed herein include methods for treating a pancreatic disease or disorder in a mammalian subject in need thereof. In some embodiments, the method comprises: a) administering to a mammalian subject at least one immune attenuating drug; b) implanting a perforated cell delivery device comprising a genetically modified cell, or population thereof, into the mammalian subject; and c) maturing the genetically modified cell, or population thereof, in the perforated cell delivery device in the mammalian subject such that the mature cell, or population thereof, produces insulin secreting cells, thereby producing insulin in the mammalian subject and treating the pancreatic disease or disorder. In some embodiments, the pancreatic disease or disorder is type I diabetes, type II diabetes, or a pancreatectomy. In some embodiments, the genetically modified cell is an allogeneic pancreatic endoderm cell.

In some embodiments, the genetically modified cell comprises (a) a nucleic acid comprising a nucleotide sequence encoding programmed death-ligand 1 (PD-L1) inserted within a gene encoding beta-2 microglobulin (B2M) and (b) a nucleic acid comprising a nucleotide sequence encoding HLA class I histocompatibility antigen, alpha chain E (HLA-E) inserted within a gene encoding thioredoxin interacting protein (TXNIP). In some embodiments, the genetically modified cell expresses PD-L1 and HLA-E and has reduced or eliminated expression of B2M and TXNIP. In some embodiments, the nucleic acid of (a) further comprises a nucleotide sequence encoding TNFAIP3. In some embodiments, the nucleic acid of (b) further comprises a nucleotide sequence encoding MANF. In some embodiments, the nucleic acid of (a) comprises the nucleotide sequence encoding TNFAIP3 linked to a nucleotide sequence encoding a P2A peptide linked to the nucleotide sequence encoding PD-L1 such that the nucleic acid of (a) comprises a TNFAIP3-P2A-PD-L1 polynucleotide sequence. In some embodiments, the TNFAIP3-P2A-PD-L1 polynucleotide sequence comprises the sequence of SEQ ID NO: 54. In some embodiments, the nucleic acid of (a) is operably linked to an exogenous promoter. In some embodiments, the exogenous promoter is a CMV, EF1α, PGK, CAG/CAGGS, or UBC promoter. In some embodiments, the nucleic acid of (b) comprises the nucleotide sequence encoding MANF linked to a nucleotide sequence encoding a P2A peptide linked to the nucleotide sequence encoding HLA-E such that the nucleic acid of (b) comprises a MANF-P2A-HLA-E polynucleotide sequence. In some embodiments, the MANF-P2A-HLA-E polynucleotide sequence comprises the sequence of SEQ ID NO: 55. In some embodiments, the nucleic acid of (b) is operably linked to an exogenous promoter. In some embodiments, the exogenous promoter is a CMV, EF1α, PGK, CAG/CAGGS, or UBC promoter.

In some embodiments, the genetically modified cell comprises: (a) a nucleic acid comprising a nucleotide sequence encoding TNFAIP3 linked to a nucleotide sequence encoding a P2A peptide linked to a nucleotide sequence encoding PD-L1 such that the nucleic acid of (a) comprises a TNFAIP3-P2A-PD-L1 polynucleotide sequence. In some embodiments, the nucleic acid of (a) is inserted within a gene encoding beta-2 microglobulin (B2M). In some embodiments, the genetically modified cell comprises: (b) a nucleic acid comprising a nucleotide sequence MANF linked to a nucleotide sequence encoding a P2A peptide linked to a nucleotide sequence encoding HLA-E such that the nucleic acid of (b) comprises a MANF-P2A-HLA-E polynucleotide sequence. In some embodiments, the nucleic acid of (b) is inserted within a gene encoding thioredoxin interacting protein (TXNIP). In some embodiments, the genetically modified cell expresses PD-L1, HLA-E, TNFAIP3 and MANF, and has reduced or eliminated expression of B2M and TXNIP. In some embodiments, the TNFAIP3-P2A-PD-L1 polynucleotide sequence comprises the sequence of SEQ ID NO: 54 and the MANF-P2A-HLA-E polynucleotide sequence comprises the sequence of SEQ ID NO: 55. In some embodiments, the nucleic acids of (a) and (b) are operably linked to an exogenous promoter. In some embodiments, the exogenous promoter is a CMV, EF1α, PGK, CAG/CAGGS, or UBC promoter.

In some embodiments, the genetically modified cell further comprises: (c) a disrupted TGFβ2 gene and the cell has reduced or eliminated expression of TGFβ2; (d) a disrupted CIITA gene and the cell has reduced or eliminated expression of CIITA; (e) an insertion of a nucleic acid encoding CD39 and the cell expresses CD39; (f) an insertion of a nucleic acid encoding CD73 and the cell expresses CD73; or (g) any combination of (c), (d), (e) and (f). In some embodiments, the nucleic acid encoding CD39 is inserted within the CIITA gene, and the cell expresses CD39 and has reduced or eliminated expression of CIITA.

The at least one immune attenuating drug can comprise a JAK1 or JAK2 inhibitor, a TNFα or TNFβ blocker, an ILIR blocker, a calcineurin inhibitor, an anti-thymocyte globulin or any combination thereof. In some embodiments, the at least one immune attenuating drug comprises ruxolitinib, etanercept, anakinra or any combination thereof. In some embodiments, the at least one immune attenuating drug comprises the calcineurin inhibitor, the anti-thymocyte globulin, or both. In some embodiments, the calcineurin inhibitor is tacrolimus or cyclosporine A. In some embodiments, the at least one immune attenuating drug comprises tacrolimus, the anti-thymocyte globulin, or both. In some embodiments, the tacrolimus is Prograf®, Astagraf XL®, Envarsus XR®, Hecoria®, or any combination thereof. In some embodiments, the anti-thymocyte globulin is Thymoglobulin®, Atgam™, Fresenius™, Tecelac™, or any combination thereof.

In some embodiments, the calcineurin inhibitor is administered at a dose of about 0.5 mg to about 100 mg. In some embodiments, the calcineurin inhibitor is administered at a dose of about 0.2 mg, 1 mg, 5 mg, 25 mg, or 100 mg. In some embodiments, the calcineurin inhibitor is administered at a dose of about 0.5 mg, 1 mg, or 5 mg. In some embodiments, the calcineurin inhibitor is administered at a dose of about 0.2 mg or about 1 mg. In some embodiments, the calcineurin inhibitor is administered orally. In some embodiments, the calcineurin inhibitor is administered at a dose of about 5 mg/ml. In some embodiments, about 1 mL of the calcineurin inhibitor is administered intravenously. In some embodiments, the anti-thymocyte globulin is administered to the subject at a dose of about 1 mg/kg to about 2 mg/kg. In some embodiments, the anti-thymocyte globulin is administered intravenously. In some embodiments, the anti-thymocyte globulin is administered to the subject at a dose of about 1.5 mg/kg. In some embodiments, each of the at least one immune attenuating drug is administered by oral administration, intravenous administration, subcutaneous administration, or any combination thereof. In some embodiments, each of the at least one immune attenuating drug is administered as a separate composition.

In some embodiments, each of the at least one immune attenuating drug is administered to the subject in a cycle of at least one week. In some embodiments, each of the at least one immune attenuating drug is administered to the subject in a cycle of at least one week, two weeks, three weeks, four weeks, five weeks, six weeks, seven weeks, eight weeks, nine weeks, ten weeks, eleven weeks, twelve weeks, or more. In some embodiments, each of the at least one immune attenuating drug is administered to the subject once or twice daily in the cycle. In some embodiments, the cycle begins prior to the implanting of b). In some embodiments, the cycle begins about 24 hours, about 12 hours, about 6 hours, about 5 hours, about 4 hours, about 3 hours, about 2 hours, about 1 hour or less, prior to the implanting of b). In some embodiments, the cycle begins after the implanting of b). In some embodiments, the cycle begins about 0.5 hour, about 1 hour, about 2 hours, about 3 hours, about 4 hours, about 5 hours, about 6 hours, about 12 hours, about 24 hours or more after the implanting of b). In some embodiments, the cycle begins concurrently with the implanting of b). The method can comprise administering at least one additional cycle of treatment to the subject.

In some embodiments, the population of genetically modified cells comprises about 1×10⁶ to about 9.5×10⁶ cells. In some embodiments, b) comprises implanting more than one perforated cell delivery device into the mammalian subject. In some embodiments, b) comprises implanting two, three, four, five, or more perforated cell delivery devices into the mammalian subject. In some embodiments, about 1.0×10⁷ to about 2.0×10⁷ genetically modified cells per kilogram are administered to the subject. In some embodiments, the mammalian subject is human.

In some embodiments, C-peptide level is increased in the serum of the subject following the implanting of b). In some embodiments, the increase is relative to (i) the C-peptide level of the subject prior to the implanting of b), (ii) the C-peptide level in one or more untreated subjects, and/or (iii) a reference C-peptide level. In some embodiments, the serum C-peptide level in the subject is increased after about 20 weeks following the implanting of b). In some embodiments, the C-peptide level in the serum of the subject is about 1.1 ng/mL to about 4.4 ng/ml after about 20 weeks following the implanting of b). In some embodiments, A1C percentage in the blood of the subject is decreased in the subject following the implanting of b). In some embodiments, the decrease is relative to (i) the A1C percentage of the subject prior to the implanting of b), (ii) the AC percentage in one or more untreated subjects, and/or (iii) a reference A1C percentage. In some embodiments, fasting plasma glucose (FPG) level in the serum and/or plasma of the subject is decreased in the subject following the implanting of b). In some embodiments, the decrease is relative to (i) the FPG level of the subject prior to the implanting of b), (ii) the FPG level in one or more untreated subjects, and/or (iii) a reference FPG level. In some embodiments, the blood A1C percentage, the serum and/or plasma FPG levels, or any combination thereof, in the subject is decreased after about 20 weeks following the implanting of b). In some embodiments, the A1C percentage in the blood of the subject is less than 6.5% after about 20 weeks following the implanting of b). In some embodiments, the FPG level in the serum and/or plasma of the subject is less than 125 mg/dL after about 20 weeks following the implanting of b).

Disclosed herein include kits and combination products. In some embodiments, the kit or combination product comprises a perforated cell delivery device, a genetically modified cell, or population thereof, and at least one immune attenuating drug. In some embodiments, the genetically modified cell is an allogeneic pancreatic endoderm cell.

In some embodiments, the genetically modified cell comprises (a) a nucleic acid comprising a nucleotide sequence encoding programmed death-ligand 1 (PD-L1) inserted within a gene encoding beta-2 microglobulin (B2M) and (b) a nucleic acid comprising a nucleotide sequence encoding HLA class I histocompatibility antigen, alpha chain E (HLA-E) inserted within a gene encoding thioredoxin interacting protein (TXNIP). In some embodiments, the genetically modified cell expresses PD-L1 and HLA-E and has reduced or eliminated expression of B2M and TXNIP. In some embodiments, the nucleic acid of (a) further comprises a nucleotide sequence encoding TNFAIP3. In some embodiments, the nucleic acid of (b) further comprises a nucleotide sequence encoding MANF. In some embodiments, the nucleic acid of (a) comprises the nucleotide sequence encoding TNFAIP3 linked to a nucleotide sequence encoding a P2A peptide linked to the nucleotide sequence encoding PD-L1 such that the nucleic acid of (a) comprises a TNFAIP3-P2A-PD-L1 polynucleotide sequence. In some embodiments, the TNFAIP3-P2A-PD-L1 polynucleotide sequence comprises the sequence of SEQ ID NO: 54. In some embodiments, the nucleic acid of (a) is operably linked to an exogenous promoter. In some embodiments, the exogenous promoter is a CMV, EF1α, PGK, CAG/CAGGS, or UBC promoter. In some embodiments, the nucleic acid of (b) comprises the nucleotide sequence encoding MANF linked to a nucleotide sequence encoding a P2A peptide linked to the nucleotide sequence encoding HLA-E such that the nucleic acid of (b) comprises a MANF-P2A-HLA-E polynucleotide sequence. In some embodiments, the MANF-P2A-HLA-E polynucleotide sequence comprises SEQ ID NO: 55. In some embodiments, the nucleic acid of (b) is operably linked to an exogenous promoter. In some embodiments, the exogenous promoter is a CMV, EF1α, PGK, CAG/CAGGS, or UBC promoter.

In some embodiments, the genetically modified cell comprises: (a) a nucleic acid comprising a nucleotide sequence encoding TNFAIP3 linked to a nucleotide sequence encoding a P2A peptide linked to a nucleotide sequence encoding PD-L1 such that the nucleic acid of (a) comprises a TNFAIP3-P2A-PD-L1 polynucleotide sequence. In some embodiments, the nucleic acid of (a) is inserted within a gene encoding beta-2 microglobulin (B2M). In some embodiments, the genetically modified cell comprises: (b) a nucleic acid comprising a nucleotide sequence MANF linked to a nucleotide sequence encoding a P2A peptide linked to a nucleotide sequence encoding HLA-E such that the nucleic acid of (b) comprises a MANF-P2A-HLA-E polynucleotide sequence. In some embodiments, the nucleic acid of (b) is inserted within a gene encoding thioredoxin interacting protein (TXNIP).

In some embodiments, the genetically modified cell expresses PD-L1, HLA-E, TNFAIP3 and MANF and has reduced or eliminated expression of B2M and TXNIP. In some embodiments, the TNFAIP3-P2A-PD-L1 polynucleotide sequence comprises the sequence of SEQ ID NO: 54 and the MANF-P2A-HLA-E polynucleotide sequence comprises the sequence of SEQ ID NO: 55. In some embodiments, the nucleic acids of (a) and (b) are operably linked to an exogenous promoter. In some embodiments, the exogenous promoter is a CMV, EF1α, PGK, CAG/CAGGS, or UBC promoter.

In some embodiments, the genetically modified cell further comprises: (c) a disrupted TGFβ2 gene and the cell has reduced or eliminated expression of TGFβ2; (d) a disrupted CIITA gene and the cell has reduced or eliminated expression of CIITA; (e) an insertion of a nucleic acid encoding CD39 and the cell expresses CD39; (f) an insertion of a nucleic acid encoding CD73 and the cell expresses CD73; or (g) any combination of (c), (d), (e) and (f). In some embodiments, the nucleic acid encoding CD39 is inserted within the CIITA gene, and the cell expresses CD39 and has reduced or eliminated expression of CIITA.

In some embodiments, the at least one immune attenuating drug comprises a JAK1 or JAK2 inhibitor, a TNFα or TNFβ blocker, an ILIR blocker, a calcineurin inhibitor, an anti-thymocyte globulin or any combination thereof. In some embodiments, the at least one immune attenuating drug comprises ruxolitinib, etanercept, anakinra or a combination of any thereof. In some embodiments, the at least one immune attenuating drug comprises the calcineurin inhibitor and the anti-thymocyte globulin. In some embodiments, the calcineurin inhibitor is tacrolimus or cyclosporine A. In some embodiments, the at least one immune attenuating drug comprises tacrolimus and the anti-thymocyte globulin. In some embodiments, the tacrolimus is Prograf®, Astagraf XL®, Envarsus XR®, Hecoria®, or any combination thereof. In some embodiments, the anti-thymocyte globulin is Thymoglobulin®, Atgam™, Fresenius™, Tecelac™, or any combination thereof.

In some embodiments, the calcineurin inhibitor is formulated for oral or intravenous administration. In some embodiments, the anti-thymocyte globulin is formulated for intravenous administration. In some embodiments, each of the at least one immune attenuating drug is formulated as a separate composition. In some embodiments, each of the at least one immune attenuating drug is formulated for intravenous administration, subcutaneous administration, oral administration, or any combination thereof. In some embodiments, the population of genetically modified cells is loaded into the perforated delivery device. In some embodiments, the perforated delivery device is loaded with about 1×10⁶ to about 9.5×10⁶ genetically modified cells.

### BRIEF DESCRIPTION OF THE DRAWINGS

FIG. 1 presents the plasmid map of B2M-CAGGS-MANF-P2A-TNFAIP3-P2A-PD-L-1 donor vector.

FIG. 2 presents the plasmid map of B2M-CAGGS-CD39-P2A-PD-L-1 donor vector.

FIG. 3 presents the plasmid map of B2M-CAGGS-TNFAIP3-P2A-PD-L-1 donor vector.

FIG. 4 presents the plasmid map of TXNIP-CAGGS-MANF-P2A-HLA-E donor vector.

FIG. 5 presents the plasmid map of CIITA-CAGGS-CD39 donor vector.

FIG. 6 shows flow cytometry of L3V003B and L3V004B cell lines for CD39 expression.

FIG. 7 shows the morphology of PEC and Stage 6 (S6) cells differentiated from wild type (WT) cells (upper panels) or X1 cells (i.e., TXNIP KO/MANF-P2A-HLA-E KI & B2M KO/TNFAIP3 (A20)-P2A-PD-L-1 KI).

FIG. 8 shows selected gene expression in PEC and Stage 6 (S6) cells differentiated from wild type (WT) cells (upper panels) or X1 (“X1”) cells (i.e., TXNIP KO/MANF-P2A-HLA-E KI & B2M KO/TNFAIP3 (A20)-P2A-PD-L-1 KI).

FIG. 9 shows flow cytometry of CHGA, PDX1 and NKX6.1 in PEC cells (PECs) differentiated from wild type (WT) cells or TXNIP KO/MANF-P2A-HLA-E KI & B2M KO/TNFAIP3 (A20)-P2A-PD-L-1 KI cells (L1V028-C3, LIV028-24).

FIG. 10A-FIG. 10B display flow cytometry data in Stage 6 (S6) cell. FIG. 10A shows flow cytometry of CHGA, PDX1 and NKX6.1 in Stage 6 (S6) cells differentiated from wild type (WT) cells. FIG. 10B shows flow cytometry of CHGA, PDX1 and NKX6.1 in Stage 6 (S6) cells differentiated from X1 cells (i.e., TXNIP KO/MANF-P2A-HLA-E KI & B2M KO/TNFAIP3 (A20)-P2A-PD-L-1 KI).

FIG. 11 shows C-peptide levels at 12, 16, 20, 24 weeks in blood samples obtained from athymic nude rats transplanted with cell aggregates of LIV009B bulk cells (GRP 1) or LIV008 clonal isolates (B2M KO/MANF-P2A-TNFAIP3-P2A-PD-L-1 KI; GRP 2 and GRP 3) or control cells (GRP 4), 90 min after intraperitoneal administration of 3 g/kg glucose for GRP 1-3 and 60 min after administration for GRP 4.

FIG. 12 shows C-peptide levels at 12, 16, 20, 24 weeks in blood samples obtained from athymic nude rats transplanted with cell aggregates from B2M KO/CD39-P2A-PD-L-1 KI or control cells 90 min (60 min for the 24-week reading) after intraperitoneal administration of 3 g/kg glucose.

FIG. 13 shows C-peptide levels at 12 and 16 weeks in blood samples obtained from NSG mice transplanted with capsules containing PEC stage or Stage 6 (S6) differentiated cell aggregates of unmodified (NCG) or B2M KO/TNFAIP3-P2A-PD-L-1 KI & TXNIP KO/MANF-P2A-HLA-E KI (X1) after glucose stimulation.

FIG. 14 presents the average C-peptide levels at 12, 16, 20, 24 weeks after glucose stimulation in the PEC-control (NCG) and PEC-X1 (B2M KO/TNFAIP3-P2A-PD-L-1 KI and TXNIP KO/MANF-P2A-HLA-E KI) groups of mice.

FIG. 15 presents C-peptide levels at 12, 16, and 20 weeks in NSG mice transplanted with capsules containing cells differentiated from unmodified (CON) or clones (i.e., 6D09, 6H07, and 5C10) of B2M KO/TNFAIP3-P2A-PD-L-1 KI & TXNIP KO/MANF-P2A-HLA-E KI (X1) cells.

FIG. 16 presents the plasmid map of B2M-CAGGS-CD39-P2A-CD73-P2A-PD-L-1 donor vector.

FIG. 17 shows flow cytometry of SOX17 and FOXA2 expression in LIV017B cells (i.e., CD39-P2A-PD-L-1 KI and B2M KO), LIV018B cells (i.e., CD39-P2A-CD73-P2A-PD-L-1 KI and B2M KO), and LIV019B cells (i.e., TNFAIP3 (A20)-P2A-PD-L-1 KI and B2M KO).

FIG. 18 shows flow cytometry of CHGA, NKX6.1, and PDX1 expression in L1V017B cells (i.e., CD39-P2A-PD-L-1 KI and B2M KO), LIV018B cells (i.e., CD39-P2A-CD73-P2A-PD-L-1 KI and B2M KO), and LIV019B cells (i.e., TNFAIP3 (A20)-P2A-PD-L-1 KI and B2M KO).

FIG. 19 presents a time course of gene expression of various markers in LIV017B cells (i.e., CD39-P2A-PD-L-1 KI and B2M KO), LIV018B cells (i.e., CD39-P2A-CD73-P2A-PD-L-1 KI and B2M KO), and LIV019B cells (i.e., TNFAIP3 (A20)-P2A-PD-L-1 KI and B2M KO).

FIG. 20A-FIG. 20B display T1DE analyses of edited cells. FIG. 20A shows T1DE analysis showing a 90% KO of the TGF-β2 gene in X1 (B2M KO/TNFAIP3-P2A-PD-L-1 KI & TXNIP KO/MANF-P2A-HLA-E KI)+TGF-β2 KO cells (“L3V002B”) with prominent edits of +1 and 7 causing a frame shift in the coding region. FIG. 20B shows T1DE analysis showing a 90% KO of the TGF-β2 gene in X4 (B2M KO/TNFAIP3-P2A-PD-L-1 KI & TXNIP KO/MANF-P2A-HLA-E KI & CIITA KO/CD39 KI)+TGF-β2 cells (“L3V004B”) with prominent edits of +1 and −7 causing a frame shift in the coding region.

FIG. 21 presents immune evasion assay data using peripheral blood mononuclear cells proliferation assay in the presence of X1 and B2M KO edited cells with or without the presence of TGF-β blockers in the medium.

FIG. 22A-FIG. 22B display ELISA assay results. FIG. 22A shows data from an ELISA assay against secreted TGF-β2 in condition media harboring differentiated wild-type, V1B (HLA-E KI, TXNIP KO, PD-L-1 KI, B2M KO) and TGF-β2 KO PECs for 72 hrs. FIG. 22B shows data from an ELISA assay against secreted TGF-β1 in condition media harboring differentiated wild-type, VIB (HLA-E KI, TXNIP KO, PD-L-1 KI, B2M KO) and TGF-β2 KO PECs for 72 hrs.

FIG. 23A-FIG. 23C display data related to TGF-β, GDF-9, and PDGF-AA secretion. FIG. 23A provides data for TGF-β secreted from VIB and TGF-β2 KO PECs. FIG. 23B provides data for GDF-9 secreted from VIB and TGF-β2 KO PEC cells. FIG. 23C provides data for PDGF-AA secreted from VIB and TGF-β2 KO PECs.

FIG. 24A-FIG. 24B show fibroblast migration assays using human lung fibroblasts (MRC-5) cells with condition media from WT, VIB, and X1 PEC cells (FIG. 24A) and WT and TGF-β2 KO PECs (FIG. 24B).

FIG. 25A-FIG. 25C show fibroblast migration assays using human fibrosarcoma (HT1080) cells with condition media from WT, VIB, and X1 PEC cells (FIG. 25A), WT and TGF-β2 KO PECs (FIG. 25B), and WT, X4 (L3V003B), and X4+TGF-β2 KO (L3V004B) PECs (FIG. 25C).

FIG. 26 shows histological evaluations of explants from 3 subjects exhibiting delayed host tissue integration (healing) of implanted devices combined with a robust inflammatory response located at the interface of the devices and the host tissue.

FIG. 27A-FIG. 27C display exemplary data related to the survival and/or immune tolerance of the disclosed cells. FIG. 27A provides data from a T-cell proliferation assay showing that the VCTX211 PECs can evade T-cell response. FIG. 27B provides data from a target showing that the VCTX211 PECs can resist NK-cell response. FIG. 27C provides PEC cell survival data in humanized mouse models showing successful evasion of both adaptive and innate immune response.

FIG. 28A-FIG. 28C display data related to C-peptide levels. FIG. 28A provides data showing that implanted PECs (12 weeks post-transplant) are capable of increased insulin production as shown using C-peptide assays. FIG. 28B provides data showing that implanted PECs (12 weeks post-transplant) are capable of maintaining glucose responsiveness as seen by rise in C-peptide levels after 30 min of glucose injection. FIG. 28C provides data showing that implanted PECs (12 weeks post-transplant) preserve insulin sensitivity as evident from decreases in C-peptide levels by 60% after insulin injection.

FIG. 29 displays histological sections confirming robust engraftment and immune evasion in nude mice model of implants comprising VCTX211 PECs.

FIG. 30A-FIG. 30B display data related to reversal of hypoglycemia by the disclosed methods. FIG. 30A provides data showing normalization of blood glucose levels within 12-16 weeks of implantation. Rats were either untreated (normoglycemic control) or treated with streptozoticin (STZ) approximately 4 weeks before being implanted with VCTX211 PECs. FIG. 30B provides data showing glucose stimulated insulin release as measured using serum C-peptide levels, when measured 90 minutes post glucose injection.

FIG. 31 shows serum C-peptide concentrations for all groups at 12- and 16-weeks after implant.

FIG. 32 displays average body weight measurements for each group over time.

FIG. 33 displays average body weight measurements over time for each group.

FIG. 34 displays average blood glucose measurements.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include methods of producing insulin in a mammalian subject. In some embodiments, the method comprises: a) administering to a mammalian subject at least one immune attenuating drug; b) implanting a perforated cell delivery device comprising a genetically modified cell, or population thereof, into the mammalian subject; and c) maturing the genetically modified cell, or population thereof, in the perforated cell delivery device in the mammalian subject such that the mature cell, or population thereof, produces insulin secreting cells, thereby producing insulin in the mammalian subject.

Disclosed herein include methods for treating a pancreatic disease or disorder in a mammalian subject in need thereof. In some embodiments, the method comprises: a) administering to a mammalian subject at least one immune attenuating drug; b) implanting a perforated cell delivery device comprising a genetically modified cell, or population thereof, into the mammalian subject; and c) maturing the genetically modified cell, or population thereof, in the perforated cell delivery device in the mammalian subject such that the mature cell, or population thereof, produces insulin secreting cells, thereby producing insulin in the mammalian subject and treating the pancreatic disease or disorder.

Disclosed herein include kits and combination products. In some embodiments, the kit or the combination product comprises a perforated cell delivery device, a genetically modified cell, or population thereof, and at least one immune attenuating drug.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, the term “deletion”, which may be used interchangeably with the terms “genetic deletion” or “knock-out”, generally refers to a genetic modification wherein a site or region of genomic DNA is removed by any molecular biology method, e.g., methods described herein, e.g., by delivering to a site of genomic DNA an endonuclease and at least one gRNA. Any number of nucleotides can be deleted. In some embodiments, a deletion involves the removal of at least one, at least two, at least three, at least four, at least five, at least ten, at least fifteen, at least twenty, or at least 25 nucleotides. In some embodiments, a deletion involves the removal of 10-50, 25-75, 50-100, 50-200, or more than 100 nucleotides. In some embodiments, a deletion involves the removal of part or all of one target gene, e.g., a B2M gene, a TXNIP gene, a CIITA gene, or TGF-β2 gene. In some embodiments, a deletion involves the removal of part or all of two target genes, three target gene, or four target genes. In some embodiments, the removal of part of a target gene refers to removal of all or part of a promoter and/or coding sequence of a gene. In some embodiments, a deletion involves the removal of a transcriptional regulator, e.g., a promoter region, of a target gene. In some embodiments, a deletion involves the removal of all or part of a coding region such that the product normally expressed by the coding region is no longer expressed, is expressed as a truncated form, or expressed at a reduced level. In some embodiments, a deletion leads to a decrease in expression of a gene relative to an unmodified cell. In some embodiments, a deletion leads to a loss of expression of a gene relative to an unmodified cell.

As used herein the terms “disruption,” “disrupting,” or “disrupted” refer to genetic modifications that alter the level of expression of a target gene. In some aspects, the disruption can be due to a deletion of at least one nucleotide within or near the target gene or a deletion of part or all of a target gene, as described above. In other aspects, the disruption also can be due to a substitution of at least one nucleotide and/or an insertion of at least one nucleotide within or near the target gene. In further aspects, the disruption can be due to an insertion of one or more exogenous polynucleotides within or near the target gene. In general, as used herein, disrupted expression refers to reduced or eliminated expression of the target gene. In some embodiments, the disruption can be a reduced level of expression (e.g., express less than 30%, less than 25%, less than 20%, less than 10%, or less than 5% of the level of an unmodified cell). In some embodiments, the disruption can be eliminated expression (e.g., no expression or an undetectable level of RNA and/or protein expression). Expression can be measured using any standard RNA-based, protein-based, and/or antibody-based detection method (e.g., RT-PCR, ELISA, flow cytometry, immunocytochemistry, and the like). Detectable levels are defined as being higher that the limit of detection (LOD), which is the lowest concentration that can be measured (detected) with statistical significance by means of a given detection method.

As used herein, the term “endonuclease” generally refers to an enzyme that cleaves phosphodiester bonds within a polynucleotide. In some embodiments, an endonuclease specifically cleaves phosphodiester bonds within a DNA polynucleotide. In some embodiments, an endonuclease is a zinc finger nuclease (ZFN), transcription activator like effector nuclease (TALEN), homing endonuclease (HE), meganuclease, MegaTAL, or a CRISPR-associated endonuclease. In some embodiments, an endonuclease is an RNA-guided endonuclease. In certain aspects, the RNA-guided endonuclease is a CRISPR nuclease, e.g., a Type II CRISPR Cas9 endonuclease or a Type V CRISPR Cpf1 endonuclease. In some embodiments, an endonuclease is a Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease, or a homolog thereof, a recombination of the naturally occurring molecule thereof, a codon-optimized version thereof, or a modified version thereof, or combinations thereof. In some embodiments, an endonuclease may introduce one or more single-stranded breaks (SSBs) and/or one or more double-stranded breaks (DSBs).

The term “exogenous” as used herein refers to a polynucleotide sequence originating outside the recipient cell or organism, a polynucleotide sequence assembled outside the recipient cell or organism, or a polynucleotide sequence originating from the recipient cell or organism but integrated into the recipient genome at a location other than the naturally occurring location. An exogenous polynucleotide sequence may comprise a gene sequence, may comprise a coding sequence (CDS) of a gene, may comprise coding sequences from more than one gene, may comprise promoter sequences, enhancer sequences, and/or other regulatory elements, may comprise ribosome skip sequences, and/or may comprise artificial sequences. An exogenous polynucleotide may be codon optimized to ensure efficient translation in the recipient cell or organism.

As used herein, the term “genetic modification” generally refers to a site of genomic DNA that has been genetically edited or manipulated using any molecular biological method, e.g., methods described herein, e.g., by delivering to a site of genomic DNA an endonuclease and at least one gRNA. Example genetic modifications include insertions, deletions, duplications, inversions, and translocations, and combinations thereof. In some embodiments, a genetic modification is a deletion. In some embodiments, a genetic modification is an insertion. In other embodiments, a genetic modification is an insertion-deletion mutation (or indel), such that the reading frame of the target gene is shifted leading to an altered gene product or no gene product.

As used herein, the term “guide RNA” or “gRNA” generally refers to short ribonucleic acid that can interact with, e.g., bind to, an endonuclease and bind, or hybridize to a target genomic site or region. In some embodiments, a gRNA is a single-molecule guide RNA (sgRNA). In some embodiments, a gRNA may comprise a spacer extension region. In some embodiments, a gRNA may comprise a tracrRNA extension region. In some embodiments, a gRNA is single-stranded. In some embodiments, a gRNA comprises naturally occurring nucleotides. In some embodiments, a gRNA is a chemically modified gRNA. In some embodiments, a chemically modified gRNA is a gRNA that comprises at least one nucleotide with a chemical modification, e.g., a 2′-O-methyl sugar modification. In some embodiments, a chemically modified gRNA comprises a modified nucleic acid backbone. In some embodiments, a chemically modified gRNA comprises a 2′-O-methyl-phosphorothioate residue. In some embodiments, a gRNA may be pre-complexed with a DNA endonuclease.

As used herein, the term “insertion,” which may be used interchangeably with the terms “genetic insertion” or “knock-in”, generally refers to a genetic modification wherein a polynucleotide is introduced or added into a site or region of genomic DNA by any molecular biological method, e.g., methods described herein, e.g., by delivering to a site of genomic DNA an endonuclease and at least one gRNA. In some embodiments, an insertion of an exogenous polynucleotide occurs within or near a target gene. In some embodiments, an insertion of an exogenous polynucleotide may occur within or near a site of genomic DNA that has been the site of a prior genetic modification, e.g., a deletion or insertion-deletion mutation. In some embodiments, an insertion occurs at a site of genomic DNA that partially overlaps, completely overlaps, or is contained within a site of a prior genetic modification, e.g., a deletion or insertion-deletion mutation. In some embodiments, an insertion simultaneously leads to a disruption of the gene at the targeted site of the insertion. In some embodiments, an insertion occurs at a safe harbor locus. In some embodiments, an insertion involves the introduction of a polynucleotide that encodes a protein of interest. In some embodiments, an insertion involves the introduction of a polynucleotide that encodes a tolerogenic factor. In some embodiments, an insertion involves the introduction of a polynucleotide that encodes a survival factor. In some embodiments, the insertion involves the introduction of a polynucleotide that encodes MANF, TNFAIP3, CD39, CD73, PD-L-1, and/or HLA-E. In some embodiments, an insertion involves the introduction of an exogenous promoter, e.g., a constitutive promoter, e.g., a CAG or CAGGS promoter. In some embodiments, an insertion involves the introduction of a polynucleotide that encodes a noncoding gene. In general, a polynucleotide to be inserted is flanked by sequences (e.g., homology arms) having substantial sequence homology with genomic DNA at or near the site of insertion.

As used herein, the terms “Major histocompatibility complex class I” or “MHC-I” generally refer to a class of biomolecules that are found on the cell surface of all nucleated cells in vertebrates, including mammals, e.g., humans; and function to display peptides of non-self or foreign antigens, e.g., proteins, from within the cell (i.e. cytosolic) to cytotoxic T cells, e.g., CD8+ T cells, in order to stimulate an immune response. In some embodiments, an MHC-I biomolecule is a MHC-I gene or a MHC-I protein. Complexation of MHC-I proteins with beta-2 microglobulin (B2M) protein is required for the cell surface expression of all MHC-I proteins. In some embodiments, decreasing the expression of an MHC-I human leukocyte antigen (HLA) relative to an unmodified cell involves a decrease (or reduction) in the expression of a MHC-I gene. In some embodiments, decreasing the expression of an MHC-I human leukocyte antigen (HLA) relative to an unmodified cell involves a decrease (or reduction) in the cell surface expression of a MHC-I protein. In some embodiments, an MHC-I biomolecule is HLA-A (NCBI Gene ID No: 3105), HLA-B (NCBI Gene ID No: 3106), HLA-C (NCBI Gene ID No: 3107), or B2M (NCBI Gene ID No: 567).

As used herein, the term “Major histocompatibility complex class II” or “MHC-II” generally refer to a class of biomolecules that are typically found on the cell surface of antigen-presenting cells in vertebrates, including mammals, e.g., humans; and function to display peptides of non-self or foreign antigens, e.g., proteins, from outside of the cell (extracellular) to cytotoxic T-cells, e.g., CD8+ T-cells, in order to stimulate an immune response. In some embodiments, an antigen-presenting cell is a dendritic cell, macrophage, or a B cell. In some embodiments, an MHC-II biomolecule is a MHC-II gene or a MHC-II protein. In some embodiments, decreasing the expression of an MHC-II human leukocyte antigen (HLA) relative to an unmodified cell involves a decrease (or reduction) in the expression of a MHC-II gene. In some embodiments, decreasing the expression of an MHC-II human leukocyte antigen (HLA) relative to an unmodified cell involves a decrease (or reduction) in the cell surface expression of a MHC-II protein. In some embodiments, a MHC-II biomolecule is HLA-DPA (NCBI Gene ID No: 3113), HLA-DPB (NCBI Gene ID No: 3115), HLA-DMA (NCBI Gene ID No: 3108), HLA-DMB (NCBI Gene ID No: 3109), HLA-DOA (NCBI Gene ID No: 3111), HLA-DOB (NCBI Gene ID No: 3112), HLA-DQA (NCBI Gene ID No: 3117), HLA-DQB (NCBI Gene ID No: 3119), HLA-DRA (NCBI Gene ID No: 3122), or HLA-DRB (NCBI Gene ID No: 3123).

As used herein, the term “polynucleotide”, which may be used interchangeably with the term “nucleic acid,” generally refers to a biomolecule that comprises two or more nucleotides. In some embodiments, a polynucleotide comprises at least two, at least five at least ten, at least twenty, at least 30, at least 40, at least 50, at least 100, at least 200, at least 250, at least 500, or any number of nucleotides. For example, the polynucleotides may include at least 500 nucleotides, at least about 600 nucleotides, at least about 700 nucleotides, at least about 800 nucleotides, at least about 900 nucleotides, at least about 1000 nucleotides, at least about 2000 nucleotides, at least about 3000 nucleotides, at least about 4000 nucleotides, at least about 4500 nucleotides, or at least about 5000 nucleotides. A polynucleotide may be a DNA or RNA molecule or a hybrid DNA/RNA molecule. A polynucleotide may be single-stranded or double-stranded. In some embodiments, a polynucleotide is a site or region of genomic DNA. In some embodiments, a polynucleotide is an endogenous gene that is comprised within the genome of an unmodified cell or universal donor cell. In some embodiments, a polynucleotide is an exogenous polynucleotide that is not integrated into genomic DNA. In some embodiments, a polynucleotide is an exogenous polynucleotide that is integrated into genomic DNA. In some embodiments, a polynucleotide is a plasmid or an adeno-associated viral vector. In some embodiments, a polynucleotide is a circular or linear molecule.

As used herein, the term “safe harbor locus” generally refers to any location, site, or region of genomic DNA that may be able to accommodate a genetic insertion into said location, site, or region without adverse effects on a cell. In some embodiments, a safe harbor locus is an intragenic or extragenic region. In some embodiments, a safe harbor locus is a region of genomic DNA that is typically transcriptionally silent. In some embodiments, a safe harbor locus is a AAVSI (PPP1 R12C), ALB, Angptl3, ApoC3, ASGR2, CCR5, FIX (F9), G6PC, Gys2, HGD, Lp(a), Pcsk9, Serpinal, TF, or TTR locus. In some embodiments, a safe harbor locus is described in Sadelain, M. et al., “Safe harbours for the integration of new DNA in the human genome,” Nature Reviews Cancer, 2012, Vol 12, pages 51-58.

As used herein, the term “safety switch” generally refers to a biomolecule that leads a cell to undergo apoptosis. In some embodiments, a safety switch is a protein or gene. In some embodiments, a safety switch is a suicide gene. In some embodiments, a safety switch, e.g., herpes simplex virus thymidine kinase (HSV-tk), leads a cell to undergo apoptosis by metabolizing a prodrug, e.g., ganciclovir. In some embodiments, the overexpressed presence of a safety switch on its own leads a cell to undergo apoptosis. In some embodiments, a safety switch is a p53-based molecule, HSV-tk, or inducible caspase-9.

As used herein, the term “subject” refers to a mammal. In some embodiments, a subject is non-human primate or rodent. In some embodiments, a subject is a human. In some embodiments, a subject has, is suspected of having, or is at risk for, a disease or disorder. In some embodiments, a subject has one or more symptoms of a disease or disorder.

As used herein, the term “survival factor” generally refers to a protein (e.g., expressed by a polynucleotide as described herein) that, when increased or decreased in a cell, enables the cell, e.g., a universal donor cell, to survive after transplantation or engraftment into a host subject at higher survival rates relative to an unmodified cell. In some embodiments, a survival factor is a human survival factor. In some embodiments, a survival factor is a member of a critical pathway involved in cell survival. In some embodiments, a critical pathway involved in cell survival has implications on hypoxia, reactive oxygen species, nutrient deprivation, and/or oxidative stress. In some embodiments, the genetic modification, e.g., deletion or insertion, of at least one survival factor enables a universal donor cell to survive for a longer time period, e.g., at least 1.05, at least 1.1, at least 1.25, at least 1.5, at least 2, at least 3, at least 4, at least 5, at least 10, at least 20, or at least 50 times longer time period, than an unmodified cell following engraftment. In some embodiments, a survival factor is MANF (NCBI Gene ID No: 7873), ZNF143 (NCBI Gene ID No: 7702), TXNIP (NCBI Gene ID No: 10628), FOXO1 (NCBI Gene ID No: 2308), or JNK (NCBI Gene ID No: 5599). In some embodiments, a survival factor is inserted into a cell, e.g., a universal donor cell. In some embodiments, a survival factor is deleted from a cell, e.g., a universal donor cell. In some embodiments, an insertion of a polynucleotide that encodes MANF enables a cell, e.g., a universal donor cell, to survive after transplantation or engraftment into a host subject at higher survival rates relative to an unmodified cell. In some embodiments, a deletion or insertion-deletion mutation within or near a TXNIP gene enables a cell, e.g., a universal donor cell, to survive after transplantation or engraftment into a host subject at higher survival rates relative to an unmodified cell.

As used herein, the term “tolerogenic factor” generally refers to a protein (e.g., expressed by a polynucleotide as described herein) that, when increased or decreased in a cell, enables the cell, e.g., a universal donor cell, to inhibit or evade immune rejection after transplantation or engraftment into a host subject at higher rates relative to an unmodified cell. In some embodiments, a tolerogenic factor is a human tolerogenic factor. In some embodiments, the genetic modification of at least one tolerogenic factor (e.g., the insertion or deletion of at least one tolerogenic factor) enables a cell, e.g., a universal donor cell. to inhibit or evade immune rejection with rates at least 1.05, at least 1.1, at least 1.25, at least 1.5, at least 2, at least 3, at least 4, at least 5, at least 10, at least 20, or at least 50 times higher than an unmodified cell following engraftment. In some embodiments, a tolerogenic factor is TNFAIP3 (NCBI Gene ID No: 7128), CD39 (NCBI Gene ID No: 953), CD73 (NCBI Gene ID No. 4907), PD-L-1 (NCBI Gene ID No: 29126), HLA-E (NCBI Gene ID No: 3133), HLA-G (NCBI Gene ID No: 3135), CTLA-4 (NCBI Gene ID No: 1493), or CD47 (NCBI Gene ID No: 961). In some embodiments, a tolerogenic factor is inserted into a cell, e.g., a universal donor cell. In some embodiments, a tolerogenic factor is deleted from a cell, e.g., a universal donor cell. In some embodiments, an insertion of a polynucleotide that encodes TNFAIP3, CD39, CD73, HLA-E, PD-L-1, HLA-G, CTLA-4, and/or CD47 enables a cell, e.g., a universal donor cell, to inhibit or evade immune rejection after transplantation or engraftment into a host subject.

As used herein, the term “transcriptional regulator of MHC-I or MHC-II” generally refers to a biomolecule that modulates, e.g., increases or decreases, the expression of an MHC-I and/or MHC-II human leukocyte antigen. In some embodiments, a biomolecule is a polynucleotide, e.g., a gene, or a protein. In some embodiments, a transcriptional regulator of MHC-I or MHC-II will increase or decrease the cell surface expression of at least one MHC-I or MHC-II protein. In some embodiments, a transcriptional regulator of MHC-I or MHC-II will increase or decrease the expression of at least one MHC-I or MHC-II gene. In some embodiments, the transcriptional regulator is CIITA (NCBI Gene ID No: 4261) or NLRC5 (NCBI Gene ID No: 84166). In some embodiments, a deletion or reduction of expression of CIITA or NLRC5 decreases expression of at least one MHC-I or MHC-II gene.

As used herein, the term “universal donor cell” generally refers to a genetically modified cell that is less susceptible to allogeneic rejection during a cellular transplant and/or demonstrates increased survival after transplantation, relative to an unmodified cell. In some embodiments, a genetically modified cell as described herein is a universal donor cell. In some embodiments, the universal donor cell has increased immune evasion and/or post-transplantation survival compared to an unmodified cell. In some embodiments, the universal donor cell has increased cell survival compared to an unmodified cell. In some embodiments, a universal donor cell may be a stem cell. In some embodiments, a universal donor cell may be an embryonic stem cell (ESC), an adult stem cell (ASC), an induced pluripotent stem cell (iPSC), or a hematopoietic stem or progenitor cell (HSPC) (also called a hematopoietic stem cell (HSC)). In some embodiments, a universal donor cell may be a differentiated cell. In some embodiments, a universal donor cell may be a somatic cell (e.g., immune system cells). In some embodiments, a universal donor cell is administered to a subject. In some embodiments, a universal donor cell is administered to a subject who has, is suspected of having, or is at risk for a disease. In some embodiments, the universal donor cell is capable of being differentiated into lineage-restricted progenitor cells or fully differentiated somatic cells. In some embodiments, the lineage-restricted progenitor cells are pancreatic endoderm progenitors, pancreatic endocrine progenitors, mesenchymal progenitor cells, muscle progenitor cells, blast cells, hematopoietic progenitor cells, or neural progenitor cells. In some embodiments, the fully differentiated somatic cells are endocrine secretory cells such as pancreatic beta cells, epithelial cells, endodermal cells, macrophages, hepatocytes, adipocytes, kidney cells, blood cells, or immune system cells. In some embodiments, the fully differentiated somatic cells are cardiomyocytes.

As used herein, the term “unmodified cell” refers to a cell that has not been subjected to a genetic modification, e.g., involving a polynucleotide or gene that encodes an MHC-I, MHC-I, transcriptional regulator of MHC-I or MHC-II, survival factor, and/or tolerogenic factor. In some embodiments, an unmodified cell may be a stem cell. In some embodiments, an unmodified cell may be an embryonic stem cell (ESC), an adult stem cell (ASC), an induced pluripotent stem cell (iPSC), or a hematopoietic stem or progenitor cell (HSPC) (also called a hematopoietic stem cell (HSC)). In some embodiments, an unmodified cell may be a differentiated cell. In some embodiments, an unmodified cell may be selected from somatic cells (e.g., immune system cells, e.g., a T-cell, e.g., a CD8+ T-cell). If a universal donor cell is compared “relative to an unmodified cell”, the universal donor cell and the unmodified cell are the same cell type or share a common parent cell line, e.g., a universal donor iPSC is compared relative to an unmodified iPSC.

As used herein, the term “within or near a gene” refers to a site or region of genomic DNA that is an intronic or exonic component of said gene or is located proximal to said gene. In some embodiments, a site of genomic DNA is within a gene if it comprises at least a portion of an intron or exon of said gene. In some embodiments, a site of genomic DNA located near a gene may be at the 5′ or 3′ end of said gene (e.g., the 5′ or 3′ end of the coding region of said gene). In some embodiments, a site of genomic DNA located near a gene may be a promoter region or repressor region that modulates the expression of said gene. In some embodiments, a site of genomic DNA located near a gene may be on the same chromosome as said gene. In some embodiments, a site or region of genomic DNA is near a gene if it is within 50 kb, 40 kb, 30 kb, 20 kb, 10 kb, 5 kb, 1 kb, or closer to the 5′ or 3′ end of said gene (e.g., the 5′ or 3′ end of the coding region of said gene).

As used herein, the term “comprising” or “comprises” is inclusive or open-ended and does not exclude additional, unrecited elements, ingredients, or method steps; the phrase “consisting of” or “consists of” is closed and excludes any element, step, or ingredient not specified; and the phrase “consisting essentially of” or “consists essentially” means that specific further components can be present, namely those not materially affecting the essential characteristics of the compound, composition, or method. When used in the context of a sequence, the phrase “consisting essentially of” or “consists essentially” means that the sequence can comprise substitutions and/or additional sequences that do not change the essential function or properties of the sequence.

As described further below, the methods and combination products provided herein advantageously enable the replacement and replenishment of insulin producing cells in an individual without chronic use of immunosuppression and without a harmful foreign body response to the delivery device. Disclosed herein include methods of producing insulin in a mammalian subject. In some embodiments, the method comprises: a) administering to a mammalian subject at least one immune attenuating drug; b) implanting a perforated cell delivery device comprising a genetically modified cell, or population thereof, into the mammalian subject; and c) maturing the genetically modified cell, or population thereof, in the perforated cell delivery device in the mammalian subject such that the mature cell, or population thereof, produces insulin secreting cells, thereby producing insulin in the mammalian subject. In some embodiments, inflammation associated with the foreign body response of the perforated cell delivery device loaded with the genetically modified cell, or population thereof in the mammalian subject is reduced compared to administering a comparative perforated cell delivery device loaded with the genetically modified cell, or population thereof, without the at least one immune attenuating drug. In some embodiments, reducing the foreign body response comprises reducing cell trafficking and/or inflammation external to the device in a host tissue, facilitating integration of the device into the host tissue, and/or improving or accelerating healing of the host tissue around the device.

Disclosed herein include methods for treating a pancreatic disease or disorder in a mammalian subject in need thereof. In some embodiments, the method comprises: a) administering to a mammalian subject at least one immune attenuating drug; b) implanting a perforated cell delivery device comprising a genetically modified cell, or population thereof, into the mammalian subject; and c) maturing the genetically modified cell, or population thereof, in the perforated cell delivery device in the mammalian subject such that the mature cell, or population thereof, produces insulin secreting cells, thereby producing insulin in the mammalian subject and treating the pancreatic disease or disorder. The pancreatic disease or disorder can be type I diabetes, type II diabetes, or a pancreatectomy. The genetically modified cell can be an allogeneic pancreatic endoderm cell.

### Genetically Modified Cells

Various methods and combination products provided herein are directed to the use and/or delivery of gene edited cells (e.g., genetically modified cells). In various aspects, the gene edited cells are edited to increase their survival or viability and/or evade immune response following engraftment into a subject. In some embodiments, these strategies enable genetically modified cells to survive and/or evade immune response at higher success rates than an unmodified cell. In some embodiments, genetically modified cells comprise the introduction of at least one genetic modification within or near at least one gene that encodes a survival factor, wherein the genetic modification comprises an insertion of a polynucleotide encoding a tolerogenic factor. The genetically modified cells may further comprise at least one genetic modification within or near a gene that encodes one or more MHC-I or MHC-II human leukocyte antigens or a component or a transcriptional regulator of an MHC-I or MHC-II complex, wherein said genetic modification comprises an insertion of a polynucleotide encoding a second tolerogenic factor.

In some embodiments, genetically modified cells comprise the introduction of at least one genetic modification within or near at least one gene that decreases the expression of one or more MHC-I and MHC-II human leukocyte antigens relative to an unmodified cell; at least one genetic modification that increases the expression of at least one polynucleotide that encodes a tolerogenic factor relative to an unmodified cell; and at least one genetic modification that alters the expression of at least one gene that encodes a survival factor relative to an unmodified cell. In other embodiments, genetically modified cells comprise at least one deletion or insertion-deletion mutation within or near at least one gene that alters the expression of one or more MHC-I and MHC-II human leukocyte antigens relative to an unmodified cell; and at least one insertion of a polynucleotide that encodes at least one tolerogenic factor at a site that partially overlaps, completely overlaps, or is contained within, the site of a deletion of a gene that alters the expression of one or more MHC-I and MHC-II HLAs. In yet other embodiments, genetically modified cells comprise at least one genetic modification that alters the expression of at least one gene that encodes a survival factor relative to an unmodified cell.

The genes that encode the major histocompatibility complex (MHC) are located on human Chr. 6p21. The resultant proteins coded by the MHC genes are a series of surface proteins that are essential in donor compatibility during cellular transplantation. MHC genes are divided into MHC class I (MHC-I) and MHC class II (MHC-II). MHC-I genes (HLA-A, HLA-B, and HLA-C) are expressed in almost all tissue cell types, presenting “non-self” antigen-processed peptides to CD8+ T-cells, thereby promoting their activation to cytolytic CD8+ T-cells. Transplanted or engrafted cells expressing “non-self”′ MHC-I molecules will cause a robust cellular immune response directed at these cells and ultimately resulting in their demise by activated cytolytic CD8+ T cells. MHC-I proteins are intimately associated with B2M in the endoplasmic reticulum, which is essential for forming functional MHC-I molecules on the cell surface. In addition, there are three non-classical MHC-Ib molecules (HLA-E, HLA-F, and HLA-G), which have immune regulatory functions. MHC-II biomolecule include HLA-DP, HLA-DM, HLA-DOA, HLA-DOB, HLA-DQ, and HLA-DR. Due to their primary function in the immune response, MHC-I and MHC-II biomolecules contribute to immune rejection following cellular engraftment of non-host cells, e.g., cellular engraftment for purposes of regenerative medicine. MHC-I cell surface molecules are composed of MHC-encoded heavy chains (HLA-A, HLA-B, or HLA-C) and the invariant subunit B2M. Thus, a reduction in the concentration of B2M within a cell allows for an effective method of reducing the cell surface expression of MHC-I cell surface molecules.

In some embodiments, a cell comprises a genomic modification of one or more MHC-I or MHC-II genes. In some embodiments, a cell comprises a genomic modification of one or more polynucleotide sequences that regulates the expression of MHC-I and/or MHC-II. In some embodiments, a genetic modification of the disclosure is performed using any gene editing method including but not limited to those methods described herein.

In some embodiments, decreasing the expression of one or more MHC-I and MHC-II human leukocyte antigens relative to an unmodified cell is accomplished by targeting, e.g., for genetic deletion and/or insertion of at least one base pair, in an MHC-I and/or MHC-II gene directly. In some embodiments, decreasing the expression of one or more MHC-I and MHC-II human leukocyte antigens relative to an unmodified cell is accomplished by targeting, e.g., for genetic deletion, a CIITA gene. In some embodiments, decreasing the expression of one or more MHC-I and MHC-II human leukocyte antigens relative to an unmodified cell is accomplished by targeting, e.g., for genetic deletion, at least one transcriptional regulator of MHC-I or MHC-II. In some embodiments, a transcriptional regulator of MHC-I or MHC-II is a NLRC5, or CIITA gene. In some embodiments, a transcriptional regulator of MHC-I or MHC-II is a RFX5, RFXAP, RFXANK, NFY-A, NFY-B, NFY-C, IRF-1, and/or TAPI gene.

In some embodiments, the genome of a cell has been modified to delete the entirety or a portion of an HLA-A, HLA-B, and/or HLA-C gene. In some embodiments, the genome of a cell has been modified to delete the entirety or a portion of a promoter region of an HLA-A, HLA-B, and/or HLA-C gene. In some embodiments, the genome of a cell has been modified to delete the entirety or a portion of a gene that encodes a transcriptional regulator of MHC-I or MHC-II. In some embodiments, the genome of a cell has been modified to delete the entirety or a portion of a promoter region of a gene that encodes a transcriptional regulator of MHC-I or MHC-II.

The present disclosure provides guide RNAs (gRNAs) that can direct the activities of an associated endonuclease to a specific target site within a polynucleotide. A guide RNA can comprise at least a spacer sequence that hybridizes to a target nucleic acid sequence of interest (e.g., hybridizes to the DNA strand that is complementary to the strand comprising a protospacer sequence of the target site), and a CRISPR repeat sequence. In CRISPR Type II systems, the gRNA also comprises a second RNA called the tracrRNA sequence. In the CRISPR Type II guide RNA (gRNA), the CRISPR repeat sequence and tracrRNA sequence hybridize to each other to form a duplex. In CRISPR Type V systems, the gRNA comprises a crRNA that forms a duplex. In some embodiments, a gRNA can bind an endonuclease, such that the gRNA and endonuclease form a complex. The gRNA can provide target specificity to the complex by virtue of its association with the endonuclease. The genome-targeting nucleic acid thus can direct the activity of the endonuclease. Disclosed herein are spacer sequences that can direct an RNA-guided nuclease system to specific genes, as described below.

In some embodiments, the genome of a cell has been modified to disrupt or decrease the expression of beta-2-microglobulin (B2M), also known as β2 microglobulin, B2 microglubulin, or IMD43. B2M is a non-polymorphic gene that encodes a common protein subunit required for surface expression of all polymorphic MHC class I heavy chains. HLA-I proteins are intimately associated with B2M in the endoplasmic reticulum, which is essential for forming functional, cell-surface expressed HLA-I molecules. Provided herein are spacer sequences for editing B2M gene. In some embodiments, the gRNA targets a site within the B2M gene comprising a 5′-GCTACTCTCTCTTTCTGGCC-3′ sequence (SEQ ID NO: 1). In some embodiments, the gRNA targets a site within the B2M gene comprising a 5′-GGCCGAGATGTCTCGCTCCG-3′ sequence (SEQ ID NO: 2). In some embodiments, the gRNA targets a site within the B2M gene comprising a 5′-CGCGAGCACAGCTAAGGCCA-3′ sequence (SEQ ID NO: 3). In alternate embodiments, the gRNA targets a site within the B2M gene comprising any of the following sequences: 5′-TATAAGTGGAGGCGTCGCGC-3′ (SEQ ID NO: 4), 5′-GAGTAGCGCGAGCACAGCTA-3′ (SEQ ID NO: 5), 5′-ACTGGACGCGTCGCGCTGGC-3′ (SEQ ID NO: 6), 5′-AAGTGGAGGCGTCGCGCTGG-3′ (SEQ ID NO: 7), 5-GGCCACGGAGCGAGACATCT-3′ (SEQ ID NO: 8), 5′-GCCCGAATGCTGTCAGCTTC-3′ (SEQ ID NO: 9). 5′-CTCGCGCTACTCTCTCTTTC-3′ (SEQ ID NO: 10), 5′-TCCTGAAGCTGACAGCATTC-3′ (SEQ ID NO: 11), 5′-TTCCTGAAGCTGACAGCATT-3′ (SEQ ID NO: 12), or 5′-ACTCTCTCTTTCTGGCCTGG-3′ (SEQ ID NO: 13). In some embodiments, the gRNA comprises an RNA version of the polynucleotide sequence of SEQ ID NO: 2. In other embodiments, the gRNA comprises an RNA version of any of SEQ ID NO: 1 or 3-13. In some embodiments, the gRNA comprises a sequence of SEQ ID NO: 62. In some embodiments, the gRNA comprises the sequence of SEQ ID NOs: 61 or 63-73. In some embodiments, the gRNA comprises a spacer sequence of SEQ ID NO: 62. In some embodiments, the gRNA comprises a spacer sequence of SEQ ID NOs: 61 or 63-73. The gRNA/CRISPR nuclease complex targets and cleaves a target site in the B2M locus. Repair of a double-stranded break by NHEJ can result in a deletion of at least on nucleotide and/or an insertion of at least one nucleotide, thereby disrupting or eliminating expression of B2M. Alternatively, the B2M locus can be targeted by at least two CRISPR systems each comprising a different gRNA, such that cleavage at two sites in the B2M locus leads to a deletion of the sequence between the two cuts, thereby eliminating expression of B2M.

In some embodiments, genetically modified cells comprise at least one genetic modification that disrupts the expression of at least one gene that encodes a survival factor, such as TXNIP, relative to an unmodified cell. In some embodiments, the genome of a cell has been modified to decrease the expression of thioredoxin interacting protein (TXNIP), which is also known as EST01027, HHCPA78, THIF, VDUP1, or ARRDC6. TXNIP is metabolic gene involved in redox regulation that can also function as a tumor suppressor. Downregulation or knockout of TXNIP can protect cells from metabolic stress. In some embodiments, the gRNA targets a site within the TXNIP gene comprising a 5′-GAAGCGTGTCTTCATAGCGC-3′ sequence (SEQ ID NO: 32). In some embodiments, the gRNA targets a site within the TXNIP gene comprising a 5′-TTACTCGTGTCAAAGCCGTT-3′ sequence (SEQ ID NO: 33). In some embodiments, the gRNA targets a site within the TXNIP gene comprising a 5′-TGTCAAAGCCGTTAGGATCC-3′ sequence (SEQ ID NO: 34). In some embodiments, the gRNA targets a site within the TXNIP gene comprising a 5′-GCCGTTAGGATCCTGGCTTG-3′ sequence (SEQ ID NO: 35). In some embodiments, the gRNA targets a site within the TXNIP gene comprising a 5′-GCGGAGTGGCTAAAGTGCTT-3′ sequence (SEQ ID NO: 36). In some embodiments, the gRNA targets a site within the TXNIP gene comprising a 5′-TCCGCAAGCCAGGATCCTAA-3′ sequence (SEQ ID NO: 37). In some embodiments, the gRNA targets a site within the TXNIP gene comprising a 5′-GTTCGGCTTTGAGCTTCCTC-3′ sequence (SEQ ID NO: 38). In some embodiments, the gRNA targets site within the TXNIP gene comprising a 5′-GAGATGGTGATCATGAGACC-3′ sequence (SEQ ID NO: 39). In some embodiments, the gRNA targets a site within the TXNIP gene comprising a 5′-TTGTACTCATATTTGTTTCC-3′ sequence (SEQ ID NO: 40). In some embodiments, the gRNA targets a site within the TXNIP gene comprising a 5′-AACAAATATGAGTACAAGTT-3′ sequence (SEQ ID NO: 41). In some embodiments, the gRNA comprises an RNA version of the polynucleotide sequence of SEQ ID NO: 37. In other embodiments, the gRNA comprises an RNA version of any one of SEQ ID NOs: 32-36 or 38-41. In some embodiments, the gRNA comprises a sequence of SEQ ID NO: 80. In some embodiments, the gRNA comprises the sequence of SEQ ID NOs: 79 or 81-84. In some embodiments, the gRNA comprises a spacer sequence of SEQ ID NO: 80. In some embodiments, the gRNA comprises a spacer sequence of SEQ ID NOs: 79 or 81-84. The gRNA/CRISPR nuclease complex targets and cleaves a target site in the TXNIP gene locus. Repair of a double-stranded break by NHEJ can result in a deletion of at least one nucleotide and/or an insertion of at least one nucleotide, thereby disrupting or eliminating expression of TXNIP. Alternatively, insertion of a polynucleotide encoding an exogenous gene into the TXNIP gene locus can disrupt or eliminate expression of TXNIP.

In some embodiments, the genome of a cell has been modified to disrupt the expression of Class II transactivator (CIITA), which is also known as C2TA, CIITAIV, MHC2TA, NLRA, or class II major histocompatibility complex transactivator. CIITA is a master regulator of major histocompatibility complex (MHC) gene expression. CIITA is a member of the nucleotide binding domain (NBD) leucine-rich repeat (LRR) family of proteins and regulates the transcription of MHC-II by associating with the MHC enhanceosome. The expression of CIITA is induced in B cells and dendritic cells as a function of developmental stage and is inducible by IFN-γ in most cell types. In some embodiments, the gRNA targets a site in the CIITA gene comprising 5′-GGTCCATCTGGTCATAGAAG-3′ (SEQ ID NO: 25). In some embodiments, the gRNA targets a site in the CIITA gene comprising 5′-GCTCCAGGTAGCCACCTTCT-3′ (SEQ ID NO: 48). In some embodiments, the gRNA targets a site in the CITA gene comprising 5′-TAGGGGCCCCAACTCCATGG-3′ (SEQ ID NO: 49). In some embodiments, the gRNA targets a site in the CIITA gene comprising 5′-GGCTTATGCCAATATCGGTG-3′ (SEQ ID NO: 50). In some embodiments, the gRNA targets a site in the CIITA gene comprising 5′-AGGTGATGAAGAGACCAGGG-3′ (SEQ ID NO: 51). In some embodiments, the gRNA comprises an RNA version of the sequence of SEQ ID NO: 25. In some embodiments, the gRNA comprises a sequence of SEQ ID NO: 74. In some embodiments, the gRNA comprises a spacer sequence of SEQ ID NO: 74. The gRNA/CRISPR nuclease complex targets and cleaves a target site in the CIITA gene locus. Repair of a double-stranded break by NHEJ can result in a deletion of at least on nucleotide and/or an insertion of at least one nucleotide, thereby disrupting or eliminating expression of CIITA. Alternatively, insertion of a polynucleotide encoding an exogenous gene into the CIITA gene locus can disrupt or eliminate expression of CIITA.

In some embodiments, the genome of the cell has been modified to disrupt the expression of TGF-β2, also known as TGFB2, Transforming Growth Factor Beta 2, Glioblastoma-Derived T-Cell Suppressor Factor, Transforming Growth Factor Beta-2 Proprotein Prepro-Transforming Growth Factor Beta-2, Cetermin, G-TSF, Transforming Growth Factor Beta-2, BSC-1 Cell Growth Inhibitor 3, TGF-Beta2, Polyergin, LDS4. The gene encodes a secreted ligand of the TGF-β2 superfamily of proteins. TGF-β2 is a key activator of fibroblasts, the central effector of fibrotic response and also promotes fibrogenic phenotype in immune and vascular cells. Disruption of TGF-β2 expression may improve long term survival of engrafted universal donor cells. In some embodiments, the genome of the cell has been modified to disrupt the TGF-82 gene. In some embodiments, a gRNA targets a site in the TGF-82 gene comprising 5′-GTTCATGCGCAAGAGGATCG-3′ (SEQ ID NO: 57). In some embodiments, the gRNA comprises a sequence of SEQ ID NO: 89. In some embodiments, the gRNA comprises a spacer sequence of SEQ ID NO: 89. The gRNA/CRISPR nuclease complex targets and cleaves a target site in the TGF-β2 gene locus. Repair of a double-stranded break by NHEJ can result in a deletion of at least on nucleotide and/or an insertion of at least one nucleotide, thereby disrupting or eliminating expression of TGF-β2. Alternatively, insertion of a polynucleotide encoding an exogenous gene into the TGF-β2 gene locus can disrupt or eliminate expression of TGF-β2.

In some embodiments, a polynucleotide encoding one or more tolerogenic factors can be inserted into cells, e.g., genetically modified or genetically unmodified cells, to create immune-privileged universal donor cells. Exemplary tolerogenic factors include, without limitation, one or more of TNFAIP3, CD39, PD-L-1, HLA-E, CD73, HLA-C, HLA-F, HLA-G, CTLA-4-Ig, CD47, C1-inhibitor (SERPING1), and IL-35. In some embodiments, the tolerogenic factor is TNFAIP3 or A20, also known as OTUD7C, TNFAIP2, AISBL, or TNF alpha induced protein 3. TNFAIP3 or A20 is a key regulator of inflammation and immunity and is known to inhibit NF-kappa B activation as well as TNF-mediated apoptosis. In some embodiments the tolerogenic factor is CD39, which is also known as ENTPD1 (ectonucleoside triphosphate diphosphohydrolase-1), NTPDasel, ATPDase, or SPG64. While CD39 is a tolerogenic factor, it may also provide benefit through increasing angiogenesis, anti-inflammatory activity, and/or other means. In some embodiments, the tolerogenic factor is PD-L-1 (programmed death ligand 1, also referred to herein as PD-L1) also known as cluster of differentiation 274 (CD274), B7 homolog (B7-H, B7H1), PDCDIL1, PDCDILG1, or PDL1. PD-L-1 appears to play a major role in suppressing the adaptive arm of immune system and is considered to be a co-inhibitory factor of the immune response. In some embodiments, the tolerogenic factor is HLA-E, also known as EA1.2, EA2.1, HLA-6.2, MHC, QA1, or major histocompatibility complex, class I, E. HLA-E is an important modulator of natural killer (NK) and cytotoxic T lymphocyte (CTL) activation and inhibitory function. In some embodiments, the tolerogenic factor is CD73, also known as 5′-nucleotidase ecto (NT5E), 5′-nucleotidase (5′-NT), ecto-5′-nucleotidase, ENT, EN, NT5, NTE, or E5NT. CD73 is a plasma membrane protein that catalyzes the conversion of AMP to adenosine. CD73-derived adenosine promotes aberrant differentiation of dendritic cells (DCs) by activating the A2b receptor on DCs which promotes a tolerogenic phenotype characterized by increased production of IL-6, IL-10, VEGF, and IL-8 and expression of immunosuppressive proteins like IDO, TGF-β, arginase 2 and COX2. In some embodiments, the genetic modification, e.g., insertion of at least one polynucleotide encoding at least one tolerogenic factor enables a universal donor cell to inhibit or evade immune rejection with rates at least 1.05, at least 1.1, at least 1.25, at least 1.5, at least 2, at least 3, at least 4, at least 5, at least 10, at least 20, or at least 50 times higher than an unmodified cell following engraftment. In some embodiments, an insertion of a polynucleotide that encodes TNFAIP3, CD39, PD-L-1, HLA-E, CD73, HLA-G, CTLA-4, and/or CD47 enables a genetically modified cell to inhibit or evade immune rejection after transplantation or engraftment into a host subject.

In some embodiments, the survival factor is MANF, which is also known as arginine-rich, mutated in early-stage tumors (ARMET), arginine-rich protein (ARP), or mesencephalic astrocyte derived neurotrophic factor. MANF is an endoplasmic reticulum (ER) stress-inducible neurotrophic factor that promotes proliferation and survival of pancreatic beta cells, as well as survival of dopaminergic neurons. In some embodiments, insertion of a polynucleotide encoding one or more survival factors, such as MANF, enables a universal donor cell to survive after transplantation or engraftment into a host subject with rates at least 1.05, at least 1.1, at least 1.25, at least 1.5, at least 2, at least 3, at least 4, at least 5, at least 10, at least 20, or at least 50 times higher than an unmodified cell following transplantation or engraftment.

The polynucleotide encoding the tolerogenic factor and/or survival factor (e.g., encoding one or more of PD-L1, TNFAIP3, MANF, and HLA-E) generally comprises left and right homology arms that flank the nucleotide sequence encoding the tolerogenic factor. The homology arms have substantial sequence homology to genomic DNA at or near the targeted insertion site. For example, the left homology arm can be a nucleotide sequence homologous with a region located to the left or upstream of the target site or cut site, and the right homology arm can be a nucleotide sequence homologous with a region located to the right or downstream of the target site or cut site. The proximal end of each homology arm can be homologous to genomic DNA sequence abutting the cut site. Alternatively, the proximal end of each homology arm can be homologous to genomic DNA sequence located up to about 10, 20, 30, 40, 50, 60, or 70 nucleobases away from the cut site. As such, the polynucleotide encoding the tolerogenic factor can be inserted into or replace the targeted gene locus within about 10, 20, 30, 40, 50, 60, or 70 base pairs of the cut site, and additional genomic DNA bordering the cut site (and having no homology to a homolog arm) can be deleted. The homology arms can range in length from about 50 nucleotides to several thousands of nucleotides. In some embodiments, the homology arms can range in length from about 500 nucleobases to about 1000 nucleobases. In some embodiments, the homology arms are about 700, about 800, or about 900 nucleobases in length. In some embodiments, the homology arms are about 800 nucleobases in length. The substantial sequence homology between the homology arms and the genomic DNA can be at least about 80%, at least about 85%, at least about 90%, at least about 95%, or at least about 99%. In some embodiments, the homology arms are identical to the genomic DNA.

In some embodiments, the homology arms are used with B2M guides (e.g., gRNAs comprising RNA version of SEQ ID NOs: 1-13). In some embodiments, the homology arms are designed to be used with any B2M guide that would eliminate the start site of the B2M gene. In some embodiments, the B2M homology arms can comprise or consist of a nucleotide sequence of SEQ ID NO: 15 or 22, or a nucleotide sequence having at least 85%, 90%, 95%, or 99% sequence identity with that of SEQ ID NO: 15 or 22. In some embodiments, the left B2M homology arm can comprise or consist of a nucleotide sequence of SEQ ID NO: 15, or a nucleotide sequence having at least 85%, 90%, 95%, or 99% sequence identity with that of SEQ ID NO: 15. In some embodiments, the right B2M homology arm can comprise or consist of a nucleotide sequence of SEQ ID NO: 22, or a nucleotide sequence having at least 85%, 90%, 95%, or 99% sequence identity with that of SEQ ID NO: 22.

In some embodiments, the homology arms are used with TXNIP guides (e.g., gRNAs comprising RNA version of SEQ ID NOs: 32-41). In some embodiments, the homology arms are designed to be used with any TXNIP guide that targets exon 1 of TXNIP (e.g., gRNAs comprising RNA version of SEQ ID NOs: 32-41). In some embodiments, the TXNIP homology arms can comprise or consist of a nucleotide sequence of SEQ ID NO: 42 or 44, or a nucleotide sequence having at least 85%, 90%, 95%, or 99% sequence identity with that of SEQ ID NO: 42 or 44. In some embodiments, the left TXNIP homology arm can comprise or consist of a nucleotide sequence of SEQ ID NO: 42, or a nucleotide sequence having at least 85%, 90%, 95%, or 99% sequence identity with that of SEQ ID NO: 42. In some embodiments, the right TXNIP homology arm can comprise or consist of a nucleotide sequence of SEQ ID NO: 44, or a nucleotide sequence having at least 85%, 90%, 95%, or 99% sequence identity with that of SEQ ID NO: 44.

In some embodiments, the homology arms are used with CIITA guides (e.g., gRNAs comprising RNA version of SEQ ID NO: 25 or 48-51). In some embodiments, the CIITA homology arms can comprise or consist of a nucleotide sequence of SEQ ID NO: 26 or 28, or a nucleotide sequence having at least 85%, 90%, 95%, or 99% sequence identity with that of SEQ ID NO: 26 or 28. In some embodiments, the left CIITA homology arm can comprise or consist of a nucleotide sequence of SEQ ID NO: 26, or a nucleotide sequence having at least 85%, 90%, 95%, or 99% sequence identity with that of SEQ ID NO: 26. In some embodiments, the right CIITA homology arm can comprise or consist of a nucleotide sequence of SEQ ID NO: 28, or a nucleotide sequence having at least 85%, 90%, 95%, or 99% sequence identity with that of SEQ ID NO: 28.

In some embodiments, the homology arms are used with TGF-82 guides (e.g., gRNAs targeting a sequence comprising SEQ ID NO: 57).

The polynucleotides disclosed herein can comprise sequence encoding one or more ribosome skips, such that, upon expression, a single transcript is produced but due to a ribosome skip during translation, two or more separate proteins are produced. In some embodiments, the ribosome skip can be a short peptide (˜20 AA) that prevents the ribosome from creating the peptide bond between a glycine and a proline at the C terminal end of the growing polypeptide chain. The ribosome pauses after the glycine, resulting in release of the nascent polypeptide chain. Translation resumes, with the proline becoming the first amino acid of a second polypeptide chain. This mechanism results in apparent co-translational cleavage of the polypeptide. A highly conserved sequence at the C-terminus of the ribosome skip peptide contributes to steric hindrance and ribosome skipping. In some embodiments, the ribosome skip peptide is a 2A sequence family member. Suitable 2A sequence family members include F2A, T2A, E2A, and P2A, wherein F2A is derived from foot-and-mouth disease virus 2A, T2A is derived from thosea asigna virus 2A, E2A is derived from equine rhinitis A virus, and P2A derived from porcine teschovirus-1 2A. In some embodiments, the ribosome skip peptide is P2A. In some embodiments, sequence encoding the ribosome skip P2A comprises or consists of a nucleotide sequence of SEQ ID NO: 18. In other embodiments, the ribosome skip can be an internal ribosome entry sequence (IRES), which is an RNA element that allows for translation initiation in a cap-independent manner. The IRES, therefore, allows for the production of two separate proteins from the single transcription unit. IRES elements are well known in the art, e.g., can be derived from viral genome (e.g., picornavirus, aphthovirus, pestivirus IRES) or from cellular mRNAs (e.g., various growth factors, transcription factors, oncogenes, and the like).

The nucleic acids encoding, e.g., one or more of PD-L1, HLA-E, TNFAIP3, and MANF as disclosed herein can be operably linked to an exogenous promoter. The exogenous promoter can be a constitutive, inducible, temporal-, tissue-, or cell type-specific promoter. In some embodiments, the exogenous promoter is a CMV, EF1α, PGK, CAG/CAGGS, or UBC promoter. In general, a CAG or CAGGS promoter comprises a CMV enhancer, a chicken β-actin promoter, and a chimeric intron. In some embodiments, a CAG or CAGGS promoter comprises or consists of a nucleotide sequence of SEQ ID NO: 16 or nucleotide sequence having at least 85%, 90%, 95%, or 99% sequence identity with that of SEQ ID NO: 16.

In certain embodiments the current disclosure envisages universal donor cells (e.g., genetically modified cells) with one or more insertions of exogenous polynucleotide corresponding to any of genes listed as knock-ins in Table 1 and/or disrupted expression of one or more of the genes listed as knock-outs in Table 1. The genetically modified cells can comprise an insertion of one polynucleotide, insertion of any two polynucleotides, insertion of any three polynucleotides, insertion of any four polynucleotides, insertion of any five polynucleotides, or insertion of all six polynucleotides corresponding to the genes listed in Table 1 in any target genomic location (e.g., a safe harbor location) and/or the engineered universal donor cells can comprise disrupted expression (e.g., reduced or eliminated expression) of one, two, three, or four of the target genes listed in Table 1. The cells can comprise any possible combination of listed gene knock-ins and gene knock-outs. In some embodiments, two or more polynucleotides to be inserted can be linked via one or more sequences encoding a ribosome skip such as a 2A peptide such that two or more separate proteins can be expressed from a single RNA transcript. In some embodiments, a polynucleotide or polynucleotides to be inserted into the genome of the cell are operably linked to an exogenous promoter.

TABLE 1
LIST OF EXEMPLARY GENE KNOCK-INS
AND GENE KNOCK-OUTS

| Gene knock-in | Gene knock-out |
|---|---|
| PD-L-1 | B2M |
| TNFAIP3 (A20) | TXNIP |
| MANF | CIITA |
| CD39 | TGF-β2 |
| HLA-E | |
| CD73 | |

In some embodiments, a polynucleotide encoding PD-L-1 is inserted at a site within or near a B2M gene locus, within or near a TXNIP gene locus, within or near a CIITA gene locus, or within or near the TGF-2 gene locus. In some embodiments, a polynucleotide encoding PD-L-1 is inserted at a site within or near a B2M gene locus. In some embodiments, a polynucleotide encoding PD-L-1 is inserted at a site within or near a B2M gene locus concurrent with or following a deletion of all or part of a B2M gene or promoter. In some embodiments, a polynucleotide encoding PD-L-1 is inserted at a site within or near a TXNIP gene locus concurrent with or following a deletion of all or part of a TXNIP gene or promoter. In some embodiments, a polynucleotide encoding PD-L-1 is inserted at a site within or near a CIITA gene locus concurrent with or following a deletion of all or part of a CIITA gene or promoter. In other embodiments, a polynucleotide encoding PD-L-1 is inserted at a site within or near a TGF-82 gene locus concurrent with or following a deletion of all or part of a TGF-β2 gene or promoter. In some embodiments, the polynucleotide encoding PD-L-1 is operably linked to an exogenous promoter. The exogenous promoter can be a CAG or CAGGS promoter. In some embodiments, the polynucleotide encoding PD-L-1 comprises a nucleotide sequence of SEQ ID NO: 20, or nucleotide sequence having at least 85%, 90%, 95%, or 99% sequence identity with that of SEQ ID NO: 20.

In some embodiments, a polynucleotide encoding TNFAIP3 is inserted at a site within or near a B2M gene locus, within or near a TXNIP gene locus, within or near a CIITA gene locus, or within or near the TGF-82 locus. In some embodiments, a polynucleotide encoding TNFAIP3 is inserted at a site within or near a B2M gene locus. In some embodiments, a polynucleotide encoding TNFAIP3 is inserted at a site within or near a B2M gene locus concurrent with or following a deletion of all or part of a B2M gene or promoter. In some embodiments, a polynucleotide encoding TNFAIP3 is inserted at a site within or near a TXNIP gene locus concurrent with or following a deletion of all or part of a TXNIP gene or promoter. In some embodiments, a polynucleotide encoding TNFAIP3 is inserted at a site within or near a CIITA gene locus concurrent with or following a deletion of all or part of a CIITA gene or promoter. In other embodiments, a polynucleotide encoding TNFAIP3 is inserted at a site within or near a TGF-82 gene locus concurrent with or following a deletion of all or part of a TGF-82 gene or promoter. In some embodiments, the polynucleotide encoding TNFAIP3 is operably linked to an exogenous promoter. The exogenous promoter can be a CAG or CAGGS promoter. In some embodiments, the polynucleotide encoding TNFAIP3 comprises a nucleotide sequence of SEQ ID NO: 19, or nucleotide sequence having at least 85%, 90%, 95%, or 99% sequence identity with that of SEQ ID NO: 19.

In some embodiments, a polynucleotide encoding MANF is inserted at a site within or near a B2M gene locus, within or near a TXNIP gene locus, within or near a CIITA gene locus, or within or near the TGF-82 locus. In some embodiments, a polynucleotide encoding MANF is inserted at a site within or near a TXNIP gene locus. In some embodiments, a polynucleotide encoding MANF is inserted at a site within or near a B2M gene locus concurrent with or following a deletion of all or part of a B2M gene or promoter. In other embodiments, a polynucleotide encoding MANF is inserted at a site within or near a TXNIP gene locus concurrent with or following a deletion of all or part of a TXNIP gene or promoter. In some embodiments, a polynucleotide encoding MANF is inserted at a site within or near a CIITA gene locus concurrent with or following a deletion of all or part of a CIITA gene or promoter. In other embodiments, a polynucleotide encoding MANF is inserted at a site within or near a TGF-82 gene locus concurrent with or following a deletion of all or part of a TGF-β2 gene or promoter. In some embodiments, the polynucleotide encoding MANF is operably linked to an exogenous promoter. The exogenous promoter can be a CAG or CAGGS promoter. In some embodiments, the polynucleotide encoding MANF comprises a nucleotide sequence of SEQ ID NO: 17, or nucleotide sequence having at least 85%, 90%, 95%, or 99% sequence identity with that of SEQ ID NO: 17.

In some embodiments, a polynucleotide encoding CD39 is inserted at a site within or near a B2M gene locus, within or near a TXNIP gene locus, within or near a CIITA gene locus, or within or near the TGF-82 locus. In some embodiments, a polynucleotide encoding CD39 is inserted at a site within or near a CIITA gene locus. In some embodiments, a polynucleotide encoding CD39 is inserted at a site within or near a B2M gene locus concurrent with or following a deletion of all or part of a B2M gene or promoter. In other embodiments, a polynucleotide encoding CD39 is inserted at a site within or near a TXNIP gene locus concurrent with or following a deletion of all or part of a TXNIP gene or promoter. In some embodiments, a polynucleotide encoding CD39 is inserted at a site within or near a CIITA gene locus concurrent with, or following a deletion of a CIITA gene or promoter. In other embodiments, a polynucleotide encoding CD39 is inserted at a site within or near a TGF-2 gene locus concurrent with or following a deletion of all or part of a TGF-82 gene or promoter. The polynucleotide encoding CD39 can be operably linked to an exogenous promoter. The exogenous promoter can be a CAG or CAGGS promoter. In some embodiments, the polynucleotide encoding CD39 comprises a nucleotide sequence of SEQ ID NO: 27, or nucleotide sequence having at least 85%, 90%, 95%, or 99% sequence identity with that of SEQ ID NO: 27.

In some embodiments, a polynucleotide encoding HLA-E is inserted at a site within or near a B2M gene locus, within or near a TXNIP gene locus, within or near a CIITA gene locus, or within or near the TGF-82 locus. In some embodiments, a polynucleotide encoding HLA-E is inserted at a site within or near a TXNIP gene locus. In some embodiments, a polynucleotide encoding HLA-E is inserted at a site within or near a B2M gene locus concurrent with or following a deletion of all or part of a B2M gene or promoter. In other embodiments, a polynucleotide encoding HLA-E is inserted at a site within or near a TXNIP gene locus concurrent with or following a deletion of all or part of a TXNIP gene or promoter. In some embodiments, a polynucleotide encoding HLA-E is inserted at a site within or near a CIITA gene locus concurrent with, or following a deletion of a CIITA gene or promoter. In other embodiments, a polynucleotide encoding HLA-E is inserted at a site within or near a TGF-82 gene locus concurrent with or following a deletion of all or part of a TGF-β2 gene or promoter. In some embodiments, the polynucleotide encoding HLA-E comprises a sequence encoding a HLA-E trimer, the HLA-E trimer comprising a B2M signal peptide fused to an HLA-G presentation peptide fused to a B2M membrane protein fused to HLA-E without its signal peptide. In some embodiments, the polynucleotide encoding HLA-E is operably linked to an exogenous promoter. The exogenous promoter can be a CMV promoter. In some embodiments, the polynucleotide encoding HLA-E comprises a nucleotide sequence of SEQ ID NO: 43, or nucleotide sequence having at least 85%, 90%, 95%, or 99% sequence identity with that of SEQ ID NO: 43.

In some embodiments, a polynucleotide encoding TNFAIP3 and PD-L-1 is inserted at a site within or near a B2M gene locus, within or near a TXNIP gene locus, within or near a CIITA gene locus, or within or near the TGF-82 locus. In some embodiments, a polynucleotide encoding TNFAIP3 and PD-L-1 is inserted at a site within or near a B2M gene locus. In some embodiments, a polynucleotide encoding TNFAIP3 and PD-L-1 is inserted at a site within or near a B2M gene locus concurrent with or following a deletion of all or part of a B2M gene or promoter. In some embodiments, a polynucleotide encoding TNFAIP3 and PD-L-1 is inserted at a site within or near a TXNIP gene locus concurrent with or following a deletion of all or part of a TXNIP gene or promoter. In some embodiments, a polynucleotide encoding TNFAIP3 and PD-L-1 is inserted at a site within or near a CIITA gene locus concurrent with or following a deletion of all or part of a CIITA gene or promoter. In other embodiments, a polynucleotide encoding TNFAIP3 and PD-L-1 is inserted at a site within or near a TGF-82 gene locus concurrent with or following a deletion of all or part of a TGF-β2 gene or promoter. In some embodiments, the polynucleotide encoding TNFAIP3 and PD-L-1 comprises sequence encoding TNFAIP3 that is linked to sequence encoding a ribosome skip that is linked to sequence encoding PD-L-1. The ribosome skip can be a 2A sequence family member, such as P2A. In some embodiments, the polynucleotide comprises TNFAIP3-P2A-PD-L-1 coding sequence. In some embodiments, the polynucleotide encoding TNFAIP3-P2A-PD-L-1 comprises or consists of a nucleotide sequence of SEQ ID NO: 54 or a nucleotide sequence having at least 85%, 90%, 95%, or 99% sequence identity with that of SEQ ID NO: 54. In some embodiments, the polynucleotide encoding TNFAIP3-P2A-PD-L-1 is operably linked to an exogenous promoter. The exogenous promoter can be a CAG or CAGGS promoter. In some embodiments, a donor plasmid encoding TNFAIP3-P2A-PD-L-1 and comprising B2M homology arms has a nucleotide sequence of SEQ ID NO: 31. In some embodiments, a donor plasmid encodes TNFAIP3-P2A-PD-L-1 and comprises TXNIP homology arms. In some embodiments, a donor plasmid encodes TNFAIP3-P2A-PD-L-1 and comprises CIITA homology arms. In some embodiments, a donor vector encodes TNFAIP3-P2A-PD-L-1 and comprises TGF-82 homology arms.

In some embodiments, a polynucleotide encoding MANF and HLA-E is inserted at a site within or near a B2M gene locus, within or near a TXNIP gene locus, or within or near a CIITA gene locus, or within or near the TGF-82 locus. In some embodiments, a polynucleotide encoding MANF and HLA-E is inserted at a site within or near a TXNIP gene locus. In some embodiments, a polynucleotide encoding MANF and HLA-E is inserted at a site within or near a B2M gene locus concurrent with, or following a deletion of all or part of a B2M gene or promoter. In some embodiments, a polynucleotide encoding MANF and HLA-E is inserted at a site within or near a TXNIP gene locus concurrent with, or following a deletion of all or part of a TXNIP gene or promoter. In some embodiments, a polynucleotide encoding MANF and HLA-E is inserted at a site within or near a CIITA gene locus concurrent with, or following a deletion of all or part of a CIITA gene or promoter. In other embodiments, a polynucleotide encoding HLA-E is inserted at a site within or near a TGF-82 gene locus concurrent with or following a deletion of all or part of a TGF-β2 gene or promoter. In some embodiments, the polynucleotide encoding MANF and HLA-E comprises sequence encoding MANF that is linked to sequence encoding a ribosome skip that is linked to sequence encoding HLA-E. The ribosome skip can be a 2A sequence family member, such as P2A. In some embodiments, the sequence encoding HLA-E comprises sequence encoding a HLA-E trimer, the HLA-E trimer comprising a B2M signal peptide fused to an HLA-G presentation peptide fused to a B2M membrane protein fused to HLA-E without its signal peptide. In some embodiments the polynucleotide comprises MANF-P2A-HLA-E coding sequence. In some embodiments, the polynucleotide encoding MANF-P2A-HLA-E comprises or consists of a nucleotide sequence of SEQ ID NO: 55 or a nucleotide sequence having at least 85%, 90%, 95%, or 99% sequence identity with that of SEQ ID NO: 55. In some embodiments, the polynucleotide encoding MANF-P2A-HLA-E is operably linked to an exogenous promoter. The exogenous promoter can be a CAG or CAGGS promoter. In some embodiments, a donor plasmid MANF-P2A-HLA-E and comprising TXNIP homology arms has a nucleotide sequence of SEQ ID NO: 45. In some embodiments, a donor plasmid encodes MANF-P2A-HLA-E and comprises B2M homology arms. In some embodiments, a donor plasmid encodes MANF-P2A-HLA-E and comprises CIITA homology arms. In some embodiments, a donor plasmid encodes MANF-P2A-HLA-E and comprises TGF-82 homology arms.

In some embodiments, the at least one polynucleotide encoding at least one tolerogenic factor and/or survival factor can be delivered to the cells as part of a vector. In some embodiments, the polynucleotide encoding MANF-P2A-HLA-E or TNFAIP3-P2A-PD-L1 can be delivered to the cells as part of a vector. For example, the vector may be a plasmid vector. In various embodiments, the amount of plasmid vector delivered to the cells may range from about 0.5 μg to about 10 μg (per about 10⁶ cells). In some embodiments, the amount of plasmid may range from about 1 μg to about 8 μg, from about 2 μg to about 6 μg, or from about 3 μg to about 5 μg. In specific embodiments, the amount of plasmid delivered to the cells may be about 4 μg.

In some embodiments, the genetically modified cell or population thereof is engineered to express PD-L1 and/or TNFAIP3 and has reduced or eliminated expression of B2M and/or TXNIP. In some embodiments, the genetically modified cell or population thereof is engineered to express TNFAIP3 and/or HLA-E (e.g., HLA-E trimer) and has reduced or eliminated expression of B2M and/or TXNIP. In some embodiments, the genetically modified cell or population thereof expresses PD-L1, HLA-E, TNFAIP3 and MANF, and has reduced or eliminated expression of B2M and TXNIP. In some embodiments, a nucleic acid encoding CD39 is inserted within the CIITA gene, and the cell or population thereof expresses CD39 and has reduced or eliminated expression of CIITA. In some embodiments, the genetically modified cell or population thereof expresses PD-L1, TNFAIP3, MANF, and HLA-E, and has reduced or eliminated expression of B2M and TXNIP. In some embodiments, the genetically modified cell or population thereof expresses PD-L1, TNFAIP3, MANF, HLA-E, and CD39, and has reduced or eliminated expression of B2M, TXNIP, and CIITA. In some embodiments, the genetically modified cell or population thereof has reduced or eliminated expression of TGFβ2.

In some embodiments, the cells further comprise increased or decreased expression, e.g., by a genetic modification, of one or more additional genes that are not necessarily implicated in either immune evasion or cell survival post-engraftment or post-transplantation. In some embodiments, the cells further comprise increased expression of one or more safety switch proteins relative to an unmodified cell. In some embodiments, the cells comprise increased expression of one or more additional genes that encode a safety switch protein. In some embodiments, a safety switch is also a suicide gene. In some embodiments, a safety switch is herpes simplex virus-1 thymidine kinase (HSV-tk) or inducible caspase-9. In some embodiments, a polynucleotide that encodes at least one safety switch is inserted into a genome, e.g., into a safe harbor locus. In some other embodiments, the one or more additional genes that are genetically modified encode one or more of safety switch proteins; targeting modalities; receptors; signaling molecules; transcription factors; pharmaceutically active proteins or peptides; drug target candidates; and proteins promoting engraftment, trafficking, homing, viability, self-renewal, persistence, and/or survival thereof integrated with the construct.

One aspect of the present disclosure provides a method of generating genetically modified cells (e.g., universal donor cells), wherein a genetically modified cell comprises at least one targeted genomic modification at one or more selected sites in genome, the method comprising genetically engineering a cell type as described herein by introducing into said cells one or more polynucleotides to allow targeted modification at selected site; introducing into said cells one or more double strand breaks at the selected sites using one or more endonucleases capable of selected site recognition; and culturing the edited cells to allow endogenous DNA repair to generate targeted insertions or deletions at the selected sites; thereby obtaining genome-modified universal donor cells. Targeted gene knockdowns or knockouts can be performed prior to, simultaneously with, or after targeted polynucleotide insertions. The genome-modified cells can undergo successive rounds of genome modification such that multiple sites are targeted and modified. The genome-modified cells are cultured, characterized, selected, and expanded using techniques well known in the art. The universal donor cells generated by this method will comprise at least one functional targeted genomic modification, and wherein the genome-modified cells, if they are stem cells, are then capable of being differentiated into progenitor cells or fully differentiated cells.

In some other embodiments, the genome-engineered universal donor cells (e.g., genetically modified cells) comprise introduced or increased expression of at least one of HLA-E, HLA-G, CD47, PD-L-1, TNFAIP3, MANF, CD73, and/or CD39. In some embodiments, the genetically modified cells comprise introduced or increased expression of HLA-E, PD-L-1, TNFAIP3, and/or MANF. In some embodiments, the genetically modified cells comprise introduced or increased expression of HLA-E, PD-L-1, TNFAIP3, MANF, and/or CD39. In some embodiments, the genetically modified cells comprise introduced or increased expression of PD-L-1 and CD39 and/or introduced or increased expression of PD-L-1, CD73, and CD39. In some embodiments, the genetically modified cells are HLA class I and/or class II deficient. In some embodiments, the genetically modified cells comprise B2M null or reduction-of-function. In some embodiments, the genetically modified cells comprise B2M null or reduction-of-function and TXNIP null or reduction-of-function. In some embodiments, the genetically modified cells comprise B2M null or reduction-of-function, TXNIP null or reduction-of-function, and CIITA null or reduction-of-function. In some embodiments, the genetically modified cells comprise B2M null or reduction-of-function, TXNIP null or reduction-of-function, CIITA null or reduction-of-function, and TGF-82 null or reduction-of-function.

In some embodiments, the genetically modified donor cells comprise integrated or non-integrated exogenous polynucleotide encoding one or more of HLA-E, HLA-G, CD47, PD-L-1, TNFAIP3, MANF, CD73, and/or CD39. In some embodiments, the genetically modified cells comprise integrated or non-integrated exogenous polynucleotide encoding one or more of HLA-E, PD-L-1, TNFAIP3, MANF, CD73, and/or CD39. In some embodiments, said introduced expression is an increased expression from either non-expressed or lowly expressed genes comprised in said cells. In some embodiments, the non-integrated exogenous polynucleotides are introduced using Sendai virus, AAV, episomal, or plasmid.

In some embodiments, the genetically modified cells are B2M null and TXNIP null with introduced expression of TNFAIP3, PD-L-1, MANF, and HLA-E. In some embodiments, the universal donor cells are CIITA null. In some embodiments the universal donor cells are TGF-82 null. In some embodiments, the genetically modified cells are (i) B2M null with a polynucleotide encoding TNFAIP3 and PD-L-1 inserted within or near the B2M gene locus, and (ii) TXNIP null with polynucleotide encoding MANF and HLA-E inserted within or near the TXNIP gene locus. In some embodiments, the genetically modified cells are (i) B2M null with a polynucleotide encoding TNFAIP3 and PD-L-1 inserted within or near the B2M gene locus, (ii) TXNIP null with polynucleotide encoding MANF and HLA-E inserted within or near the TXNIP gene locus, and (iii) CIITA null with polynucleotide encoding CD39 inserted into or near the CIITA gene locus. In some embodiments, the genetically modified cells are (i) B2M null with a polynucleotide encoding TNFAIP3 and PD-L-1 inserted within or near the B2M gene locus, (ii) TXNIP null with polynucleotide encoding MANF and HLA-E inserted within or near the TXNIP gene locus, and (iii) CIITA null with polynucleotide encoding CD39 inserted into or near the CIITA gene locus, and (iv) TGF-β2 null.

The genetically modified cell can comprise a nucleic acid comprising a nucleotide sequence encoding TNFAIP3 linked to a nucleotide sequence encoding a P2A peptide linked to a nucleotide sequence encoding PD-L1 such that the nucleic acid of (a) comprises a TNFAIP3-P2A-PD-L1 polynucleotide sequence. The nucleic acid of (a) can be inserted within a gene encoding beta-2 microglobulin (B2M). The genetically modified cell can comprise: a nucleic acid comprising a nucleotide sequence MANF linked to a nucleotide sequence encoding a P2A peptide linked to a nucleotide sequence encoding HLA-E such that the nucleic acid of (b) comprises a MANF-P2A-HLA-E polynucleotide sequence. The nucleic acid of (b) can be inserted within a gene encoding thioredoxin interacting protein (TXNIP). In some embodiments, genetically modified cell expresses PD-L1, HLA-E, TNFAIP3 and MANF, and has reduced or eliminated expression of B2M and TXNIP.

In certain embodiments, said universal donor cells further comprise increased or decreased expression of at least one safety switch protein. Methods of generating any of the genetically modified cells described herein are contemplated to be performed using at least any of the gene editing methods described herein.

### Genome Editing Methods

Genome editing generally refers to the process of modifying the nucleotide sequence of a genome, preferably in a precise or pre-determined manner. In some embodiments, genome editing methods as described herein, e.g., the CRISPR-endonuclease system, may be used to genetically modify a cell as described herein, e.g., to create a universal donor cell. In some embodiments, genome editing methods as described herein, e.g., the CRISPR-endonuclease system, may be used to genetically modify a cell as described herein, e.g., to introduce at least one genetic modification within or near at least one gene that decreases the expression of one or more MHC-I and/or MHC-II human leukocyte antigens or other components of the MHC-I or MHC-II complex relative to an unmodified cell; to introduce at least one genetic modification that increases the expression of at least one polynucleotide that encodes a tolerogenic factor relative to an unmodified cell; and/or to introduce at least one genetic modification that increases or decreases the expression of at least one gene that encodes a survival factor relative to an unmodified cell.

Examples of methods of genome editing described herein include methods of using site-directed nucleases to cut deoxyribonucleic acid (DNA) at precise target locations in the genome, thereby creating single-strand or double-strand DNA breaks at particular locations within the genome. Such breaks can be and regularly are repaired by natural, endogenous cellular processes, such as homology-directed repair (HDR) and non-homologous end joining (NHEJ), as described in Cox et al., “Therapeutic genome editing: prospects and challenges,” Nature Medicine, 2015, 21 (2), 121-31. These two main DNA repair processes consist of a family of alternative pathways. NHEJ directly joins the DNA ends resulting from a double-strand break, sometimes with the loss or addition of nucleotide sequence, which may disrupt or enhance gene expression. HDR utilizes a homologous sequence, or donor sequence, as a template for inserting a defined DNA sequence at the break point. The homologous sequence can be in the endogenous genome, such as a sister chromatid. Alternatively, the donor sequence can be an exogenous polynucleotide, such as a plasmid, a single-strand oligonucleotide, a double-stranded oligonucleotide, a duplex oligonucleotide or a virus, that has regions (e.g., left and right homology arms) of high homology with the nuclease-cleaved locus, but which can also contain additional sequence or sequence changes including deletions that can be incorporated into the cleaved target locus. A third repair mechanism can be microhomology-mediated end joining (MMEJ), also referred to as “Alternative NHEJ,” in which the genetic outcome is similar to NHEJ in that small deletions and insertions can occur at the cleavage site. MMEJ can make use of homologous sequences of a few base pairs flanking the DNA break site to drive a more favored DNA end joining repair outcome, and recent reports have further elucidated the molecular mechanism of this process; see, e.g., Cho and Greenberg, Nature, 2015, 518, 174-76; Kent et al., Nature Structural and Molecular Biology, 2015, 22 (3): 230-7; Mateos-Gomez et al., Nature, 2015, 518, 254-57; Ceccaldi et al., Nature, 2015, 528, 258-62. In some instances, it may be possible to predict likely repair outcomes based on analysis of potential microhomologies at the site of the DNA break.

Each of these genome editing mechanisms can be used to create desired genetic modifications. A step in the genome editing process can be to create one or two DNA breaks, the latter as double-strand breaks or as two single-stranded breaks, in the target locus as near the site of intended mutation. This can be achieved via the use of endonucleases, as described and illustrated herein.

In general, the genome editing methods described herein can be in vitro or ex vivo methods. In some embodiments, the genome editing methods disclosed herein are not methods for treatment of the human or animal body by therapy and/or are not processes for modifying the germ line genetic identity of human beings.

### CRISPR Endonuclease System

The CRISPR-endonuclease system is a naturally occurring defense mechanism in prokaryotes that has been repurposed as an RNA-guided DNA-targeting platform used for gene editing. CRISPR systems include Types I, II, III, IV, V, and VI systems. In some aspects, the CRISPR system is a Type II CRISPR/Cas9 system. In other aspects, the CRISPR system is a Type V CRISPR/Cpf system. CRISPR systems rely on a DNA endonuclease, e.g., Cas9, and two noncoding RNAs, crisprRNA (crRNA) and trans-activating RNA (tracrRNA), to target the cleavage of DNA.

The crRNA drives sequence recognition and specificity of the CRISPR-endonuclease complex through Watson-Crick base pairing, typically with a ˜20 nucleotide (nt) sequence in the target DNA. Changing the sequence of the 5′ 20 nt in the crRNA (e.g., the spacer) allows targeting of the CRISPR-endonuclease complex to specific loci. The CRISPR-endonuclease complex only binds DNA sequences that contain a sequence match to the first 20 nt of the single-guide RNA (sgRNA) if the target sequence (e.g., protospacer) is followed by a specific short DNA motif (with the sequence NGG) referred to as a protospacer adjacent motif (PAM). In some embodiments, the PAM can serve as a binding signal for CRISPR-endonucleases, e.g., Cas9. In some embodiments, the spacer sequence of the gRNA will hybridize to a sequence commentary to the target sequence (e.g., sequence comprising the PAM, e.g., “PAM strand”), and Cas9 cleaves the DNA.

TracrRNA hybridizes with the 3′ end of crRNA to form an RNA-duplex structure that is bound by the endonuclease to form the catalytically active CRISPR-endonuclease complex, which can then cleave the target DNA. Once the CRISPR-endonuclease complex is bound to DNA at a target site, two independent nuclease domains within the endonuclease each cleave one of the DNA strands three bases upstream of the PAM site, leaving a double-strand break (DSB) where both strands of the DNA terminate in a base pair (a blunt end).

In some embodiments, the endonuclease is a Cas9 (CRISPR associated protein 9) endonuclease. In some embodiments, the Cas9 endonuclease is from Streptococcus pyogenes, although other Cas9 homologs may be used, e.g., S. aureus Cas9, N. meningitidis Cas9, S. thermophilus CRISPR 1 Cas9, S. thermophilus CRISPR 3 Cas9, or T. denticola Cas9. In other instances, the CRISPR endonuclease is Cpf1, e.g., L. bacterium ND2006 Cpf1 or Acidaminococcus sp. BV3L6 Cpf1. In some embodiments, the endonuclease is Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease. In some embodiments, wild-type variants may be used. In some embodiments, modified versions (e.g., a homolog thereof, a recombination of the naturally occurring molecule thereof, codon-optimized thereof, or modified versions thereof) of the preceding endonucleases may be used. The CRISPR nuclease can be linked to at least one nuclear localization signal (NLS). The at least one NLS can be located at or within 50 amino acids of the amino-terminus of the CRISPR nuclease and/or at least one NLS can be located at or within 50 amino acids of the carboxy-terminus of the CRISPR nuclease.

Exemplary CRISPR/Cas polypeptides include the Cas9 polypeptides as published in Fonfara et al., “Phylogeny of Cas9 determines functional exchangeability of dual-RNA and Cas9 among orthologous type II CRISPR-Cas systems,” Nucleic Acids Research, 2014, 42:2577-2590. The CRISPR/Cas gene naming system has undergone extensive rewriting since the Cas genes were discovered. Fonfara et al. also provides PAM sequences for the Cas9 polypeptides from various species.

dCas9-FokI or dCpf1-Fok1 and Other Nucleases

Combining the structural and functional properties of the nuclease platforms described above offers a further approach to genome editing that can potentially overcome some of the inherent deficiencies. As an example, the CRISPR genome editing system typically uses a single Cas9 endonuclease to create a DSB. The specificity of targeting is driven by a 20 or 24 nucleotide sequence in the guide RNA that undergoes Watson-Crick base-pairing with the target DNA (plus an additional 2 bases in the adjacent NAG or NGG PAM sequence in the case of Cas9 from S. pyogenes). Such a sequence is long enough to be unique in the human genome, however, the specificity of the RNA/DNA interaction is not absolute, with significant promiscuity sometimes tolerated, particularly in the 5′ half of the target sequence, effectively reducing the number of bases that drive specificity. One solution to this has been to completely deactivate the Cas9 or Cpf1 catalytic function-retaining only the RNA-guided DNA binding function- and instead fusing a FokI domain to the deactivated Cas9; see, e.g., Tsai et al., Nature Biotech, 2014, 32:569-76; and Guilinger et al., Nature Biotech., 2014, 32:577-82. Because FokI must dimerize to become catalytically active, two guide RNAs are required to tether two FokI fusions in close proximity to form the dimer and cleave DNA. This essentially doubles the number of bases in the combined target sites, thereby increasing the stringency of targeting by CRISPR-based systems.

As further example, fusion of the TALE DNA binding domain to a catalytically active HE, such as I-TevI, takes advantage of both the tunable DNA binding and specificity of the TALE, as well as the cleavage sequence specificity of I-TevI, with the expectation that off-target cleavage can be further reduced.

### Base Editing

In some embodiments, a gene is edited in a cell using base editing. Base Editing is a technique enabling the conversion of one nucleotide into another without double-stranded breaks in the DNA. Base editing allows for conversion of a C to T, G to A, or vice versa. An example editor for cytosine includes rAPOBEC1 which is fused to a catalytically inactive form of Cas9. The Cas9 helps to bind a site of interest and the rAPOBEC1 cytidine deaminase induces the point mutation. Conversion of adenine requires a mutant transfer RNA adenosine deaminase (TadA), a Cas9 nickase, and an sgRNA, as described herein. The construct is able to introduce the site-specific mutation without introducing a strand break. In some embodiments, Base Editing is used to introduce one or more mutations in a cell described herein.

### RNA-Guided Endonucleases

The RNA-guided endonuclease systems as used herein can comprise an amino acid sequence having at least 10%, at least 15%, at least 20%, at least 30%, at least 40%, at least 50%, at least 60%, at least 70%, at least 75%, at least 80%, at least 85%, at least 90%, at least 95%, at least 99%, or 100% amino acid sequence identity to a wild-type exemplary endonuclease, e.g., Cas9 from S. pyogenes, US2014/0068797 SEQ ID NO. 8 or Sapranauskas et al., Nucleic Acids Res, 39 (21): 9275-9282 (2011). The endonuclease can comprise at least 70, 75, 80, 85, 90, 95, 97, 99, or 100% identity to a wild-type endonuclease (e.g., Cas9 from S. pyogenes, supra) over 10 contiguous amino acids. The endonuclease can comprise at most: 70, 75, 80, 85, 90, 95, 97, 99, or 100% identity to a wild-type endonuclease (e.g., Cas9 from S. pyogenes, supra) over 10 contiguous amino acids. The endonuclease can comprise at least: 70, 75, 80, 85, 90, 95, 97, 99, or 100% identity to a wild-type endonuclease (e.g., Cas9 from S. pyogenes, supra) over 10 contiguous amino acids in a HNH nuclease domain of the endonuclease. The endonuclease can comprise at most: 70, 75, 80, 85, 90, 95, 97, 99, or 100% identity to a wild-type endonuclease (e.g., Cas9 from S. pyogenes, supra) over 10 contiguous amino acids in a HNH nuclease domain of the endonuclease. The endonuclease can comprise at least: 70, 75, 80, 85, 90, 95, 97, 99, or 100% identity to a wild-type endonuclease (e.g., Cas9 from S. pyogenes, supra) over 10 contiguous amino acids in a RuvC nuclease domain of the endonuclease. The endonuclease can comprise at most: 70, 75, 80, 85, 90, 95, 97, 99, or 100% identity to a wild-type endonuclease (e.g., Cas9 from S. pyogenes, supra) over 10 contiguous amino acids in a RuvC nuclease domain of the endonuclease.

The endonuclease can comprise a modified form of a wild-type exemplary endonuclease. The modified form of the wild-type exemplary endonuclease can comprise a mutation that reduces the nucleic acid-cleaving activity of the endonuclease. The modified form of the wild-type exemplary endonuclease can have less than 90%, less than 80%, less than 70%, less than 60%, less than 50%, less than 40%, less than 30%, less than 20%, less than 10%, less than 5%, or less than 1% of the nucleic acid-cleaving activity of the wild-type exemplary endonuclease (e.g., Cas9 from S. pyogenes, supra). The modified form of the endonuclease can have no substantial nucleic acid-cleaving activity. When an endonuclease is a modified form that has no substantial nucleic acid-cleaving activity, it is referred to herein as “enzymatically inactive.”

Mutations contemplated can include substitutions, additions, and deletions, or any combination thereof. The mutation can convert the mutated amino acid to alanine. The mutation can convert the mutated amino acid to another amino acid (e.g., glycine, serine, threonine, cysteine, valine, leucine, isoleucine, methionine, proline, phenylalanine, tyrosine, tryptophan, aspartic acid, glutamic acid, asparagine, glutamine, histidine, lysine, or arginine). The mutation can convert the mutated amino acid to a non-natural amino acid (e.g., selenomethionine). The mutation can convert the mutated amino acid to amino acid mimics (e.g., phosphomimics). The mutation can be a conservative mutation. For example, the mutation can convert the mutated amino acid to amino acids that resemble the size, shape, charge, polarity, conformation, and/or rotamers of the mutated amino acids (e.g., cysteine/serine mutation, lysine/asparagine mutation, histidine/phenylalanine mutation). The mutation can cause a shift in reading frame and/or the creation of a premature stop codon. Mutations can cause changes to regulatory regions of genes or loci that affect expression of one or more genes.

### Guide RNAs

The present disclosure provides guide RNAs (gRNAs) that can direct the activities of an associated endonuclease to a specific target site within a polynucleotide. A guide RNA can comprise at least a spacer sequence that hybridizes to a target nucleic acid sequence of interest (e.g., hybridizes to the DNA strand that is complementary to the strand comprising a protospacer sequence of the target site), and a CRISPR repeat sequence. In CRISPR Type II systems, the gRNA also comprises a second RNA called the tracrRNA sequence. In the CRISPR Type II guide RNA (gRNA), the CRISPR repeat sequence and tracrRNA sequence hybridize to each other to form a duplex. In CRISPR Type V systems, the gRNA comprises a crRNA that forms a duplex. In some embodiments, a gRNA can bind an endonuclease, such that the gRNA and endonuclease form a complex. The gRNA can provide target specificity to the complex by virtue of its association with the endonuclease. The genome-targeting nucleic acid thus can direct the activity of the endonuclease.

Exemplary guide RNAs include a spacer sequences that comprises 15-200 nucleotides wherein the gRNA targets a genome location based on the GRCh38 human genome assembly. As is understood by the person of ordinary skill in the art, each gRNA can be designed to include a spacer sequence complementary to its genomic target site or region, i.e., the “target sequence.” The “target sequence” is in a target gene that is adjacent to a PAM sequence and is the sequence to be modified by Cas9. The “target sequence” is on the so-called PAM-strand in a “target nucleic acid,” which is a double-stranded molecule containing the PAM-strand and a complementary non-PAM strand. One of skill in the art recognizes that the gRNA spacer sequence hybridizes to the complementary sequence located in the non-PAM strand of the target nucleic acid of interest. Thus, the gRNA spacer sequence is the RNA equivalent of the target sequence. See Jinek et al., Science, 2012, 337, 816-821 and Deltcheva et al., Nature, 2011, 471, 602-607.

The gRNA can be a double-molecule guide RNA. The gRNA can be a single-molecule guide RNA. A double-molecule guide RNA can comprise two strands of RNA. The first strand can comprise in the 5′ to 3′ direction, an optional spacer extension sequence, a spacer sequence and a minimum CRISPR repeat sequence. The second strand can comprise a minimum tracrRNA sequence (complementary to the minimum CRISPR repeat sequence), a 3′ tracrRNA sequence and an optional tracrRNA extension sequence. A single-molecule guide RNA (sgRNA) can comprise, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension can comprise elements that contribute additional functionality (e.g., stability) to the guide RNA. The single-molecule guide linker can link the minimum CRISPR repeat and the minimum tracrRNA sequence to form a hairpin structure. The optional tracrRNA extension can comprise one or more hairpins.

In some embodiments, an sgRNA comprises a 20 nucleotide spacer sequence at the 5′ end of the sgRNA sequence. In some embodiments, an sgRNA comprises a less than a 20 nucleotide spacer sequence at the 5′ end of the sgRNA sequence. In some embodiments, an sgRNA comprises a more than 20 nucleotide spacer sequence at the 5′ end of the sgRNA sequence. In some embodiments, an sgRNA comprises a variable length spacer sequence with 17-30 nucleotides at the 5′ end of the sgRNA sequence. In some embodiments, an sgRNA comprises a spacer extension sequence with a length of more than 1, 5, 10, 15, 20, 25, 30, 35, 40, 45, 50, 60, 70, 80, 90, 100, 120, 140, 160, 180, or 200 nucleotides. In some embodiments, an sgRNA comprises a spacer extension sequence with a length of less than 3, 5, 10, 15, 20, 25, 30, 35, 40, 45, 50, 60, 70, 80, 90, or 100 nucleotides.

In some embodiments, an sgRNA comprises a spacer extension sequence that comprises another moiety (e.g., a stability control sequence, an endoribonuclease binding sequence, or a ribozyme). The moiety can decrease or increase the stability of a nucleic acid targeting nucleic acid. The moiety can be a transcriptional terminator segment (i.e., a transcription termination sequence). The moiety can function in a eukaryotic cell. The moiety can function in a prokaryotic cell. The moiety can function in both eukaryotic and prokaryotic cells. Non-limiting examples of suitable moieties include: a 5′ cap (e.g., a 7-methylguanylate cap (m7 G)), a riboswitch sequence (e.g., to allow for regulated stability and/or regulated accessibility by proteins and protein complexes), a sequence that forms a dsRNA duplex (i.e., a hairpin), a sequence that targets the RNA to a subcellular location (e.g., nucleus, mitochondria, chloroplasts, and the like), a modification or sequence that provides for tracking (e.g., direct conjugation to a fluorescent molecule, conjugation to a moiety that facilitates fluorescent detection, a sequence that allows for fluorescent detection, etc.), and/or a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like).

In some embodiments, an sgRNA comprises a spacer sequence that hybridizes to a sequence in a target polynucleotide. The spacer of a gRNA can interact with a target polynucleotide in a sequence-specific manner via hybridization (i.e., base pairing). The nucleotide sequence of the spacer can vary depending on the sequence of the target nucleic acid of interest.

In a CRISPR-endonuclease system, a spacer sequence can be designed to hybridize to a target polynucleotide that is located 5′ of a PAM of the endonuclease used in the system. The spacer may perfectly match the target sequence or may have mismatches. Each endonuclease, e.g., Cas9 nuclease, has a particular PAM sequence that it recognizes in a target DNA. For example, S. pyogenes Cas9 recognizes a PAM that comprises the sequence 5′-NRG-3′, where R comprises either A or G, where Nis any nucleotide and Nis immediately 3′ of the target nucleic acid sequence targeted by the spacer sequence.

A target polynucleotide sequence can comprise 20 nucleotides. The target polynucleotide can comprise less than 20 nucleotides. The target polynucleotide can comprise more than 20 nucleotides. The target polynucleotide can comprise at least: 5, 10, 15, 16, 17, 18, 19, 20, 21, 22, 23, 24, 25, 30 or more nucleotides. The target polynucleotide can comprise at most: 5, 10, 15, 16, 17, 18, 19, 20, 21, 22, 23, 24, 25, 30 or more nucleotides. The target polynucleotide sequence can comprise 20 bases immediately 5′ of the first nucleotide of the PAM.

A spacer sequence that hybridizes to a target polynucleotide can have a length of at least about 6 nucleotides (nt). The spacer sequence can be at least about 6 nt, at least about 10 nt, at least about 15 nt, at least about 18 nt, at least about 19 nt, at least about 20 nt, at least about 25 nt, at least about 30 nt, at least about 35 nt or at least about 40 nt, from about 6 nt to about 80 nt, from about 6 nt to about 50 nt, from about 6 nt to about 45 nt, from about 6 nt to about 40 nt, from about 6 nt to about 35 nt, from about 6 nt to about 30 nt, from about 6 nt to about 25 nt, from about 6 nt to about 20 nt, from about 6 nt to about 19 nt, from about 10 nt to about 50 nt, from about 10 nt to about 45 nt, from about 10 nt to about 40 nt, from about 10 nt to about 35 nt, from about 10 nt to about 30 nt, from about 10 nt to about 25 nt, from about 10 nt to about 20 nt, from about 10 nt to about 19 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. In some examples, the spacer sequence can comprise 20 nucleotides. In some examples, the spacer can comprise 19 nucleotides. In some examples, the spacer can comprise 18 nucleotides. In some examples, the spacer can comprise 22 nucleotides.

In some examples, the percent complementarity between the spacer sequence and the target nucleic acid is at least about 30%, at least about 40%, at least about 50%, at least about 60%, at least about 65%, at least about 70%, at least about 75%, at least about 80%, at least about 85%, at least about 90%, at least about 95%, at least about 97%, at least about 98%, at least about 99%, or 100%. In some examples, the percent complementarity between the spacer sequence and the target nucleic acid is at most about 30%, at most about 40%, at most about 50%, at most about 60%, at most about 65%, at most about 70%, at most about 75%, at most about 80%, at most about 85%, at most about 90%, at most about 95%, at most about 97%, at most about 98%, at most about 99%, or 100%. In some examples, the percent complementarity between the spacer sequence and the target nucleic acid is 100% over the six contiguous 5′-most nucleotides of the target sequence of the complementary strand of the target nucleic acid. The percent complementarity between the spacer sequence and the target nucleic acid can be at least 60% over about 20 contiguous nucleotides. The length of the spacer sequence and the target nucleic acid can differ by 1 to 6 nucleotides, which may be thought of as a bulge or bulges.

A tracrRNA sequence can comprise nucleotides that hybridize to a minimum CRISPR repeat sequence in a cell. A minimum tracrRNA sequence and a minimum CRISPR repeat sequence may form a duplex, i.e. a base-paired double-stranded structure. Together, the minimum tracrRNA sequence and the minimum CRISPR repeat can bind to an RNA-guided endonuclease. At least a part of the minimum tracrRNA sequence can hybridize to the minimum CRISPR repeat sequence. The minimum tracrRNA sequence can be at least about 30%, about 40%, about 50%, about 60%, about 65%, about 70%, about 75%, about 80%, about 85%, about 90%, about 95%, or 100% complementary to the minimum CRISPR repeat sequence.

The minimum tracrRNA sequence can have a length from about 7 nucleotides to about 100 nucleotides. For example, the minimum tracrRNA sequence can be from about 7 nucleotides (nt) to about 50 nt, from about 7 nt to about 40 nt, from about 7 nt to about 30 nt, from about 7 nt to about 25 nt, from about 7 nt to about 20 nt, from about 7 nt to about 15 nt, from about 8 nt to about 40 nt, from about 8 nt to about 30 nt, from about 8 nt to about 25 nt, from about 8 nt to about 20 nt, from about 8 nt to about 15 nt, from about 15 nt to about 100 nt, from about 15 nt to about 80 nt, from about 15 nt to about 50 nt, from about 15 nt to about 40 nt, from about 15 nt to about 30 nt or from about 15 nt to about 25 nt long. The minimum tracrRNA sequence can be approximately 9 nucleotides in length. The minimum tracrRNA sequence can be approximately 12 nucleotides. The minimum tracrRNA can consist of tracrRNA nt 23-48 described in Jinek et al., supra.

The minimum tracrRNA sequence can be at least about 60% identical to a reference minimum tracrRNA (e.g., wild type, tracrRNA from S. pyogenes) sequence over a stretch of at least 6, 7, or 8 contiguous nucleotides. For example, the minimum tracrRNA sequence can be at least about 65% identical, about 70% identical, about 75% identical, about 80% identical, about 85% identical, about 90% identical, about 95% identical, about 98% identical, about 99% identical or 100% identical to a reference minimum tracrRNA sequence over a stretch of at least 6, 7, or 8 contiguous nucleotides. The duplex between the minimum CRISPR RNA and the minimum tracrRNA can comprise a double helix. The duplex between the minimum CRISPR RNA and the minimum tracrRNA can comprise at least about 1, 2, 3, 4, 5, 6, 7, 8, 9, or 10 or more nucleotides. The duplex between the minimum CRISPR RNA and the minimum tracrRNA can comprise at most about 1, 2, 3, 4, 5, 6, 7, 8, 9, or 10 or more nucleotides. The duplex can comprise a mismatch (i.e., the two strands of the duplex are not 100% complementary). The duplex can comprise at least about 1, 2, 3, 4, or 5 or mismatches. The duplex can comprise at most about 1, 2, 3, 4, or 5 or mismatches. The duplex can comprise no more than 2 mismatches.

In some embodiments, a tracrRNA is a 3′ tracrRNA. In some embodiments, a 3′ tracrRNA sequence can comprise a sequence with at least about 30%, about 40%, about 50%, about 60%, about 65%, about 70%, about 75%, about 80%, about 85%, about 90%, about 95%, or 100% sequence identity to a reference tracrRNA sequence (e.g., a tracrRNA from S. pyogenes).

In some embodiments, an gRNA may comprise a tracrRNA extension sequence. A tracrRNA extension sequence can have a length from about 1 nucleotide to about 400 nucleotides. The tracrRNA extension sequence can have a length of more than 1, 5, 10, 15, 20, 25, 30, 35, 40, 45, 50, 60, 70, 80, 90, 100, 120, 140, 160, 180, or 200 nucleotides. The tracrRNA extension sequence can have a length from about 20 to about 5000 or more nucleotides. The tracrRNA extension sequence can have a length of less than 5, 10, 15, 20, 25, 30, 35, 40, 45, 50, 60, 70, 80, 90, or 100 nucleotides. The tracrRNA extension sequence can comprise less than 10 nucleotides in length. The tracrRNA extension sequence can be 10-30 nucleotides in length. The tracrRNA extension sequence can be 30-70 nucleotides in length.

The tracrRNA extension sequence can comprise a functional moiety (e.g., a stability control sequence, ribozyme, endoribonuclease binding sequence). The functional moiety can comprise a transcriptional terminator segment (i.e., a transcription termination sequence). The functional moiety can have a total length from about 10 nucleotides (nt) to about 100 nucleotides, from about 10 nt to about 20 nt, from about 20 nt to about 30 nt, from about 30 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt, from about 15 nt to about 80 nt, from about 15 nt to about 50 nt, from about 15 nt to about 40 nt, from about 15 nt to about 30 nt, or from about 15 nt to about 25 nt.

In some embodiments, an sgRNA may comprise a linker sequence with a length from about 3 nucleotides to about 100 nucleotides. In Jinek et al., supra, for example, a simple 4 nucleotide “tetraloop” (-GAAA-) was used (Jinek et al., Science, 2012, 337 (6096): 816-821). An illustrative linker has a length from about 3 nucleotides (nt) to about 90 nt, from about 3 nt to about 80 nt, from about 3 nt to about 70 nt, from about 3 nt to about 60 nt, from about 3 nt to about 50 nt, from about 3 nt to about 40 nt, from about 3 nt to about 30 nt, from about 3 nt to about 20 nt, from about 3 nt to about 10 nt. For example, the linker can have a length from about 3 nt to about 5 nt, from about 5 nt to about 10 nt, from about 10 nt to about 15 nt, from about 15 nt to about 20 nt, from about 20 nt to about 25 nt, from about 25 nt to about 30 nt, from about 30 nt to about 35 nt, from about 35 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. The linker of a single-molecule guide nucleic acid can be between 4 and 40 nucleotides. The linker can be at least about 100, 500, 1000, 1500, 2000, 2500, 3000, 3500, 4000, 4500, 5000, 5500, 6000, 6500, or 7000 or more nucleotides. The linker can be at most about 100, 500, 1000, 1500, 2000, 2500, 3000, 3500, 4000, 4500, 5000, 5500, 6000, 6500, or 7000 or more nucleotides.

Linkers can comprise any of a variety of sequences, although in some examples the linker will not comprise sequences that have extensive regions of homology with other portions of the guide RNA, which might cause intramolecular binding that could interfere with other functional regions of the guide. In Jinek et al., supra, a simple 4 nucleotide sequence-GAAA was used (Jinek et al., Science, 2012, 337 (6096): 816-821), but numerous other sequences, including longer sequences can likewise be used. The linker sequence can comprise a functional moiety. For example, the linker sequence can comprise one or more features, including an aptamer, a ribozyme, a protein-interacting hairpin, a protein binding site, a CRISPR array, an intron, or an exon. The linker sequence can comprise at least about 1, 2, 3, 4, or 5 or more functional moieties. In some examples, the linker sequence can comprise at most about 1, 2, 3, 4, or 5 or more functional moieties.

In some embodiments, an sgRNA does not comprise a uracil, e.g., at the 3′end of the sgRNA sequence. In some embodiments, an sgRNA does comprise one or more uracils, e.g., at the 3′end of the sgRNA sequence. In some embodiments, a sgRNA comprises 1, 2, 3, 4, 5, 6, 7, 8, 9, or 10 uracils (U) at the 3′ end of the sgRNA sequence. An sgRNA may be chemically modified. In some embodiments, a chemically modified gRNA is a gRNA that comprises at least one nucleotide with a chemical modification, e.g., a 2′-O-methyl sugar modification. In some embodiments, a chemically modified gRNA comprises a modified nucleic acid backbone. In some embodiments, a chemically modified gRNA comprises a 2′-O-methyl-phosphorothioate residue. In some embodiments, chemical modifications enhance stability, reduce the likelihood or degree of innate immune response, and/or enhance other attributes, as described in the art. In some embodiments, a modified gRNA may comprise modified backbones, for example, phosphorothioates, phosphotriesters, morpholinos, methyl phosphonates, short chain alkyl or cycloalkyl intersugar linkages or short chain heteroatomic or heterocyclic intersugar linkages. Morpholino-based compounds are described in Braasch and David Corey, Biochemistry, 2002, 41 (14): 4503-4510; Genesis, 2001, Volume 30, Issue 3; Heasman, Dev. Biol., 2002, 243:209-214; Nasevicius et al., Nat. Genet., 2000, 26:216-220; Lacerra et al., Proc. Natl. Acad. Sci., 2000, 97:9591-9596.; and U.S. Pat. No. 5,034,506, issued Jul. 23, 1991. Cyclohexenyl nucleic acid oligonucleotide mimetics are described in Wang et al., J. Am. Chem. Soc., 2000, 122:8595-8602.

In some embodiments, a modified gRNA may comprise one or more substituted sugar moieties, e.g., one of the following at the 2′ position: OH, SH, SCH₃, F, OCN, OCH₃, OCH₃ O(CH₂)ₙ CH₃, O(CH₂)ₙ NH₂, or O(CH₂)ₙ CH₃, where n is from 1 to about 10; C1 to C10 lower alkyl, alkoxyalkoxy, substituted lower alkyl, alkaryl or aralkyl; Cl; Br; CN; CF₃; OCF₃; O-, S-, or N-alkyl; O-, S-, or N-alkenyl; SOCH₃; SO₂ CH₃; ONO₂; N₃; NH₂; heterocycloalkyl; heterocycloalkaryl; aminoalkylamino; polyalkylamino; substituted silyl; an RNA cleaving group; a reporter group; an intercalator; 2′-O-(2-methoxyethyl); 2′-methoxy (2′-O—CH₃); 2′-propoxy (2′—OCH₂ CH₂CH₃); and 2′-fluoro (2′-F). Similar modifications may also be made at other positions on the gRNA, particularly the 3′ position of the sugar on the 3′ terminal nucleotide and the 5′ position of 5′ terminal nucleotide. In some examples, both a sugar and an internucleoside linkage, i.e., the backbone, of the nucleotide units can be replaced with novel groups.

Guide RNAs can also include, additionally or alternatively, nucleobase (often referred to in the art simply as “base”) modifications or substitutions. As used herein, “unmodified” or “natural” nucleobases include adenine (A), guanine (G), thymine (T), cytosine (C), and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino) adenine, 2-(imidazolylalkyl) adenine, 2-(aminoalklyamino) adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl) adenine, and 2,6-diaminopurine. Kornberg, A., DNA Replication, W. H. Freeman & Co., San Francisco, pp. 75-77, 1980; Gebeyehu et al., Nucl. Acids Res. 1997, 15:4513. A “universal” base known in the art, e.g., inosine, can also be included. 5-Me-C substitutions have been shown to increase nucleic acid duplex stability by 0.6-1.2° C. (Sanghvi, Y. S., in Crooke, S. T. and Lebleu, B., eds., Antisense Research and Applications, CRC Press, Boca Raton, 1993, pp. 276-278) and are aspects of base substitutions.

Modified nucleobases can comprise other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine, and 3-deazaguanine and 3-deazaadenine.

### Complexes of a Genome-targeting Nucleic Acid and an Endonuclease

A gRNA interacts with an endonuclease (e.g., a RNA-guided nuclease such as Cas9), thereby forming a complex. The gRNA guides the endonuclease to a target polynucleotide.

The endonuclease and gRNA can each be administered separately to a cell or a subject. In some embodiments, the endonuclease can be pre-complexed with one or more guide RNAs, or one or more crRNA together with a tracrRNA. The pre-complexed material can then be administered to a cell or a subject. Such pre-complexed material is known as a ribonucleoprotein particle (RNP). The endonuclease in the RNP can be, for example, a Cas9 endonuclease or a Cpf1 endonuclease. The endonuclease can be flanked at the N-terminus, the C-terminus, or both the N-terminus and C-terminus by one or more nuclear localization signals (NLSs). For example, a Cas9 endonuclease can be flanked by two NLSs, one NLS located at the N-terminus and the second NLS located at the C-terminus. The NLS can be any NLS known in the art, such as a SV40 NLS. The molar ratio of genome-targeting nucleic acid to endonuclease in the RNP can range from about 1:1 to about 10:1. For example, the molar ratio of sgRNA to Cas9 endonuclease in the RNP can be 3:1.

### Nucleic Acids Encoding System Components

The present disclosure provides a nucleic acid comprising a nucleotide sequence encoding a genome-targeting nucleic acid of the disclosure, an endonuclease of the disclosure, and/or any nucleic acid or proteinaceous molecule necessary to carry out the aspects of the methods of the disclosure. The encoding nucleic acids can be RNA, DNA, or a combination thereof.

The nucleic acid encoding a genome-targeting nucleic acid of the disclosure, an endonuclease of the disclosure, and/or any nucleic acid or proteinaceous molecule necessary to carry out the aspects of the methods of the disclosure can comprise a vector (e.g., a recombinant expression vector). The term “vector” refers to a nucleic acid molecule capable of transporting another nucleic acid to which it has been linked. One type of vector is a “plasmid”, which refers to a circular double-stranded DNA loop into which additional nucleic acid segments can be ligated. Another type of vector is a viral vector, wherein additional nucleic acid segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome.

In some examples, vectors can be capable of directing the expression of nucleic acids to which they are operatively linked. Such vectors are referred to herein as “recombinant expression vectors”, or more simply “expression vectors”, which serve equivalent functions. The term “operably linked” means that the nucleotide sequence of interest is linked to regulatory sequence(s) in a manner that allows for expression of the nucleotide sequence. The term “regulatory sequence” is intended to include, for example, promoters, enhancers and other expression control elements (e.g., polyadenylation signals). Such regulatory sequences are well known in the art and are described, for example, in Goeddel; Gene Expression Technology: Methods in Enzymology, 1990, 185, Academic Press, San Diego, CA. Regulatory sequences include those that direct constitutive expression of a nucleotide sequence in many types of host cells, and those that direct expression of the nucleotide sequence only in certain host cells (e.g., tissue-specific regulatory sequences). It will be appreciated by those skilled in the art that the design of the expression vector can depend on such factors as the choice of the target cell, the level of expression desired, and the like.

Expression vectors contemplated include, but are not limited to, viral vectors based on vaccinia virus, poliovirus, adenovirus, adeno-associated virus, SV40, herpes simplex virus, human immunodeficiency virus, retrovirus (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus) and other recombinant vectors. Other vectors contemplated for eukaryotic target cells include, but are not limited to, the vectors pXT1, pSG5, pSVK3, pBPV, pMSG, and pSVLSV40 (Pharmacia). Other vectors can be used so long as they are compatible with the host cell.

In some examples, a vector can comprise one or more transcription and/or translation control elements. Depending on the host/vector system utilized, any of a number of suitable transcription and translation control elements, including constitutive and inducible promoters, transcription enhancer elements, transcription terminators, etc. can be used in the expression vector. The vector can be a self-inactivating vector that either inactivates the viral sequences or the components of the CRISPR machinery or other elements.

Non-limiting examples of suitable eukaryotic promoters (i.e., promoters functional in a eukaryotic cell) include those from cytomegalovirus (CMV) immediate early, herpes simplex virus (HSV) thymidine kinase, early and late SV40, long terminal repeats (LTRs) from retrovirus, human elongation factor-1α promoter (EF1α), chicken beta-actin promoter (CBA), ubiquitin C promoter (UBC), a hybrid construct comprising the cytomegalovirus enhancer fused to the chicken beta-actin promoter, a hybrid construct comprising the cytomegalovirus enhancer fused to the promoter, the first exon, and the first intron of chicken beta-actin gene (CAG or CAGGS), murine stem cell virus promoter (MSCV), phosphoglycerate kinase-1 locus promoter (PGK), and mouse metallothionein-I promoter.

A promoter can be an inducible promoter (e.g., a heat shock promoter, tetracycline-regulated promoter, steroid-regulated promoter, metal-regulated promoter, estrogen receptor-regulated promoter, etc.). The promoter can be a constitutive promoter (e.g., CMV promoter, UBC promoter, CAG or CAGGS promoter). In some cases, the promoter can be a spatially restricted and/or temporally restricted promoter (e.g., a tissue specific promoter, a cell type specific promoter, etc.).

Introduction of the complexes, polypeptides, and nucleic acids of the disclosure into cells can occur by viral or bacteriophage infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

### Cell Types

In various aspects of the disclosure, the gene edited cells comprise a terminally differentiated or lineage restricted cell. In various aspects, the gene edited cell comprise a pancreatic cell or progenitor cells. In some embodiments, the lineage-restricted progenitor cells are pancreatic endoderm progenitors, pancreatic endocrine progenitors, mesenchymal progenitor cells, muscle progenitor cells, blast cells, hematopoietic progenitor cells, or neural progenitor cells. In some embodiments, the terminally differentiated cells are somatic cells. In some embodiments, the terminally differentiated cells are endocrine secretory cells such as pancreatic beta cells, epithelial cells, endodermal cells, macrophages, hepatocytes, adipocytes, kidney cells, blood cells, or immune system cells. In some embodiments, the terminally differentiated cells are beta cells.

A “differentiated cell” is a cell that has progressed further down the developmental pathway than the cell to which it is being compared. Thus, stem cells can differentiate into lineage-restricted precursor cells (such as a pancreatic endoderm cell), which in turn can differentiate into other types of precursor cells further down the pathway (such as a myocyte precursor), and then to an end-stage (e.g., terminally) differentiated cell, such as a myocyte or pancreatic beta cell, which plays a characteristic role in a certain tissue type and may or may not retain the capacity to proliferate further. In some embodiments, the differentiated cell may be a pancreatic beta cell.

Another step of the methods of the present disclosure may comprise differentiating cells into differentiated cells. The differentiating step may be performed according to any method known in the art. For example, human iPSCs are differentiated into definitive endoderm using various treatments, including activin and B27 supplement (Life Technologies). The definitive endoderm is further differentiated into hepatocyte, the treatment includes: FGF4, HGF, BMP2, BMP4, Oncostatin M, Dexamethasone, etc. (Duan et al, Stem Cells, 2010; 28:674-686; Ma et al, Stem Cells Translational Medicine, 2013; 2:409-419). In another embodiment, the differentiating step may be performed according to Sawitza et al, Sci Rep. 2015; 5:13320. A differentiated cell may be any somatic cell of a mammal, e.g., a human. In some embodiments, a somatic cell may be an exocrine secretory epithelial cells (e.g., salivary gland mucous cell, prostate gland cell), a hormone-secreting cell (e.g., anterior pituitary cell, gut tract cell, pancreatic islet), a keratinizing epithelial cell (e.g., epidermal keratinocyte), a wet stratified barrier epithelial cell, a sensory transducer cell (e.g., a photoreceptor), an autonomic neuron cell, a sense organ and peripheral neuron supporting cell (e.g., Schwann cell), a central nervous system neuron, a glial cell (e.g., astrocyte, oligodendrocyte), a lens cell, an adipocyte, a kidney cell, a barrier function cell (e.g., a duct cell), an extracellular matrix cell, a contractile cell (e.g., skeletal muscle cell, heart muscle cell, smooth muscle cell), a blood cell (e.g., erythrocyte), an immune system cell (e.g., megakaryocyte, microglial cell, neutrophil, Mast cell, a T-cell, a B-cell, a Natural Killer cell), a germ cell (e.g., spermatid), a nurse cell, or an interstitial cell.

In general, populations of the genetically modified cells disclosed herein maintain expression of the inserted one or more nucleotide sequences over time. For example, at least about 50%, at least about 55%, at least about 60%, at least about 65%, at least about 70%, at least about 75%, at least about 80%, at least about 85%, at least about 90%, at least about 95%, or at least about 99% of the universal donor cells express the inserted one or more tolerogenic factors and/or survival factors. Moreover, populations of lineage-restricted or fully differentiated cells derived from the universal donor cells disclosed herein maintain expression of the inserted one or more nucleotide sequences over time. For example, at least about 50%, at least about 55%, at least about 60%, at least about 65%, at least about 70%, at least about 75%, at least about 80%, at least about 85%, at least about 90%, at least about 95%, or at least about 99% of the lineage-restricted or fully differentiated cells express the one or more tolerogenic factors and/or survival factors.

### Formulation and Delivery for Gene Editing

Guide RNAs, polynucleotides, e.g., polynucleotides that encode a tolerogenic factor and/or survival factor, or polynucleotides that encode an endonuclease, and endonucleases as described herein may be formulated and delivered to cells in any manner known in the art.

Guide RNAs and/or polynucleotides may be formulated with pharmaceutically acceptable excipients such as carriers, solvents, stabilizers, adjuvants, diluents, etc., depending upon the particular mode of administration and dosage form. Guide RNAs and/or polynucleotides compositions can be formulated to achieve a physiologically compatible pH, and range from a pH of about 3 to a pH of about 11, about pH 3 to about pH 7, depending on the formulation and route of administration. In some cases, the pH can be adjusted to a range from about pH 5.0 to about pH 8. In some cases, the compositions can comprise a therapeutically effective amount of at least one compound as described herein, together with one or more pharmaceutically acceptable excipients. Optionally, the compositions can comprise a combination of the compounds described herein, or can include a second active ingredient useful in the treatment or prevention of bacterial growth (for example and without limitation, anti-bacterial or anti-microbial agents), or can include a combination of reagents of the present disclosure.

Suitable excipients include, for example, carrier molecules that include large, slowly metabolized macromolecules such as proteins, polysaccharides, polylactic acids, polyglycolic acids, polymeric amino acids, amino acid copolymers, and inactive virus particles. Other exemplary excipients can include antioxidants (for example and without limitation, ascorbic acid), chelating agents (for example and without limitation, EDTA), carbohydrates (for example and without limitation, dextrin, hydroxyalkylcellulose, and hydroxyalkylmethylcellulose), stearic acid, liquids (for example and without limitation, oils, water, saline, glycerol and ethanol), wetting or emulsifying agents, pH buffering substances, and the like.

Guide RNA polynucleotides (provided as RNA or DNA that can be transcribed into RNA) and/or endonuclease polynucleotide(s) (RNA or DNA) can be delivered by viral or non-viral delivery vehicles known in the art. Alternatively, endonuclease polypeptide(s) can be delivered by viral or non-viral delivery vehicles known in the art, such as electroporation or lipid nanoparticles. In further alternative aspects, the DNA endonuclease can be delivered as one or more polypeptides, either alone or pre-complexed with one or more guide RNAs, or one or more crRNA together with a tracrRNA.

Polynucleotides can be delivered by non-viral delivery vehicles including, but not limited to, nanoparticles, liposomes, ribonucleoproteins, positively charged peptides, small molecule RNA-conjugates, aptamer-RNA chimeras, and RNA-fusion protein complexes. Some exemplary non-viral delivery vehicles are described in Peer and Lieberman, Gene Therapy, 2011, 18:1127-1133 (which focuses on non-viral delivery vehicles for siRNA that are also useful for delivery of other polynucleotides). For polynucleotides of the disclosure, the formulation may be selected from any of those taught, for example, in International Application PCT/US2012/069610.

Polynucleotides, such as guide RNA, sgRNA, and mRNA encoding an endonuclease, may be delivered to a cell or a subject by a lipid nanoparticle (LNP). An LNP refers to any particle having a diameter of less than 1000 nm, 500 nm, 250 nm, 200 nm, 150 nm, 100 nm, 75 nm, 50 nm, or 25 nm. Alternatively, a nanoparticle may range in size from 1-1000 nm, 1-500 nm, 1-250 nm, 25-200 nm, 25-100 nm, 35-75 nm, or 25-60 nm.

LNPs may be made from cationic, anionic, or neutral lipids. Neutral lipids, such as the fusogenic phospholipid DOPE or the membrane component cholesterol, may be included in LNPs as “helper lipids” to enhance transfection activity and nanoparticle stability. Limitations of cationic lipids include low efficacy owing to poor stability and rapid clearance, as well as the generation of inflammatory or anti-inflammatory responses. LNPs may also be comprised of hydrophobic lipids, hydrophilic lipids, or both hydrophobic and hydrophilic lipids.

Any lipid or combination of lipids that are known in the art can be used to produce a LNP. Examples of lipids used to produce LNPs are: DOTMA, DOSPA, DOTAP, DMRIE, DC-cholesterol, DOTAP-cholesterol, GAP-DMORIE-DPyPE, and GL67A-DOPE-DMPE-polyethylene glycol (PEG). Examples of cationic lipids are: 98N12-5, C12-200, DLin-KC2-DMA (KC2), DLin-MC3-DMA (MC3), XTC, MD1, and 7C1. Examples of neutral lipids are: DPSC, DPPC, POPC, DOPE, and SM. Examples of PEG-modified lipids are: PEG-DMG, PEG-CerC14, and PEG-CerC20. The lipids can be combined in any number of molar ratios to produce a LNP. In addition, the polynucleotide(s) can be combined with lipid(s) in a wide range of molar ratios to produce a LNP.

A recombinant adeno-associated virus (rAAV) vector can be used for delivery. Techniques to produce rAAV particles, in which an AAV genome to be packaged that includes the polynucleotide to be delivered, rep and cap genes, and helper virus functions are provided to a cell are standard in the art. Production of rAAV typically requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived from and may be from a different AAV serotype than the rAAV genome ITRs, including, but not limited to, AAV serotypes described herein. Production of pseudotyped rAAV is disclosed in, for example, international patent application publication number WO 01/83692.

### Immune Attenuating Drugs

Methods provided herein comprise administering at least one immune attenuating drug to a subject. Further, combination products provided herein also comprise at least one immune attenuating drug. In some embodiments, inflammation associated with the foreign body response of the perforated cell delivery device loaded with the genetically modified cell, or population thereof in the mammalian subject is reduced compared to administering a comparative perforated cell delivery device loaded with the genetically modified cell, or population thereof, without the at least one immune attenuating drug. In some embodiments, reducing the foreign body response comprises reducing cell trafficking and/or inflammation external to the device in a host tissue, facilitating integration of the device into the host tissue, and/or improving or accelerating healing of the host tissue around the device.

As used herein, “immune attenuating drugs” refer to a drug agent or biologic that modulates an immune response when administered to a subject. In certain aspects, the at least one immune attenuating drug comprises a JAK inhibitor (e.g., a JAK 1 or 2 inhibitor). In some aspects, the at least one immune attenuating drug comprises an IFNγ inhibitor and/or an anti-fibrotic. In other aspects, the at least one immune attenuating drug may comprise a TNFα or TNFβ blocker. In other aspects, the at least one immune attenuating drug may comprise an ILIR blocker. In some embodiments the at least one immune attenuating drug comprises or is a calcineurin inhibitor. In some embodiments, the at least one immune attenuating drug comprises or is an anti-thymocyte globulin (ATG).

In various aspects, the at least one immune attenuating drug may comprise more than one drug, agent or biologic, that together modulate an immune response in a subject. For example, the at least one immune attenuating drug may comprise at least one IFNγ blocker, a JAK inhibitor and a TNFα/β blocker. For example, the at least one immune attenuating drug may comprise at least one JAK inhibitor and at least one TNFα/β blocker. In other examples, the at least one immune attenuating drug may comprise at least one JAK inhibitor and at least one IFNγ blocker. In still other examples, the at least one immune attenuating drug may comprise at least one TNFα/β blocker and at least one IFNγ blocker. In some embodiments, the at least one immune attenuating drug comprises a calcineurin inhibitor and an anti-thymocyte globulin.

In some aspects the at least one immune attenuating drug comprises a JAK inhibitor (e.g., a JAK 1 or 2 inhibitor). Janus Kinases (JAKs) are a subgroup of non-receptor tyrosine kinases that transduce signals specifically from cytokine receptors, and whose enzymatic activity is essential for the biological activity of cytokines. In some aspects, the at least one immune attenuating drug may comprise a JAK 1 inhibitor (e.g., but not limited to, Tofacitinib, GLPG-0634, Ruxolitinib, Baricitinib, CYT387, AZD1480, and/or GSK2586184). In still other aspects, the at least one immune attenuating drug may comprise a JAK 2 inhibitor (e.g., but not limited to, Tofacitinib, Ruxolitinib, Baricitinib, TG101348, AC-430, CEP-337709, Lestaurtinib, Pacritinib, CYT387, BMS-911543, AZD1480, and/or SB1518).

In some aspects, the at least one immune attenuating drug comprises a TNFα or TNFβ blocker. TNF receptors are involved in the immune response to invading pathogens. In some aspects, the at least one immune attenuating drug can comprise a TNFα or TNFβ blocker (e.g., but not limited to infliximab (REMICADE), etanercept (ENBREL), adalimumab (HUMIRA), certolizumab pegol (CIMZIA), and golimumab (SIMPONI)).

In some aspects, the at least one immune attenuating drug comprises ruxolitinib (JAKAVI), etenercept (ENBREL), anakinra (KINERET) or a combination of any thereof. In some aspects, the at least one immune attenuating drug comprises ruxolitinib. In some aspects, the at least one immune attenuating drug comprises etenercept. In some aspects, the at least one immune attenuating drug comprises anakinra. In still other aspects the at least one immune attenuating drug comprises ruxolitinib and anakinra. In other aspects, the at least one immune attenuating drug comprises ruxolitinib and eternercept. In other aspects, the at least one immune attenuating drug comprises etenercept and anakinra. In other aspects, the at least one immune attenuating drug comprises ruxolitinib, anakinra and etenercept.

In some embodiments, the at least one immune attenuating drug comprises a lymphocyte-depleting agent such as an anti-lymphocyte antibody, e.g., anti-T cell antibodies, e.g., anti-thymocyte globulin (ATG), such as, e.g., Thymoglobulin®, Atgam™, Fresenius™, and Tecelac™ ATG is a polyclonal antibody directed against thymocytes. Currently marketed ATG products are produced by injecting thymocytes from one species (e.g., human) into another species (e.g., rabbit or horse). ATG binds to cell surface proteins such as lymphocyte surface antigens CD2, CD3, CD4, CD8, CD11a, CD18, CD25, HLA DR, and HLA class I. Without being bound by any particular theory, ATG is believed to induce immunosuppression primarily as a result of T cell depletion and has been used for pretreating transplant patients to reduce the risk of rejection in the context of organ transplantation.

In some embodiments, the at least one immune attenuating drug comprises a calcineurin inhibitor. Calcineurin (CaN) is a calcium and calmodulin dependent serine/threonine protein phosphatase (also known as protein phosphatase 3, and calcium-dependent serine-threonine phosphatase). CaN activates the T cells of the immune system and can be blocked by drugs. Calcineurin activates nuclear factor of activated T cell cytoplasmic (NFATc), a transcription factor, by dephosphorylating it. The activated NFATc is then translocated into the nucleus, where it upregulates the expression of interleukin 2 (IL-2), which, in turn, stimulates the growth and differentiation of the T cell response. Calcineurin is the target of a class of drugs called calcineurin inhibitors, which include ciclosporin, voclosporin, pimecrolimus and tacrolimus.

The at least one immune attenuating drug can comprise the calcineurin inhibitor, the anti-thymocyte globulin, or both. The calcineurin inhibitor can be tacrolimus or cyclosporine A. The at least one immune attenuating drug can comprise tacrolimus, the anti-thymocyte globulin, or both. In some embodiments, the at least one immune attenuating drug comprises or is a calcineurin inhibitor. In some embodiments, the at least one immune attenuating drug comprises or is tacrolimus. In some embodiments, the at least one immune attenuating drug comprises or is an anti-thymocyte globulin. The tacrolimus can be Prograf®, Astagraf XL®, Envarsus XR®, Hecoria®, or any combination thereof. The anti-thymocyte globulin can be Thymoglobulin®, Atgam™, Fresenius™, Tecelac™, or any combination thereof.

Dosing and administration regimens for many immune attenuating drugs are known in the art. Each of the at least one immune attenuating drug can be administered by oral administration, intravenous administration, subcutaneous administration, or any combination thereof. Each of the at least one immune attenuating drug can be administered as a separate composition. For example, when two immune attenuating drugs are administered, one can be formulated for oral administration and the other for intravenous administration. In some embodiments, the at least one immune attenuating drug comprises tacrolimus and ATG. In some embodiments, the tacrolimus is administered orally and the ATG is administered intravenously. Each of the least one immune attenuating drug can be administered at the same time of day (e.g., concurrently), or at different times (e.g., asynchronously).

In some embodiments, the at least one immune attenuating drug is administered at a dose of about 0.5 mg to about 100 mg (e.g., about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 mg, a number or a range between any two of these values). The calcineurin inhibitor can be administered at a dose of about 0.5 mg to about 100 mg (e.g., about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 mg, a number or a range between any two of these values). The calcineurin inhibitor can be administered at a dose of about, at least, or at least about, 0.2 mg, 1 mg, 5 mg, 25 mg, or 100 mg. The calcineurin inhibitor can be administered at a dose of about 0.5 mg, 1 mg, or 5 mg. The calcineurin inhibitor can be administered at a dose of about 0.2 mg or about 1 mg. The calcineurin inhibitor can be administered orally. The calcineurin inhibitor can be administered intravenously. The calcineurin inhibitor can be administered at a dose of about 5 mg/ml. About 1 mL of the calcineurin inhibitor of about 5 mg/mL can be administered intravenously.

The anti-thymocyte globulin can be administered to the subject at a dose of about, at least, or at least about 1 mg/kg to about 2 mg/kg (e.g., about 1, 1.1, 1.2, 1.3, 1.4, 1.5, 1.6, 1.7, 1.8, 1.9, 2 mg/kg, or a number or a range between any two of the values). The anti-thymocyte globulin can be administered intravenously. The anti-thymocyte globulin can be administered to the subject at a dose of about 1.5 mg/kg.

In various aspects, the methods provided herein comprise administering the at least one immune attenuating drug pre-, peri-, or post-surgical implant of a suitable perforated cell delivery device (discussed further below). In various aspects, a short-term preparatory drug regimen (e.g., a cycle of treatment) including immune-attenuation medications may be used during a pre-, peri-, and post-surgical implant period to modulate the inflammation associated with the FBR and enhance the healing and integration of the perforated cell delivery device (PD) with the surrounding tissue. In some aspects, the preparatory regimen of immune attenuating medications include a calcineurin inhibitor (e.g., tacrolimus) and anti-thymocyte globulin.

Each of the at least one immune attenuating drug can be administered to the subject in a cycle of at least one week. Each of the at least one immune attenuating drug can be administered to the subject in a cycle of at least one week, two weeks, three weeks, four weeks, five weeks, six weeks, seven weeks, eight weeks, nine weeks, ten weeks, eleven weeks, twelve weeks, or more. Each of the at least one immune attenuating drug can be administered to the subject once or twice daily in the cycle. In some embodiments, for example, when the at least one immune attenuating drug comprises two immune attenuating drugs (e.g., tacrolimus and ATG), one drug can be administered once daily, and the other drug can be administered twice daily. In some embodiments, when the at least one immune attenuating drug comprises two immune attenuating drugs, both drugs can be administered twice daily. In some embodiments, when the at least one immune attenuating drug comprises two immune attenuating drugs, both drugs can be administered once daily.

In some embodiments, the cycle begins prior to the implanting of the perforated cell delivery device. In some embodiments, the cycle begins about, at least, or at least about 24 hours, about 12 hours, about 6 hours, about 5 hours, about 4 hours, about 3 hours, about 2 hours, about 1 hour or less, prior to the implanting of the perforated cell delivery device. In some embodiments, the cycle beings about 1 month, 4 weeks, 3 weeks, 2 weeks, 7 days, 6 days, 5 days, 4 days, 3 days, or 2 days prior to the implanting of the perforated delivery device.

In some embodiments, the cycle begins after the implanting of the perforated cell delivery device. In some embodiments, the cycle begins about 0.5 hour, about 1 hour, about 2 hours, about 3 hours, about 4 hours, about 5 hours, about 6 hours, about 12 hours, about 24 hours or more after the implanting of the perforated cell delivery device. In some embodiments, the cycle begins about 1 day, 2 days, 3 days, 4 days, 5 days, 6 days, 1 week, 2 weeks, 3 weeks, 4 weeks, one month or more after the implanting of the perforated delivery device. In some embodiments, the cycle begins concurrently with the implanting of the perforated cell delivery device. The method can comprise administering at least one additional cycle of treatment to the subject. A skilled practitioner, such as a medical doctor, can administer at least one additional cycle of treatment to the subject as needed to, e.g., reduce foreign body response in the subject.

### Cell Delivery Devices

Methods provided are directed to administering gene edited cells to a subject using a perforated cell delivery device. Also provided are combination products comprising a perforated cell delivery device, a gene edited cell, or population thereof, and at least one immune attenuating drug. In various aspects, the administering comprises implanting a device, such as a perforated cell delivery device, comprising a population of genetically modified cells into the subject. The population of genetically modified cells can comprise about 1×10⁶ to about 9.5×10⁶ cells (e.g., about 1×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, 2×10⁶, 2.1×10⁶, 2.2×10⁶, 2.3×10⁶, 2.4×10⁶, 2.5×10⁶, 2.6×10⁶, 2.7×10⁶, 2.8×10⁶, 2.9×10⁶, 3×10⁶, 3.1×10⁶, 3.2×10⁶, 3.3×10⁶, 3.4×10⁶, 3.5×10⁶, 3.6×10⁶, 3.7×10⁶, 3.8×10⁶, 3.9×10⁶, 4×10⁶, 4.1×10⁶, 4.2×10⁶, 4.3×10⁶, 4.4×10⁶, 4.5×10⁶, 4.6×10⁶, 4.7×10⁶, 4.8×10⁶, 4.9×10⁶, 5×10⁶, 5.1×10⁶, 5.2×10⁶, 5.3×10⁶, 5.4×10⁶, 5.5×10⁶, 5.6×10⁶, 5.7×10⁶, 5.8×10⁶, 5.9×10⁶, 6×10⁶, 6.1×10⁶, 6.2×10⁶, 6.3×10⁶, 6.4×10⁶, 6.5×10⁶, 6.6×10⁶, 6.7×10⁶, 6.8×10⁶, 6.9×10⁶, 7×10⁶, 7.1×10⁶, 7.2×10⁶, 7.3×10⁶, 7.4×10⁶, 7.5×10⁶, 7.6×10⁶, 7.7×10⁶, 7.8×10⁶, 7.9×10⁶, 8×10⁶, 8.1×10⁶, 8.2×10⁶, 8.3×10⁶, 8.4×10⁶, 8.5×10⁶, 8.6×10⁶, 8.7×10⁶, 8.8×10⁶, 8.9×10⁶, 9×10⁶, 9.1×10⁶, 9.2×10⁶, 9.3×10⁶, 9.4×10⁶, 9.5×10⁶ cells, or a number or a range between any two of these values). Suitable devices are known in the art and described in more detail below.

In some embodiments, the method comprises implanting more than one perforated cell delivery device into the mammalian subject. In some embodiments, the method comprises implanting two, three, four, five, or more perforated cell delivery devices into the mammalian subject. About 1.0×10⁷ to about 2.0×10⁷ genetically modified cells per kilogram can be administered to the subject (e.g., about 1×10⁷, 1.1×10⁷, 1.2×10⁷, 1.3×10⁷, 1.4×10⁷, 1.5×10⁷, 1.6×10⁷, 1.7×10⁷, 1.8×10⁷, 1.9×10⁷, 2×10⁷ cells per kilogram, or a number or a range between any two of these values).

In some embodiments, hypoimmunogenic cells are implanted in a perforated cell delivery device which provides direct cell-to-cell contact between host vasculature and the encapsulated cells. In some embodiments, perforated means a hole or pore in the device. In some embodiments not all the layers of the device are perforated. For example, see PCT Application No. PCT/US2016/0061442 which is herein incorporated by reference in its entirety which discusses perforated cell delivery devices with perforations in just one layer, for example, the cell-excluding membrane; or, in just the cell-excluding membrane and the non-woven fabric layer. In some embodiments, hypoimmunogenic cells are encapsulated in a perforated device surrounded by a non-woven fabric. In these embodiments, the non-woven fabric is on the outside of the cell delivery device. Rather than affecting implanted cells, the non-woven fabric enhances host vascularization surrounding the cell housing. See, e.g., PCT/US2016/061442 and U.S. Pat. No. 8,278,106 (both of which are herein incorporated by reference in their entirety) which describe perforated devices and device polymers.

In some embodiments, the holes/perforations are smaller than cell aggregates contained in the device, such as the hPSC-derived aggregates, e.g. definitive endoderm lineage cell aggregates, contained therein. In some embodiments, a perforated cell delivery device implanted into a mammalian subject (e.g., a human) contains perforations in just the cell-excluding membrane (the other layers of the device are not perforated) and wherein the holes are separated by about 2 mm or more and wherein the hole diameter is less than about 100 microns is provided.

### Perforated Cell Delivery Devices

To promote vascularization shortly after implant, cells are implanted in a perforated cell delivery device which provides direct cell-to-cell contact between host vasculature and the encapsulated cells. In some embodiments, not all the layers of the device are perforated. For example, in some embodiments, a perforated cell delivery device is provided with perforations in just one layer, for example, the cell-excluding membrane; or, in just the cell-excluding membrane and the non-woven fabric layer. This helps retain the implanted cells/tissue while at the same time allowing exchanges with the host such as ingress of the vasculature, macrophages and the like.

By laser drilling the perforations, the perforation size, number and location can be selected. The perforations are of sufficient size to allow host vascular tissue (such as capillaries) and stromal cells that support pancreatic cell types to enter the device lumen. In some embodiments, the perforations are sized such that host macrophages and other phagocytes can also enter the device and remove necrotic debris from the perforated device lumen. In some embodiments, the perforations are also sized to allow therapeutic agents such as insulin produced by the graft to exit the cell delivery device. Perforations allowing for vascular structures to grow into the device lumen help anchor the device to the host and inhibit movement of the device. In some embodiments, the perforations are also sized based on cell aggregate diameter to maximize cell retention.

In some embodiments, the device includes a cell housing made of a biocompatible material adapted to be implanted in a host, and to substantially contain therapeutic agents which can be immunologically compatible or incompatible with the host (e.g., the genetically modified cell or population thereof as provided herein), the chamber having a wall comprising cell-excluding membrane and optionally a mesh layer or layers and film weld, said wall having holes traversing just the cell-excluding membrane; where the holes have an inner diameter at the narrowest point large enough to permit a host capillary to traverse the thickness of the wall, and where said holes are numerous enough to permit said host capillary to support the viability of the therapeutic agents which may be contained therein.

In some embodiments, a perforated delivery device is provided wherein one or more layers of the delivery device are perforated. In some embodiments, a perforated delivery device is provided wherein one or more layers of the delivery device are not perforated. In some embodiments, only the cell-excluding membrane is perforated. In some embodiments, a cell delivery device comprises holes which do not traverse each wall of the device. In some embodiments, perforations in the cell delivery device consist of holes which do not traverse each wall of the device but host vasculature growth into the inner lumen of the cell delivery device still occurs. In some embodiments, a cell delivery device that does not comprise a non-woven fabric is disclosed. In some embodiments, a cell delivery device that does not comprise a non-woven fabric but the cell-excluding membrane is perforated is disclosed. In such embodiments, the hole diameter in the cell-excluding membrane is used to retain the cells, e.g., the holes in the device are smaller than the cell aggregates contained therein.

In some embodiments, the cells in the perforated delivery device comprise PDX1/NKX6.1 co-positive pancreatic progenitor cells. In one embodiment, the cells in the perforated delivery devices comprise immature beta cells expressing insulin (INS) and NKX6.1 or immature beta cells expressing INS (e.g., insulin), NKX6.1 and MAFB. In some embodiments, the cells in the perforated delivery device comprise mature beta cells expressing INS and MAFA or INS, NKX6.1 and MAFA. In some embodiments, the cells in the perforated delivery device comprise pancreatic endocrine cells. In some embodiments, the cells in the perforated delivery device comprise pancreatic insulin secreting cells. In some embodiments, cells in the perforated delivery devices comprise pancreatic beta or insulin cells capable of secreting insulin in response to blood glucose levels. In some embodiments, the cells are genetically modified cells, and comprise one or more of the genetic modifications described herein.

### Perforated Devices Surrounded by a Non-Woven Fabric

In some embodiments, the non-woven fabric is on the outside of the cell delivery device. Rather than affecting implanted cells, the non-woven fabric enhances host vascularization surrounding the cell housing. In some embodiments, a cell delivery device comprising a non-woven fabric is disclosed. In some embodiments, a cell delivery device comprising a non-woven polyester fabric (NWPF) is disclosed. Polypropylene, polyethylene, nylon, polyurethane, polyamide are some examples of a non-woven polyester fabric that can be used. In some embodiments, the cell-excluding membrane is surrounded (or coated) with a non-woven fabric, e.g., the non-woven fabric is external to the cell-excluding membrane. Stated another way, the non-woven fabric faces the host not the implanted cells. In some embodiments, the non-woven fabric forms a jacket around the cell excluding membrane. In some embodiments, only the cell-excluding membrane is perforated, and the other layers of the device including the non-woven fabric are not perforated. In some embodiments, just the cell-excluding membrane and the non-woven fabric are perforated and the other layers of the device are not perforated.

In some embodiments, the holes/perforations are smaller than cell aggregates contained in the device, such as the hPSC-derived aggregates, e.g. definitive endoderm lineage cell aggregates, e.g., the genetically modified cell or population thereof, contained therein. In some embodiments, the holes are smaller than the genetically modified cell aggregates (e.g., populations) contained therein. In some embodiments, the holes are smaller than the cell aggregates contained therein. In some embodiments, the holes are smaller than the genetically modified cell aggregates contained therein. In some embodiments, the holes are smaller than the mature beta cell aggregates contained therein that mature from genetically modified PECs. In some embodiments, the hole diameter is small enough to retain the cells but large enough to ensure that the desired therapeutic effect is achieved. For example, in the case of a diabetic patient the hole diameter is determined by the ability of the implanted cells to mature and/or produce insulin in response to blood glucose levels.

In some embodiments, a perforated cell delivery device is implanted into a mammalian subject (e.g., a human). In some embodiments, a perforated cell delivery device implanted into a mammalian subject contains perforations in just the cell-excluding membrane and the non-woven polyester fabric (the other layers of the device are not perforated) and wherein the holes are separated by about 2 mm (measuring center to center from the holes) or more and wherein the hole diameter is less than about 100 microns is provided. In some embodiments, a perforated cell delivery device implanted into a mammalian subject contains perforations in just the cell-excluding membrane and the non-woven polyester fabric (the other layers of the device are not perforated) and wherein the holes are separated by about 2 mm or more. In some embodiments, a perforated cell delivery device implanted into a mammalian subject contains perforations in just the cell-excluding membrane and the non-woven polyester fabric (the other layers of the device are not perforated) and wherein the hole diameter is less than about 100 microns is provided. In some embodiments, a perforated cell delivery device implanted into a mammalian subject contains perforations in just the cell-excluding membrane (the other layers of the device are not perforated) and wherein the holes are separated by about 2 mm or more and wherein the hole diameter is less than about 100 microns is provided.

In some embodiments, a cell delivery device comprises a perforated cell-excluding membrane and PDX1-positive pancreatic endoderm cells (PECs), which can be implanted into a human subject wherein the PDX1-positive pancreatic endoderm cells mature in vivo into insulin-producing cells. In some embodiments, the PDX1-positive PECs comprise one or more genetic modifications disclosed herein (e.g., are genetically modified). In some embodiments, a cell delivery device comprises just a perforated cell-excluding membrane and perforated NWF layer and PDX1-positive genetically modified pancreatic endoderm cells, wherein the cell delivery device can be implanted into a human patient wherein the PDX-positive genetically modified pancreatic endoderm cells mature in vivo to insulin-producing cells. In some embodiments, the PECs are NKX6.1-positive, PDX1-positive, INS-positive, CHGA-negative, or any combination thereof.

### Laminated Devices

In some embodiments, the cell-excluding membrane is laminated to the non-woven fabric. In some embodiments, the cell-excluding membrane is laminated to the non-woven fabric. When the cell-excluding membrane is laminated to a non-woven fabric the cell-excluding membrane remains flat. Without lamination, the cell-excluding membrane can deform out of plane which creates dams which may lead to an uneven distribution of cells. An uneven distribution of cells can lead to necrotic regions, cell death and otherwise may reduce efficacy. Control of cell distribution within the chamber or lumen of the cell delivery device is also referred to as the spatial location of cells within the cell delivery device. In some embodiments, a method for controlling the distribution of cells (cell location) within a cell delivery device is provided comprising laminating the cell-excluding membrane to a non-woven fabric. In some embodiments, a method for controlling the distribution of cells (cell location) within a cell delivery device is provided comprising laminating the cell-excluding membrane to a NWF. By achieving an even distribution of cells inside the lumen of the cell delivery device fewer cells need to be implanted. The use of fewer cells results in less cellular debris. Another benefit is the enhanced diffusion of nutrients to the cells because the cells are evenly distributed and in closer contact with the membrane. Enhanced diffusion of nutrients to the cells leads to improved cell survival.

Complete filling of the lumen can be achieved consistently with devices incorporating laminated membranes resulting in maximizing therapeutic efficacy of the implanted cells. Longer and larger lumens can be filled with devices incorporating laminated membranes.

### Perforated and Laminated Devices

Perforations can also be referred to as holes, pores, openings, punctures, apertures or channels. It is to be understood that the foregoing devices are non-limiting disclosures and that other devices in keeping with the embodiments described herein are embodied by this disclosure. This disclosure envisions combinations of the above-described devices. For example, in some embodiments, a cell-excluding membrane, and non-woven fabric are laminated together and then perforated. In some embodiments, a perforated cell-excluding membrane and a non-perforated, non-woven fabric are laminated together.

In some embodiments, the perforations are of circular shape or oval shape or elliptical shape. It should be noted that the perforations can have other shapes such as rectangular or hexagonal or polygonal, or slits. In some embodiments, the perforations have a uniform shape. In some embodiments, the perforations do not have a uniform shape. In some embodiments, the perforations are uniformly distributed on the cell excluding membrane. In some embodiments, the perforations are variably spaced on the cell excluding membrane, for example, they may be clustered at the center of the device or at the ends of the device. In some embodiments, the plurality of perforations is spaced in a series of rows and columns forming a grid arrangement or concentric circles or any other geometric configuration or combinations of such configurations. In some embodiments, the plurality of perforations is randomly distributed. In one embodiment, perforations are not on each cell-excluding membrane but only on one side of the device.

In some embodiments, a cell delivery device comprises layers wherein only the cell-excluding membrane is perforated with holes. In some embodiments, a cell delivery device comprises a film ring, a mesh and cell-excluding membrane wherein only the cell-excluding membrane is perforated with holes. In some embodiments, a cell delivery device comprises a film ring, a mesh, non-woven fabric and cell-excluding membrane wherein only the cell-excluding membrane and non-woven fabric layer are perforated with holes. In some embodiments, a cell delivery device comprises non-woven fabric and cell-excluding layers wherein only the cell-excluding membrane and non-woven fabric layer are perforated with holes. In some embodiments, a cell delivery device comprises a non-woven fabric external to the cell-excluding membrane wherein only the cell-excluding membrane and non-woven fabric layer are perforated with holes. In some embodiments, a cell delivery device comprises non-woven fabric and cell-excluding layers laminated to each other wherein only the cell-excluding membrane and non-woven fabric layer are perforated with holes. In some embodiments, a cell delivery device comprises a non-woven fabric external to the cell-excluding layer and laminated to the cell-excluding membrane wherein only the cell-excluding membrane and non-woven fabric layer are perforated with holes. In some embodiments, a cell delivery device comprises layers wherein only the cell-excluding membrane and non-woven fabric layer are perforated with holes wherein the holes are made with a laser.

### Diameter of the Perforation

The aperture of the perforations can enable the cell-excluding membrane to retain the encapsulated elements, while at the same time allowing exchanges with the host such as ingress of vasculature, macrophages and other phagocytes that can remove necrotic debris from the perforated device lumen and stromal cells that support pancreatic cell types. In some embodiments, the perforations are less than about 100 μm in diameter to allow capillary ingrowth. Pancreatic progenitor cell aggregates can average approximately 180 μm in diameter with quartile range approximately 100-200 μm, therefore hole diameters of about 100 μm or less can provide substantial retention of the cell product, while still achieving the other benefits described above and, thus, facilitate both delivery and retrieval of the cells as well as allow capillary ingrowth. Hence, the cells are exposed to the host tissue, e.g., host blood vessels, but due to their larger size, the risk of cell escape is low. In some embodiments, the holes have an inner diameter large enough to allow the ingrowth and egress of host capillaries and large enough to allow the hormone produced by the therapeutic agent to exit the device lumen/chamber.

The hole size (diameter) may be varied depending on the cell function. For example, if complete cell containment is not necessary, then there is less restriction with regard to hole diameter and density. The holes in a particular device may have the same diameter, or may have different diameters in different parts of the device. For example, if the majority of the encapsulated cells, cell aggregates, organoids, clusters, clumps, and tissues tend to be located approximately in the center of the device, then more holes may be necessary for cell survival in that region of the device as compared to the proximal and distal ends of the device which may have fewer and/or smaller holes. As such, there is flexibility in the size, density and distribution of perforations, so long as host-implant cell-to-cell vascularization is established shortly after transplantation.

In some embodiments, pancreatic progenitor cell aggregates are larger in size as compared to the average hole diameter of the perforation in a device. In some embodiments, the cell delivery device is perforated with holes less than about 300 microns, less than about 200 microns, less than about 150 microns, less than about 100 microns, or less than about 75 microns, or less than about 60 microns, or less than about 50 microns in diameter. In some embodiments, the cell delivery device is perforated with holes between about 300-50 microns or about 200-50 microns, or about 200-75 microns or about 70-80 microns in diameter. In some embodiments, the hole diameter is greater than about 200 microns. In some embodiments, the hole diameter is about 200-400 microns. In some embodiments, the hole diameter is about 70-90 microns.

In some embodiments, the perforations have a diameter between about 40 and 150 microns. In some embodiments, the perforations have a uniform diameter. In some embodiments, the perforations do not have a uniform diameter.

### Density of Perforations

In some embodiments, less than 0.4% of the device's surface area is perforated and the holes are separated by about 2 mm (measuring center to center of the holes); however, they can be separated by less or more than 2 mm and still promote host-implant cell-to-cell vascularization. In some embodiments, less than about 5.0%, less than about 4.0%, less than about 3.0%, less than about 2.0%, less than about 1.0%, less than about 0.8%, less than about 0.3%, less than about 0.2%, less than about 0.1%, less than about 0.05% of the device's surface area is perforated. In some embodiments about 5.0-0.5%, about 5.0-3.5%, about 4.0-2.0% of the device's surface area is perforated.

In some embodiments perforations are avoided by replacing the cell-excluding membrane with a highly permeable membrane. For example, a membrane that consists of 80-120 micron pores in the membrane, such pores occurring at a density much like that described herein. In some embodiments, perforations can be relatively few and still provide the desired benefit of direct host vascularization while enhancing cell survival.

In some embodiments, a cell delivery device comprises a perforated cell-excluding membrane with holes separated by about 0.5 mm, 1.0 mm, 1.5 mm, 2 mm, 4 mm, 8 mm or more (measuring center to center of the holes). In some embodiments, a cell delivery device comprises a perforated cell-excluding membrane and perforated non-woven fabric layer with holes separated by about 0.5 mm, 1.0 mm, 1.5 mm, 2 mm, 4 mm, 8 mm or more. In some embodiments, a cell delivery device comprises a perforated cell-excluding membrane laminated to a perforated non-woven fabric layer with holes separated by about 0.5 mm, 1.0 mm, 1.5 mm, 2 mm, 4 mm, 8 mm or more. In one embodiment, a cell delivery device consisting of holes or perforations, wherein the holes are separated by about 0.5 mm-4 mm, or by about 0.5 mm-2 mm, or by about 1.0 mm-2 mm is provided.

The number/density of holes can be from 5-200 or from 20-100 holes per device and will depend in part of the size of the device (lumen surface area). Indeed, the number/density of holes can be from 20 to 50 to 100 holes per device lumen. The number/density of holes can be from 5-200 or from 20-100 holes per device lumen. A skilled artisan can determine the number/density of holes to achieve the desired effect. In the case of a diabetic patient, the number/density of holes is determined by the ability of the implanted cells to mature and/or produce insulin in response to blood glucose levels.

In some embodiments, a cell delivery device comprising holes or perforations, wherein the holes are separated by about 0.5 mm, 1.0 mm, 1.5 mm, 2 mm or more is provided and wherein the hole diameter is less than about 200 microns, less than about 150 microns, less than about 100 microns, or less than about 75 microns. In some embodiments, a cell delivery device comprising perforations, wherein the holes are separated by about 2 mm or more and wherein the hole diameter is less than about 200 microns is provided. In some embodiments, a cell delivery device comprising holes or perforations, wherein the holes are separated by about 2 mm or more and wherein the hole diameter is less than about 100 microns (measuring center to center of the holes) is provided.

### Manufacture of Perforated Cell Delivery Devices

Manufacturing methods known in the art can be used to produce the disclosed perforated devices. Historically, devices were assembled, loaded with cells, and then a needle was used to manually add perforations to an intact device. As such, all layers of the device were perforated. See U.S. application Ser. No. 12/618,659 and PCT Application No. WO/1993/002635.

Additionally, because the cells were inside the device when the perforations were made some portion of encapsulated cells are in the path of the needle upon perforation and the needle could damage some of the encapsulated cells. This method can also lead to inadvertent contamination (cells leaving the device) as the needle is inserted to make the hole and then removed.

Embodiments herein describe using a laser that provides control over hole size and distribution and does not perforate each layer of the device; and does not perforate the device after the cells are loaded. In this way, no cells are injured or destroyed by forming the perforations, potential contamination is reduced and just the cell-excluding membrane (or just the cell-excluding membrane and non-woven fabric layer) is perforated so that the other layers can help retain the encapsulated cells in the delivery device upon implant. In some embodiments, the device is perforated prior to loading of the cells.

Perforated cell delivery devices can be constructed in multiple size configurations and can comprise smaller devices (with nominal 20 μl capacity) and larger devices (with greater than about 200 μl capacity). Perforated and unperforated cell delivery devices share identical materials, manufacturing techniques and thickness.

By using lasers instead of a needle, disclosed is the manufacture of perforated cell delivery devices wherein only the cell-excluding membrane is perforated. In some embodiments, the non-woven fabric is laminated to the cell-excluding membrane and only these two layers are perforated. In some embodiments, the manufacture of holes in the device layers is automated. In some embodiments, the perforations are of circular shape or oval shape or elliptical shape. It should be noted that the perforations can have other shapes such as rectangular or hexagonal or polygonal, or slits. In one embodiment, the perforations have a uniform shape. In some embodiments, the perforations do not have a uniform shape. In some embodiments, the perforations are uniformly distributed on the cell excluding membrane. In some embodiments, the perforations are variably spaced on the cell excluding membrane, for example, they may be clustered at the center of the device or at the ends of the device. In some embodiments, the plurality of perforations is spaced in a series of rows and columns forming a grid arrangement or concentric circles or any other geometric configuration or combinations of such configurations. In some embodiments, the plurality of perforations is randomly distributed. In some embodiments, perforations are not on each cell-excluding membrane but only on one side of the device.

In some embodiments, there are a plurality of different cell populations in the device. In one embodiment, there are a plurality of chambers in the device and each chamber is separated by a cell-free zone or island and each chamber is perforated. In some embodiments, there are a plurality of chambers in the device and each chamber is separated by a cell free zone and not all chambers are perforated. In some embodiments, pancreatic progenitors are encapsulated in one chamber and a different therapeutic agent is encapsulated in another chamber. In this instance, only the chamber comprising the pancreatic progenitors will be perforated.

### Kit and Combination Product

Disclosed herein includes kits and combination products. The kits or combination products can comprise at least one of an immune attenuating drug and a device loaded with hypoimmunogenic (e.g., genetically modified) cells, i.e., each alone may be a candidate medical device or cell product, but used together they make a combination product. In various aspects, the combination product refers to an immune attenuating drug (described above) and a perforated device loaded with hypoimmunogenic cells (e.g., a gene edited cell provided above or population thereof). This can be referred to as a “combination product”, or “perforated combination product.” The at least one immune attenuating drug does not need to be packed inside the device to form part of the combination product. In other words, the at least one immune attenuating drug may be administered separately from the device loaded with hypoimmunogenic cells. In other aspects, the device may comprise the hypoimmunogenic cells and the at least one immune attenuating drug. The device (perforated or not) can be any macro cell delivery device described herein including but not limited to those cell encapsulation devices as described in U.S. Pat. Nos. 8,278,10⁶ and 9,526,880, PCT Application No. PCT/US2016/0061442 and U.S. Design Patent Nos. D714956, D718472, D718467, D718466, D718468, D718469, D718470, D718471, D720469, D726306, D726307, D728095, D734166, D734847, D747467, D747468, D747798, D750769, D750770, D755986, D760399, D761423, D761424 (incorporated by reference in their entirety). The cells loaded into the device (perforated or not) may be any genetically modified cells discussed above including but not limited to definitive endoderm, PDX1-positive endoderm, PDX1-positive foregut endoderm, pancreatic endoderm, pancreatic endoderm cells expressing PDX1 and NKX6.1, endocrine progenitors, endocrine progenitors expressing NKX6.1 and INS, immature beta cell, immature beta cells expressing NKX6.1, INS and MAFB, mature endocrine cells, mature endocrine cells expressing INS, GCG, SST and PP, and mature beta cells and mature beta cells expressing INS and MAFA.

Perforated delivery devices loaded with pancreatic endoderm or pancreatic progenitor hypoimmunogenic cells which mature when implanted in vivo are intended to reduce insulin dependence and/or reduce hypoglycemia in patients with diabetes. This includes, but is not limited to high-risk type I diabetic patients who are hypoglycemia unaware, labile (brittle), or have received an organ transplant and who can tolerate, or are already on, immune suppression therapy. As substantially described in PCT Application No. PCT/US2016/0061442 (incorporated by reference in its entirety), the primary method of action is via human pancreatic endoderm cells (PEC) or pancreatic progenitor hypoimmunogenic cells, contained in a permeable, durable, implantable medical device that facilitates direct host vascularization. The PEC hypoimmunogenic cells differentiate and mature into therapeutic glucose-responsive, insulin-releasing hypoimmunogenic cells after implantation. As such, the perforated combination product supports secretion of human insulin. The perforated combination product limits distribution (egress) of PEC hypoimmunogenic cells in vivo. The perforated combination product will be implanted in a location that permits sufficient vascular engraftment to sustain the population of therapeutic hypoimmunogenic cells within the device and facilitate distribution of insulin and other pancreatic products to the bloodstream. The perforated combination product is intended to be implanted and explanted with conventional surgical tools, and to provide a therapeutic dose for two years or more. The device is intended to retain an adequate dose of the PEC hypoimmunogenic cells product during formulation, shelf-life, handling and surgical implant to achieve clinical efficacy and ensure the cell product is located within the tissue capsule to meet safety requirements.

The FBR is an immune-mediated reaction to implanted materials where a cascade of inflammatory events and wound-healing processes can result in fibrosis, or the cellular and collagenous deposition that encapsulates implants (Anderson et al., 2008; Wick et al., 2013; Wynn and Ramalingam, 2012). While the FBR is necessary to enable subsequent vascularization, these events can compromise the performance and durability of implantable devices that require use over extended periods. Implant isolation by fibrosis often interferes with function, as a thick fibrotic layer can cut off the nourishment for cell-based implants, and ultimately lead to impaired graft survival and function.

A myriad of approaches have been adopted to modulate the inflammation associated with the FBR and enhance the integration of the implanted device with the surrounding tissue. These approaches include the use of novel biomaterials with reduced propensity to trigger the FBR and short-term administration of pharmaceutical agents to target specific fibroblast migration and suppress cytokine production by invading inflammatory cells (Ward, 2008).

Examples of pharmaceutical intervention include: nonsteroidal anti-inflammatory drugs (NSAIDs) with the implants (Kastellorizios et al., 2015), corticosteroids and tyrosine kinase inhibitors (Avula et al., 2014), dexamethasone (a potent anti-inflammatory), incorporated into drug-releasing devices engineered to release over extended periods of time and reduce device fibrosis (Bhardwaj et al., 2010), antifibrotic agents used to treat fibrosis of lung, liver, and kidney have been used to reduce the FBR (Gancedo et al., 2008; Gu et al., 2016; Taguchi et al., 2021), as there are many mechanistic similarities between the FBR and fibrosis of vital organs (Love and Jones, 2009).

The terms “administering,” “introducing,” “implanting,” “engrafting” and “transplanting” are used interchangeably in the context of the placement of cells, e.g., progenitor cells, into a subject, by a method or route that results in at least partial localization of the introduced cells at a desired site. The cells, e.g., progenitor cells, or their differentiated progeny can be administered by any appropriate route that results in delivery to a desired location in the subject where at least a portion of the implanted cells or components of the cells remain viable. The period of viability of the cells after administration to a subject can be as short as a few hours, e.g., twenty-four hours, to a few days, to as long as several years, or even the lifetime of the subject, i.e., long-term engraftment.

A genetically modified cell, e.g., universal donor cell, as described herein may be viable after administration to a subject for a period that is longer than that of an unmodified cell.

In some embodiments, a combination product of a gene edited cell, immune attenuating agent and perforated device may be administered to a subject, e.g., a human subject, who has, is suspected of having, or is at risk for a disease or disorder. In some embodiments, the subject is suffering or is at risk of developing symptoms indicative of a disease or disorder. In some embodiments, the disease is diabetes, e.g., type I diabetes or type II diabetes. In some embodiments, the disorder is a pancreatectomy.

Disclosed herein are combination products. In some embodiments, the combination product comprises a perforated cell delivery device, a genetically modified cell, or population thereof, and at least one immune attenuating drug. The genetically modified cell can be an allogeneic pancreatic endoderm cell.

The genetically modified cell can comprise (a) a nucleic acid comprising a nucleotide sequence encoding programmed death-ligand 1 (PD-L1) inserted within a gene encoding beta-2 microglobulin (B2M) and (b) a nucleic acid comprising a nucleotide sequence encoding HLA class I histocompatibility antigen, alpha chain E (HLA-E) inserted within a gene encoding thioredoxin interacting protein (TXNIP). In some embodiments, the genetically modified cell expresses PD-L1 and HLA-E and has reduced or eliminated expression of B2M and TXNIP. The nucleic acid of (a) can comprise a nucleotide sequence encoding TNFAIP3. The nucleic acid of (b) can comprise a nucleotide sequence encoding MANF. The nucleic acid of (a) can comprise the nucleotide sequence encoding TNFAIP3 linked to a nucleotide sequence encoding a P2A peptide linked to the nucleotide sequence encoding PD-L1 such that the nucleic acid of (a) comprises a TNFAIP3-P2A-PD-L1 polynucleotide sequence. The TNFAIP3-P2A-PD-L1 polynucleotide sequence can comprise the sequence of SEQ ID NO: 54 or a sequence that is at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 54. The nucleic acid of (a) can be operably linked to an exogenous promoter. The exogenous promoter can be a CMV, EF1α, PGK, CAG/CAGGS, or UBC promoter.

The nucleic acid of (b) can comprise the nucleotide sequence encoding MANF linked to a nucleotide sequence encoding a P2A peptide linked to the nucleotide sequence encoding HLA-E such that the nucleic acid of (b) comprises a MANF-P2A-HLA-E polynucleotide sequence. The MANF-P2A-HLA-E polynucleotide sequence can comprise SEQ ID NO: 55 or a sequence that is at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 55. The nucleic acid of (b) can be operably linked to an exogenous promoter. The exogenous promoter can be a CMV, EF1α, PGK, CAG/CAGGS, or UBC promoter.

The genetically modified cell can comprise: (a) a nucleic acid comprising a nucleotide sequence encoding TNFAIP3 linked to a nucleotide sequence encoding a P2A peptide linked to a nucleotide sequence encoding PD-L1 such that the nucleic acid of (a) comprises a TNFAIP3-P2A-PD-L1 polynucleotide sequence. The nucleic acid of (a) can be inserted within a gene encoding beta-2 microglobulin (B2M). and In some embodiments, the genetically modified cell can comprise (b) a nucleic acid comprising a nucleotide sequence MANF linked to a nucleotide sequence encoding a P2A peptide linked to a nucleotide sequence encoding HLA-E such that the nucleic acid of (b) comprises a MANF-P2A-HLA-E polynucleotide sequence. The nucleic acid of (b) can be inserted within a gene encoding thioredoxin interacting protein (TXNIP).

In some embodiments, the genetically modified cell expresses PD-L1, HLA-E, TNFAIP3 and MANF and has reduced or eliminated expression of B2M and TXNIP. The TNFAIP3-P2A-PD-L1 polynucleotide sequence can comprise the sequence of SEQ ID NO: 54 or a sequence that is at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 54; and the MANF-P2A-HLA-E polynucleotide sequence can comprise the sequence of SEQ ID NO: 55 or a sequence that is at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 55. The nucleic acids of (a) and (b) can be operably linked to an exogenous promoter. The exogenous promoter can be a CMV, EF1α, PGK, CAG/CAGGS, or UBC promoter.

In some embodiments, the genetically modified cell further comprises: (c) a disrupted TGFβ2 gene and the cell has reduced or eliminated expression of TGFβ2; (d) a disrupted CIITA gene and the cell has reduced or eliminated expression of CIITA; (e) an insertion of a nucleic acid encoding CD39 and the cell expresses CD39; (f) an insertion of a nucleic acid encoding CD73 and the cell expresses CD73; or (g) any combination of (c), (d), (e) and (f). In some embodiments, nucleic acid encoding CD39 is inserted within the CIITA gene, and the cell expresses CD39 and has reduced or eliminated expression of CIITA.

The at least one immune attenuating drug can comprise a JAK1 or JAK2 inhibitor, a TNFα or TNFβ blocker, an ILIR blocker, a calcineurin inhibitor, an anti-thymocyte globulin or any combination thereof. The at least one immune attenuating drug can comprise ruxolitinib, etanercept, anakinra or a combination of any thereof. The at least one immune attenuating drug can comprise the calcineurin inhibitor and the anti-thymocyte globulin. The calcineurin inhibitor can be tacrolimus or cyclosporine A. The at least one immune attenuating drug can comprise tacrolimus and the anti-thymocyte globulin. The tacrolimus can be Prograf®, Astagraf XL®, Envarsus XR®, Hecoria®, or any combination thereof. The anti-thymocyte globulin can be Thymoglobulin®, Atgam™ Fresenius™, Tecelac™, or any combination thereof.

The calcineurin inhibitor can be formulated for oral or intravenous administration. The anti-thymocyte globulin can be formulated for intravenous administration. Each of the at least one immune attenuating drug can be formulated as a separate composition. Each of the at least one immune attenuating drug can be formulated for intravenous administration, subcutaneous administration, oral administration, or any combination thereof. The population of genetically modified cells can be loaded into the perforated delivery device.

The perforated delivery device can be loaded with about 1×10⁶ to about 9.5×10⁶ genetically modified cells (e.g., about 1×10⁶, 1.1×10⁶, 1.2×10⁶, 1.3×10⁶, 1.4×10⁶, 1.5×10⁶, 1.6 ×10⁶, 1.7×10⁶, 1.8×10⁶, 1.9×10⁶, 2×10⁶, 2.1×10⁶, 2.2×10⁶, 2.3×10⁶, 2.4×10⁶, 2.5×10⁶, 2.6 ×10⁶, 2.7×10⁶, 2.8×10⁶, 2.9×10⁶, 3×10⁶, 3.1×10⁶, 3.2×10⁶, 3.3×10⁶, 3.4×10⁶, 3.5×10⁶, 3.6 ×10⁶, 3.7×10⁶, 3.8×10⁶, 3.9×10⁶, 4×10⁶, 4.1×10⁶, 4.2×10⁶, 4.3×10⁶, 4.4×10⁶, 4.5×10⁶, 4.6 ×10⁶, 4.7×10⁶, 4.8×10⁶, 4.9×10⁶, 5×10⁶, 5.1×10⁶, 5.2×10⁶, 5.3×10⁶, 5.4×10⁶, 5.5×10⁶, 5.6 ×10⁶, 5.7×10⁶, 5.8×10⁶, 5.9×10⁶, 6×10⁶, 6.1×10⁶, 6.2×10⁶, 6.3×10⁶, 6.4×10⁶, 6.5×10⁶, 6.6 ×10⁶, 6.7×10⁶, 6.8×10⁶, 6.9×10⁶, 7×10⁶, 7.1×10⁶, 7.2×10⁶, 7.3×10⁶, 7.4×10⁶, 7.5×10⁶, 7.6 ×10⁶, 7.7×10⁶, 7.8×10⁶, 7.9×10⁶, 8×10⁶, 8.1×10⁶, 8.2×10⁶, 8.3×10⁶, 8.4×10⁶, 8.5×10⁶, 8.6 ×10⁶, 8.7×10⁶, 8.8×10⁶, 8.9×10⁶, 9×10⁶, 9.1×10⁶, 9.2×10⁶, 9.3×10⁶, 9.4×10⁶, 9.5×10⁶ cells, or a number or a range between any two of these values).

In some embodiments, a kit is provided comprising any of the combination product described herein.

### Methods of Treatment

Various aspects of the present disclosure are directed to methods of producing insulin in a subject and reducing foreign body response in the subject. Disclosed herein include methods of producing insulin in a mammalian subject. In some embodiments, the method comprises: a) administering to a mammalian subject at least one immune attenuating drug; b) implanting a perforated cell delivery device comprising a genetically modified cell, or population thereof, into the mammalian subject; and c) maturing the genetically modified cell, or population thereof, in the perforated cell delivery device in the mammalian subject such that the mature cell, or population thereof, produces insulin secreting cells, thereby producing insulin in the mammalian subject.

In some embodiments, inflammation associated with the foreign body response of the perforated cell delivery device loaded with the genetically modified cell, or population thereof in the mammalian subject is reduced compared to administering a comparative perforated cell delivery device loaded with the genetically modified cell, or population thereof, without the at least one immune attenuating drug. In some embodiments, reducing the foreign body response comprises reducing cell trafficking and/or inflammation external to the device in a host tissue, facilitating integration of the device into the host tissue, and/or improving or accelerating healing of the host tissue around the device. In some aspects, as discussed above, the inflammation (e.g., a foreign body response) can comprise fibrosis, or cellular and collagenous deposition that encapsulates implants. Thus, reducing the inflammation can comprise reducing cell trafficking and/or inflammation external to the device in a host tissue, facilitating integration of the device into the host tissue, and/or improving or accelerating healing of the host tissue around the device.

Disclosed herein include methods for treating a pancreatic disease or disorder in a mammalian subject in need thereof. In some embodiments, the method comprises: a) administering to a mammalian subject at least one immune attenuating drug; b) implanting a perforated cell delivery device comprising a genetically modified cell, or population thereof, into the mammalian subject; and c) maturing the genetically modified cell, or population thereof, in the perforated cell delivery device in the mammalian subject such that the mature cell, or population thereof, produces insulin secreting cells, thereby producing insulin in the mammalian subject and treating the pancreatic disease or disorder.

In various aspects, the pancreatic disease or disorder can comprise type I diabetes, type II diabetes, or a pancreatectomy. In various aspects, the subject is a mammal. In further aspects, the subject is human. In various aspects, the at least one immune attenuating drug is administered before implantation of the cell delivery device. In some aspects, the at least one immune attenuating drug is administering simultaneously with implantation of the cell delivery device. In other aspects, the at least one immune attenuating drug is administered after implantation of the cell delivery device. In some cases, the at least one immune attenuating drug is administered before and during implantation, during or after implantation, or before and after implantation. In some aspects, the at least one immune attenuating drug is administered before, during and after implantation of the cell delivery device.

The pancreatic disease or disorder can be type I diabetes, type II diabetes, or a pancreatectomy. The genetically modified cell can be an allogeneic pancreatic endoderm cell. The genetically modified cell can comprise (a) a nucleic acid comprising a nucleotide sequence encoding programmed death-ligand 1 (PD-L1) inserted within a gene encoding beta-2 microglobulin (B2M) and (b) a nucleic acid comprising a nucleotide sequence encoding HLA class I histocompatibility antigen, alpha chain E (HLA-E) inserted within a gene encoding thioredoxin interacting protein (TXNIP). In some embodiments, the genetically modified cell expresses PD-L1 and HLA-E and has reduced or eliminated expression of B2M and TXNIP. The nucleic acid of (a) can comprise a nucleotide sequence encoding TNFAIP3. The nucleic acid of (b) can comprise a nucleotide sequence encoding MANF. The nucleic acid of (a) can comprise the nucleotide sequence encoding TNFAIP3 linked to a nucleotide sequence encoding a P2A peptide linked to the nucleotide sequence encoding PD-L1 such that the nucleic acid of (a) comprises a TNFAIP3-P2A-PD-L1 polynucleotide sequence. The TNFAIP3-P2A-PD-L1 polynucleotide sequence can comprise the sequence of SEQ ID NO: 54 or a sequence that is at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 54. The nucleic acid of (a) can be operably linked to an exogenous promoter. The exogenous promoter can be a CMV, EF1α, PGK, CAG/CAGGS, or UBC promoter. The nucleic acid of (b) can comprise the nucleotide sequence encoding MANF linked to a nucleotide sequence encoding a P2A peptide linked to the nucleotide sequence encoding HLA-E such that the nucleic acid of (b) comprises a MANF-P2A-HLA-E polynucleotide sequence. The MANF-P2A-HLA-E polynucleotide sequence can comprise the sequence of SEQ ID NO: 55 or a sequence that is at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 55. The nucleic acid of (b) can be operably linked to an exogenous promoter. The exogenous promoter can be a CMV, EF1α, PGK, CAG/CAGGS, or UBC promoter.

The genetically modified cell can comprise: (a) a nucleic acid comprising a nucleotide sequence encoding TNFAIP3 linked to a nucleotide sequence encoding a P2A peptide linked to a nucleotide sequence encoding PD-L1 such that the nucleic acid of (a) comprises a TNFAIP3-P2A-PD-L1 polynucleotide sequence. The nucleic acid of (a) can be inserted within a gene encoding beta-2 microglobulin (B2M). The genetically modified cell can comprise: (b) a nucleic acid comprising a nucleotide sequence MANF linked to a nucleotide sequence encoding a P2A peptide linked to a nucleotide sequence encoding HLA-E such that the nucleic acid of (b) comprises a MANF-P2A-HLA-E polynucleotide sequence. The nucleic acid of (b) can be inserted within a gene encoding thioredoxin interacting protein (TXNIP). In some embodiments, genetically modified cell expresses PD-L1, HLA-E, TNFAIP3 and MANF, and has reduced or eliminated expression of B2M and TXNIP. The TNFAIP3-P2A-PD-L1 polynucleotide sequence can comprise the sequence of SEQ ID NO: 54 or a sequence that is at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 54; and the MANF-P2A-HLA-E polynucleotide sequence can comprise the sequence of SEQ ID NO: 55 or a sequence that is at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 55. The nucleic acids of (a) and (b) can be operably linked to an exogenous promoter.

The exogenous promoter can be a CMV, EF1α, PGK, CAG/CAGGS, or UBC promoter. In some embodiments, the genetically modified cell further comprises: (c) a disrupted TGFβ2 gene and the cell has reduced or eliminated expression of TGFβ2; (d) a disrupted CIITA gene and the cell has reduced or eliminated expression of CIITA; (e) an insertion of a nucleic acid encoding CD39 and the cell expresses CD39; (f) an insertion of a nucleic acid encoding CD73 and the cell expresses CD73; or (g) any combination of (c), (d), (e) and (f). The nucleic acid encoding CD39 can be inserted within the CIITA gene, and the cell expresses CD39 and has reduced or eliminated expression of CIITA.

The at least one immune attenuating drug can comprise a JAK1 or JAK2 inhibitor, a TNFα or TNFβ blocker, an ILIR blocker, a calcineurin inhibitor, an anti-thymocyte globulin or any combination thereof. The at least one immune attenuating drug can comprise ruxolitinib, etanercept, anakinra or any combination thereof. The at least one immune attenuating drug can comprise the calcineurin inhibitor, the anti-thymocyte globulin, or both. The calcineurin inhibitor can be tacrolimus or cyclosporine A. The at least one immune attenuating drug can comprise tacrolimus, the anti-thymocyte globulin, or both. The tacrolimus can be Prograf®, Astagraf XL®, Envarsus XR®, Hecoria®, or any combination thereof. The anti-thymocyte globulin can be Thymoglobulin®, Atgam™, Fresenius™, Tecelac™, or any combination thereof.

The calcineurin inhibitor can be administered at a dose of about 0.5 mg to about 100 mg (e.g., about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 mg, a number or a range between any two of these values). The calcineurin inhibitor can be administered at a dose of about 0.2 mg, 1 mg, 5 mg, 25 mg, or 100 mg. The calcineurin inhibitor can be administered at a dose of about 0.5 mg, 1 mg, or 5 mg. The calcineurin inhibitor can be administered at a dose of about 0.2 mg or about 1 mg. The calcineurin inhibitor can be administered orally. The calcineurin inhibitor can be administered at a dose of about 5 mg/ml. About 1 mL of the calcineurin inhibitor can be administered intravenously. The anti-thymocyte globulin can be administered to the subject at a dose of about 1 mg/kg to about 2 mg/kg (e.g., about 1, 1.1, 1.2, 1.3, 1.4, 1.5, 1.6, 1.7, 1.8, 1.9, 2 mg/kg, or a number or a range between any two of the values). The anti-thymocyte globulin can be administered intravenously. The anti-thymocyte globulin can be administered to the subject at a dose of about 1.5 mg/kg. Each of the at least one immune attenuating drug can be administered by oral administration, intravenous administration, subcutaneous administration, or any combination thereof. Each of the at least one immune attenuating drug can be administered as a separate composition. Each of the at least one immune attenuating drug can be administered to the subject in a cycle of at least one week. Each of the at least one immune attenuating drug can be administered to the subject in a cycle of at least one week, two weeks, three weeks, four weeks, five weeks, six weeks, seven weeks, eight weeks, nine weeks, ten weeks, eleven weeks, twelve weeks, or more. Each of the at least one immune attenuating drug can be administered to the subject once or twice daily in the cycle. In some embodiments, the cycle begins prior to the implanting of the perforated cell delivery device. In some embodiments, the cycle begins about, at least, or at least about 24 hours, about 12 hours, about 6 hours, about 5 hours, about 4 hours, about 3 hours, about 2 hours, about 1 hour or less, prior to the implanting of the perforated cell delivery device. In some embodiments, the cycle beings about 1 month, 4 weeks, 3 weeks, 2 weeks, 7 days, 6 days, 5 days, 4 days, 3 days, or 2 days prior to the implanting of the perforated delivery device. In some embodiments, the cycle begins after the implanting of the perforated cell delivery device. In some embodiments, the cycle begins about 0.5 hour, about 1 hour, about 2 hours, about 3 hours, about 4 hours, about 5 hours, about 6 hours, about 12 hours, about 24 hours or more after the implanting of the perforated cell delivery device. In some embodiments, the cycle begins about 1 day, 2 days, 3 days, 4 days, 5 days, 6 days, 1 week, 2 weeks, 3 weeks, 4 weeks, one month or more after the implanting of the perforated delivery device. In some embodiments, the cycle begins concurrently with the implanting of the perforated delivery device. The method can comprise administering at least one additional cycle of treatment to the subject.

The population of genetically modified cells can comprise about 1×10⁶ to about 9.5×10⁶ cells (e.g., about 1×10⁶, 1.1×10⁶, 1.2×10⁶, 1.3×10⁶, 1.4×10⁶, 1.5×10⁶, 1.6×10⁶, 1.7 ×10⁶, 1.8×10⁶, 1.9×10⁶, 2×10⁶, 2.1×10⁶, 2.2×10⁶, 2.3×10⁶, 2.4×10⁶, 2.5×10⁶, 2.6×10⁶, 2.7 ×10⁶, 2.8×10⁶, 2.9×10⁶, 3×10⁶, 3.1×10⁶, 3.2×10⁶, 3.3×10⁶, 3.4×10⁶, 3.5×10⁶, 3.6×10⁶, 3.7 ×10⁶, 3.8×10⁶, 3.9×10⁶, 4×10⁶, 4.1×10⁶, 4.2×10⁶, 4.3×10⁶, 4.4×10⁶, 4.5×10⁶, 4.6×10⁶, 4.7 ×10⁶, 4.8×10⁶, 4.9×10⁶, 5×10⁶, 5.1×10⁶, 5.2×10⁶, 5.3×10⁶, 5.4×10⁶, 5.5×10⁶, 5.6×10⁶, 5.7 ×10⁶, 5.8×10⁶, 5.9×10⁶, 6×10⁶, 6.1×10⁶, 6.2×10⁶, 6.3×10⁶, 6.4×10⁶, 6.5×10⁶, 6.6×10⁶, 6.7 ×10⁶, 6.8×10⁶, 6.9×10⁶, 7×10⁶, 7.1×10⁶, 7.2×10⁶, 7.3×10⁶, 7.4×10⁶, 7.5×10⁶, 7.6×10⁶, 7.7 ×10⁶, 7.8×10⁶, 7.9×10⁶, 8×10⁶, 8.1×10⁶, 8.2×10⁶, 8.3×10⁶, 8.4×10⁶, 8.5×10⁶, 8.6×10⁶, 8.7 ×10⁶, 8.8×10⁶, 8.9×10⁶, 9×10⁶, 9.1×10⁶, 9.2×10⁶, 9.3×10⁶, 9.4×10⁶, 9.5×10⁶ cells, or a number or a range between any two of these values). In some embodiments, b) comprises implanting more than one perforated cell delivery device into the mammalian subject. In some embodiments, b) comprises implanting two, three, four, five, or more perforated cell delivery devices into the mammalian subject. About 1.0×10⁷ to about 2.0×10⁷ genetically modified cells per kilogram (e.g., about 1×10⁷, 1.1×10⁷, 1.2×10⁷, 1.3×10⁷, 1.4×10⁷, 1.5×10⁷, 1.6×10⁷, 1.7×10⁷, 1.8×10⁷, 1.9 ×10⁷, 2×10⁷ cells per kilogram, or a number or a range between any two of these values) can be administered to the subject. The mammalian subject can be human.

Several methods are available and known in the art for diagnosing and/or monitoring diabetes in a subject. In some embodiments, C-peptide level is increased in the serum of the subject following the implanting of b). The increase can be relative to (i) the C-peptide level of the subject prior to the implanting of b), (ii) the C-peptide level in one or more untreated subjects, and/or (iii) a reference C-peptide level.

In some embodiments, the serum C-peptide level in the subject is increased after about 20 weeks following the implanting of b). In some embodiments, the serum C-peptide level in the subject is increased after less than 20 weeks (e.g., after 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, or 3 months) following the implanting of b). In some embodiments, the serum C-peptide level in the subject is increased after more than 20 weeks (e.g., after 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 55 years, 60 years, 65 years, or more than 65 years) following the implanting of b). The C-peptide level in the serum of the subject can be about 1.1 ng/mL to about 4.4 ng/ml (e.g., about 1.1, 1.5, 2.0, 2.5, 3.0, 2.5, 3.0, 3.5, 4.0, 4.4 ng/mL or a number or a range between any two of these values) following the implanting of b). The C-peptide level in the serum of the subject can be about 1.1 ng/ml to about 4.4 ng/ml (e.g., about 1.1, 1.5, 2.0, 2.5, 3.0, 2.5, 3.0, 3.5, 4.0, 4.4 ng/mL or a number or a range between any two of these values) after about 20 weeks following the implanting of b). In some embodiments, the C-peptide level in the serum of the subject is greater than 4.4 ng/mL prior to the implanting of the perforated cell delivery device.

In some embodiments, A1C percentage in the blood of the subject is decreased in the subject following the implanting of b). The decrease can be relative to (i) the A1C percentage of the subject prior to the implanting of b), (ii) the A1C percentage in one or more untreated subjects, and/or (iii) a reference A1C percentage. In some embodiments, fasting plasma glucose (FPG) level in the serum and/or plasma of the subject is decreased in the subject following the implanting of b). The decrease can be relative to (i) the FPG level of the subject prior to the implanting of b), (ii) the FPG level in one or more untreated subjects, and/or (iii) a reference FPG level.

The blood AlC percentage, the serum and/or plasma FPG levels, or any combination thereof, in the subject can be decreased after about 20 weeks following the implanting of b). The blood A1C percentage, the serum and/or plasma FPG levels, or any combination thereof, in the subject can be decreased after less than 20 weeks (e.g., after 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, or 3 months) following the implanting of b). The blood AlC percentage, the serum and/or plasma FPG levels, or any combination thereof, in the subject can be decreased after more than 20 weeks (e.g., after 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 55 years, 60 years, 65 years, or more than 65 years) following the implanting of b). The A1C percentage in the blood of the subject can be less than 6.5% following the implanting of b). The A1C percentage in the blood of the subject can be less than 6.5% after about 20 weeks following the implanting of b). In some embodiments, the A1C percentage is less than 5.7% after about 20 weeks following the implanting of the perforated delivery device. In some embodiments, the A1C percentage is 6.5% or greater in the blood of the subject prior to the implanting of the perforated cell delivery device.

The FPG level in the serum and/or plasma of the subject can be less than 125 mg/dL after about 20 weeks following the implanting of b). The FPG level in the serum and/or plasma of the subject can be less than 125 mg/dL after less than 20 weeks (e.g., after 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, or 3 months) following the implanting of b). The FPG level in the serum and/or plasma of the subject can be less than 125 mg/dL after more than 20 weeks (e.g., after 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 55 years, 60 years, 65 years, or more than 65 years) following the implanting of b). The FPG level in the serum and/or plasma of the subject can be less than 99 mg/dL following the implanting of the perforated delivery device. The FPG level in the serum and/or plasma of the subject can be less than 99 mg/dL after about 20 weeks following the implanting of the perforated delivery device. In some embodiments, the FPG level in the serum and/or plasma of the subject is greater than 126 mg/dL prior to the implanting of the perforated cell delivery device.

### EXAMPLES

Some aspects of the embodiments discussed above are disclosed in further detail in the following examples, which are not in any way intended to limit the scope of the present disclosure.

### Example 1

### Cell Maintenance and Expansion

Maintenance of hESC/hiPSCs

Human embryonic stem cells (hESC) and human induced pluripotent stem cells (hiPSCs) were maintained in StemFlex Complete (Life Technologies, A3349401) on BIOLAMININ 521 CTG (BioLamina Cat #CT521) or laminin 511 coated tissue culture plates. The cells were fed daily with StemFlex media. For plating of the cells as single cells, the cells were plated with 1% RevitaCell™ Supplement (100X) (ThermoFisher Cat #A2644501) in StemFlex on BIOLAMININ or laminin 511 coated plates. For passaging, 1% REVITACELL™ Supplement (100×) was added.

Single Cell Cloning of hPSCs

For single cell cloning, hPSCs (hESCs or hiPSCs) were fed with StemFlex Complete (Life Technologies, A3349401) with 1% RevitaCell™ Supplement (100×) (ThermoFisher Cat #A2644501). Following dissociation with ACCUTASE®, the cells were sorted as a single cell per well of a pre-coated plate. The 96-well plates were pre-coated with a 1:10 or a 1:20 dilution of BIOLAMININ 521 CTG (BioLamina Cat #CT521) in DPBS, calcium, magnesium (Life Technologies, 14040133) for 2 hours at 37° C. The WOLF FACS-sorter (Nanocellect) was used to sort single cells into the wells. The plates were pre-filled with 100-200 μL of StemFlex Complete with RevitaCell™ and 4 μL/mL of Recombinant Laminin iMatrix-511 E8 (AMSBIO, AMS.892 011). Three days post cell seeding, the cells were fed with fresh StemFlex and continued to be fed every other day with 100-200 μL of media. After 10 days of growth, the cells were fed daily with StemFlex until day 12-14. At this time, the plates were dissociated with ACCUTASE® and the collected cell suspensions were split 1:2 with half going into a new 96-well plate for maintenance and half going into a DNA extraction solution QuickExtract™ DNA Extraction Solution (Lucigen). Following DNA extraction, PCR was performed to assess presence or absence of desired gene edits at the targeted DNA locus. Sanger sequencing was used to verify desired edits.

Expansion of Single Cell Derived hPSCs Clones

For hESCs, successfully targeted clones were passaged from 96-well plates to 24-well plates using StemFlex and BIOLAMININ 521 or Recombinant Laminin iMatrix-511 E8. Following expansion in 24-well plates, the cells were passaged onto 6-well plates and a transition to KSR A10H10 media was begun the day after plating in StemFlex. The first day post plating, the cells were fed with a 50:50 mix of KSR A10H10 and StemFlex. The next day the cells were fed with 100% KSR A10H10. After 2 days in 100% KSR A10H10, the cells could be passaged using 10% XF in KSR A10H10. If the cells had not had 2 days of 100% KSR A10H10, the cells received BIOLAMININ 521 or Recombinant Laminin iMatrix-511 E8 to enable attachment and survival, followed by additional growth in KSR A10H10 and full transition to culture with laminin. Following the full transition to KSR A10H10, hESCs clones were passaged as described in Schulz et al. (2012) PLOS ONE 7 (5): e37004.

For hiPSCs, cells are maintained in StemFlex Complete throughout the cloning and regular maintenance processes on BIOLAMININ-coated plates with RevitaCell™ at the passaging stages.

### Example 2

Generation of B2M Knock-Out (KO) with MANF-P2A-TNFAIP3-P2A-PD-L-1 Knock In (KI)

### Human Pluripotent Stem Cells

This example describes the generation and characterization of specific universal donor cells with additional edits to improve survival (MANF) and immune evasion (TNFAIP3, also known as A20) according to the present disclosure. Cells were generated in which a transgene encoding MANF-P2A-TNFAIP3-P2A-PD-L-1 was inserted into the B2M gene locus, thereby knocking out the B2M gene.

B2M targeting gRNAs were designed for targeting exon 1 of the B2M coding sequence. These gRNAs had predicted low off-target scores based on sequence homology prediction using gRNA design software. The target sequences of the gRNAs are presented in Table 2. A gRNA comprises spacer sequence corresponding to the target DNA sequence.

TABLE 2
B2M gRNA TARGET SEQUENCES

| | | Target | | Spacer |
|---|---|---|---|---|
| | Target Sequence | SEQ ID | | SEQ ID |
| Name | (5′-3′) | NO: | PAM | NO: † |
| B2M-1 gRNA | GCTACTCTCTCTTTCTGGCC | 1 | TGG | 61 |
| (Exon_1_T12) | | | | |
| B2M-2 gRNA | GGCCGAGATGTCTCGCTCCG | 2 | TGG | 62 |
| (Exon_1_T2) | | | | |
| B2M-3 gRNA | CGCGAGCACAGCTAAGGCCA | 3 | CGG | 63 |
| (Exon_1_T8) | | | | |
| Exon_1_T1 | TATAAGTGGAGGCGTCGCGC | 4 | TGG | 64 |
| Exon_1_T3 | GAGTAGCGCGAGCACAGCTA | 5 | AGG | 65 |
| Exon_1_T4 | ACTGGACGCGTCGCGCTGGC | 6 | GGG | 66 |
| Exon_1_T5 | AAGTGGAGGCGTCGCGCTGG | 7 | CGG | 67 |
| Exon_1_T6 | GGCCACGGAGCGAGACATCT | 8 | CGG | 68 |
| Exon_1_T7 | GCCCGAATGCTGTCAGCTTC | 9 | AGG | 69 |
| Exon_1_T9 | CTCGCGCTACTCTCTCTTTC | 10 | TGG | 70 |
| Exon_1_T10 | TCCTGAAGCTGACAGCATTC | 11 | GGG | 71 |
| Exon_1_T11 | TTCCTGAAGCTGACAGCATT | 12 | CGG | 72 |
| Exon_1_T13 | ACTCTCTCTTTCTGGCCTGG | 13 | AGG | 73 |

† Spacer sequence is the RNA version of the target sequence, upstream of the PAM site.

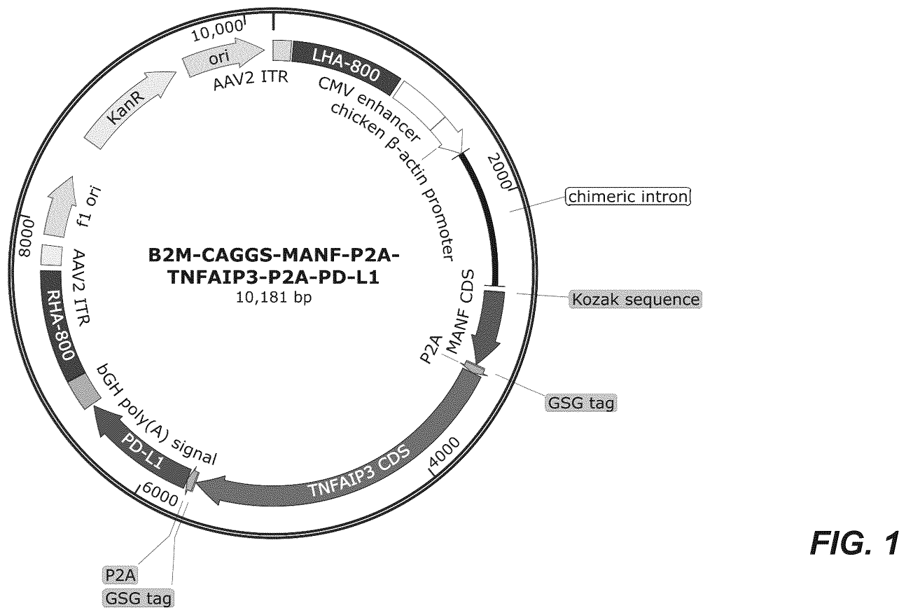

Plasmid design to insert a transgene encoding MANF-P2A-TNFAIP3-P2A-PD-L-1 into the B2M locus was made such that the starting codon of B2M was removed after undergoing homology directed repair (HDR) to insert the transgene, nullifying any chance of partial B2M expression. Successful HDR resulted in the insertion of the 3 genes of MANF, TNFAIP3, and PD-L-1 (CD274) into the genome. The three coding sequences were linked by P2A peptide coding sequences to allow for expression of the three separate proteins from a single transcript. The coding sequence of MANF-P2A-TNFAIP3-P2A-PD-L-1 comprises the nucleotide sequence of SEQ ID NO: 52. FIG. 1 presents a schematic of the B2M-CAGGS-MANF-P2A-TNFAIP3-P2A-PD-L-1 donor plasmid and Table 3 identifies the elements and locations therein. The donor plasmid contained a CAGGS promoter (i.e., comprising a CMV enhancer, a chicken β-actin promoter, and a chimeric intron) driven cDNA of MANF-P2A-TNFAIP3-P2A-PD-L-1 flanked by 800 base pair homology arms with identical sequence to the B2M locus around exon 1. The complete sequence of the plasmid comprises the nucleotide sequence of SEQ ID NO: 24.

TABLE 3
ELEMENTS OF B2M-CAGGS-MANF-P2A-
TNFAIP3-P2A-PD-L-1 DONOR PLASMID

| Element | Location (size in bp) | | SEQ ID NO: |
|---|---|---|---|
| Left ITR | 1-130 | (130) | 14 |
| LHA-B2M | 145-944 | (800) | 15 |
| CAGGS promoter | 973-2639 | (1667) | 16 |
| MANF | 2684-3229 | (546) | 17 |
| P2A | 3239-3295 | (57) | 18 |
| TNFAIP3 | 3296-5665 | (2370) | 19 |
| P2A | 5675-5731 | (57) | 18 |
| PD-L-1 | 5732-6604 | (873) | 20 |
| bGH poly(A) signal | 6622-6846 | (225) | 21 |
| RHA-B2M | 6853-7652 | (800) | 22 |
| Right ITR | 7694-7834 | (141) | 23 |
| Entire plasmid | 10,181 | bp | 24 |

Human ESCs were electroporated using the Neon Electroporator (Neon Transfection System ThermoFisher Cat #MPK5000) with 4 μg of plasmid DNA per million hESCs, along with a ribonucleoprotein (RNP) mixture of Cas9 protein and B2M-2 gRNA (comprising a spacer sequence of SEQ ID NO: 62). To form the RNP complex, gRNA and Cas9 were combined in one vessel with R-buffer (Neon Transfection System 100 μL Kit ThermoFisher Cat #MPK10096) to a total volume of 25-50 μL and incubated for 15 min at RT. Cells were dissociated using ACCUTASE®, then resuspended in DMEM/F12 media (Gibco, cat #11320033), counted using an NC-200 (Chemometec) and centrifuged. A total of 1×10⁶ cells were resuspended with the plasmid, the RNP complex, and R-buffer. This mixture was then electroporated. Following electroporation, the cells were pipetted out into an Eppendorf tube or a well of a 6-well plate filled with StemFlex media with RevitaCell™. This cell suspension was then plated into pre-coated tissue culture dishes. Cells were cultured in a normoxia incubator (37° C., 8% C₀2).

Seven to ten days post electroporation, the cells were enriched for PD-L-1 expressing cells via magnetic assisted cell sorting (MACS) using anti-mouse IgG Dynabeads (ThermoFisher, CELLection™ Pan Mouse IgG Kit, 11531D. These enriched cells (LIV008 cell line) represented a bulk KI population that was highly PD-L-1 positive. The enriched cells were then FACS-sorted for PD-L-1 surface expression using the WOLF FACS-sorter (Nanocellect) into BIOLAMININ 521 CTG coated 96-well plates with StemFlex and RevitaCell™. To detect the PD-L-1 surface expression, anti-PD-L-1 fluorescent antibodies were used (see Table 4). For FACS-sorting, unedited cells served as a negative control. PD-L-1 positive cells were selected for sorting and single cell cloning.

TABLE 4
ANTIBODIES FOR FLOW CYTOMETRY

| Antigen | Clone | Fluorophore | Manufacturer | Catalog # |
|---|---|---|---|---|
| B2M | 2M2 | PE | Biolegend | 316305 |
| HLA-ABC | W6/32 | Alexa 488 | Biolegend | 311415 |
| mIgG1 kappa | N/A | PE | BD Bioscience | 555749 |
| PD-L-1 | B7-H1 | Alexa-488 | ThermoFisher | 53-5983-42 |
| HLA-E | 3D12 | PE | ThermoFisher | 12-9953-42 |

Plated single cells were grown in a normoxia incubator (37° C., 8% C₀2) with every other day media changes until colonies were large enough to be re-seeded as single cells. When confluent, samples were split for maintenance and genomic DNA extraction. Correctly targeted clones were identified via PCR for the MANF-TNFAIP3-PD-L-1 KI insertion using primers that amplify from outside the plasmid homology arms at the site of insertion into the B2M locus, enabling amplification of the KI integrated DNA only. The B2M KO state of clones was confirmed via PCR and Sanger sequencing. The correct KI and KO clones were expanded in increasing tissue culture formats until a population size of 30 million cells was reached.

### Example 3

Generation of B2M KO with CD39-P2A-PD-L-1 KI Human Pluripotent Stem Cells

Cells were generated in which a transgene encoding CD39-P2A-PD-L-1 was inserted into the B2M gene locus, thereby knocking out the B2M gene.

Human pluripotent stem cells were electroporated essentially as described above in Example 2 with a B2M-CAGGS-CD39-P2A-PD-L-1 donor plasmid, as detailed below in Table 5, and an RNP comprising Cas9 and a B2M-2 gRNA (comprising a spacer sequence of SEQ ID NO: 62).

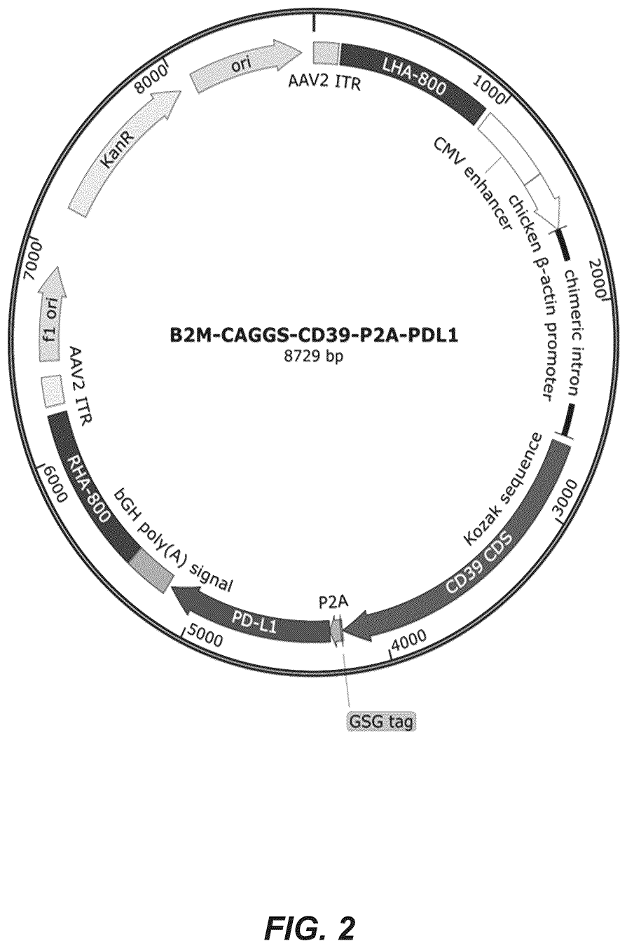

FIG. 2 presents a schematic of the B2M-CAGGS-CD39-P2A-PD-L-1 donor plasmid and Table 5 identifies the elements and locations therein. The B2M-CAGGS-CD39-P2A-PD-L-1 donor plasmid comprises a CAGGS promoter (comprising a CMV enhancer, a chicken β-actin promoter, and a chimeric intron) to drive expression of cDNA of CD39-P2A-PD-L-1 (SEQ ID NO: 53) flanked by 800 base pair homology arms with identical sequence to the B2M locus around exon 1. The complete sequence of the B2M-CAGGS-CD39-P2A-PD-L-1 donor plasmid comprises the nucleotide sequence of SEQ ID NO: 30.

TABLE 5
ELEMENTS OF B2M-CAGGS-CD39-
P2A-PD-L-1 DONOR PLASMID

| Element | Location (size in bp) | | SEQ ID NO: |
|---|---|---|---|
| Left ITR | 1-130 | (130) | 14 |
| LHA-B2M | 145-944 | (800) | 15 |
| CAGGS promoter | 973-2639 | (1667) | 16 |
| CD39 | 2684-4213 | (1530) | 27 |
| P2A | 4223-4279 | (57) | 18 |
| PD-L-1 | 4280-5152 | (873) | 20 |
| bGH poly(A) signal | 5170-5394 | (225) | 21 |
| RHA-B2M | 5401-6200 | (800) | 22 |
| Right ITR | 6242-6382 | (141) | 23 |
| Entire plasmid | 8729 | bp | 30 |

Seven to ten days post electroporation, the cells were enriched for PD-L-1 expressing cells via magnetic assisted cell sorting (MACS) using anti-mouse IgG Dynabeads (ThermoFisher, CELLection™ Pan Mouse IgG Kit, 11531D. These enriched cells represented a bulk KI population that was highly PD-L-1 positive. The enriched cells were then FACS-sorted for PD-L-1 surface expression using the WOLF FACS-sorter (Nanocellect) into BIOLAMININ 521 CTG coated 96-well plates with StemFlex and RevitaCell™. To detect the PD-L-1 surface expression, anti-PD-L-1 fluorescent antibodies were used (see Table 4). For FACS-sorting, unedited cells served as a negative control. PD-L-1 positive cells were selected for sorting and single cell cloning.

Plated single cells were grown in a normoxia incubator (37° C., 8% C₀2) with every other day media changes until colonies were large enough to be re-seeded as single cells. When confluent, samples were split for maintenance and genomic DNA extraction. Correctly targeted clones were identified via PCR for the CD39-PD-L-1 KI insertion using primers that amplify from outside the plasmid homology arms at the site of insertion into the B2M locus, enabling amplification of the KI integrated DNA only. The B2M KO state of clones was confirmed via PCR and Sanger sequencing. The correct KI and KO clones (LIV017 cell line) were expanded in increasing tissue culture formats until a population size of 30 million cells was reached.

### Example 4

Generation of B2M KO with TNFAIP3-P2A-PD-L-1 KI and TXNIP KO with MANF-P2A-HLA-E KI Human Pluripotent Stem Cells (“X1” cells)

Cells were generated in which a transgene encoding TNFAIP3-P2A-PD-L-1 was inserted into the B2M gene locus and a transgene encoding MANF-P2A-HLA-E was inserted into a TXNIP gene locus, thereby knocking out the B2M and TXNIP genes.

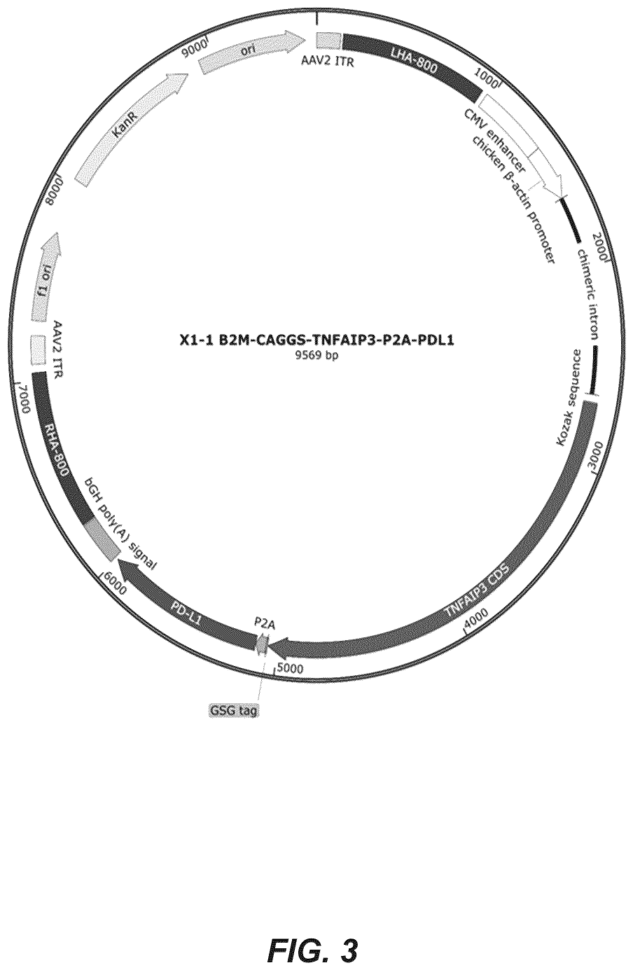

Human pluripotent stem cells were electroporated essentially as described above in Example 2 with a B2M-CAGGS-TNFAIP3-P2A-PD-L-1 donor plasmid (see below) and an RNP comprising Cas9 and B2M-2 gRNA (comprising a spacer sequence of SEQ ID NO: 62). FIG. 3 presents a schematic of the B2M-CAGGS-TNFAIP3-P2A-PD-L-1 donor plasmid (also called X1-1 cassette) and Table 6 identifies the elements and locations therein. The B2M-CAGGS-TNFAIP3-P2A-PD-L-1 donor plasmid comprises a CAGGS promoter (comprising a CMV enhancer, a chicken β-actin promoter, and a chimeric intron) to drive expression of cDNA of TNFAIP3-P2A-PD-L-1 (SEQ ID NO: 54) flanked by 800 base pair homology arms with identical sequence to the B2M locus around exon 1. The complete sequence of the B2M-CAGGS-TNFAIP3-P2A-PD-L-1 donor plasmid comprises the nucleotide sequence of SEQ ID NO: 31.

TABLE 6
ELEMENTS OF B2M-CAGGS-TNFAIP3-
P2A-PD-L-1 DONOR PLASMID

| Element | Location (size in bp) | | SEQ ID NO: |
|---|---|---|---|
| Left ITR | 1-130 | (130) | 14 |
| LHA-B2M | 145-944 | (800) | 15 |
| CAGGS promoter | 973-2639 | (1667) | 16 |
| TNFAIP3 | 2684-5053 | (2370) | 19 |
| P2A | 5063-5119 | (57) | 18 |
| PD-L-1 | 5120-5992 | (873) | 20 |
| bGH poly(A) signal | 6010-6234 | (225) | 21 |
| RHA-B2M | 6241-7040 | (800) | 22 |
| Right ITR | 7082-7222 | (141) | 23 |
| Entire plasmid | 9569 | bp | 31 |

Seven to ten days post electroporation, the cells were enriched for PD-L-1 expressing cells via MACS essentially as described in Example 2. Post PD-L-1 enrichment, the enriched cells were electroporated with a TXNIP-CAGGS-MANF-P2A-HLA-E donor plasmid, as detailed below, and an RNP comprising Cas9 and a gRNA targeting exon 1 of the TXNIP gene (i.e., TXNIP_Exon 1_T5 gRNA, comprising a spacer sequence of SEQ ID NO: 80). Table 7 presents the target sequences of additional gRNAs that target exon 1 or exon 2 of the TXNIP gene. These gRNAs had predicted low off-target scores based on sequence homology prediction using gRNA design software. A gRNA comprises an RNA spacer sequence corresponding to the target DNA sequence.

TABLE 7
TXNIP gRNA TARGET SEQUENCES

| | | Target | | Spacer |
|---|---|---|---|---|
| | Target Sequence | SEQ | | SEQ ID |
| Name | (5′-3′) | ID NO: | PAM | NO: † |
| TXNIP_Exon_ | GAAGCGTGTCTTCATAGCGC | 32 | AGG | 75 |
| 1_T1 | | | | |
| TXNIP_Exon_ | TTACTCGTGTCAAAGCCGTT | 33 | AGG | 76 |
| 1_T21 | | | | |
| TXNIP_Exon_ | TGTCAAAGCCGTTAGGATCC | 34 | TGG | 77 |
| 1_T22 | | | | |
| TXNIP_Exon_ | GCCGTTAGGATCCTGGCTTG | 35 | CGG | 78 |
| 1_T23 | | | | |
| TXNIP_Exon_ | GCGGAGTGGCTAAAGTGCTT | 36 | TGG | 79 |
| 1_T25 | | | | |
| TXNIP_Exon_ | TCCGCAAGCCAGGATCCTAA | 37 | CGG | 80 |
| 1_T5 | | | | |
| TXNIP_Exon_ | GTTCGGCTTTGAGCTTCCTC | 38 | AGG | 81 |
| 2_T4 | | | | |
| TXNIP_Exon_ | GAGATGGTGATCATGAGACC | 39 | TGG | 82 |
| 2_T2 | | | | |
| TXNIP_Exon_ | TTGTACTCATATTTGTTTCC | 40 | AGG | 83 |
| 2_T1 | | | | |
| TXNIP_Exon_ | AACAAATATGAGTACAAGTT | 41 | CGG | 84 |
| 2_T3 | | | | |

† Spacer sequence is the RNA version of the target sequence upstream of the PAM site.

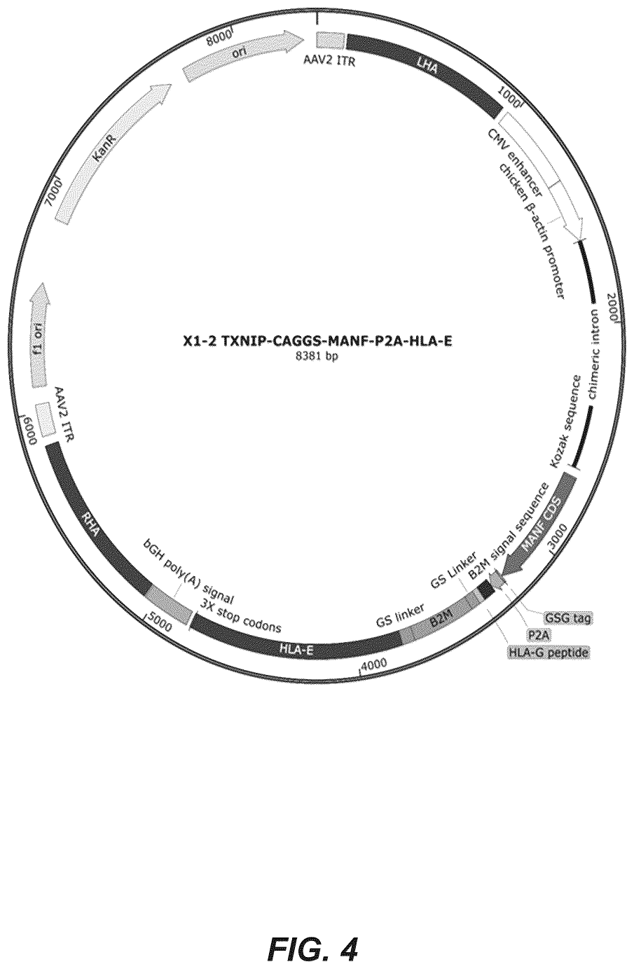

FIG. 4 presents a schematic of the TXNIP-CAGGS-MANF-P2A-HLA-E donor plasmid (also called X1-2 cassette) and Table 8 identifies the elements and locations therein. The TXNIP-CAGGS-MANF-P2A-HLA-E donor plasmid comprises a CAGGS promoter (comprising a CMV enhancer, a chicken β-actin promoter, and a chimeric intron) to drive expression of cDNA of MANF-P2A-HLA-E (SEQ ID NO: 55) flanked by 800 base pair homology arms with identical sequence to the TXNIP locus around exon 1. The HLA-E sequence (SEQ ID NO: 43) encodes an HLA-E trimer, which comprises a B2M signal peptide fused to an HLA-G presentation peptide fused to a GS linker fused to the B2M membrane protein fused to a GS linker fused to the HLA-E protein without its signal peptide. This trimer design has been previously published (Gornalusse et al. (2017) Nat. Biotechnol. 35 (8): 765-772). The complete sequence of the TXNIP-CAGGS-MANF-P2A-HLA-E donor plasmid comprises the nucleotide sequence of SEQ ID NO: 45.

TABLE 8
ELEMENTS OF TXNIP-CAGGS-MANF-P2A-HLA-E
DONOR PLASMID

| Element | Location (size in bp) | | SEQ ID NO: |
|---|---|---|---|
| Left ITR | 1-130 | (130) | 14 |
| LHA-TXNIP | 145-944 | (800) | 42 |
| CAGGS promoter | 973-2639 | (1667) | 16 |
| MANF | 2684-3229 | (546) | 17 |
| P2A | 3239-3295 | (57) | 18 |
| HLA-E | 3296-4795 | (1500) | 43 |
| bGH poly(A) signal | 4822-5046 | (225) | 21 |
| RHA-TXNIP | 5053-5852 | (800) | 44 |
| Right ITR | 5894-6034 | (141) | 23 |
| Entire plasmid | 8381 | bp | 45 |

Seven to ten days post electroporation, the cells were enriched for HLA-E expressing cells via MACS using Miltenyi reagents or ThermoFisher reagents. These enriched cells were then FACS sorted using the WOLF FACS-sorter (Nanocellect) into BIOLAMININ 521 CTG coated 96-well plates with StemFlex and RevitaCell™ with gating set for PD-L-1 and HLA-E double positive cells. To detect the PD-L-1 surface expression and HLA-E surface expression, anti-PD-L-1 and anti-HLA-E fluorescent antibodies were used (see Table 4). For FACS-sorting, unedited cells served as a negative control. PD-L-1 and HLA-E double positive cells (LIV028 cell line) were selected for sorting and single cell cloning.

Plated single cells were grown in a normoxia incubator (37° C., 8% C₀2) with every other day media changes until colonies were large enough to be re-seeded as single cells. When confluent, samples were split for maintenance and genomic DNA extraction. Correctly targeted clones were identified via PCR for the PD-L-1 KI insertion and the HLA-E KI insertion using primers that amplify from outside the plasmid homology arms at each insertion site, thereby enabling amplification of the KI integrated DNA only. The B2M and TXNIP KO state of clones were confirmed via PCR and Sanger sequencing. The correct KI and KO clones were expanded in increasing tissue culture formats until a population size of 30 million cells was reached. These cells are referred to as X1 cells hereafter.

### Example 5

Generation of B2M KO with TNFAIP3-P2A-PD-L-1 KI, TXNIP KO with MANF-P2A-HLA-E KI, and CIITA KO with CD39 KI Human Pluripotent Stem Cells

Cells were generated in which a transgene encoding TNFAIP3-P2A-PD-L-1 was inserted into the B2M gene locus, a transgene encoding MANF-P2A-HLA-E was inserted into the TXNIP gene locus, and a transgene encoding CD39 was inserted into the CIITA gene locus, thereby knocking out the B2M, TXNIP, and CIITA genes.

Human pluripotent stem cells were electroporated essentially as described above in Example 2 with the B2M-CAGGS-TNFAIP3-P2A-PD-L-1 donor plasmid (SEQ ID NO: 31, Table 6) and an RNP comprising Cas9 and B2M-2 gRNA comprising a spacer sequence of SEQ ID NO: 62). Seven to ten days post electroporation, the cells were enriched for PD-L-1 expressing (positive) cells via MACS essentially as described in Example 2. After the enriched PD-L-1 positive population was expanded, the cells were electroporated essentially as described above in Example 2 with the TXNIP-CAGGS-MANF-P2A-HLA-E donor plasmid (SEQ ID NO: 45, Table 8) and an RNP comprising Cas9 and TXNIP_Exon 1_T5 gRNA (comprising a spacer sequence of SEQ ID NO: 80). After enrichment for HLA-E positive cells and expansion of PD-L-1 and HLA-E cells, the double positive cells were used for further insertion of CD39 into the CIITA locus.

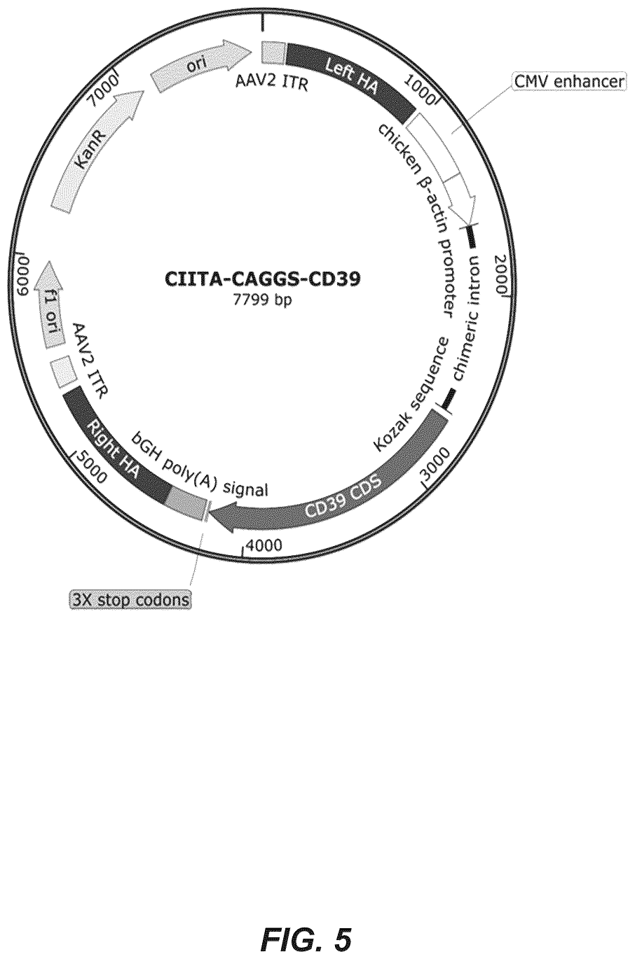

The CIITA-CAGGS-CD39 donor plasmid (SEQ ID NO: 29, Table 9) was introduced along with the ribonucleoprotein (RNP) complex made up of the CIITA targeting gRNA (CIITA Ex3_T6 gRNA (comprising a spacer sequence of SEQ ID NO: 74)) and Cas9 protein. FIG. 5 presents a schematic of the CIITA-CAGGS-CD39 donor plasmid and Table 9 identifies the elements and locations therein. The CIITA-CAGGS-CD39 donor plasmid comprises a CAGGS promoter (comprising a CMV enhancer, a chicken-actin promoter, and a chimeric intron) to drive expression of cDNA of CD39 flanked by 800 base pair homology arms with identical sequence to the CIITA locus around exon 3. The complete sequence of the plasmid comprises the nucleotide sequence of SEQ ID NO: 29.

TABLE 9
ELEMENTS OF CIITA-CAGGS-CD39 DONOR PLASMID

| Element | Location (size in bp) | | SEQ ID NO: |
|---|---|---|---|
| Left ITR | 1-130 | (130) | 14 |
| LHA-CIITA | 145-944 | (800) | 26 |
| CAGGS promoter | 973-2639 | (1667) | 16 |
| CD39 | 2684-4213 | (1530) | 27 |
| bGH poly(A) signal | 4240-4464 | (225) | 21 |
| RHA-CIITA | 4471-5270 | (800) | 28 |
| Right ITR | 5312-5452 | (141) | 23 |

| | | |
|---|---|---|
| Entire plasmid | 7799 | 29 |

In particular, a clone of X1, described in Example 4, was transfected with the CIITA-CAGGS-CD39 donor plasmid along with the RNP made up of the CIITA targeting gRNA (CIITA Ex3_T6 gRNA (comprising a spacer sequence of SEQ ID NO: 74)) and Cas9 protein. Per 2 million of hESC cells, 4 μg of plasmid DNA was delivered along with the RNP via electroporation. Electroporation was carried out in hESC cells using the Neon Electroporator with the RNP mixture of Cas9 protein (Biomay) and guide RNA (Biospring) at a molar ratio of 5:1 (gRNA:Cas9) with absolute values of 125 pmol Cas9 and 625 pmol gRNA per 2 million cells. To form the RNP complex, gRNA and Cas9 were combined in one vessel with R-buffer (Neon Transfection Kit) to a total volume of 25-50 μL and incubated for 15 min at room temperature (RT). Cells were dissociated using ACCUTASE®, then resuspended in StemFlex media, counted using an NC-200 (Chemometec) and centrifuged. A total of 2×10⁶ cells were resuspended with the RNP complex and R-buffer was added to a total volume of ˜115 μL. This mixture was then electroporated with 3 pulses for 30 ms at 1000 V. Two electroporations were performed. Following electroporation, the cells were pipetted out into a well of a 6 well plate filled with StemFlex media with RevitaCell and laminin 511. The plates were pre-coated with BIOLAMININ 521 CTG at 1:10 dilution. Cells were cultured in a normoxia incubator (37° C., 8% CO₂).

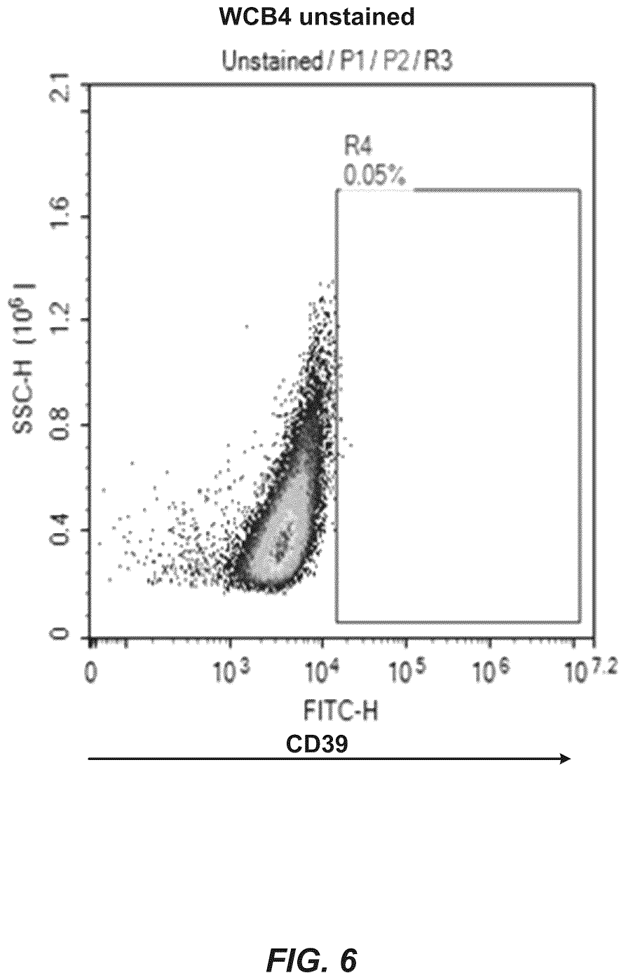

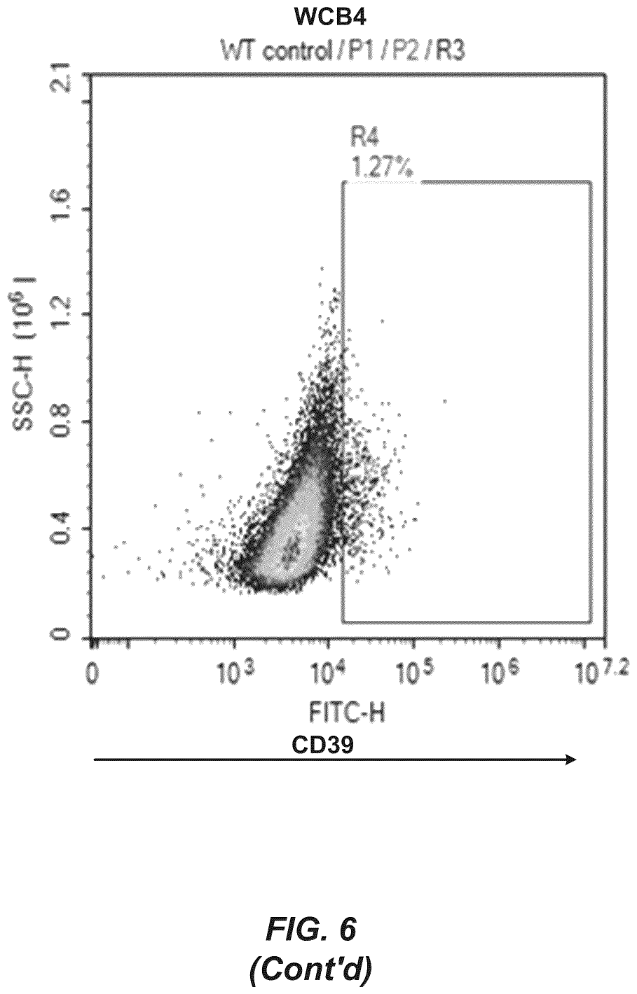

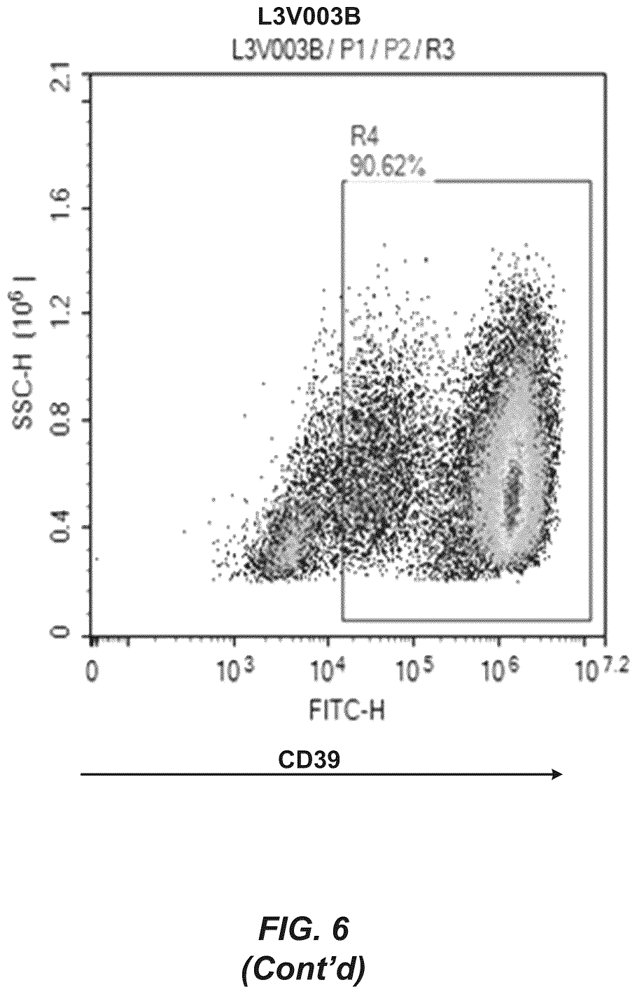

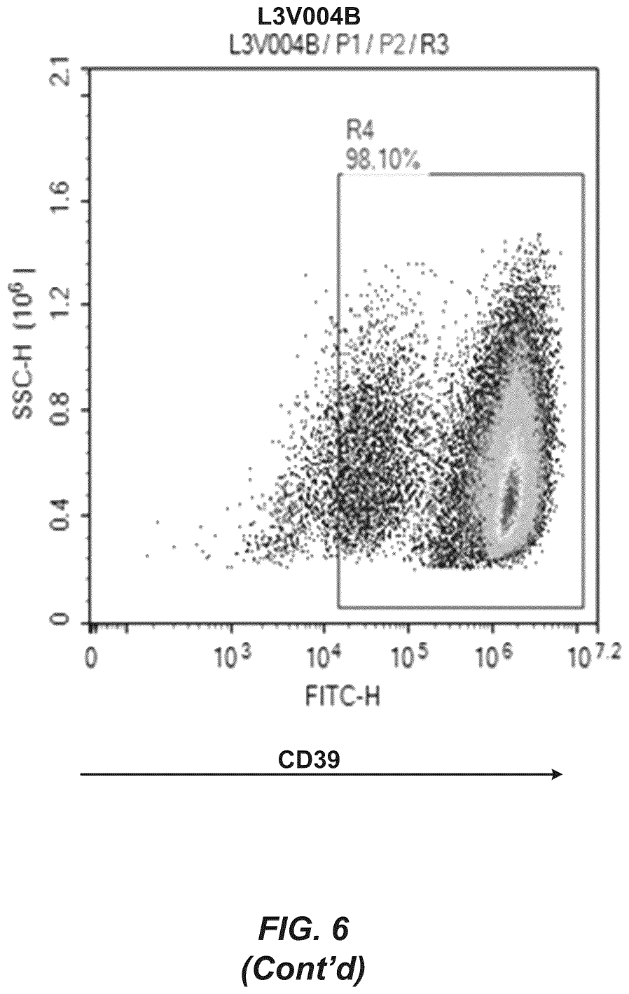

Two days post electroporation, the cells were enriched for transfected CD39 expressing cells using an antibody against CD39 via fluorescence assisted cell sorting (FACS). These enriched cells were then expanded and sorted again 7 to 10 days post electroporation to enrich for CD39 knock-in. These enriched cells, generated from the clone of X1, represent a bulk transfected population of CD39 positive cells (“L3V003B,” also referred to as “X4”). A guide targeting the TGF-β2 gene was also used to edit the clone of X1 having the CD39 KI to generate a bulk transfected population of CD39 positive cells and TGF-β2 negative cells (“L3V004B,” also referred to as “X4+TGF-β2 KO.” These populations were assessed for CD39 expression by flow cytometry, however the overall percentage was lower than expected so the bulk cells were enriched a third time for CD39 expressing cells and showed >90% CD39 expression by flow cytometry (FIG. 6).

### Example 6

### G-Band Karyotype Analysis of Edited Clones

1 million edited ES cells (see Examples 2 and 4) were passaged into a T-25 culture flask with culture media (DMEM/F12+10% Xeno-free KSR with 10 ng/ml Activin and 10 ng/ml Heregulin). After culturing overnight, three T25 culture flasks were shipped to Cytogenetics Laboratory (Cell Line Genetics, Inc.) for Karyotyping analysis; FISH analysis for Chromosome 1, 12, 17, 20; and array comparative genomic hybridization (aCGH) analysis with standard 8×60K array. The G-banding results of selected B2M KO with MANF-TNFAIP3 (A20)-PD-L-1 KI clones (LIV008 cell lines; Example 2) and TXNIP KO with MANF-P2A-HLA-E KI/B2M KO with TNFAIP3 (A20)-P2A-PD-L-1 KI clones (LIV028 cell lines; Example 4) are shown in Table 10.

TABLE 10
G-BAND KARYOTYPING RESULTS

| | | | | | aCGH |
|---|---|---|---|---|---|
| | | | Karyotyping | FISH | array |
| Cell Line | Type | Passage | analysis | analysis | analysis |
| LIV008-C1 | B2M KO with MANF- | P34 | Normal | 3.5% | PASS |
| | TNFAIP3(A20)-PD-L-1 KI | | | Trisomy | |
| | | | | 1 qp 32.3 | |
| LIV008-C3 | B2M KO with MANF- | P34 | Normal | Normal | PASS |
| | TNFAIP3(A20)-PD-L-1 KI | | | | |
| LIV028-C2 | TXNIP KO with MANF- | P37 | Normal | Normal | PASS |
| | P2A-HLA-E KI; B2M KO | | | | |
| | with A20-P2A-PD-L-1 KI | | | | |
| LIV028-C3 | TXNIP KO with MANF- | P36 | Normal | 8.5% | PASS |
| | P2A-HLA-E KI; B2M KO | | | duplication | |
| | with A20-P2A-PD-L-1 KI | | | of MDM4 | |
| LIV028-C17 | TXNIP KO with MANF- | P36 | Normal | Normal | PASS |
| | P2A-HLA-E KI; B2M KO | | | | |
| | with A20-P2A-PD-L-1 KI | | | | |
| LIV028-C18 | TXNIP KO with MANF- | P38 | Normal | Normal | PASS |
| | P2A-HLA-E KI; B2M KO | | | | |
| | with A20-P2A-PD-L-1 KI | | | | |
| LIV028-C21 | TXNIP KO with MANF- | P38 | Normal | 8.5% | PASS |
| | P2A-HLA-E KI; B2M KO | | | duplication | |
| | with A20-P2A-PD-L-1 KI | | | of MDM4 | |
| LIV028-C24 | TXNIP KO with MANF- | P36 | Normal | Normal | PASS |
| | P2A-HLA-E KI; B2M KO | | | | |
| | with A20-P2A-PD-L-1 KI | | | | |

### Example 7

### Differentiation of Edited Human Embryonic Stem Cells to Pancreatic Endoderm Cells (PECs)

### Maintenance of Edited Human Embryonic Stem Cells(ES)

The edited human pluripotent stem cells at various passages (P38-42) were maintained by seeding at 33,000 cells/cm² for a 4-day passage or 50,000 cells/cm² for a 3-day passage with hESM medium (DMEM/F12+10% KSR+10 ng/ml Activin A and 10 ng/mL Heregulin) and final 10% human AB serum.

### Aggregation of Edited Human Embryonic Stem Cells for PECs Differentiation

The edited cells were dissociated into single cells with ACCUTASE® and then centrifuged and resuspended in 2% StemPro (Cat #A1000701, Invitrogen, CA) in DMEM/F12 medium at 1 million cells per ml, and total 350-400 million of cells were seeded in one 850 cm² roller bottle (Cat #431198, Corning, NY) with rotation speed at 8 RPM=0.5 RPM for 18-20 hours before differentiation. The aggregates from edited human pluripotent stem cells were differentiated into pancreatic lineages using in roller bottles as described in Schulz et al. (2012) PLOS ONE 7 (5): e37004. Aggregates from edited human pluripotent stems cells were differentiated into pancreatic lineages as described in Rezania et al. (2014) Nat. Biotechnol. 32 (11): 1121-1133 and US20200208116.

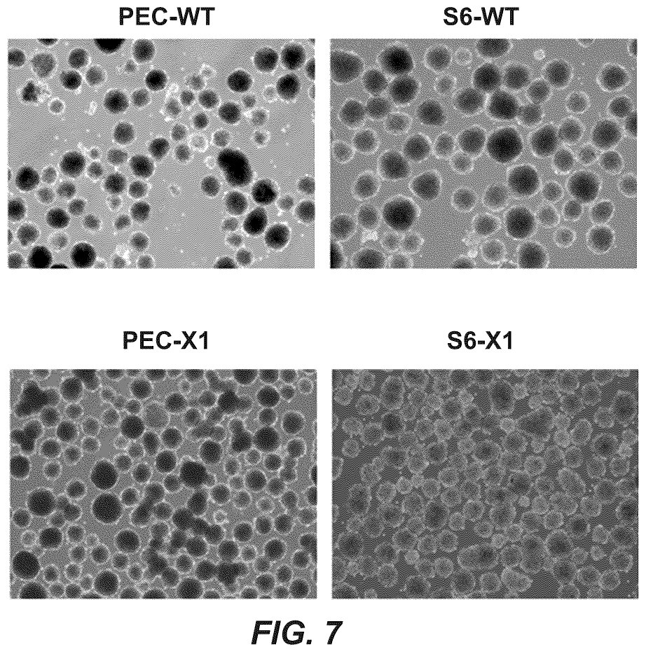

FIG. 7 shows similar morphology among TXNIP KO/MANF-P2A-HLA-E KI & B2M KO/TNFAIP3 (A20)-P2A-PD-L-1 KI clones (“X1” or LIV028 cell line) at PEC stage and Stage 6 (S6) and those differentiated from wild-type cells.

### Example 8

### Gene Expression at PEC Stage and Stage 6

Targeted RNAseq for gene expression analysis was performed using Illumina TruSeq and a custom panel of oligos targeting 111 genes. The panel primarily contained genes that are markers of the developmental stages during pancreatic differentiation. At end of PEC stage and Stage 6, 10 μL APV (aggregated pellet volume) was collected and extracted using the Qiagen RNeasy or RNeasy 96 spin column protocol, including on-column DNase treatment. Quantification and quality control were performed using either the TapeStation combined with Qubit, or by using the Qiagen QIAxcel. 50-200 ng of RNA was processed according to the Illumina TruSeq library preparation protocol, which consists of cDNA synthesis, hybridization of the custom oligo pool, washing, extension, ligation of the bound oligos, PCR amplification of the libraries, and clean-up of the libraries, prior to quantification and quality control of the resulting dsDNA libraries using either the TapeStation combined with Qubit, or by using the Qiagen QIAxcel. The libraries were subsequently diluted to a concentration of 4 nM and pooled, followed by denaturing, spike in of PhiX control, and further dilution to 10-12 pM prior to loading on the Illumina MiSeq sequencer. Following the sequencing run, initial data analysis was performed automatically through BaseSpace, generating raw read counts for each of the custom probes. For each gene, these read counts were then summed for all probes corresponding to that gene, with the addition of 1 read count (to prevent downstream divisions by 0). Normalization was performed to the gene SF3B2, and the reads were typically visualized as fold change vs. Stage 0. When the data was processed for principal component analysis, normalization was performed using the DEseq method.

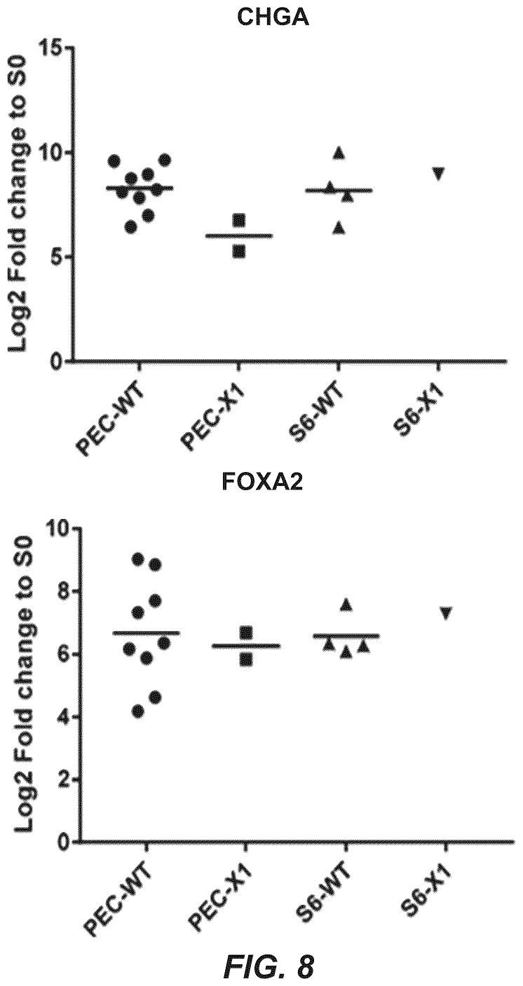

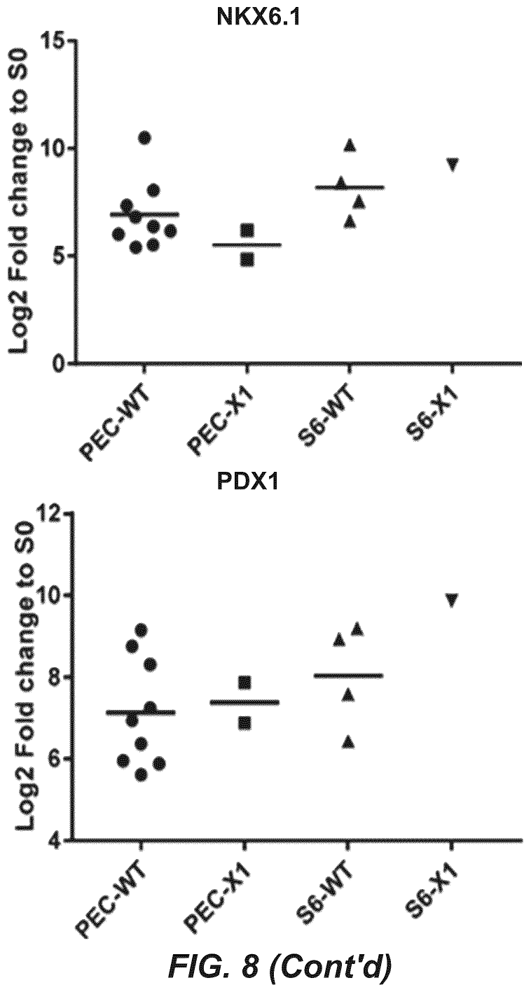

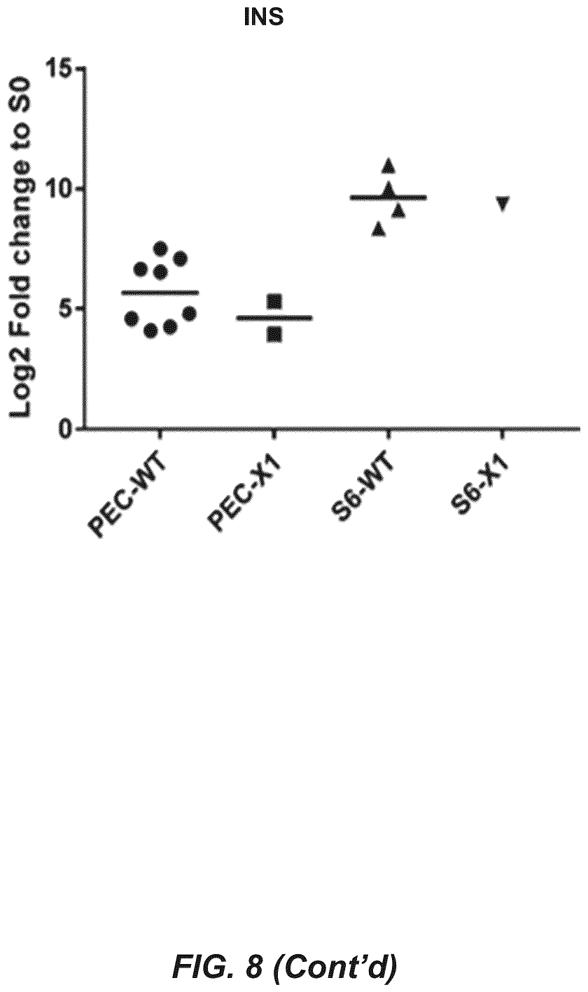

Selected gene expression is shown in FIG. 8. The expression pattern of CHGA, FOXA2, NKX6.1, PDX1 and INS from the “X1” clones, i.e., TXNIP KO/MANF-P2A-HLA-E KI & B2M KO/TNFAIP3 (A20)-P2A-PD-L-1 KI (X1), at PEC stage and Stage 6 (S6) was similar to that of cells differentiated from wild-type cells.

### Example 9

### Flow Cytometry for CHGA, PDX1 and NKX6.1 at PEC Stage and Stage 6

PEC stage and stage 6 aggregates were washed with PBS and then enzymatically dissociated to single cells suspension at 37° C. using ACCUMAX™ (Catalog #A7089, Sigma, MO). MACS Separation Buffer (Cat #130-091-221, Miltenyi Biotec, North Rhine-Westphalia, Germany) was added and the suspension was passed through a 40 μm filter and pelleted. For intracellular marker staining, cells were fixed for 30 mins in 4% (wt/v) paraformaldehyde, washed in FACS Buffer (PBS, 0.1% (wt/v) BSA, 0.1% (wt/v) NaN3) and then cells were permeabilized with Perm Buffer (PBS, 0.2% (v/v) Triton X-100 (Cat #A16046, Alfa Aesar, MA), 5% (v/v)normal donkey serum, 0.1% (wt/v) NaN3) for 30 mins on ice and then washed with washing buffer (PBS, 1% (wt/v) BSA, 0.1% (wt/v) NaN3). Cells were incubated with primary antibodies (Table 11) diluted with Block Buffer (PBS, 0.1% (v/v) Triton X-100, 5% (v/v)normal donkey serum, 0.1% (wt/v) NaN3) overnight at 4° C. Cells were washed in IC buffer and then incubated with appropriate secondary antibodies for 60 mins at 4° C. Cells were washed in IC buffer and then in FACS Buffer. Flow cytometry data were acquired with NovoCyte Flow Cytometer (ACEA Biosciences, Brussels). Data were analyzed using FlowJo software (Tree Star, Inc.). Intact cells were identified based on forward (low angle) and side (orthogonal,) 90° light scatter. Background was estimated using antibody controls and undifferentiated cells. In the figures, a representative flow cytometry plot is shown for one of the sub-populations. Numbers reported in the figures represent the percentage of total cells from the intact cells gate.

TABLE 11
ANTIBODIES FOR FLOW CYTOMETRY

| Antigen | Fluorophore | Source | Dilution |
|---|---|---|---|
| PDX1 | PE | BD Bioscience (Cat#562161) | 1:2.5 |
| NKX6.1 | AF647 | BD Bioscience (Cat#563338) | 1:2.5 |
| CHGA | AF405 | Novus (Cat#NBP2-33198AF405) | 1:1000 |

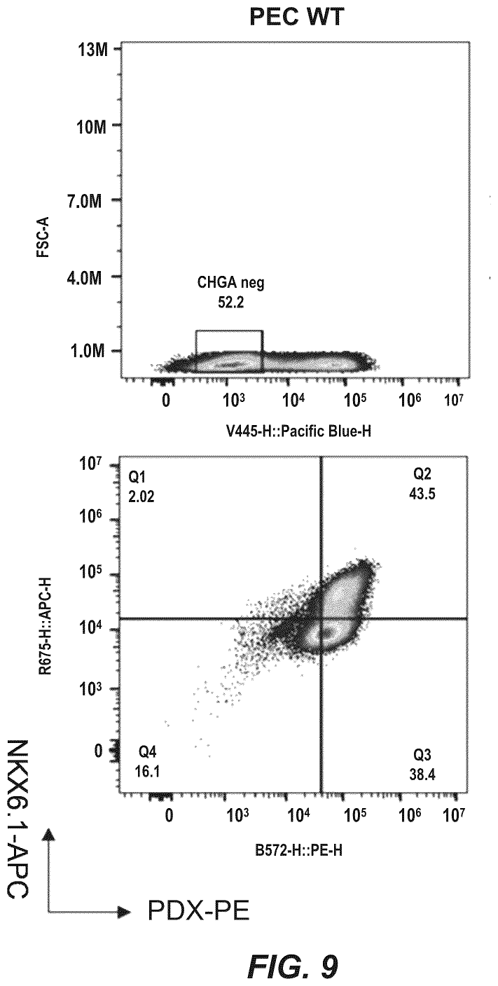

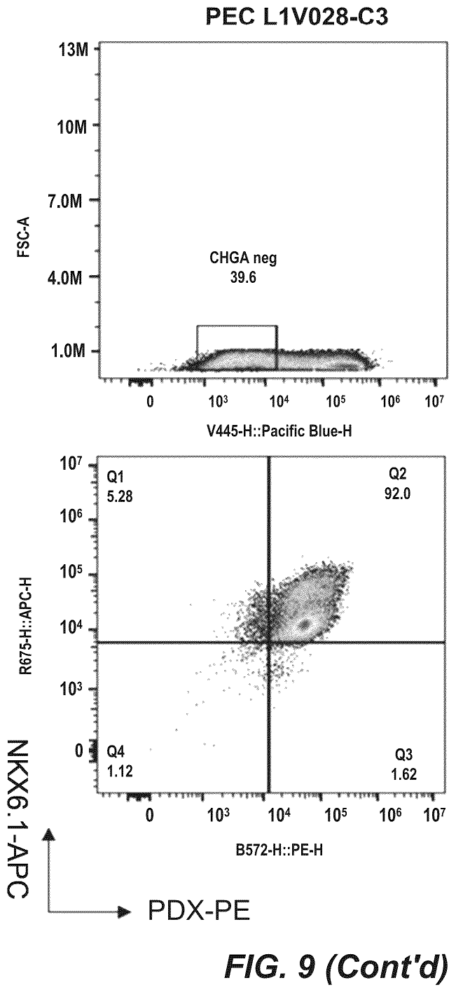

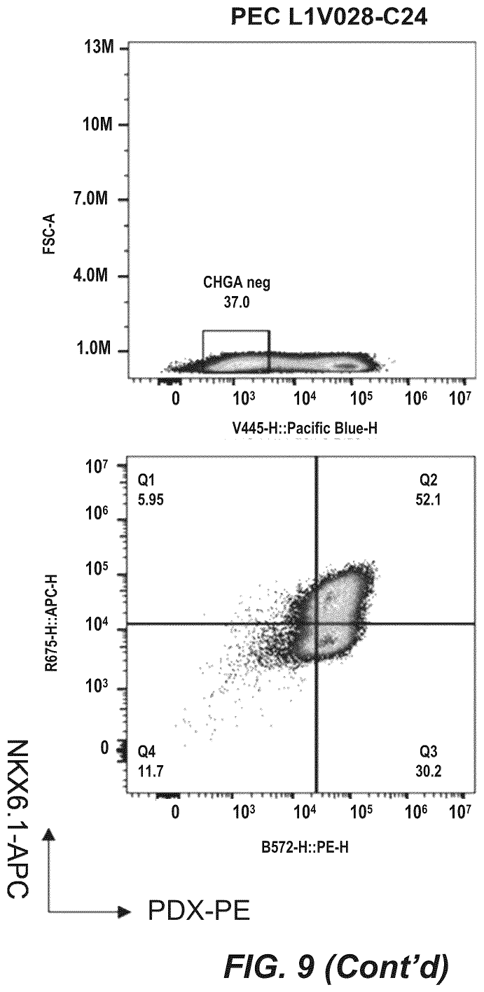

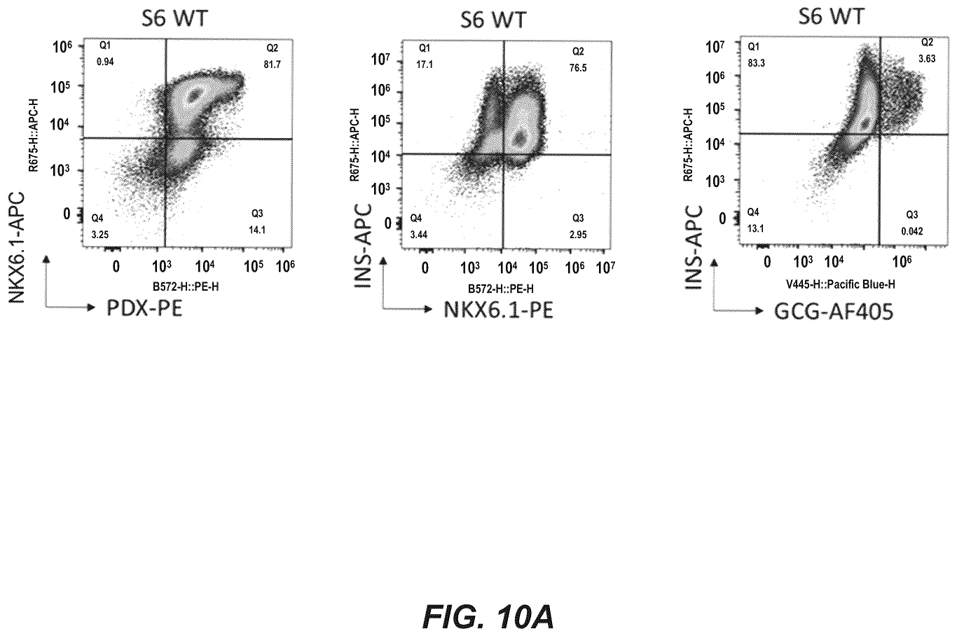

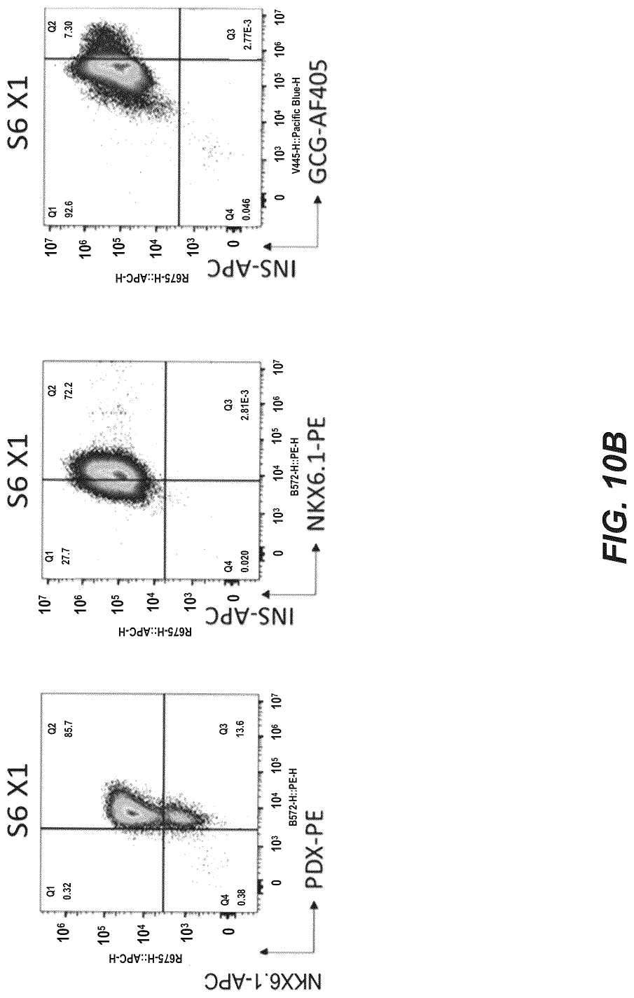

FIG. 9 presents flow cytometry for CHGA, PDX1 and NKX6.1 in PECs differentiated from wild type cells or two LIV028 clones generated in Example 4 (i.e., TXNIP KO/MANF-P2A-HLA-EKI & B2M KO/TNFAIP3 (A20)-P2A-PD-L-1 KI). FIG. 10A and FIG. 10B presents flow cytometry for CHGA, PDX1 and NKX6.1 in Stage 6 (S6) cells differentiated from wild type cells (FIG. 10A) or X1 cells (i.e., TXNIP KO/MANF-P2A-HLA-E KI & B2M KO/TNFAIP3 (A20)-P2A-PD-L-1 KI) (FIG. 10B).

### Example 10

In vivo Efficacy Study of B2M KO/MANF-P2A-TNFAIP3-P2A-PD-L-1 KI Cells

Pancreatic endoderm cells were generated from the B2M KO/MANF-P2A-TNFAIP3 (A20)-P2A-PD-L-1 KI (L1V008) cell line described above in Example 2 and a clonal unmodified cell line obtained from transfection with a non-cutting guide-RNA (NCG).

Pancreatic endoderm aggregates derived from the indicated clonal lines were loaded into perforated cell delivery devices (PD) to produce test or control articles. The PDs permitted direct vascularization upon subcutaneous transplantation, and the encapsulated pancreatic progenitor cells matured in vivo into functional pancreatic endocrine cells including glucose-responsive, insulin-producing cells.

As summarized in Table 12, the LIV008 and control cells were tested in four groups of athymic nude rats in which each was implanted subcutaneously with two articles, each containing approximately 7×10⁶ cells.

TABLE 12
STUDY DESIGN

| | | Genetic Modification | | | GSIS |
|---|---|---|---|---|---|
| | | Knock-out | | Number | Test |
| Group | Group | (Loss of | Knock-in | of | Time |
| Number | ID | Function) | (Gain of Function) | Animals | Points |
| 1 | LIV009B | B2M | MANF, TNFAIP3, | 8 per | Week |
| | (Bulk) | | PD-L-1 | Group | 12, 16, |
| 2 | L1V008-C1 | B2M | MANF, TNFAIP3, | | 20, 24 |
| | | | PD-L-1 | | |
| 3 | L1V008-C3 | B2M | MANF, TNFAIP3, | | |
| | | | PD-L-1 | | |
| 4 | Control | None | None | | |

Starting at 12 weeks all surviving animals were subjected to efficacy evaluation through glucose stimulated insulin secretion (GSIS) testing. Blood samples were obtained from non-fasted animals prior to and after intraperitoneal administration of 3 g/kg glucose. Serum concentrations of human C-peptide were determined through standard enzyme linked immunosorbent assays. The C-peptide reading for the control group (GRP 4) was taken 60 min after intraperitoneal administration of glucose, while the readings for the experimental groups were taken 90 min post administration.

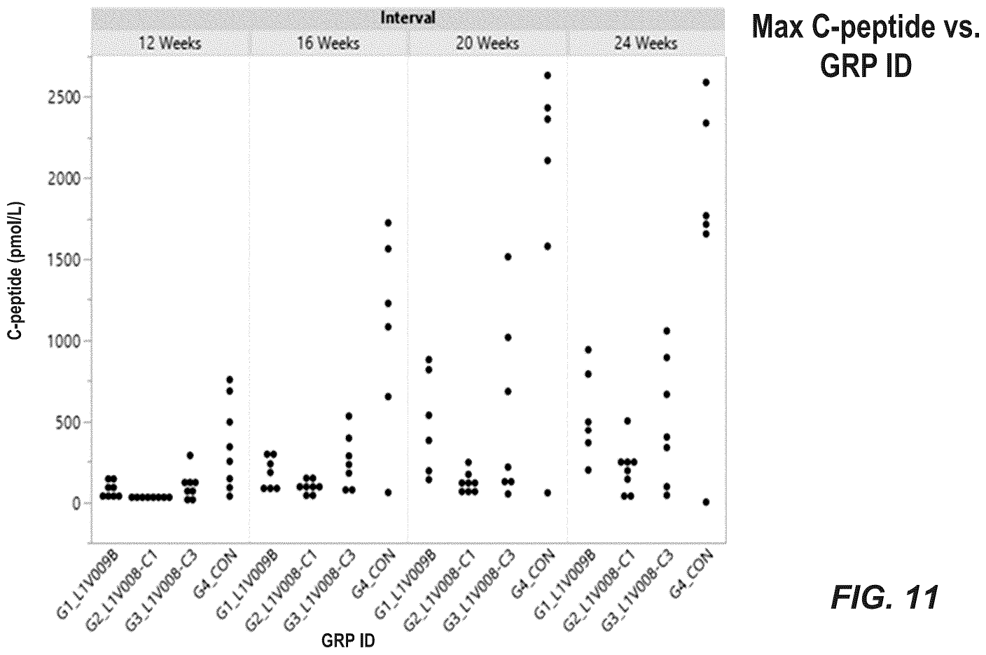

FIG. 11 presents the C-peptide levels for the four groups at 12, 16, 20 and 24 weeks. Results indicated there were no substantial differences between experimental groups. These results indicated that neither the genetic modifications that were introduced nor the manipulations required to generate clonal lines affected the ability for the cell lines in question to differentiate into pancreatic endoderm cells in vitro and subsequently generate functional beta cells in vivo.

### Example 11

In vivo Efficacy Study of B2M KO/CD39-P2A-PD-L-1 KI Cells

Pancreatic endoderm aggregates derived from the B2M KO/CD39-P2A-PD-L-1 KI (L1V017) cell line prepared in Example 3 or from control cells were loaded into perforated devices and implanted into animals for GSIS testing as described above in Example 10. Table 13 presents the study design.

TABLE 13
STUDY DESIGN

| | | Genetic Modification | | | |
|---|---|---|---|---|---|
| | | Knock-out | Knock-in | Number | |
| Group | | (Loss of | (Gain of | of | GSIS Test |
| Number | Group ID | Function) | Function | Animals | Time Points |
| 1 | L1V017B | B2M | CD39, | 6 per | Week |
| | (Bulk) | | PD-L-1 | group | 12, 16, |
| 2 | Control | None | None | | 20, 24 |

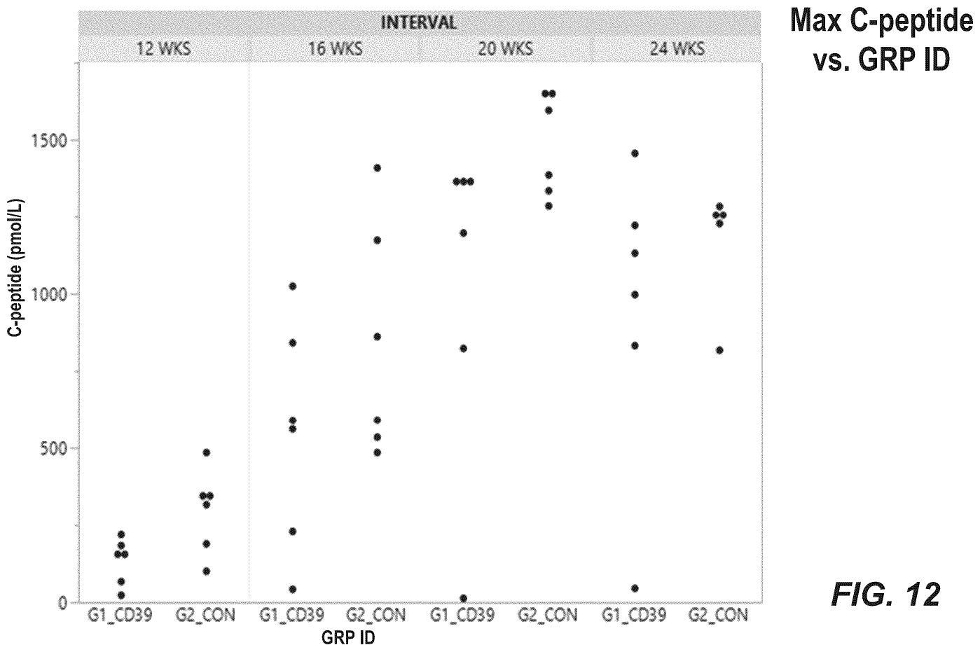

As shown in FIG. 12, the genetic modifications and manipulations required to generate this cell line did not affect the cells' ability cells to differentiate into pancreatic endoderm cells in vitro and subsequently generate functional beta cells in vivo.

### Example 12

In vivo Efficacy Study of TXNIP KO/MANF-P2A-HLA-E KI & B2M KO/TNFAIP3 (A20)-P2A-PD-L-1 KI Cells

PEC stage and stage 6 cells differentiated from control cells (NCG) or a LIV028 clone generated in Example 4 (i.e., TXNIP KO/MANF-P2A-HLA-E KI & B2M KO/TNFAIP3 (A20)-P2A-PD-L-1 KI; “X1”) were tested for in vivo efficacy. Test or control capsules were transplanted into the left kidney of NSG mice (Jackson Laboratory Stock No: 005557). Table 14 presents the study design.

TABLE 14
STUDY DESIGN

| | | Genetic Modification | | | | | Read out |
|---|---|---|---|---|---|---|---|
| | | Knock-out | Knock-in | | Number | Trans- | of C- |
| Group | Group | (Loss of | (Gain of | | of | plantation | peptide |
| Number | ID | Function) | Function) | Stage | Animals | site | serum |
| 1 | NCG | None | None | PEC | 5 per | Kidney | Week 12, |
| 2 | L1V028- | TXNIP | PD-L-1 | PEC | Group | capsule | 16, 20, 24 |
| | C24 | B2M | HLA-E | | | (left Side) | |
| | (X1) | | MANF | | | | |
| | | | TNFAIP3 | | | | |
| 3 | NCG | None | None | S6 | | | |
| 4 | L1V028- | TXNIP | PD-L-1 | S6 | | | |
| | C24 | B2M | HLA-E | | | | |
| | (X1) | | MANF | | | | |
| | | | TNFAIP3 | | | | |
| 5 | V1B-H9 | TXNIP | PD-L-1 | S6 | | | |
| | | B2M | HLA-E | | | | |

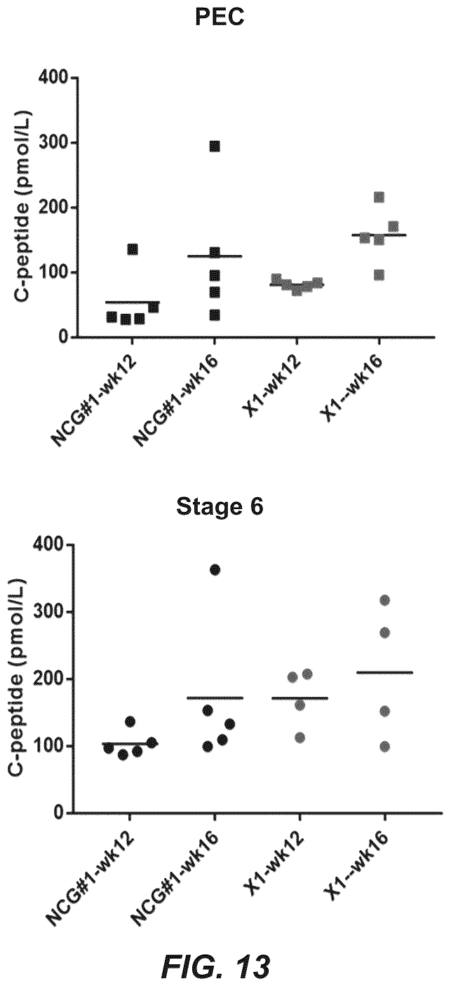

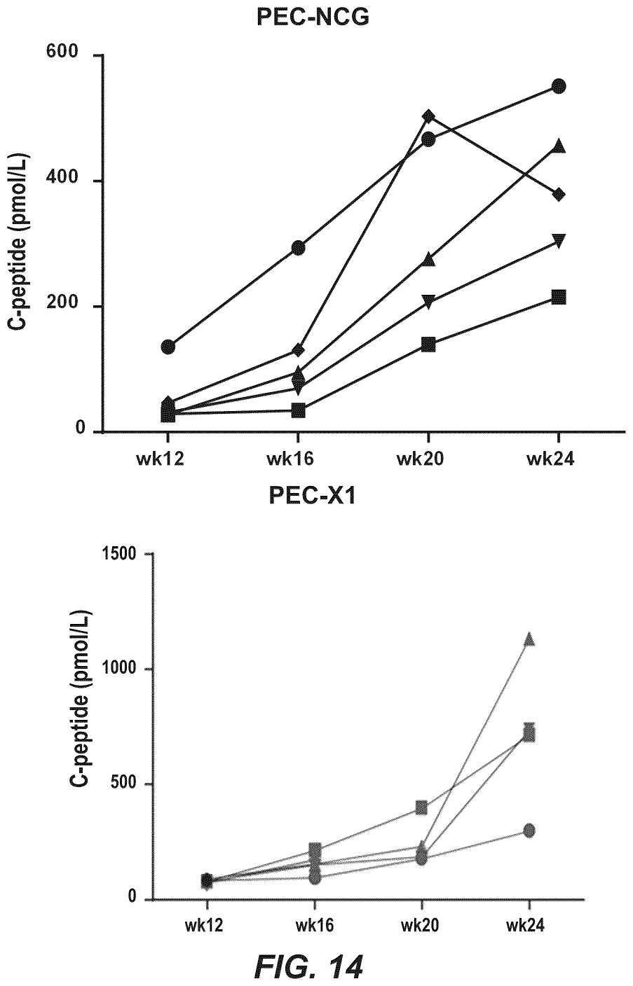

GSIS testing was performed at 12, 16, 20 and 24 weeks. FIG. 13 presents the C-peptide levels at weeks 12 and 16 for individual animals in the PEC-control, PEC-X1, S6-control, and S6-X1 groups. FIG. 14 presents a time course of the mean C-peptide levels from week 12 to week 24 for PEC-control and PEC-X1 groups. These results show that the X1 cells are able to differentiate into pancreatic endoderm cells in vitro and subsequently generate functional beta cells in vivo.

At 26 weeks, after GSIS testing, animals were euthanized and explanted test articles were fixed in neutral buffered formalin, processed to slides, and stained with H&E and by immunohistochemistry for insulin and glucagon.

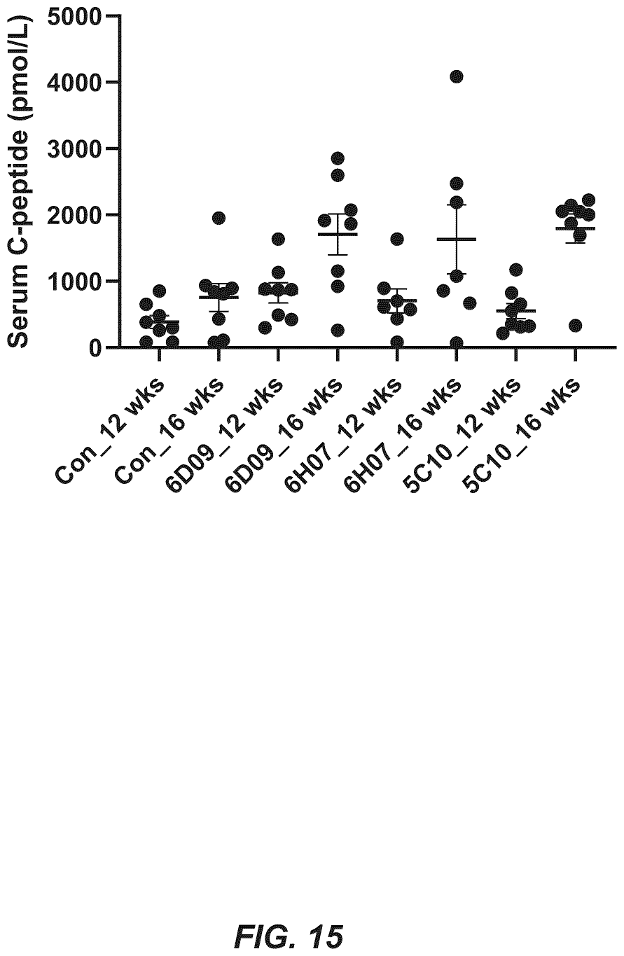

Several seed run clones from the “X1” cell line (i.e., LIV028) were also tested in vivo. The clones were selected based on whole genome sequencing. They had Het/Hom on-site genotypes, exhibited no unintended plasmid insertions, and did not exhibit any variants that may have functionally altered oncogenes. Clone 6D09 had no putative off-target insertions, whereas clones 6H07 and 5C10 has at least one putative off-target insertion. GSIS testing was performed at weeks 12 and 16. FIG. 15 presents C-peptide levels for each animal and group mean levels at 12 weeks, 16 weeks, and 20 weeks. Clones 6D09, 6H07, and 5C10 exhibited good in vivo efficiency.

### Example 13

Generation of B2M Knock Out (KO) with CD39-P2A-CD73-P2A-PD-L-1 KI Human Pluripotent Stem Cells

Cells were generated in which a transgene encoding CD39-P2A-CD73-P2A-PD-L-1 was inserted into the B2M gene locus thereby knocking out the B2M gene.

Human pluripotent stem cells were electroporated essentially as described above in Example 2 with a B2M-CAGGS-CD39-P2A-CD73-P2A-PD-L-1 donor plasmid, as detailed below in Table 15, and an RNP comprising Cas9 and a B2M-2 gRNA (comprising a spacer sequence of SEQ ID NO: 62).

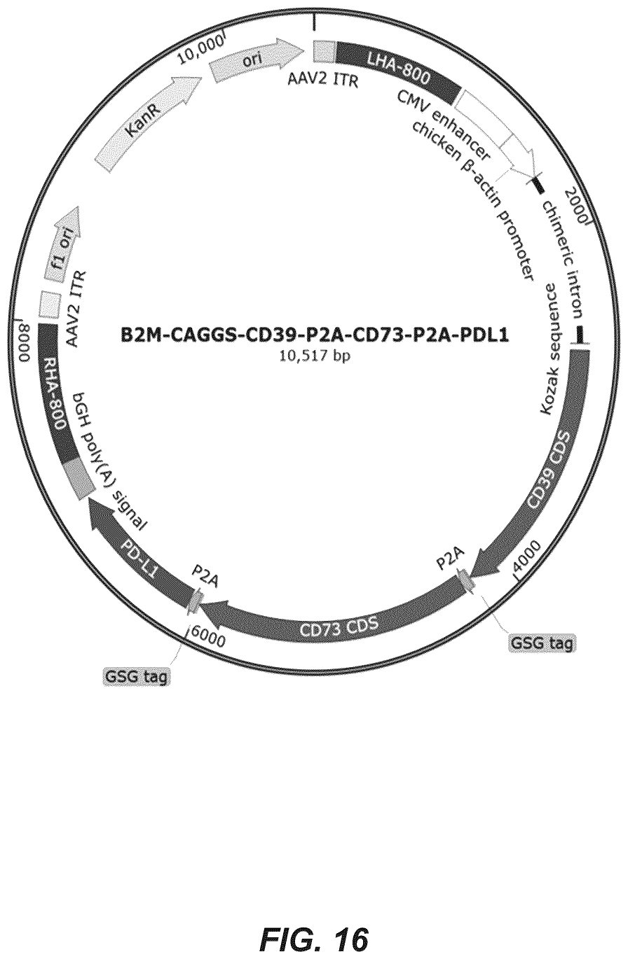

FIG. 16 presents a schematic of the B2M-CAGGS-CD39-P2A-CD73-P2A-PD-L-1 plasmid and Table 15 identifies the elements and locations therein. The B2M-CAGGS-CD39-P2A-CD73-P2A-PD-L-1 donor plasmid comprises a CAGGS promoter to drive expression of cDNA of CD39-P2A-CD73-P2A-PD-L-1 (SEQ ID NO: 56) flanked by 800 base pair homology arms with identical sequence to the B2M locus around exon 1. The complete sequence of the B2M-CAGGS-CD39-P2A-CD73-P2A-PD-L-1 donor plasmid comprises the nucleotide sequence of SEQ ID NO: 47.

TABLE 15
ELEMENTS OF B2M-CAGGS-CD39-P2A-CD73-P2A-PD-L-1
DONOR PLASMID

| Element | Location (size in bp) | | SEQ ID NO: |
|---|---|---|---|
| Left ITR | 1-130 | (130) | 14 |
| LHA-B2M | 145-944 | (800) | 15 |
| CAGGS promoter | 973-2639 | (1667) | 16 |
| CD39 | 2684-4213 | (1530) | 27 |
| P2A | 4223-4279 | (57) | 18 |
| CD73 | 4280-6001 | (1722) | 46 |
| P2A | 6011-6067 | (57) | 18 |
| PD-L-1 | 6068-6940 | (873) | 20 |
| bGH poly(A) signal | 6958-7182 | (225) | 21 |
| RHA-B2M | 7189-7988 | (800) | 22 |
| Right ITR | 8030-8170 | (141) | 23 |
| Entire plasmid | 10,517 | bp | 47 |

Seven to ten days post electroporation, the cells were enriched for PD-L-1 expressing cells via magnetic assisted cell sorting (MACS) using anti-mouse IgG Dynabeads (ThermoFisher, CELLection™ Pan Mouse IgG Kit, 11531D. These enriched cells represented a bulk KI population that was highly PD-L-1 positive. The enriched cells were then FACS-sorted for PD-L-1 surface expression using the WOLF FACS-sorter (Nanocellect) into BIOLAMININ 521 CTG coated 96-well plates with StemFlex and RevitaCell™. To detect the PD-L-1 surface expression, anti-PD-L-1 fluorescent antibodies were used (see Table 4). For FACS-sorting, unedited cells served as a negative control. PD-L-1 positive cells were selected for sorting and single cell cloning.

Plated single cells were grown in a normoxia incubator (37° C., 8% CO₂) with every other day media changes until colonies were large enough to be re-seeded as single cells. When confluent, samples were split for maintenance and genomic DNA extraction. Correctly targeted clones were identified via PCR for the CD39-P2A-CD73-P2A-PD-L-1 KI insertion using primers that amplify from outside the plasmid homology arms at the site of insertion into the B2M locus, enabling amplification of the KI integrated DNA only. The B2M KO state of clones was confirmed via PCR and Sanger sequencing. The correct KI and KO clones (LIV018B cell line) were expanded in increasing tissue culture formats until a population size of 30 million cells was reached.

### Example 14

Generation of B2M KO with TNFAIP3 (A20)-P2A-PD-L-1 KI Human Pluripotent Stem Cells

Human pluripotent stem cells were electroporated essentially as described above in Example 2 with a B2M-CAGGS-TNFAIP3 (A20)-P2A-PD-L-1 donor plasmid (SEQ ID NO: 31, Table 6) and an RNP comprising Cas9 and B2M-2 gRNA (comprising a spacer sequence of SEQ ID NO: 62) to generate a LIV019B cell line. FIG. 3 presents a schematic of the B2M-CAGGS-TNFAIP3-P2A-PD-L-1 donor plasmid (also called X1-1 cassette).

Seven to ten days post electroporation, the cells were enriched for PD-L-1 expressing cells via magnetic assisted cell sorting (MACS) using anti-mouse IgG Dynabeads (ThermoFisher, CELLection™ Pan Mouse IgG Kit, 11531D. These enriched cells represented a bulk KI population that was highly PD-L-1 positive. The enriched cells were then FACS-sorted for PD-L-1 surface expression using the WOLF FACS-sorter (Nanocellect) into BIOLAMININ 521 CTG coated 96-well plates with StemFlex and RevitaCell™. To detect the PD-L-1 surface expression, anti-PD-L-1 fluorescent antibodies were used (see Table 4). For FACS-sorting, unedited cells served as a negative control. PD-L-1 positive cells were selected for sorting and single cell cloning.

Plated single cells were grown in a normoxia incubator (37° C., 8% CO₂) with every other day media changes until colonies were large enough to be re-seeded as single cells. When confluent, samples were split for maintenance and genomic DNA extraction. Correctly targeted clones were identified via PCR for the A20-P2A-PD-L-1 KI insertion using primers that amplify from outside the plasmid homology arms at the site of insertion into the B2M locus, enabling amplification of the KI integrated DNA only. The B2M KO state of clones was confirmed via PCR and Sanger sequencing. The correct KI and KO clones (LIV019B cell line) were expanded in increasing tissue culture formats until a population size of 30 million cells was reached.

### Example 15

### Differentiation and Characterization of Additional Edited Cell Lines

Cells from the LIV017B cell line (i.e., CD39-P2A-PD-L-1 KI and B2M KO) prepared above in Example 3, the LIV018B cell line (i.e., CD39-P2A-CD73-P2A-PD-L-1 KI and B2M KO) prepared above in Example 13, and the LIV019B cell line (i.e., TNFAIP3 (A20)-P2A-PD-L-1 KI and B2M KO) prepared above in Example 14 were differentiated essentially as described above in Example 7.

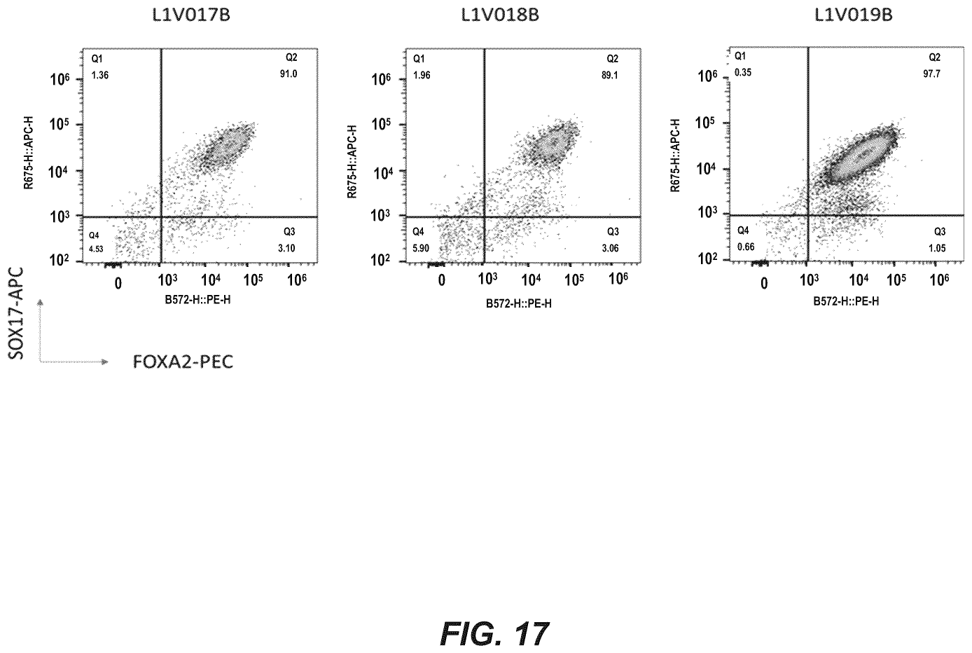

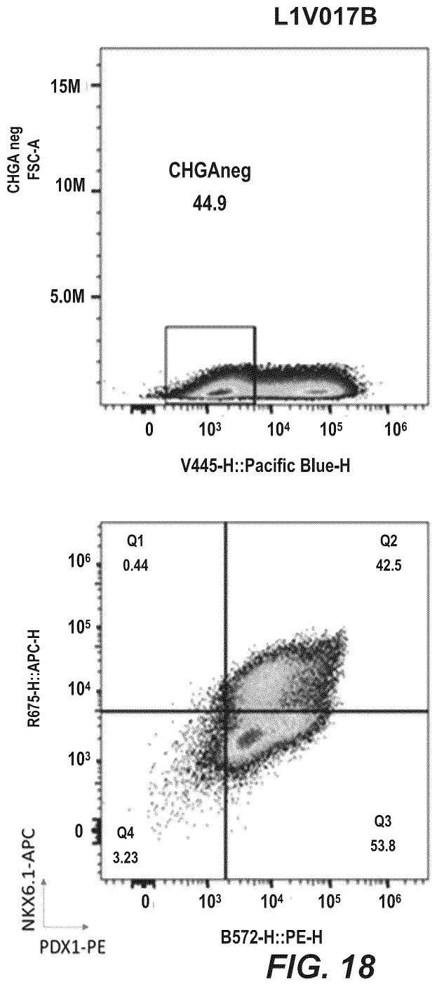

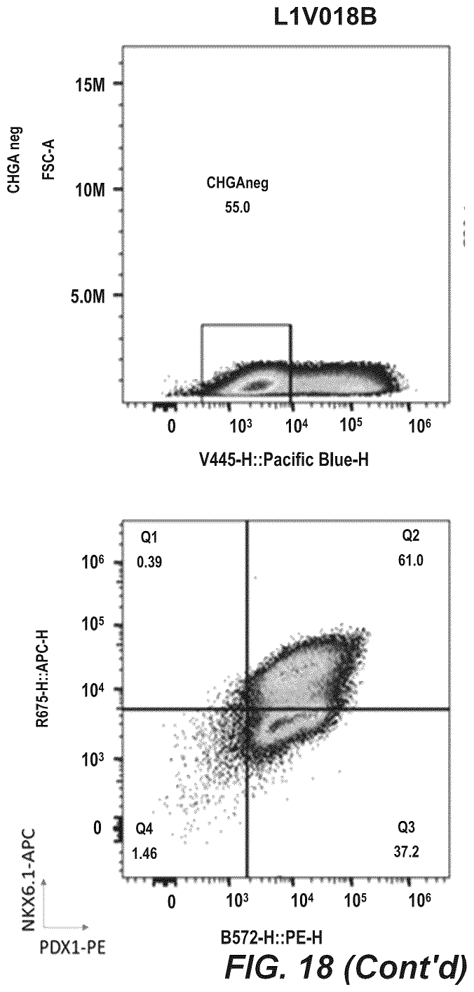

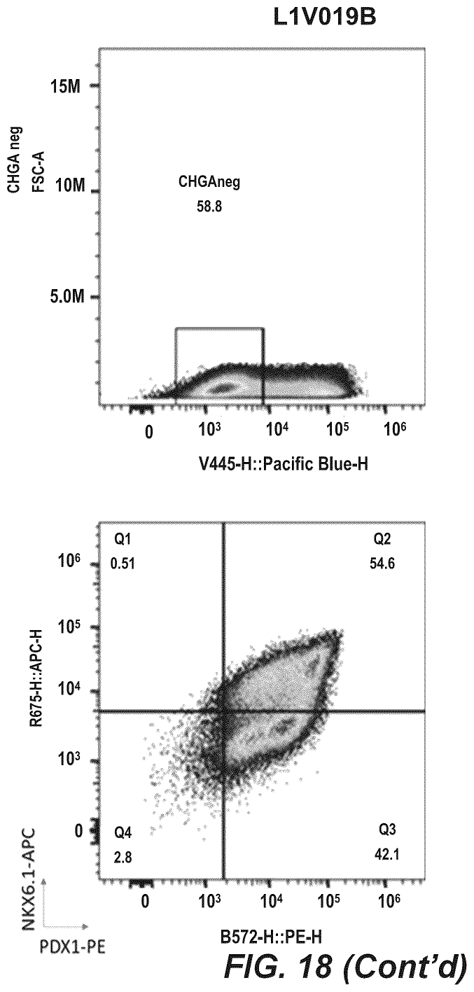

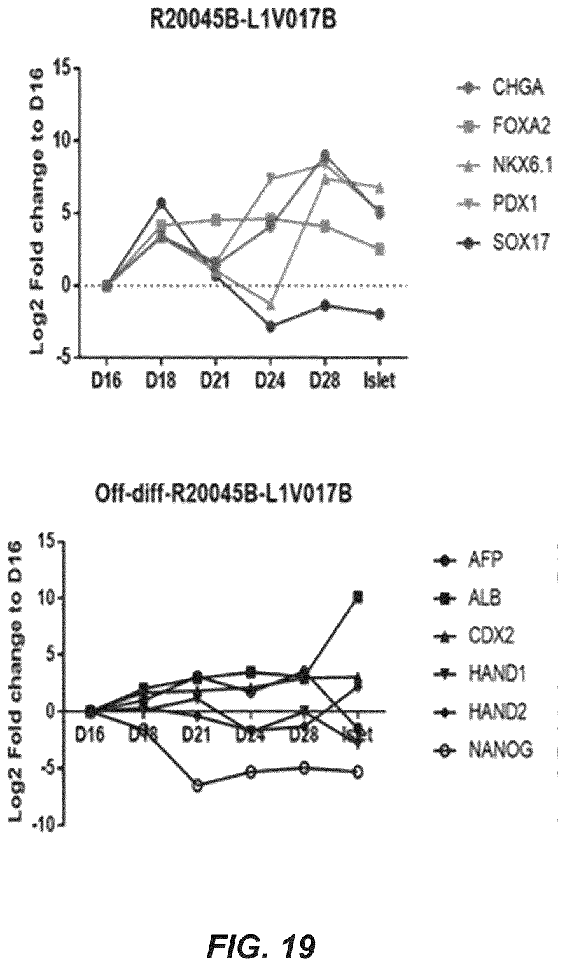

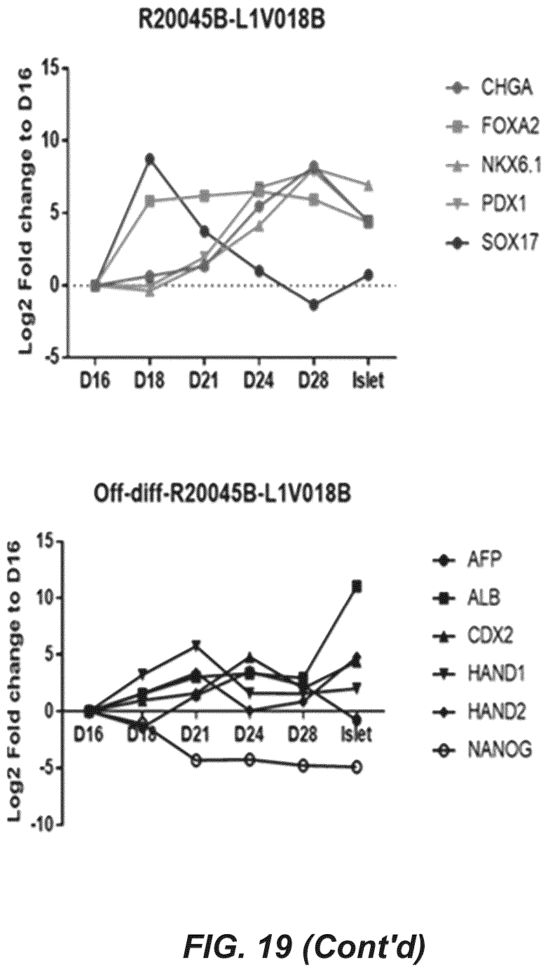

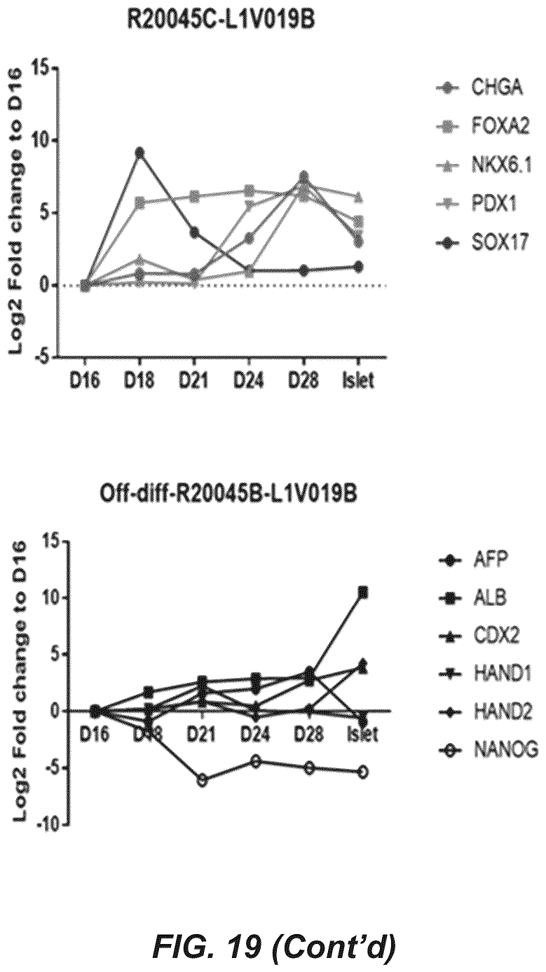

Gene expression was examined at various time points during the differentiation process essentially described above in Examples 11 and 12. FIG. 17 presents flow cytometry for SOX17 and FOXA2 expression at day 18 to confirm presence of DE (definitive endoderm) cells. Presence of differentiated pancreatic endodermal cells (PEC) was further confirmed by flow cytometry by the presence of a CHGA negative and PDX1 and NKX6.1 positive predominant population (see FIG. 18). The time course of expression from day 16 to islets cells of various makers (e.g., CHGA, FOXA2, NKX6.1, PDX1, SOX17, AFP, ALB, CDX2, HAND1, HAND2, NANOG) is shown in FIG. 19.

### Example 16

### Differentiation of Edited Human Embryonic Stem Cells to Pancreatic Endoderm Cells (PECs) Maintenance of Edited Human Embryonic Stem Cells(ES)

The edited human pluripotent stem cells comprising a B2M KO with TNFAIP3-P2A-PD-L-1 KI, TXNIP KO with MANF-P2A-HLA-E KI, CIITA KO with CD39 KI (“X4”; see Example 7) at various passages (P38-42) were maintained by seeding at about 33,000 cells/cm² for a 4-day passage or about 50,000 cells/cm² for a 3-day passage with hESM medium (DMEM/F12+10% KSR+10 ng/ml Activin A and 10 ng/mL Heregulin) and final 10% human AB serum.

### Aggregation of Edited Human Embryonic Stem Cells for PECs Differentiation

The edited cells were dissociated into single cells with ACCUTASE® and then centrifuged and resuspended in 2% StemPro (Cat #A1000701, Invitrogen, CA) in DMEM/F12 medium at 1 million cells per ml, and total 350-400 million of cells were seeded in one 850 cm² roller bottle (Cat #431198, Corning, NY) with rotation speed at 8 RPM+0.5 RPM for 18-20 hours before differentiation. The aggregates from edited human pluripotent stem cells were differentiated into pancreatic lineages using in roller bottles as described in Schulz et al. (2012) PLOS ONE 7 (5): e37004 and shown for X1 cells. Aggregates from edited human pluripotent stems cells were differentiated into pancreatic lineages as described in Rezania et al. (2014) Nat. Biotechnol. 32 (11): 1121-1133 and US20200208116.

The expression pattern of CHGA, FOXA2, NKX6.1, PDX1 and INS from the “X4” clones, i.e., TXNIP KO/MANF-P2A-HLA-E KI, B2M KO/TNFAIP3 (A20)-P2A-PD-L-1 KI, and CIITA KO/CD39 KI, at PEC stage and Stage 6 (S6) was determined to confirm differentiation.

### Example 17

Generation of X1 Human Pluripotent Stem Cells with TGF-β2 KO

Cells were generated in which a transgene encoding TNFAIP3-P2A-PD-L-1 was inserted into the B2M gene locus, a transgene encoding MANF-P2A-HLA-E was inserted into the TXNIP gene locus, a transgene encoding CD39 was inserted into the CIITA gene locus, and the TGF-β2 gene was knocked out thereby the cells had knock outs of the B2M, TXNIP, CIITA and TGF-β2 genes.

Human pluripotent stem cells were electroporated essentially as described above in Example 2 with the B2M-CAGGS-TNFAIP3-P2A-PD-L-1 donor plasmid (SEQ ID NO: 31, Table 6) and an RNP comprising Cas9 and B2M-2 gRNA (comprising a spacer sequence of SEQ ID NO: 62). Seven to ten days post electroporation, the cells were enriched for PD-L-1 expressing (positive) cells via MACS essentially as described in Example 2. After the enriched PD-L-1 positive population was expanded, the cells were electroporated essentially as described above in Example 2 with the TXNIP-CAGGS-MANF-P2A-HLA-E donor plasmid (SEQ ID NO: 45, Table 8) and an RNP comprising Cas9 and TXNIP_Exon 1_T5 gRNA (comprising a spacer sequence of SEQ ID NO: 80). After enrichment for HLA-E positive cells and expansion of PD-L-1 and HLA-E cells, the double positive cells were electroporated with the CIITA-CAGGS-CD39 donor plasmid (SEQ ID NO: 29, Table 9) and an RNP comprising Cas9 and CIITA Ex3_T6 gRNA (comprising a spacer sequence of SEQ ID NO: 74). The cells were enriched for CD39 expressing cells, expanded, and selected for PD-L-1, HLA-E, and CD39 triple positive cells, which were characterized as described above.

Confirmed triple positive cells, which also had B2M, TXNIP, and CIITA genes knocked out, were electroporated with RNP comprising Cas9 and a TGF-β2 gRNA to generate a TGF-β2 knock out. The TGF-β2 gRNA1 (comprising a spacer sequence comprising an RNA sequence corresponding to 5′-GTTCATGCGCAAGAGGATCG-3′ (SEQ ID NO: 57), the PAM is AGG) was used to knock-out the TGF-β2 protein in an X1 clone and an X4 bulk cell lines by causing a frameshift mutation in the TGF-β2 gene exon 1. Electroporation was carried out in these enriched hESC cells using the Neon Electroporator with the RNP mixture of Cas9 protein (Biomay) and guide RNA (IDT) at a molar ratio of 5:1 (gRNA:Cas9) with absolute values of 125 pmol Cas9 and 625 pmol gRNA per 1 million cells. To form the RNP complex, gRNA and Cas9 were combined in one vessel with R-buffer (Neon Transfection Kit) to a total volume of 25-50 μL and incubated for 15 min at room temperature (RT). This mixture was then combined with the cells to a total volume of ˜115 μL using R-buffer. This mixture was then electroporated with 1 pulse for 20 ms at 1500 V. Following electroporation, the cells were pipetted out into a 6 well plate filled with STEMFLEX™ media with REVITACELL™ Supplement (100×) and laminin 511. Cells were cultured in a normoxia incubator (37° C., 8% CO₂). The L3V003B (“X4”) population targeted with the TGF-β2 gRNA was named L3V004B (“X4+TGF-β2 KO”) while the X1 clone population targeted with the TGF-β2 gRNA was named L3V002B (“X1+TGF-β2 KO”). This process was repeated once more for L3V004B population and two times for L3V002B to ensure a high efficiency of TGF-β2 KO.

Plated single cells were grown in a normoxia incubator (37° C., 8% CO₂) with every other day media changes until colonies were large enough to be re-seeded as single cells. When confluent, samples were split for maintenance and genomic DNA extraction. Correctly targeted clones were confirmed by PCR and Sanger sequencing.

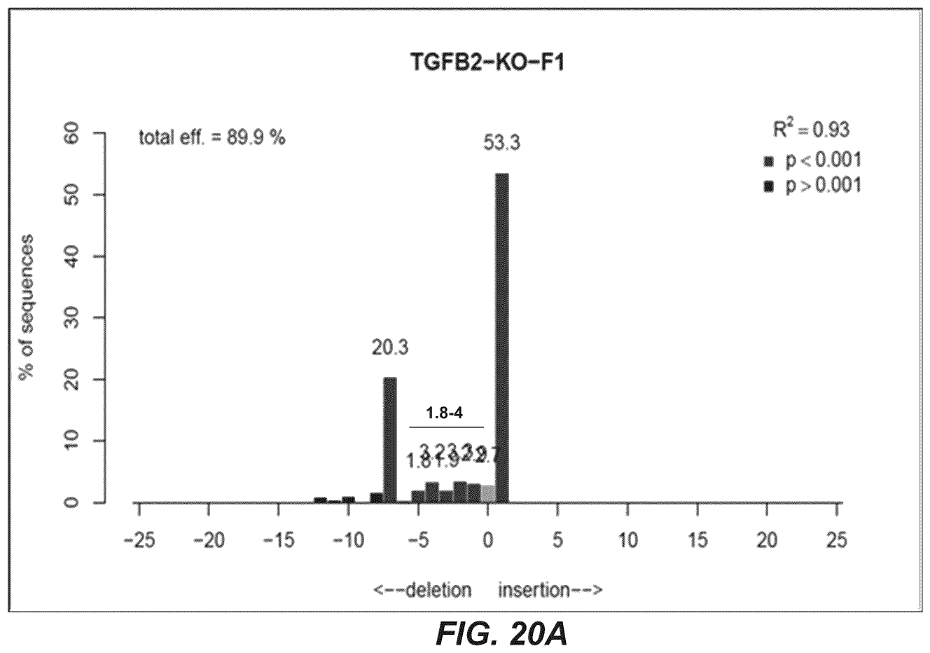

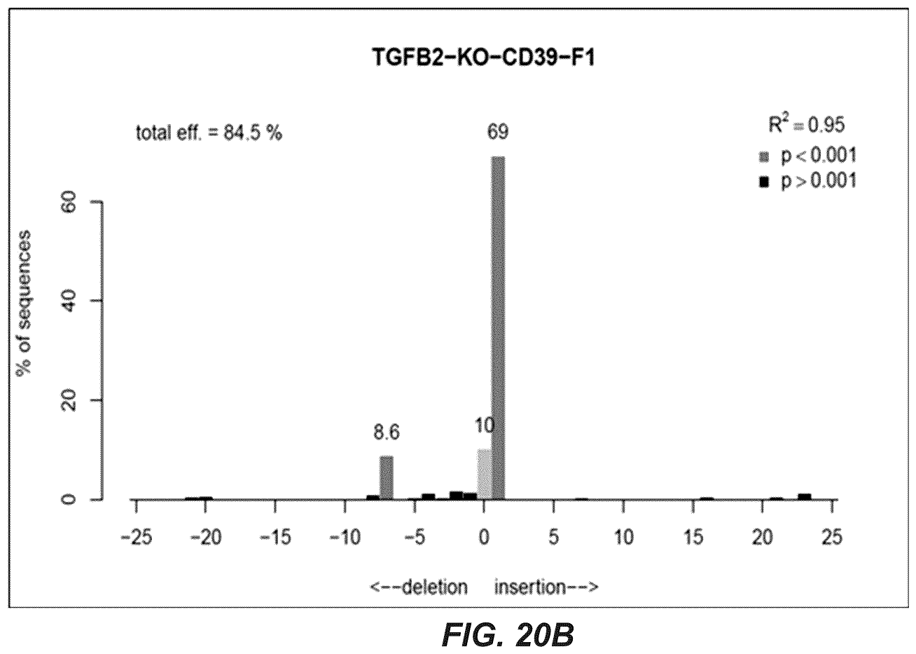

PCR for the target TGF-β2 sequence was performed and the resulting amplified DNA was assessed for cutting efficiency by T1DE analysis. PCR for relevant regions was performed using Platinum Taq Supermix (Invitrogen, cat #125320176 and Cat #11495017). The sequence of the PCR primers are presented in Table 16. FIG. 20A and FIG. 20B show the TGF-β2 KO editing efficiencies for two bulk edited lines L3V002 (“TGFB2-KO-F1”) and L3V004 (“TGFB2-KO-CD39-F1”). Both populations had over 80% KO which was above the desired threshold with+1 and −7 indels being the most prominent edits.

TABLE 16
TGFB2 KO PRIMERS

| | | | SEQ |
|---|---|---|---|
| | | | ID |
| Name | Type | Sequence (5′-3′) | NO: |
| TGF-β2 F1 | forward | AGGATACGTTTTTCTGTTGGGC | 59 |
| TGF-β2 R1 | reverse | GGAGAACGGGAAAAGAGCGA | 60 |

### Example 18

### Differentiation of Edited Human Embryonic Stem Cells to Pancreatic Endoderm Cells (PECs) Maintenance of Edited Human Embryonic Stem Cells(ES)

The edited human pluripotent stem cells comprising a B2M KO with TNFAIP3-P2A-PD-L-1 KI, TXNIP KO with MANF-P2A-HLA-E KI, CIITA KO with CD39 KI, and TGF-β2 KO (“X4+TGF-β2 KO”) at various passages (P38-42) were maintained by seeding at about 33,000 cells/cm² for a 4-day passage or about 50,000 cells/cm² for a 3-day passage with hESM medium (DMEM/F12+10% KSR+10 ng/ml Activin A and 10 ng/mL Heregulin) and final 10% human AB serum.

### Aggregation of Edited Human Embryonic Stem Cells for PECs Differentiation

The edited cells were dissociated into single cells with ACCUTASE® and then centrifuged and resuspended in 2% StemPro (Cat #A1000701, Invitrogen, CA) in DMEM/F12 medium at 1 million cells per ml, and total 350-400 million of cells were seeded in one 850 cm² roller bottle (Cat #431198, Corning, NY) with rotation speed at 8 RPM+0.5 RPM for 18-20 hours before differentiation. The aggregates from edited human pluripotent stem cells were differentiated into pancreatic lineages using in roller bottles as described in Schulz et al. (2012) PLOS ONE 7 (5): e37004 and shown for X1 cells. Aggregates from edited human pluripotent stems cells were differentiated into pancreatic lineages as described in Rezania et al. (2014) Nat. Biotechnol. 32 (11): 1121-1133 and US20200208116.

The expression pattern of CHGA, FOXA2, NKX6.1, PDX1 and INS from the “X4+TGF-β2 KO” clones, i.e., TXNIP KO/MANF-P2A-HLA-E KI & B2M KO/TNFAIP3 (A20)-P2A-PD-L-1 KI (X1) CIITA KO/CD39 KI and TGF-β2 KO, at PEC stage and Stage 6 (S6) was determined to confirm differentiation.

### Example 19

Immune Evasion Assay with B2M KO and X1 PECs

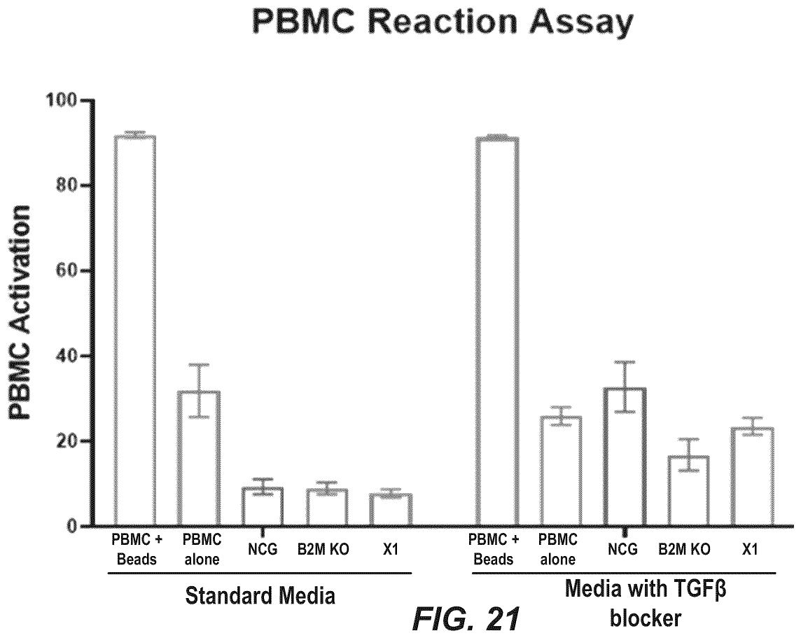

The capacity of B2M KO and TXNIP KO/MANF-P2A-HLA-E KI & B2M KO/TNFAIP3 (A20)-P2A-PD-L-1 KI (“X1”) cells to evade the immune response with and in the absence of TGF-β signaling in the media was tested using an immune evasion assay using peripheral blood mononuclear cell (PBMC) proliferation assay. The assay was conducted as per the manufacturer's instruction provided for the CellTrace™ CFSE Cell Proliferation Kit. Briefly fluorescently labelled PBMCs were added to X-VIVO-15 media comprising edited or non-cutting control PEC cells, IL-2 and human serum with or without TGF-β blocking antibodies. Antibodies were used against TGF-β1, TGF-β2 and TGF-β3 to block the proteins from signaling in the media and inhibit TGFB-mediated immune evasion. PBMC cell proliferation was monitored using the dye-dilution CFSE Cell Proliferation kit over a period of 5-days. The PBMC activation data without or with the TGF-β blocker is provided in FIG. 21. The results show that without TGF-β blocking, all PECs was “immune evasive” as no T-cell activation was induced for any of the samples. With TGF-β blocking, there was more T-cell activation. The NCG (non-cutting control that had normal B2M) drove T-cell activation responses above the PBMCs alone control, but both B2M KO and X1 (which also has a B2M KO) PECs were below the baseline, which suggested that X1 and B2M KO PECs were immune evasive while NCG PECs were mildly immunogenic to allogenic PBMCs.

### Example 20

### Characterization of Edited and Differentiated PECs for TGF-β2 Secretion

The TGF-β1 and TGF-β2 secretion level profiles in edited and differentiated cells were tested in 72 hr condition media using an ELISA based assay using anti-TGF-β1 and anti-TGF-β2 antibodies. The antibodies used are provided in the Table 17 below.

TABLE 17
ANTIBODIES

| ELISA target | Vendor | Cat# |
|---|---|---|
| TGF-β1 | ThermoFisher | BMS249 |
| TGF-β2 | R&D | DB250 |
| S100A8/A9- | FisherScientific | 501656476 |
| Calprotein 9 | | |
| GDF9 | LifeSpan | LS-526-1 |
| PDGF-AA | ThermoFisher | EHPDGFA |
| PDGF-BB | ThermoFisher | EHPDGFB |

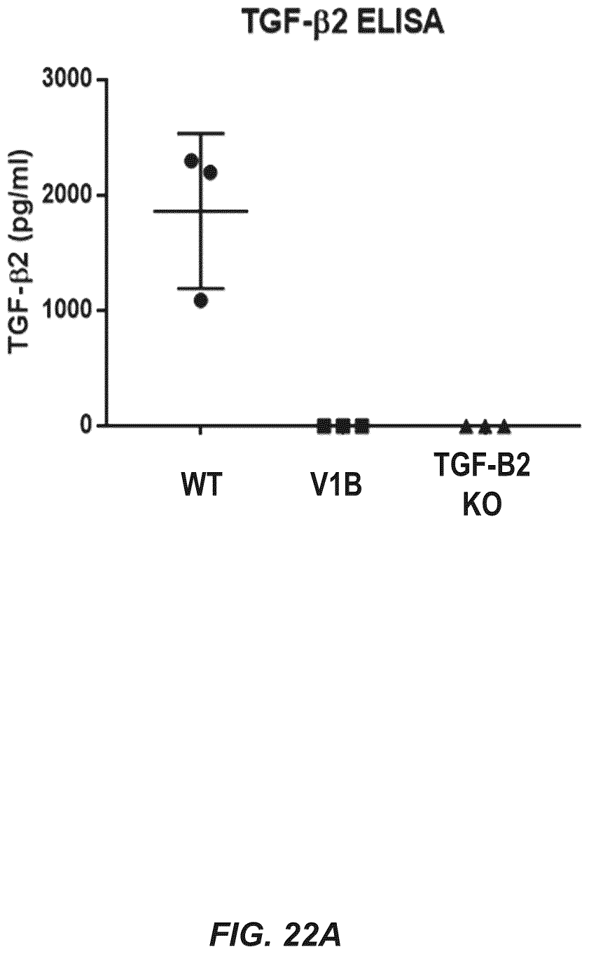

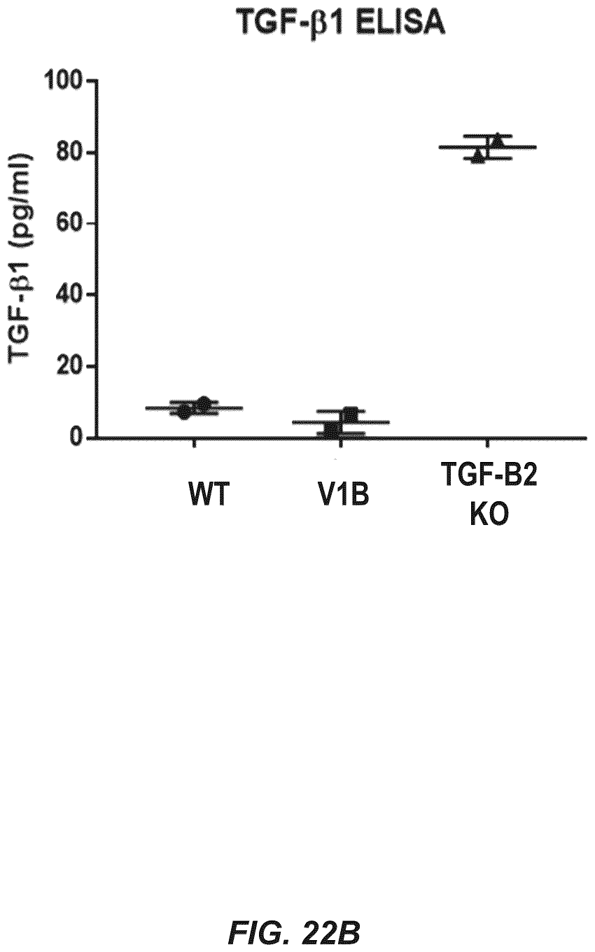

The TGF-β2 and TGF-β1 secretion profiles were determined in a TGF-β2 KO cell and an edited cell having HLA-E KI, TXNIP KO, PD-L-1 KI, and B2M KO (“VIB”). Results show that both VIB and TGF-β2 KO cells exhibited undetectable levels of TGF-β2 in the condition media (see FIG. 22A). However, interestingly conditioned media from TGF-β2 KO cells exhibited higher levels of TGF-β1 secretion (see FIG. 22B).

### Example 21

### Characterization of Chemoattractants Secreted by TGF-β2 KO Cells

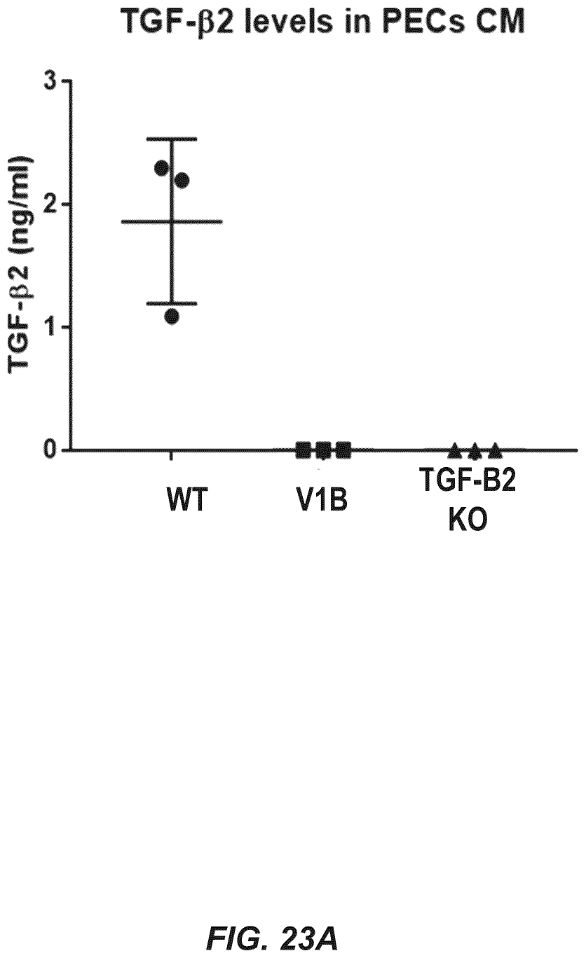

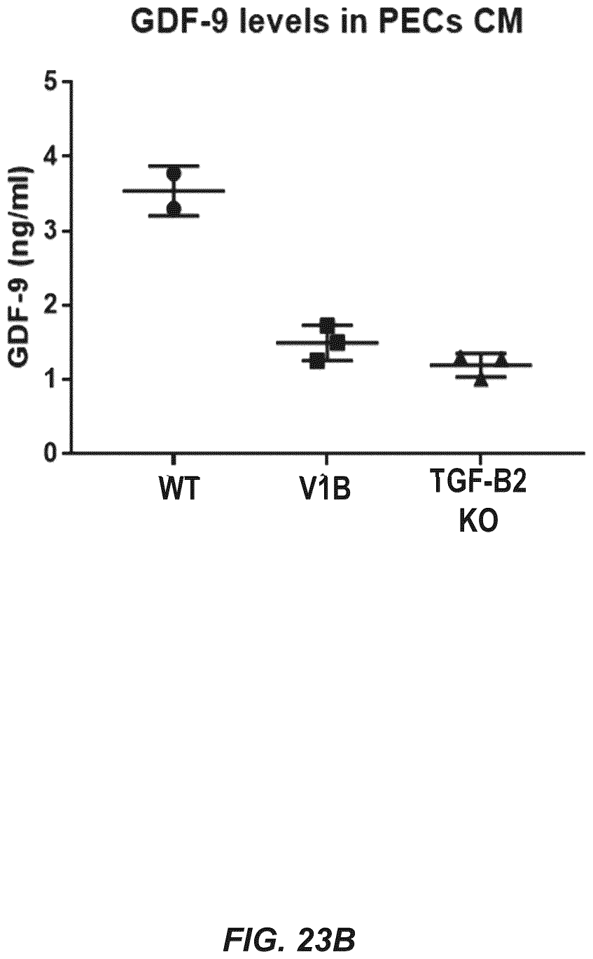

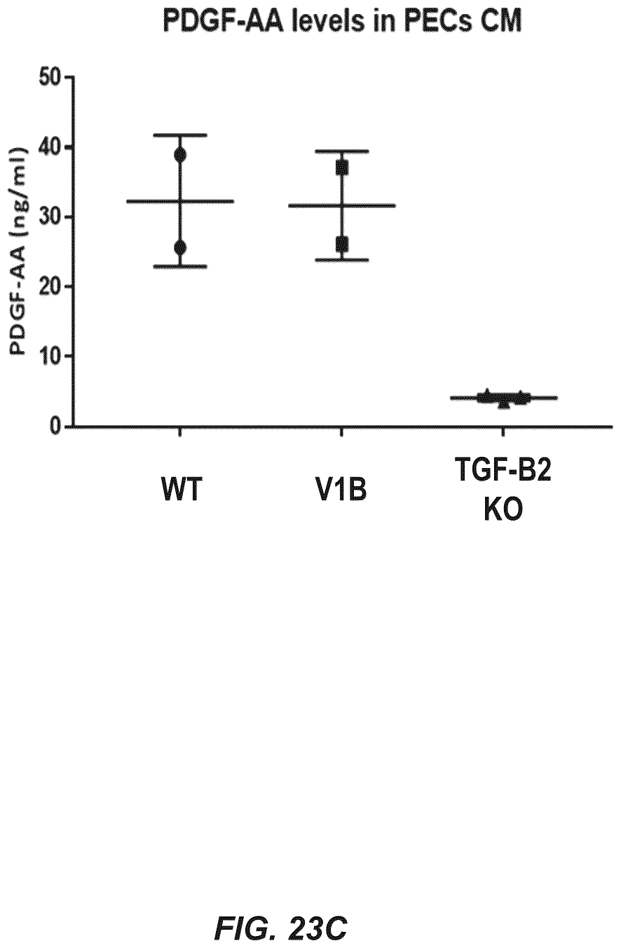

Fibroblast migration and resulting fibrosis is directed by chemoattractants secreted by the engrafted cells. An ELISA based approach was used to ask if the TGF-β2 KO cells have reduced secretion of chemoattractants as compared to VIB cells (HLA-E KI, TXNIP KO, PD-L-1 KI, B2M KO) and X1 (antibodies provided in Table 17 in Example 20). The tested chemoattractants included TGF-β2 (see FIG. 23A), growth differentiation factor (GDF-9, see FIG. 23B), and platelet derived growth factor-AA (PDGF-AA, see FIG. 23C).

Results suggest that both VIB and TGF-β2 KO cells showed greatly reduced secretion of TGF-β2 and GDF-9. However, only TGF-β2 KO cells showed reduced secretion of PDGF-AA.

### Example 22

### In Vitro Fibroblast Migration Assay

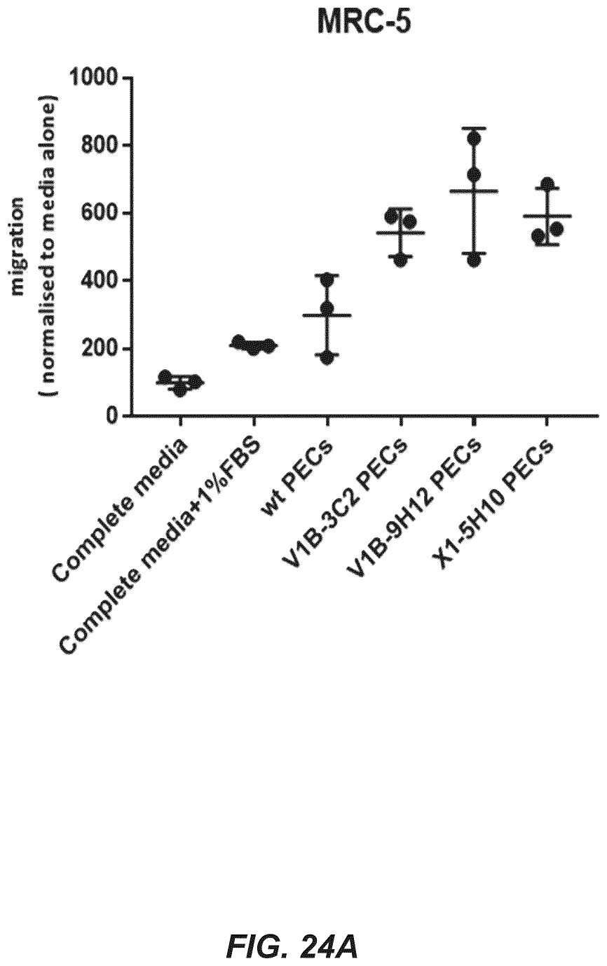

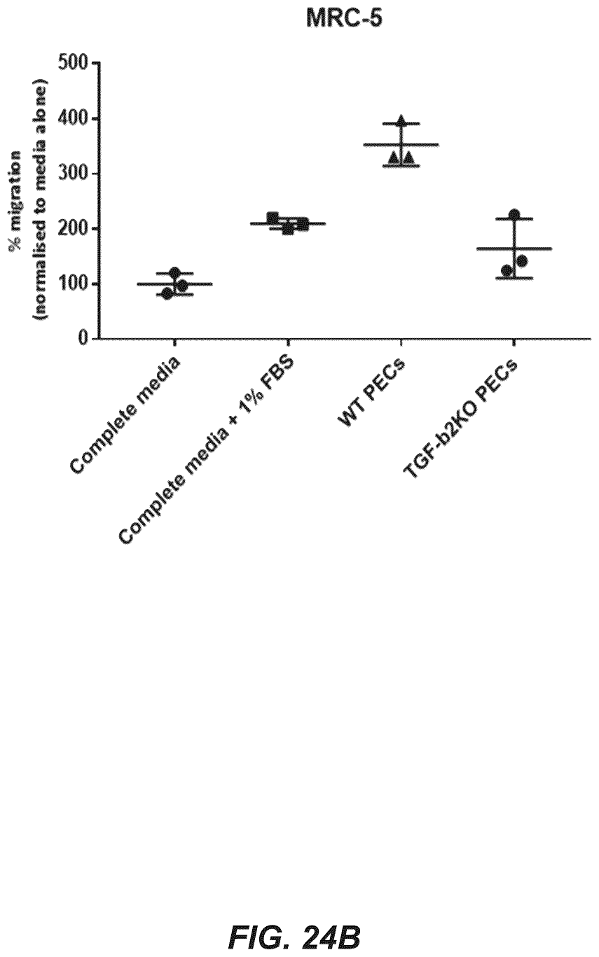

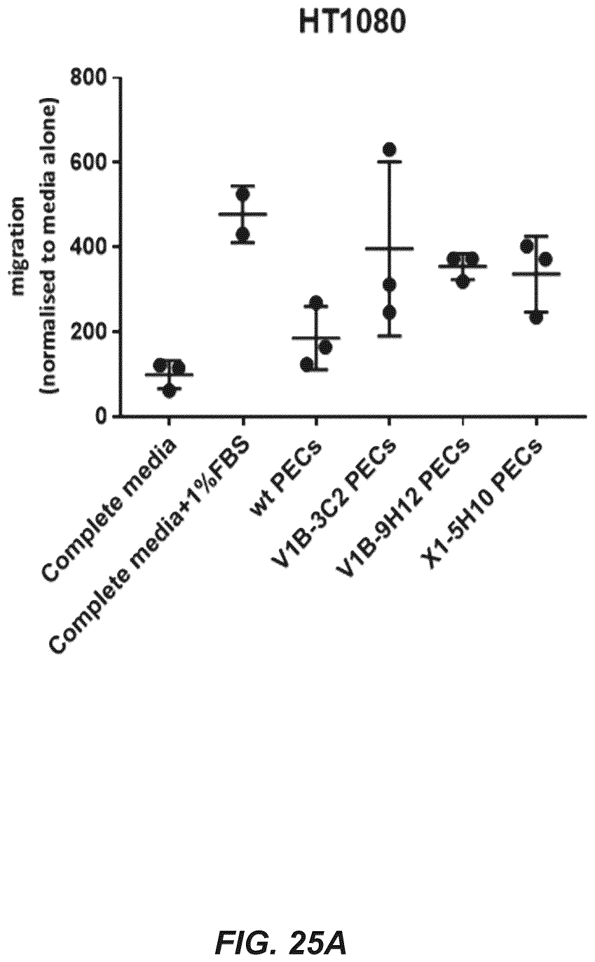

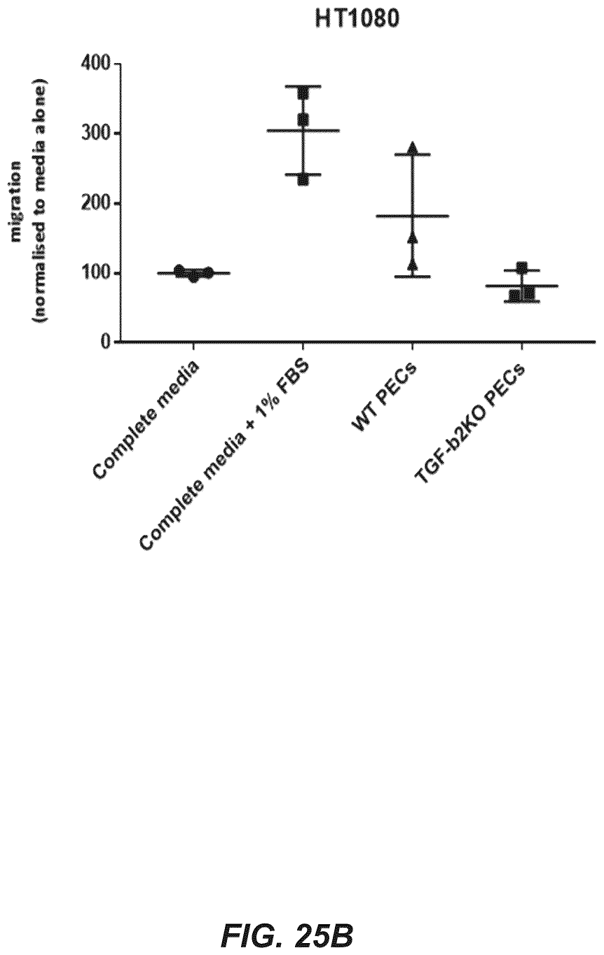

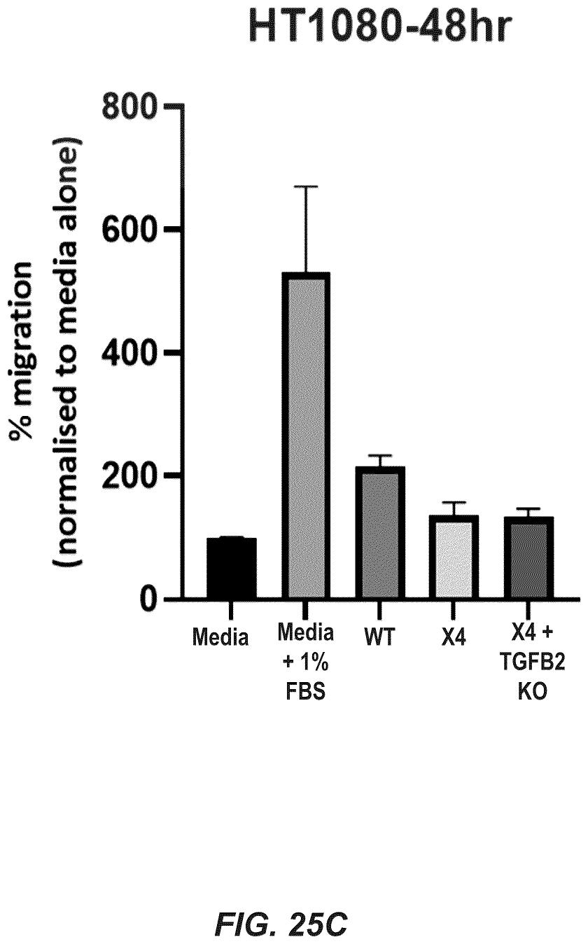

In vitro fibroblast migration assays were conducted using the QCM chemotaxis cell migration assay kit from Millipore/Sigma (cat #ECM509) as per the manufacturer's instructions. Briefly, cell suspensions comprising MRC-5 (human lung fibroblast) or HT1080 (human fibrosarcoma) cells were placed in the upper chamber of an assay cell that is separated from the outer chamber comprising 72-hr condition media from wild-type, V1B, TGF-β2 KO, X1, X4, and/or X4+TGF-β2 KO PECs by a 8 μm pore size polycarbonate membrane. Cells were allowed to migrate through the polycarbonate membrane for 2-24 hrs. Migrated cells clung to the bottom of membrane. Migrated cells were dissociated from the membrane and lysed. The cells were quantified using the CyQuant GR Dye. FIG. 24A-FIG. 24B show fibroblast migration assay results performed using human lung fibroblasts (MRC-5) cells with condition media from WT, VIB, and X1 cells (FIG. 24A) and WT and TGF-β2 KO cells (FIG. 24B). FIG. 25A-FIG. 25C show fibroblast migration assay results performed using human fibrosarcoma (HT1080) cells with condition media from WT, VIB, and X1 cells (FIG. 25A), WT and TGF-β2 KO cells (FIG. 25B), and WT, X4, and X4+TGF-β2 KO cells (FIG. 25C). As seen from the data presented, both the TGF-β2 KO edited PEC condition media supported reduced migration of fibroblasts compared to wild-type.

### Example 23

Patient Trials to Study Implantation of Perforated Devices with Edited Cells

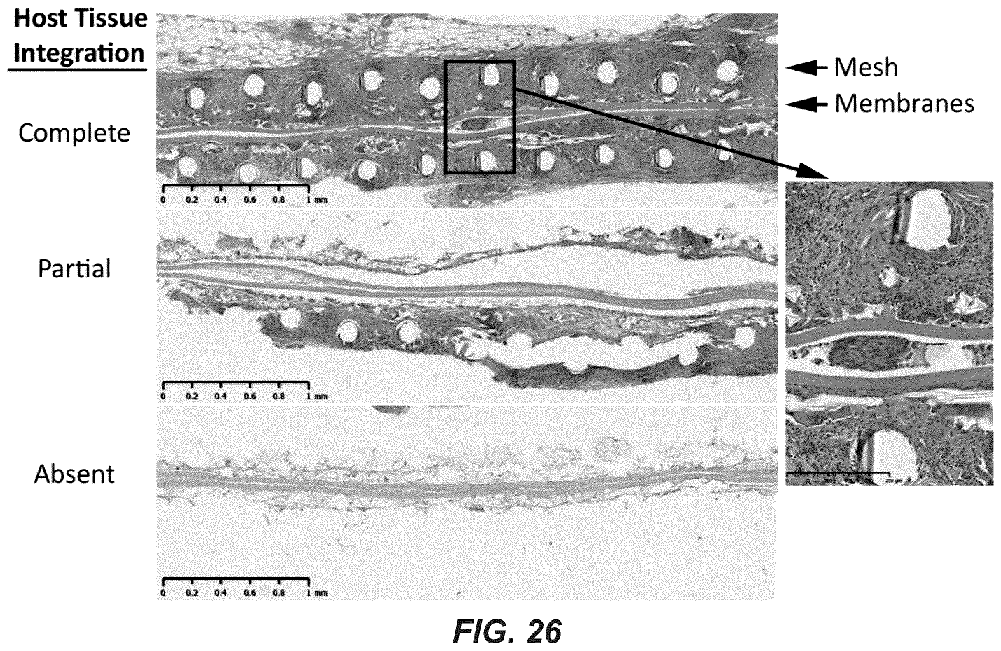

Subjects were implanted with perforated devices comprising genetically modified (B2M knock-out, TXNIP KO, PD-L1 knock-in KI, and HLA-E trimer KI) PECs. Units were explanted at various time points and examined histologically ex vivo to evaluate if the gene edited cells were viable and had differentiated adequately. Exploratory biomarker analysis was also conducted that included but were not limited to RNA sequencing and spatial transcriptomics. Implants were usually well tolerated but foreign body response (FBR) and delayed healing were seen in a few cases when implanted with the perforated devices with gene edited cells, although with varied intensity (see FIG. 26).

### Example 24

### Genetically Modified Cells Evade Immunity in Multiple Assays

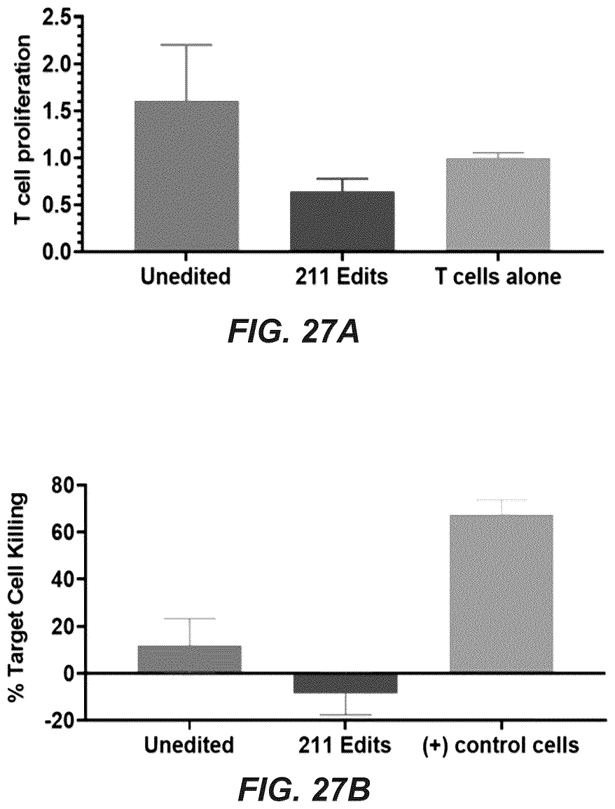

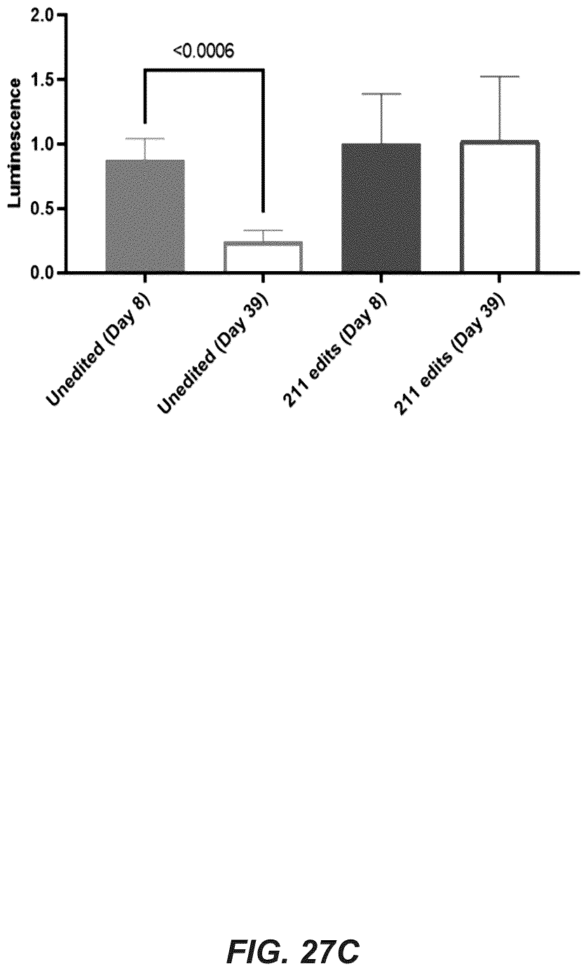

Genetically modified pancreatic endoderm cells comprising TXNIP KO/MANF-P2A-HLA-E KI & B2M KO/TNFAIP3 (A20)-P2A-PD-L-1 KI edits, were tested for the ability to evade immunity in multiple assays. As shown in FIG. 27A-FIG. 27C, 211 cells were able to eliminate T-cell response (FIG. 27A), resist NK cell attack (FIG. 27B) and survive in a humanized mouse model (FIG. 27C). The humanized mouse model contained human DC, B-cells, T-cells, NK cells and monocytes, and the ability for these genetically modified pancreatic cells to survive demonstrates the broad immune evasive potential for these cells.

### Example 25

### Genetically Modified Cells Improved Stimuli-Responsive Insulin Production

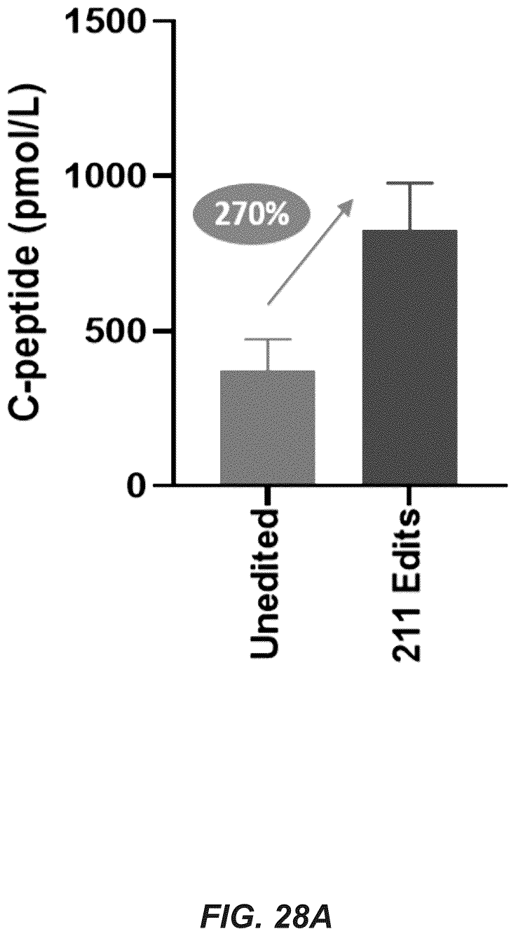

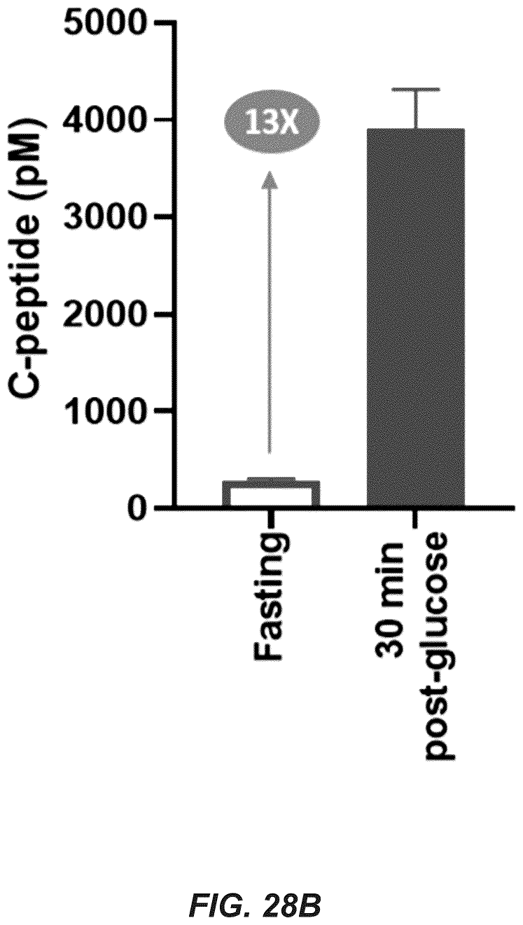

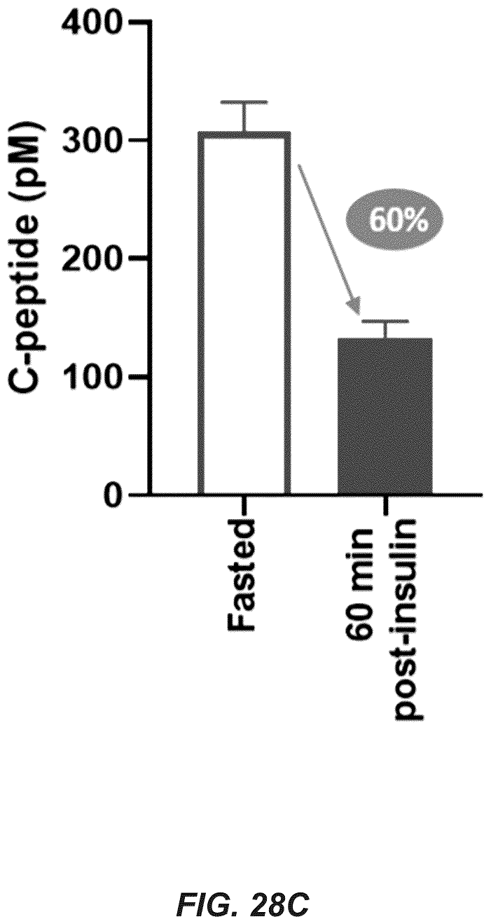

Genetically modified pancreatic endoderm cells (TXNIP KO/MANF-P2A-HLA-E KI & B2M KO/TNFAIP3 (A20)-P2A-PD-L-1 KI) were transplanted into a recipient animal and measures of insulin production, glucose responsiveness and insulin sensitivity were all taken 12 weeks post-transplant. Control, unedited cells, were also transplanted. The edits lead to increased insulin production (FIG. 28A). maintained glucose responsiveness (FIG. 28B), and preserved insulin sensitivity (FIG. 28C).

### Example 26

### Robust Engraftment of Genetically Modified Cells in Nude Rat Model

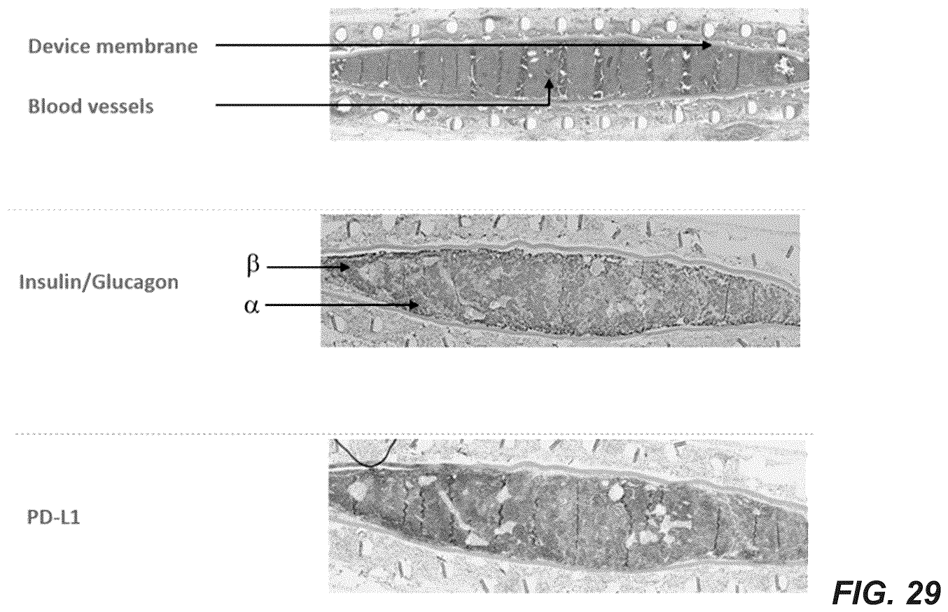

Nude rats were implanted with perforated devices comprising genetically modified pancreatic endoderm cells (TXNIP KO/MANF-P2A-HLA-E KI & B2M KO/TNFAIP3 (A20)-P2A-PD-L-1 KI). Units were explanted at various time points and examined histologically ex vivo to evaluate if the gene edited cells were viable and have differentiated adequately. H&E sections of the implant at 24 weeks post implantation showed good vascularization, implanted cells showed favorable differentiation with a β/α ratio of approximately 2 and retention of PD-L1 expression was observed in long-term grafts (FIG. 29).

### Example 27

### Genetically Modified Cells Reversed Hypoglycemia in Diabetic Rat Model

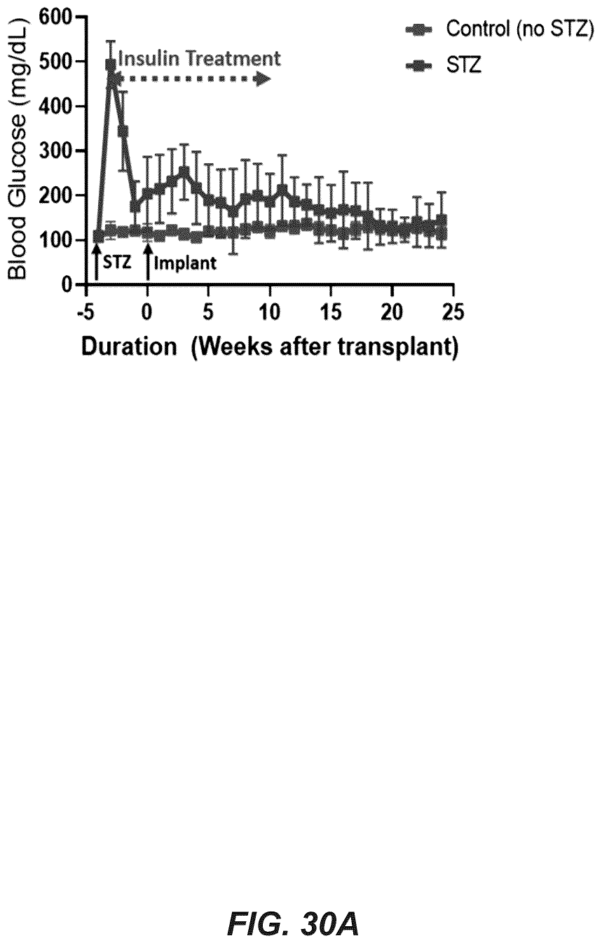

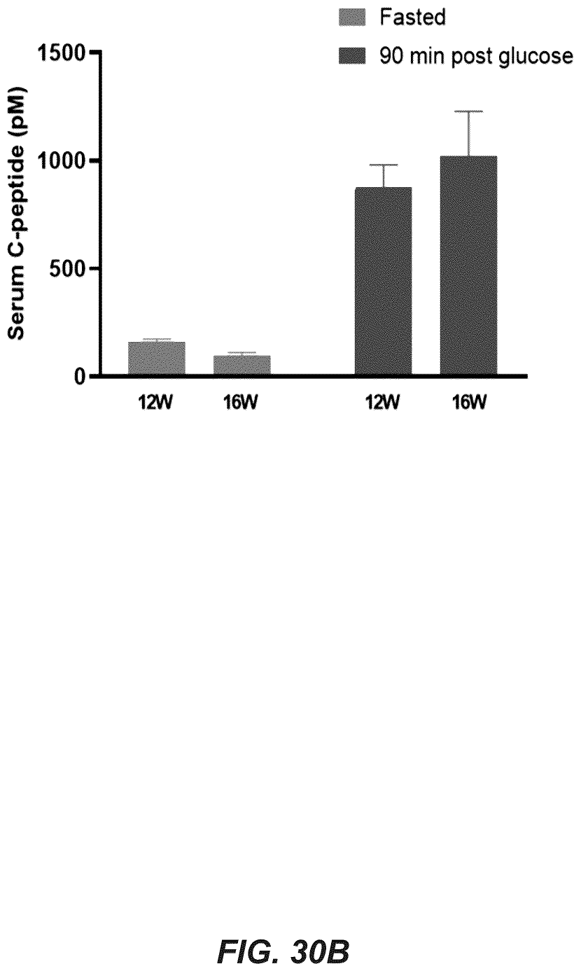

Rats were treated with streptozotocin or left untreated (normoglycemic control) four weeks before implantation with genetically modified pancreatic endoderm cells (TXNIP KO/MANF-P2A-HLA-E KI & B2M KO/TNFAIP3 (A20)-P2A-PD-L-1 KI). Blood glucose was measured up to 25 weeks post transplantation with insulin treatment occurring before implantation. FIG. 30A shows how blood glucose levels normalized by 12-16 weeks. FIG. 30B shows how glucose stimulated insulin release increased 90 minutes post glucose.

### Example 28

### Effects of Immune Modulating Drugs on Function of Genetically Modified Cells

Provided in this Example are in vivo efficacy evaluations of VCTX211 combination products (e.g., genetically modified cells loaded into a perforated delivery device) to assess potential sensitivity of exposure to calcineurin inhibitors. In this study, athymic nude rats each received two implants of compositions comprising genetically modified cells disclosed herein (e.g., VCTX211) each. The animals were otherwise untreated or received chow formulated to contain calcineurin inhibitors cyclosporine A or tacrolimus. Regulated insulin secretion was assessed at 12- and 16-weeks following implant.

VCTX211 comprises pancreatic endoderm cells (PEC) derived from genetically modified human embryonic stem cells loaded into perforated delivery devices that are implanted subcutaneously. The cellular component of VCTX211 differentiates in vivo to generate pancreatic endocrine cells that secrete hormones such as insulin in a glucose-responsive manner. The perforated membranes of the device retain the PEC-derived cells within the lumen, but also allow entry to host-derived cells such as endothelial cells that promote the formation of blood vessels within the device lumen. The advantage of improved perfusion through direct vascularization of the grafted tissue comes with the disadvantage of the graft also being directly exposed to the host immune system. Benefits of the genetic modifications of VCTX211 include avoidance of alloimmune targeting of the graft by human immune cells.

In studies of a combination product similar to VCTX211, but produced with unmodified cells, testing was performed for in vivo sensitivity to tacrolimus and cyclosporine A. In these studies, the calcineurin inhibitors did not appear to affect the development or function of beta cells derived from the implants. The administration of the calcineurin inhibitors was achieved through formulation into the chow fed to the animals (athymic rats). In these previous studies, exposure levels of cyclosporine A measured in rats achieved or exceeded target levels for the prophylactic use in clinical organ transplantation settings, while they were below target levels for tacrolimus. Increased tacrolimus exposure could not be achieved due to toxicity resulting in inappetence and severe weight loss.

Overall study design of the study provided in this example is outlined in Table 18.

TABLE 18
STUDY OUTLINE

| Group | ¹Group ID | Number Of | ²GSIS | Termination & Ex Vivo |
|---|---|---|---|---|
| Number | (Diet) | Animals | Testing | Analyses |
| 1 | CsA-250 | 10 | 12 & 16 | 18 Weeks Histology |
| 2 | TAC-150 | 10 | weeks | |
| 3 | Control | 10 | | |

¹Each animal received two subcutaneous VCTX211 implants consisting of PD20 devices loaded with approximately 9 × 10⁶ PEC-211 cells.
CsA-250, chow containing 250 mg/kg cyclosporine A was administered through the GSIS testing at 12 weeks; TAC-150, chow containing 150 mg/kg tacrolimus was administered for 3 weeks.
²GSIS, Glucose-stimulated insulin secretion

VCTX211 test articles (e.g., perforated delivery devices loaded with genetically modified cells disclosed herein) were first generated with pancreatic endoderm cell (PEC-211; e.g., comprising B2M KO and KI of TNFAIP3 and PD-L1; and KO of TXNIP and KI of MANF and HLA-E) aggregates derived from VCTX211 stem cell progenitor cells. To do so, the thaw and culture of PEC-211 aggregates was first performed in roller bottles. On the third day post-thaw, 20 μL aliquots of settled pellet volume (approximately 9×10⁶ cells) were prepared and loaded into sterilized PD20 devices (e.g., perforated cell delivery device) to produce VCTX test articles. In some embodiments, about 9.1×10⁶ cells were loaded per device. In some embodiments, multiple 10 μL aliquots were used to determine the dose of cells loaded per test article. After formulation, test articles were maintained at room temperature in 10 mL storage tubes with media for 2 days.

Prior to implant, 30 male athymic nude rats (described in Table 19) were randomly assigned to three groups of ten animals each, and unbiased weight distribution was confirmed. At the time of group randomization, each group received either regular chow (Group 3), or chow containing cyclosporine A (Group 1) or tacrolimus (Group 2) (chow formulations described in Tables 19A-C) ad libitum starting from just prior to implant. At implant, each animal was surgically implanted subcutaneously with two VCTX test articles. The animals then continued to receive their respective chow. Group 1 and 3 animals received their respective chow for 12 weeks, while Group 2 animals only tolerated the tacrolimus-containing chow for 3 weeks. At that time, due to diminished appetence and reduced body weight, the Group 2 animals were switched to the control chow (i.e. Table 19C). At 12 weeks, all animals were switched to standard rodent chow (Teklad 2920X, Envigo).

TABLE 19
TEST SYSTEM

| Species | Rat (Rattus norvegicus) |
|---|---|
| Strain/Stock | Athymic nude; 265I-Hsd:RH-Foxn I^{rnu} |
| Vendor | Envigo (formerly Harlan), Indianapolis, IN |
| Sex | Male |
| Age at implant | 19 weeks (older animals were shown to be more |
| | tolerant of the TAC-150 and CsA-150 diets) |
| Body weight range | 313-460 grams, randomized across study groups |
| Justification for use | Immune-compromised animals are necessary to |
| | accept xenografts of human cells. |
| Number used | 30 |
| for study | |

All surviving animals were subjected to glucose-stimulated insulin secretion testing through measurements of serum C-peptide after glucose administration at 12- and 16-weeks after implant. The study was terminated after 18 weeks. At necropsy, VCTX211 explants were harvested and preserved for histological examination.

TABLE 20A
CHOW FORMULATIONS

| Chow formulation | |
|---|---|
| name | CsA-250 (Group 1) |
| Bio-Serve Diet Code | F10357 |
| Drug content | 250 mg cyclosporine A per kg chow |
| Drug source | CycloSPORINE ORAL SOLUTION USP |
| information | MODIFIED. 100mg/mL. NDC 0172-7313-20 |
| Description | Rodent Diet, AIN-76A, cyclosporine (250 mg/kg)- |
| | CSA-Viacyte, Bacon Flavor, ½ inch Pellets |
| Exposure duration | 12 weeks from 3 days pre-implant |

TABLE 20B
CHOW FORMULATIONS

| Chow formulation | |
|---|---|
| name | TAC-150 (Group 2) |
| Bio-Serve Diet Code | F10356 |
| Drug content | 150 mg tacrolimus per kg chow |
| Drug source | Prograf tacrolimus, 5 mg/capsule, |
| information | NDC 0469-0657-73 |
| Description | Rodent Diet, AIN-76A, tacrolimus(150 mg/kg)- |
| | CSA-Viacyte, Bacon Flavor, ½ inch Pellets |
| Exposure duration | 3 weeks from 3 days pre-implant |

TABLE 20C
CHOW FORMULATIONS

| Chow formulation name | Control (Group 3) |
|---|---|
| Bio-Serve Diet Code | F10355 |
| Drug content | None |
| Drug source information | Not applicable |
| Description | Rodent Diet, AIN-76A, Bacon Flavor, |
| | ½ inch Pellets |
| Exposure duration | 12 weeks from 3 days pre-implant |

In response to persistent low body weight and moderate hyperglycemia among animals in Group 1 (CsA), the study was concluded at 18 weeks

### Results

Test articles achieved the target dose of approximately 9×10⁶ PEC cells per device. Details of the characterization of the prepared test articles are presented in Table 21.

TABLE 21
TEST ARTICLE CHARACTERIZATION

| | | Viable PEC-211 Cells (×10⁶) | | |
|---|---|---|---|---|
| PEC-211 Lot# | Viability | Per 10 μL | Per 20 μL | Per Animal |
| E3008 | 89% | 4.56 | 9.12 | 18.2 |

### Animals and Diet

Twelve of the 30 animals on study died or were euthanized prior to the study termination at 18 weeks.

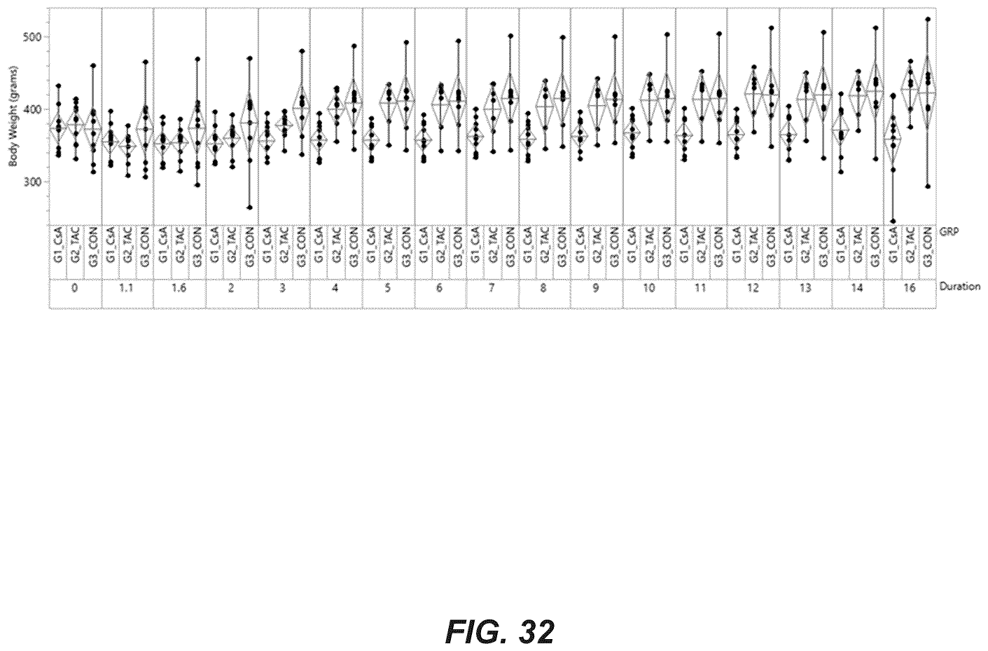

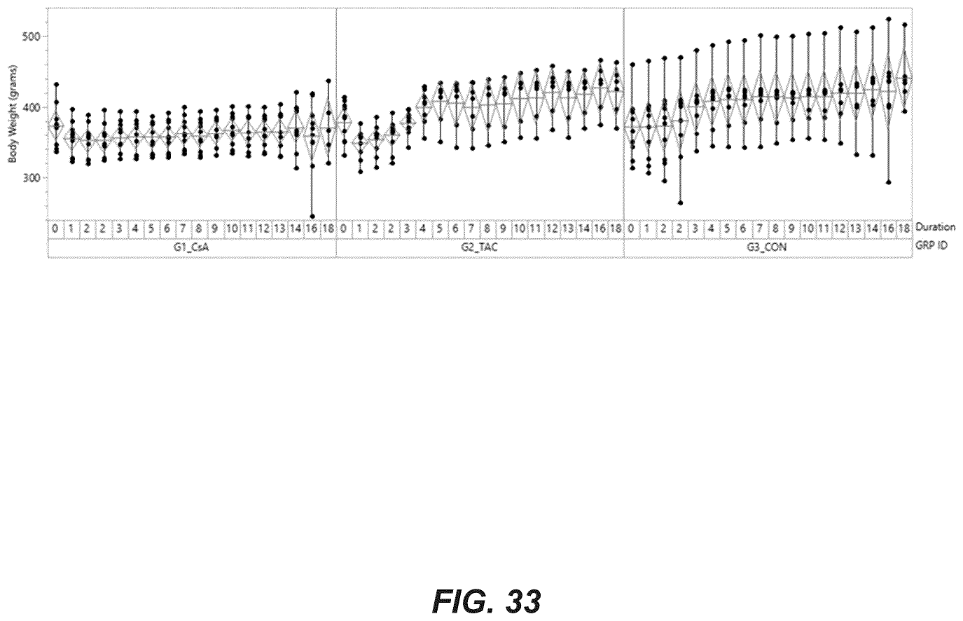

Animals fed with the control diet (Group 3) gradually gained weight, however the test group animals lost on average 6% (Group 1, CsA) and 8% (Group 2, TAC) of body weight within two weeks of receiving the drug-containing diets (FIG. 32-FIG. 33). By 3 weeks, 3 animals on the TAC diet (Group 2) had died or were euthanized in extremis. The remaining animals were then switched to the control diet as they appeared not to tolerate the diet formulation. These remaining animals gained weight and their average body weight caught up with the average of the control Group 3. One additional Group 2 animal was euthanized at 7 weeks, after presenting with severe hyperglycemia. This was an expected possible outcome as clinical transplantation-associated hyperglycemia has been described for immunosuppression regimes that include calcineurin inhibitors.

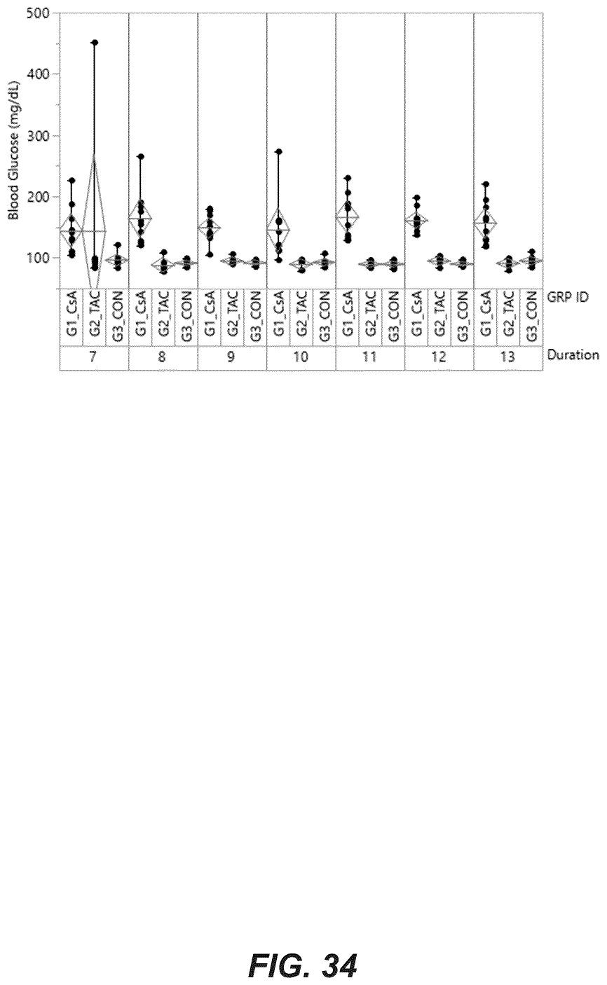

The body weight of Group 1 (CsA) animals stabilized after the initial decline at 2 weeks, but these animals remained underweight compared to the control group. Evaluation of blood glucose showed elevated levels in most Group 1 animals by 7 weeks, but generally remained below 250 mg/dL (FIG. 34). Elevated blood glucose levels below 250 mg/dL can be tolerated in nude rats without intervention, but, without being bound by any particular theory, likely contributed to the lack of weight gain in Group 1 animals. After 15-16 weeks, 4 Group 1 animals were euthanized in poor health. Three of these animals had maloccluded teeth that may have affected their ability to eat. The fourth animal also presented with sudden weight loss.

For control Group 3, 4 animals were also terminated prematurely. Two control animals presented with significant weight loss within 2 weeks of the implant surgery and one animal presented with rapid weight loss at 16 weeks, but no cause for their condition was identified. One animal presented with swollen hind limb and impaired mobility at 15 weeks. Tibiofemoral joint inflammation is a background condition identified in nude rats and the suspected diagnosis for this animal. Additional details for all animals are presented in Table 22-Table 23.

### Glucose-Stimulated Insulin Secretion (GSIS)

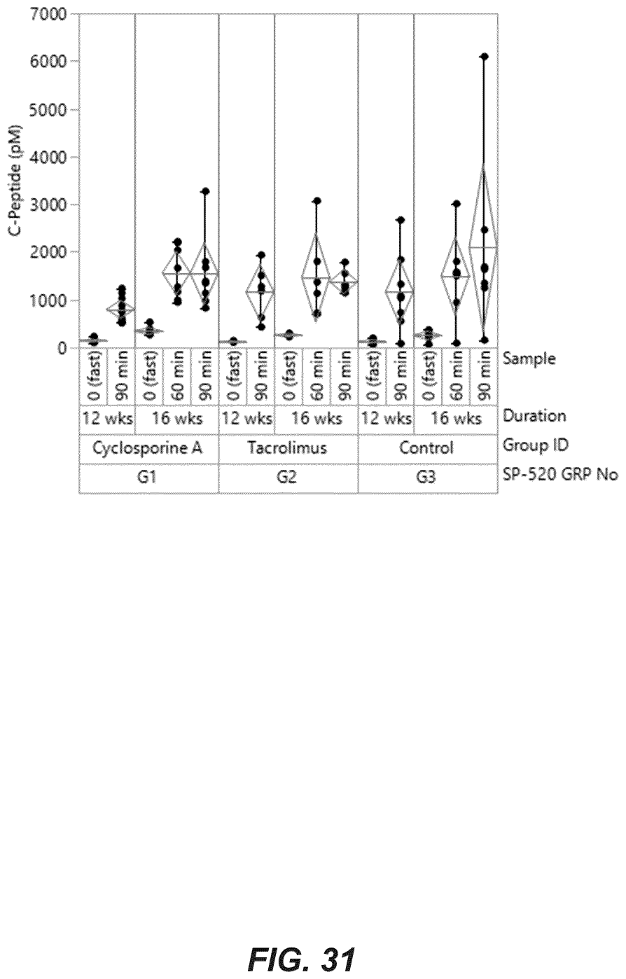

The GSIS test was performed on all surviving animals at 12 and 16 weeks and these data are presented in FIG. 31, while individual animal C-peptide measurements are tabulated in Table 23. C-peptide levels were measured in the fasted state and after 90-minutes (at 12 weeks) or 60- and 90-minutes (at 16 weeks) after glucose administration. Comparing fasted versus stimulated states, the results show glucose-responsive increases in serum C-peptide concentrations. The magnitude of the response was similar between groups, indicating the drug treatments did not appear to impact the functional development of the VCTX211 implants.

### CONCLUSION

Animals implanted with VCTX211 were exposed to calcineurin inhibitors cyclosporine A for 12 weeks or tacrolimus for 3 weeks. Treatment group animals demonstrated reduced body weight and elevated blood glucose. However, compared to control animals, functional development of VCTX211 grafts, as evaluated with GSIS tests, were not affected in animals treated with cyclosporin A or tacrolimus.

TABLE 22
INDIVIDUAL ANIMAL WEIGHT, FATE, AND BLOOD GLUCOSE

| Group | | Body Weight (gram) | | | | Timing of |
|---|---|---|---|---|---|---|
| ID | Rat ID | Study Start | 12 wks | 16 wks | Termination | Termination (weeks) |
| 1 | C4525 | 373 | 358 | 360 | 367 | 18 |
| CsA | C4526 | 383 | 360 | 316 | 316 | 16 |
| | C4527 | 376 | 389 | 388 | 388 | 16 |
| | C4528 | 407 | 388 | 417 | 392 | 18 |
| | C4529 | 336 | 336 | † | 245 | 15 |
| | C4530 | 340 | 336 | 350 | 346 | 18 |
| | C4531 | 371 | 368 | 370 | 320 | 18 |
| | C4532 | 432 | 400 | 419 | 437 | 18 |
| | C4533 | 346 | 345 | 377 | 367 | 18 |
| | C4534 | 374 | 357 | 349 | 349 | 16 |
| 2 | C4535 | 387 | † | | 380 | 3 |
| TAC | C4536 | 414 | † | | ND | 7 |
| | C4537 | 385 | 442 | 451 | 445 | 18 |
| | C4538 | 409 | 436 | 433 | 425 | 18 |
| | C4539 | 366 | † | | 388 | 3 |
| | C4540 | 398 | 433 | 438 | 436 | 18 |
| | C4541 | 350 | 368 | 375 | 370 | 18 |
| | C4542 | 351 | 395 | 400 | 397 | 18 |
| | C4543 | 331 | † | | 357 | 3 |
| | C4544 | 402 | 462 | 466 | 463 | 18 |
| 3 | C4545 | 343 | † | | 295 | 1 |
| Control | C4546 | 394 | 410 | | 400 | 15 |
| | C4547 | 366 | 432 | 448 | 443 | 18 |
| | C4548 | 460 | 515 | 524 | 516 | 18 |
| | C4549 | 383 | 428 | 436 | 434 | 18 |
| | C4550 | 393 | 427 | 443 | 438 | 18 |
| | C4551 | 323 | † | | 264 | 2 |
| | C4552 | 350 | 396 | 403 | 394 | 18 |
| | C4553 | 313 | 339 | 293 | 293 | 16 |
| | C4554 | 397 | 427 | 437 | 422 | 18 |

† These animals died or were euthanized early due to poor health,.

TABLE 23
INDIVIDUAL ANIMAL WEIGHT,
FATE, AND BLOOD GLUCOSE

| | | C-peptide (pM) | | | | |
|---|---|---|---|---|---|---|
| | | 12 Weeks | | 16 Weeks | | |
| Group ID | Rat ID | Fast | 90 min | Fast | 60 min | 90 min |
| 1 | C4525 | 134 | 747 | 308 | 2197 | 825 |
| CsA | C4526 | 114 | 607 | † | | |
| | C4527 | 139 | 1023 | 302 | 990 | 969 |
| | C4528 | 139 | 876 | 533 | 2222 | 1669 |
| | C4529 | 144 | 1237 | † | | |
| | C4530 | 233 | 772 | 322 | 1266 | 1344 |
| | C4531 | 164 | 530 | 339 | 2033 | 3274 |
| | C4532 | 117 | 1138 | 390 | 1659 | 1791 |
| | C4533 | 157 | 510 | 263 | 943 | 1381 |
| | C4534 | 99 | 545 | 291 | 1165 | 1134 |
| 2 | C4535 | † | | | | |
| TAC | C4536 | † | | | | |
| | C4537 | 113 | 1933 | 282 | 1131 | 1312 |
| | C4538 | 123 | 1504 | 253 | 1798 | 1545 |
| | C4539 | † | | | | |
| | C4540 | 119 | 627 | 273 | 720 | 1271 |
| | C4541 | 107 | 428 | 227 | 1365 | 1207 |
| | C4542 | 147 | 1178 | 302 | 3073 | 1782 |
| | C4543 | † | | | | |
| | C4544 | 109 | 1271 | 219 | 700 | 1140 |
| 3 | C4545 | † | | | | |
| Control | C4546 | 202 | 552 | † | | |
| | C4547 | 130 | 1833 | 313 | 943 | 2464 |
| | C4548 | 106 | 1322 | 207 | 1796 | 1247 |
| | C4549 | 135 | 1033 | 250 | 1553 | 1343 |
| | C4550 | 139 | 730 | 304 | 1575 | 1633 |
| | C4551 | † | | | | |
| | C4552 | 140 | 2670 | 370 | 3013 | 6095 |
| | C4553 | 62 | 84 | 71 | 92 | 155 |
| | C4554 | 103 | 1075 | 254 | 1494 | 1684 |

† These animals died or were euthanized early due to poor health.

### Example 29

### Evaluation of PEC-211 in the Presence of Tacrolimus

Evaluation of the PEC-211 cells in the presence of Tacrolimus was performed in vitro. Stage 4 Pancreatic progenitor PEC-211 cells were further differentiated to stage 6 to produce matured islet like cell population in presence of 0.1 μM of Tacrolimus for 10 days. Control PEC-211 cells were not treated with Tacrolimus. Table 24 below shows flow cytometry data of CHGA, PDX1 and NKX6.1 in Stage 6 (S6) cells. The results shows that 0.1 μM Tacrolimus did not affect in vitro differentiation to islet cells.

TABLE 24
EVALUATION OF PEC-211 DIFFERENTIATION TO ISLET
LIKE CELLS IN THE PRESENCE OF 0.1 μM TACROLIMUS

| | | | | | CHGA-NKX6.1+ |
|---|---|---|---|---|---|
| | Total | Total | Total | Total | PDX1+ or- |
| Sample ID | CHGA+ | NKX6.1+ | INS+ | GCG+ | (Pancreatic Progenitor) |
| E3007_S6 | 71.7 | 49.2 | 69.5 | 34.7 | 19.9 |
| PEC-211 | | | | | |
| control | | | | | |
| E3007_S6 | 76.3 | 48.7 | 70.4 | 36.6 | 16.9 |
| PEC-211 | | | | | |
| (0.1 μM) | | | | | |
| Tacrolimus | | | | | |

Table 25 below presents additional sequences of the disclosure.

TABLE 25
SEQUENCES

| SEQ ID | |
|---|---|
| NO: | Sequence |
| 14 | CCTGCAGGCAGCTGCGCGCTCGCTCGCTCACTGAGGCCGCCCGGGCGTCGGG |
| | CGACCTTTGGTCGCCCGGCCTCAGTGAGCGAGCGAGCGCGCAGAGAGGGAGT |
| | GGCCAACTCCATCACTAGGGGTTCCT |
| 15 | gttctagggtggaaactaagagaatgatgtacctagagggcgctggaagctctaaagccctagcagttactgcttttactattagtg |
| | gtcgtttttttctcccccccgccccccgacaaatcaacagaacaaagaaaattacctaaacagcaaggacatagggaggaacttc |
| | ttggcacagaactttccaaacactttttcctgaagggatacaagaagcaagaaaggtactctttcactaggaccttctctgagctgtc |
| | ctcaggatgcttttgggactatttttcttacccagagaatggagaaaccctgcagggaattcccaagctgtagttataaacagaagtt |
| | ctccttctgctaggtagcattcaaagatcttaatcttctgggtttccgttttctcgaatgaaaaatgcaggtccgagcagttaactggct |
| | ggggcaccattagcaagtcacttagcatctctggggccagtctgcaaagcgagggggcagccttaatgtgcctccagcctgaag |
| | tcctagaatgagcgcccggtgtcccaagctggggcgcgcaccccagatcggagggcgccgatgtacagacagcaaactcac |
| | ccagtctagtgcatgccttcttaaacatcacgagactctaagaaaaggaaactgaaaacgggaaagtccctctctctaacctggca |
| | ctgcgtcgctggcttggagacaggtgacggtccctgcgggccttgtcctgattggctgggcacgcgtttaatataagtggaggcg |
| | tcgcgctggcgggcattcctgaagct |
| 16 | gacattgattattgactagtTATTAATAGTAATCAATTACGGGGTCATTAGTTCATAGCCC |
| | ATATATGGAGTTCCGCGTTACATAACTTACGGTAAATGGCCCGCCTGGCTGAC |
| | CGCCCAACGACCCCCGCCCATTGACGTCAATAATGACGTATGTTCCCATAGTA |
| | ACGCCAATAGGGACTTTCCATTGACGTCAATGGGTGGACTATTTACGGTAAAC |
| | TGCCCACTTGGCAGTACATCAAGTGTATCATATGCCAAGTACGCCCCCTATTG |
| | ACGTCAATGACGGTAAATGGCCCGCCTGGCATTATGCCCAGTACATGACCTTA |
| | TGGGACTTTCCTACTTGGCAGTACATCTACGTATTAGTCATCGCTATTACCAT |
| | GGGTCGAGGTGAGCCCCACGTTCTGCTTCACTCTCCCCATCTCCCCCCCCTCC |
| | CCACCCCCAATTTTGTATTTATTTATTTTTTAATTATTTTGTGCAGCGATGGGG |
| | GCGGGGGGGGGGGGGGCGCGCGCCAGGCGGGGCGGGGCGGGGCGAGGGGC |
| | GGGGCGGGGCGAGGCGGAGAGGTGCGGCGGCAGCCAATCAGAGCGGCGCGC |
| | TCCGAAAGTTTCCTTTTATGGCGAGGCGGCGGCGGCGGCGGCCCTATAAAAA |
| | GCGAAGCGCGCGGCGGGCGGGAGTCGCTGCGTTGCCTTCGCCCCGTGCCCCG |
| | CTCCGCGCCGCCTCGCGCCGCCCGCCCCGGCTCTGACTGACCGCGTTACTCCC |
| | ACAGGTGAGCGGGCGGGACGGCCCTTCTCCTCCGGGCTGTAATTAGCGCTTG |
| | GTTTAATGACGGCTCGTTTCTTTTCTGTGGCTGCGTGAAAGCCTTAAAGGGCT |
| | CCGGGAGGGCCCTTTGTGCGGGGGGGAGCGGCTCGGGGGGTGCGTGCGTGTG |
| | TGTGTGCGTGGGGAGCGCCGCGTGCGGCCCGCGCTGCCCGGCGGCTGTGAGC |
| | GCTGCGGGCGCGGCGCGGGGCTTTGTGCGCTCCGCGTGTGCGCGAGGGGAGC |
| | GCGGCCGGGGGCGGTGCCCCGCGGTGCGGGGGGGCTGCGAGGGGAACAAAG |
| | GCTGCGTGCGGGGTGTGTGCGTGGGGGGGTGAGCAGGGGGTGTGGGCGCGGC |
| | GGTCGGGCTGTAACCCCCCCCTGCACCCCCCTCCCCGAGTTGCTGAGCACGGC |
| | CCGGCTTCGGGTGCGGGGCTCCGTGCGGGGCGTGGCGCGGGGCTCGCCGTGC |
| | CGGGCGGGGGGTGGCGGCAGGTGGGGGTGCCGGGCGGGGCGGGGCCGCCTC |
| | GGGCCGGGGAGGGCTCGGGGGAGGGGCGCGGCGGCCCCGGAGCGCCGGCGG |
| | CTGTCGAGGCGCGGCGAGCCGCAGCCATTGCCTTTTATGGTAATCGTGCGAG |
| | AGGGCGCAGGGACTTCCTTTGTCCCAAATCTGGCGGAGCCGAAATCTGGGAG |
| | GCGCCGCCGCACCCCCTCTAGCGGGCGCGGGCGAAGCGGTGCGGCGCCGGCA |
| | GGAAGGAAATGGGCGGGGAGGGCCTTCGTGCGTCGCCGCGCCGCCGTCCCCT |
| | TCTCCATCTCCAGCCTCGGGGCTGCCGCAGGGGGACGGCTGCCTTCGGGGGG |
| | GACGGGGCAGGGCGGGGTTCGGCTTCTGGCGTGTGACCGGCGGCTCTAGAGC |
| | CTCTGCTAACCATGTTCATGCCTTCTTCTTTTTCCTACAG |
| 17 | atgaggaggatgtgggccacgcaggggctggcggtggcgctggctctgagcgtgctgccgggcagccgggcgctgcggcc |
| | gggcgactgcgaagtttgtatttcttatctgggaagattttaccaggacctcaaagacagagatgtcacattctcaccagccactatt |
| | gaaaacgaacttataaagttctgccgggaagcaagaggcaaagagaatcggttgtgctactatatcggggccacagatgatgca |
| | gccaccaaaatcatcaatgaggtatcaaagcctctggcccaccacatccctgtggagaagatctgtgagaagcttaagaagaag |
| | gacagccagatatgtgagcttaagtatgacaagcagatcgacctgagcacagtggacctgaagaagctccgagttaaagagct |
| | gaagaagattctggatgactggggggagacatgcaaaggctgtgcagaaaagtctgactacatccggaagataaatgaactgat |
| | gcctaaatatgcccccaaggcagccagtgcacggaccgatttg |
| 18 | GCTACTAACTTCAGCCTGCTGAAGCAGGCTGGAGACGTGGAGGAGAACCCTG |
| | GACCT |
| 19 | atggctgaacaagtccttcctcaggctttgtatttgagcaatatgcggaaagctgtgaagatacgggagagaactccagaagaca |
| | tttttaaacctactaatgggatcattcatcattttaaaaccatgcaccgatacacactggaaatgttcagaacttgccagttttgtcctca |
| | gtttcgggagatcatccacaaagccctcatcgacagaaacatccaggccaccctggaaagccagaagaaactcaactggtgtc |
| | gagaagtccggaagcttgtggcgctgaaaacgaacggtgacggcaattgcctcatgcatgccacttctcagtacatgtggggcg |
| | ttcaggacacagacttggtactgaggaaggcgctgttcagcacgctcaaggaaacagacacacgcaactttaaattccgctggc |
| | aactggagtctctcaaatctcaggaatttgttgaaacggggctttgctatgatactcggaactggaatgatgaatgggacaatcttat |
| | caaaatggcttccacagacacacccatggcccgaagtggacttcagtacaactcactggaagaaatacacatatttgtcctttgca |
| | acatcctcagaaggccaatcattgtcatttcagacaaaatgctaagaagtttggaatcaggttccaatttcgcccctttgaaagtggg |
| | tggaatttacttgcctctccactggcctgcccaggaatgctacagataccccattgttctcggctatgacagccatcattttgtaccct |
| | tggtgaccctgaaggacagtgggcctgaaatccgagctgttccacttgttaacagagaccggggaagatttgaagacttaaaagt |
| | tcactttttgacagatcctgaaaatgagatgaaggagaagctcttaaaagagtacttaatggtgatagaaatccccgtccaaggctg |
| | ggaccatggcacaactcatctcatcaatgccgcaaagttggatgaagctaacttaccaaaagaaatcaatctggtagatgattactt |
| | tgaacttgttcagcatgagtacaagaaatggcaggaaaacagcgagcaggggaggagagaggggcacgcccagaatcccat |
| | ggaaccttccgtgccccagctttctctcatggatgtaaaatgtgaaacgcccaactgccccttcttcatgtctgtgaacacccagcc |
| | tttatgccatgagtgctcagagaggcggcaaaagaatcaaaacaaactcccaaagctgaactccaagccgggccctgaggggc |
| | tccctggcatggcgctcggggcctctcggggagaagcctatgagcccttggcgtggaaccctgaggagtccactggggggcc |
| | tcattcggccccaccgacagcacccagcccttttctgttcagtgagaccactgccatgaagtgcaggagccccggctgccccttc |
| | acactgaatgtgcagcacaacggattttgtgaacgttgccacaacgcccggcaacttcacgccagccacgccccagaccacac |
| | aaggcacttggatcccgggaagtgccaagcctgcctccaggatgttaccaggacatttaatgggatctgcagtacttgcttcaaaa |
| | ggactacagcagaggcctcctccagcctcagcaccagcctccctccttcctgtcaccagcgttccaagtcagatccctcgcggct |
| | cgtccggagcccctccccgcattcttgccacagagctggaaacgacgcccctgctggctgcctgtctcaagctgcacggactcc |
| | tggggacaggacggggacgagcaagtgcagaaaagccggctgcgtgtattttgggactccagaaaacaagggcttttgcaca |
| | ctgtgtttcatcgagtacagagaaaacaaacattttgctgctgcctcagggaaagtcagtcccacagcgtccaggttccagaacac |
| | cattccgtgcctggggagggaatgcggcacccttggaagcaccatgtttgaaggatactgccagaagtgtttcattgaagctcag |
| | aatcagagatttcatgaggccaaaaggacagaagagcaactgagatcgagccagcgcagagatgtgcctcgaaccacacaaa |
| | gcacctcaaggcccaagtgcgcccgggcctcctgcaagaacatcctggcctgccgcagcgaggagctctgcatggagtgtca |
| | gcatcccaaccagaggatgggccctggggcccaccggggtgagcctgcccccgaagacccccccaagcagcgttgccggg |
| | cccccgcctgtgatcattttggcaatgccaagtgcaacggctactgcaacgaatgctttcagttcaagcagatgtatggc |
| 20 | atgaggatatttgctgtctttatattcatgacctactggcatttgctgaacgcatttactgtcacggttcccaaggacctatatgtggta |
| | gagtatggtagcaatatgacaattgaatgcaaattcccagtagaaaaacaattagacctggctgcactaattgtctattgggaaatg |
| | gaggataagaacattattcaatttgtgcatggagaggaagacctgaaggttcagcatagtagctacagacagagggcccggctg |
| | ttgaaggaccagctctccctgggaaatgctgcacttcagatcacagatgtgaaattgcaggatgcaggggtgtaccgctgcatga |
| | tcagctatggtggtgccgactacaagcgaattactgtgaaagtcaatgccccatacaacaaaatcaaccaaagaattttggttgtg |
| | gatccagtcacctctgaacatgaactgacatgtcaggctgagggctaccccaaggccgaagtcatctggacaagcagtgaccat |
| | caagtcctgagtggtaagaccaccaccaccaattccaagagagaggagaaacttttcaatgtgaccagcacactgagaatcaac |
| | acaacaactaatgagattttctactgcacttttaggagattagatcctgaggaaaaccatacagctgaattggtcatcccagaactac |
| | ctctggcacatcctccaaatgaaaggactcacttggtaattctgggagccatcttattatgccttggtgtagcactgacattcatcttc |
| | cgtttaagaaaagggagaatgatggatgtgaaaaaatgtggcatccaagatacaaactcaaagaagcaaagtgatacacatttgg |
| | aggagacgtaa |
| 21 | GTGCCTTCTAGTTGCCAGCCATCTGTTGTTTGCCCCTCCCCCGTGCCTTCCTTG |
| | ACCCTGGAAGGTGCCACTCCCACTGTCCTTTCCTAATAAAATGAGGAAATTGC |
| | ATCGCATTGTCTGAGTAGGTGTCATTCTATTCTGGGGGGTGGGGTGGGGCAGG |
| | ACAGCAAGGGGGAGGATTGGGAAGACAATAGCAGGCATGCTGGGGATGCGG |
| | TGGGCTCTATGGGT |
| 22 | Ccagcgtgagtctctcctaccctcccgctctggtccttcctctcccgctctgcaccctctgtggccctcgctgtgctctctcgctccg |
| | tgacttcccttctccaagttctccttggtggcccgccgtggggctagtccagggctggatctcggggaagcggcggggtggcct |
| | gggagtggggaagggggtgcgcacccgggacgcgcgctacttgcccctttcggcggggagcaggggagacctttggcctac |
| | ggcgacgggagggtcgggacaaagtttagggcgtcgataagcgtcagagcgccgaggttgggggagggtttctcttccgctct |
| | ttcgcggggcctctggctcccccagcgcagctggagtgggggacgggtaggctcgtcccaaaggcgcggcgctgaggtttgt |
| | gaacgcgtggaggggcgcttggggtctgggggaggcgtcgcccgggtaagcctgtctgctgcggctctgcttcccttagactg |
| | gagagctgtggacttcgtctaggcgcccgctaagttcgcatgtcctagcacctctgggtctatgtggggccacaccgtggggag |
| | gaaacagcacgcgacgtttgtagaatgcttggctgtgatacaaagcggtttcgaataattaacttatttgttcccatcacatgtcactt |
| | ttaaaaaattataagaactacccgttattgacatctttctgtgtgccaaggactttatgtgctttgcgtcatttaattttgaaaacagttatc |
| | ttccgccatagataactactatggttatcttct |
| 23 | AGGAACCCCTAGTGATGGAGTTGGCCACTCCCTCTCTGCGCGCTCGCTCGCTC |
| | ACTGAGGCCGGGCGACCAAAGGTCGCCCGACGCCCGGGCTTTGCCCGGGCGG |
| | CCTCAGTGAGCGAGCGAGCGCGCAGCTGCCTGCAGG |
| 24 | CCTGCAGGCAGCTGCGCGCTCGCTCGCTCACTGAGGCCGCCCGGGCGTCGGG |
| | CGACCTTTGGTCGCCCGGCCTCAGTGAGCGAGCGAGCGCGCAGAGAGGGAGT |
| | GGCCAACTCCATCACTAGGGGTTCCTGCGGCCGCACGCGTgttctagggtggaaactaag |
| | agaatgatgtacctagagggcgctggaagctctaaagccctagcagttactgcttttactattagtggtcgtttttttctcccccccgc |
| | cccccgacaaatcaacagaacaaagaaaattacctaaacagcaaggacatagggaggaacttcttggcacagaactttccaaac |
| | actttttcctgaagggatacaagaagcaagaaaggtactctttcactaggaccttctctgagctgtcctcaggatgcttttgggacta |
| | tttttcttacccagagaatggagaaaccctgcagggaattcccaagctgtagttataaacagaagttctccttctgctaggtagcatt |
| | caaagatcttaatcttctgggtttccgttttctcgaatgaaaaatgcaggtccgagcagttaactggctggggcaccattagcaagtc |
| | acttagcatctctggggccagtctgcaaagcgagggggcagccttaatgtgcctccagcctgaagtcctagaatgagcgcccgg |
| | tgtcccaagctggggcgcgcaccccagatcggagggcgccgatgtacagacagcaaactcacccagtctagtgcatgccttctt |
| | aaacatcacgagactctaagaaaaggaaactgaaaacgggaaagtccctctctctaacctggcactgcgtcgctggcttggaga |
| | caggtgacggtccctgcgggccttgtcctgattggctgggcacgcgtttaatataagtggaggcgtcgcgctggcgggcattcct |
| | gaagctaagcttgtggacgatatcgaattcgcacgacattgattattgactagtTATTAATAGTAATCAATTAC |
| | GGGGTCATTAGTTCATAGCCCATATATGGAGTTCCGCGTTACATAACTTACGG |
| | TAAATGGCCCGCCTGGCTGACCGCCCAACGACCCCCGCCCATTGACGTCAAT |
| | AATGACGTATGTTCCCATAGTAACGCCAATAGGGACTTTCCATTGACGTCAAT |
| | GGGTGGACTATTTACGGTAAACTGCCCACTTGGCAGTACATCAAGTGTATCAT |
| | ATGCCAAGTACGCCCCCTATTGACGTCAATGACGGTAAATGGCCCGCCTGGC |
| | ATTATGCCCAGTACATGACCTTATGGGACTTTCCTACTTGGCAGTACATCTAC |
| | GTATTAGTCATCGCTATTACCATGGGTCGAGGTGAGCCCCACGTTCTGCTTCA |
| | CTCTCCCCATCTCCCCCCCCTCCCCACCCCCAATTTTGTATTTATTTATTTTTTA |
| | ATTATTTTGTGCAGCGATGGGGGCGGGGGGGGGGGGGGCGCGCGCCAGGCG |
| | GGGCGGGGCGGGGCGAGGGGCGGGGCGGGGCGAGGCGGAGAGGTGCGGCG |
| | GCAGCCAATCAGAGCGGCGCGCTCCGAAAGTTTCCTTTTATGGCGAGGCGGC |
| | GGCGGCGGCGGCCCTATAAAAAGCGAAGCGCGCGGCGGGCGGGAGTCGCTG |
| | CGTTGCCTTCGCCCCGTGCCCCGCTCCGCGCCGCCTCGCGCCGCCCGCCCCGG |
| | CTCTGACTGACCGCGTTACTCCCACAGGTGAGCGGGCGGGACGGCCCTTCTCC |
| | TCCGGGCTGTAATTAGCGCTTGGTTTAATGACGGCTCGTTTCTTTTCTGTGGCT |
| | GCGTGAAAGCCTTAAAGGGCTCCGGGAGGGCCCTTTGTGCGGGGGGGAGCGG |
| | CTCGGGGGGTGCGTGCGTGTGTGTGTGCGTGGGGAGCGCCGCGTGCGGCCCG |
| | CGCTGCCCGGCGGCTGTGAGCGCTGCGGGCGCGGCGCGGGGCTTTGTGCGCT |
| | CCGCGTGTGCGCGAGGGGAGCGCGGCCGGGGGCGGTGCCCCGCGGTGCGGG |
| | GGGGCTGCGAGGGGAACAAAGGCTGCGTGCGGGGTGTGTGCGTGGGGGGGT |
| | GAGCAGGGGGTGTGGGCGCGGCGGTCGGGCTGTAACCCCCCCCTGCACCCCC |
| | CTCCCCGAGTTGCTGAGCACGGCCCGGCTTCGGGTGCGGGGCTCCGTGCGGG |
| | GCGTGGCGCGGGGCTCGCCGTGCCGGGCGGGGGGTGGCGGCAGGTGGGGGT |
| | GCCGGGCGGGGCGGGGCCGCCTCGGGCCGGGGAGGGCTCGGGGGAGGGGCG |
| | CGGCGGCCCCGGAGCGCCGGCGGCTGTCGAGGCGCGGCGAGCCGCAGCCATT |
| | GCCTTTTATGGTAATCGTGCGAGAGGGCGCAGGGACTTCCTTTGTCCCAAATC |
| | TGGCGGAGCCGAAATCTGGGAGGCGCCGCCGCACCCCCTCTAGCGGGCGCGG |
| | GCGAAGCGGTGCGGCGCCGGCAGGAAGGAAATGGGCGGGGAGGGCCTTCGT |
| | GCGTCGCCGCGCCGCCGTCCCCTTCTCCATCTCCAGCCTCGGGGCTGCCGCAG |
| | GGGGACGGCTGCCTTCGGGGGGGACGGGGCAGGGCGGGGTTCGGCTTCTGGC |
| | GTGTGACCGGCGGCTCTAGAGCCTCTGCTAACCATGTTCATGCCTTCTTCTTTT |
| | TCCTACAGgggggatccgtttatctgcagaattcgcccttgacgtcgccaccatgaggaggatgtgggccacgcagggg |
| | ctggcggtggcgctggctctgagcgtgctgccgggcagccgggcgctgcggccgggcgactgcgaagtttgtatttcttatctg |
| | ggaagattttaccaggacctcaaagacagagatgtcacattctcaccagccactattgaaaacgaacttataaagttctgccggga |
| | agcaagaggcaaagagaatcggttgtgctactatatcggggccacagatgatgcagccaccaaaatcatcaatgaggtatcaaa |
| | gcctctggcccaccacatccctgtggagaagatctgtgagaagcttaagaagaaggacagccagatatgtgagcttaagtatga |
| | caagcagatcgacctgagcacagtggacctgaagaagctccgagttaaagagctgaagaagattctggatgactggggggag |
| | acatgcaaaggctgtgcagaaaagtctgactacatccggaagataaatgaactgatgcctaaatatgcccccaaggcagccagt |
| | gcacggaccgatttgGGAAGCGGAGCTACTAACTTCAGCCTGCTGAAGCAGGCTGGA |
| | GACGTGGAGGAGAACCCTGGACCTatggctgaacaagtccttcctcaggctttgtatttgagcaatatgcg |
| | gaaagctgtgaagatacgggagagaactccagaagacatttttaaacctactaatgggatcattcatcattttaaaaccatgcaccg |
| | atacacactggaaatgttcagaacttgccagttttgtcctcagtttcgggagatcatccacaaagccctcatcgacagaaacatcca |
| | ggccaccctggaaagccagaagaaactcaactggtgtcgagaagtccggaagcttgtggcgctgaaaacgaacggtgacggc |
| | aattgcctcatgcatgccacttctcagtacatgtggggcgttcaggacacagacttggtactgaggaaggcgctgttcagcacgct |
| | caaggaaacagacacacgcaactttaaattccgctggcaactggagtctctcaaatctcaggaatttgttgaaacggggctttgct |
| | atgatactcggaactggaatgatgaatgggacaatcttatcaaaatggcttccacagacacacccatggcccgaagtggacttca |
| | gtacaactcactggaagaaatacacatatttgtcctttgcaacatcctcagaaggccaatcattgtcatttcagacaaaatgctaaga |
| | agtttggaatcaggttccaatttcgcccctttgaaagtgggtggaatttacttgcctctccactggcctgcccaggaatgctacagat |
| | accccattgttctcggctatgacagccatcattttgtacccttggtgaccctgaaggacagtgggcctgaaatccgagctgttccac |
| | ttgttaacagagaccggggaagatttgaagacttaaaagttcactttttgacagatcctgaaaatgagatgaaggagaagctcttaa |
| | aagagtacttaatggtgatagaaatccccgtccaaggctgggaccatggcacaactcatctcatcaatgccgcaaagttggatga |
| | agctaacttaccaaaagaaatcaatctggtagatgattactttgaacttgttcagcatgagtacaagaaatggcaggaaaacagcg |
| | agcaggggaggagagaggggcacgcccagaatcccatggaaccttccgtgccccagctttctctcatggatgtaaaatgtgaa |
| | acgcccaactgccccttcttcatgtctgtgaacacccagcctttatgccatgagtgctcagagaggcggcaaaagaatcaaaaca |
| | aactcccaaagctgaactccaagccgggccctgaggggctccctggcatggcgctcggggcctctcggggagaagcctatga |
| | gcccttggcgtggaaccctgaggagtccactggggggcctcattcggccccaccgacagcacccagcccttttctgttcagtga |
| | gaccactgccatgaagtgcaggagccccggctgccccttcacactgaatgtgcagcacaacggattttgtgaacgttgccacaa |
| | cgcccggcaacttcacgccagccacgccccagaccacacaaggcacttggatcccgggaagtgccaagcctgcctccaggat |
| | gttaccaggacatttaatgggatctgcagtacttgcttcaaaaggactacagcagaggcctcctccagcctcagcaccagcctcc |
| | ctccttcctgtcaccagcgttccaagtcagatccctcgcggctcgtccggagcccctccccgcattcttgccacagagctggaaa |
| | cgacgcccctgctggctgcctgtctcaagctgcacggactcctggggacaggacggggacgagcaagtgcagaaaagccgg |
| | ctgcgtgtattttgggactccagaaaacaagggcttttgcacactgtgtttcatcgagtacagagaaaacaaacattttgctgctgcc |
| | tcagggaaagtcagtcccacagcgtccaggttccagaacaccattccgtgcctggggagggaatgcggcacccttggaagcac |
| | catgtttgaaggatactgccagaagtgtttcattgaagctcagaatcagagatttcatgaggccaaaaggacagaagagcaactg |
| | agatcgagccagcgcagagatgtgcctogaaccacacaaagcacctcaaggcccaagtgcgcccgggcctcctgcaagaac |
| | atcctggcctgccgcagcgaggagctctgcatggagtgtcagcatcccaaccagaggatgggccctggggcccaccggggtg |
| | agcctgcccccgaagacccccccaagcagcgttgccgggcccccgcctgtgatcattttggcaatgccaagtgcaacggctac |
| | tgcaacgaatgctttcagttcaagcagatgtatggcGGAAGCGGAGCTACTAACTTCAGCCTGCTGA |
| | AGCAGGCTGGAGACGTGGAGGAGAACCCTGGACCTatgaggatatttgctgtctttatattcatg |
| | acctactggcatttgctgaacgcatttactgtcacggttcccaaggacctatatgtggtagagtatggtagcaatatgacaattgaat |
| | gcaaattcccagtagaaaaacaattagacctggctgcactaattgtctattgggaaatggaggataagaacattattcaatttgtgca |
| | tggagaggaagacctgaaggttcagcatagtagctacagacagagggcccggctgttgaaggaccagctctccctgggaaatg |
| | ctgcacttcagatcacagatgtgaaattgcaggatgcaggggtgtaccgctgcatgatcagctatggtggtgccgactacaagcg |
| | aattactgtgaaagtcaatgccccatacaacaaaatcaaccaaagaattttggttgtggatccagtcacctctgaacatgaactgac |
| | atgtcaggctgagggctaccccaaggccgaagtcatctggacaagcagtgaccatcaagtcctgagtggtaagaccaccacca |
| | ccaattccaagagagaggagaaacttttcaatgtgaccagcacactgagaatcaacacaacaactaatgagattttctactgcactt |
| | ttaggagattagatcctgaggaaaaccatacagctgaattggtcatcccagaactacctctggcacatcctccaaatgaaaggact |
| | cacttggtaattctgggagccatcttattatgccttggtgtagcactgacattcatcttccgtttaagaaaagggagaatgatggatgt |
| | gaaaaaatgtggcatccaagatacaaactcaaagaagcaaagtgatacacatttggaggagacgtaaCCGCTGATCA |
| | GCCTCGACTGTGCCTTCTAGTTGCCAGCCATCTGTTGTTTGCCCCTCCCCCGTG |
| | CCTTCCTTGACCCTGGAAGGTGCCACTCCCACTGTCCTTTCCTAATAAAATGA |
| | GGAAATTGCATCGCATTGTCTGAGTAGGTGTCATTCTATTCTGGGGGGTGGGG |
| | TGGGGCAGGACAGCAAGGGGGAGGATTGGGAAGACAATAGCAGGCATGCTG |
| | GGGATGCGGTGGGCTCTATGGGTCGACccagcgtgagtctctcctaccctcccgctctggtccttcctct |
| | cccgctctgcaccctctgtggccctcgctgtgctctctcgctccgtgacttcccttctccaagttctccttggtggcccgccgtggg |
| | gctagtccagggctggatctcggggaagcggcggggggcctgggagtggggaagggggtgcgcacccgggacgcgcgc |
| | tacttgcccctttcggggggagcaggggagacctttggcctacggcgacgggagggtcgggacaaagtttagggcgtcgata |
| | agcgtcagagcgccgaggttgggggagggtttctcttccgctctttcgcggggcctctggctcccccagcgcagctggagtgg |
| | gggacgggtaggctcgtcccaaaggcgcggcgctgaggtttgtgaacgcgtggaggggcgcttggggtctgggggaggcgt |
| | cgcccgggtaagcctgtctgctgcggctctgcttcccttagactggagagctgtggacttcgtctaggcgcccgctaagttcgcat |
| | gtcctagcacctctgggtctatgtggggccacaccgtggggaggaaacagcacgcgacgtttgtagaatgcttggctgtgatac |
| | aaagcggtttcgaataattaacttatttgttcccatcacatgtcacttttaaaaaattataagaactacccgttattgacatctttctgtgt |
| | gccaaggactttatgtgctttgcgtcatttaattttgaaaacagttatcttccgccatagataactactatggttatcttctGGTAAC |
| | CACGTGCGGACCGAGGCTGCAGCGTCGTCCTCCCTAGGAACCCCTAGTGATG |
| | GAGTTGGCCACTCCCTCTCTGCGCGCTCGCTCGCTCACTGAGGCCGGGCGACC |
| | AAAGGTCGCCCGACGCCCGGGCTTTGCCCGGGCGGCCTCAGTGAGCGAGCGA |
| | GCGCGCAGCTGCCTGCAGGGGCGCCTGATGCGGTATTTTCTCCTTACGCATCT |
| | GTGCGGTATTTCACACCGCATACGTCAAAGCAACCATAGTACGCGCCCTGTA |
| | GCGGCGCATTAAGCGCGGCGGGTGTGGTGGTTACGCGCAGCGTGACCGCTAC |
| | ACTTGCCAGCGCCCTAGCGCCCGCTCCTTTCGCTTTCTTCCCTTCCTTTCTCGC |
| | CACGTTCGCCGGCTTTCCCCGTCAAGCTCTAAATCGGGGGCTCCCTTTAGGGT |
| | TCCGATTTAGTGCTTTACGGCACCTCGACCCCAAAAAACTTGATTTGGGTGAT |
| | GGTTCACGTAGTGGGCCATCGCCCTGATAGACGGTTTTTCGCCCTTTGACGTT |
| | GGAGTCCACGTTCTTTAATAGTGGACTCTTGTTCCAAACTGGAACAACACTCA |
| | ACCCTATCTCGGGCTATTCTTTTGATTTATAAGGGATTTTGCCGATTTCGGCCT |
| | ATTGGTTAAAAAATGAGCTGATTTAACAAAAATTTAACGCGAATTTTAACAA |
| | AATATTAACGTTTACAATTTTATGGTGCACTCTCAGTACAATCTGCTCTGATG |
| | CCGCATAGTTAAGCCAGCCCCGACACCCGCCAACACCCGCTGACGCGCCCTG |
| | ACGGGCTTGTCTGCTCCCGGCATCCGCTTACAGACAAGCTGTGACCGTCTCCG |
| | GGAGCTGCATGTGTCAGAGGTTTTCACCGTCATCACCGAAACGCGCGAGACG |
| | AAAGGGCCTCGTGATACGCCTATTTTTATAGGTTAATGTCATGAACAATAAAA |
| | CTGTCTGCTTACATAAACAGTAATACAAGGGGTGTTATGAGCCATATTCAACG |
| | GGAAACGTCGAGGCCGCGATTAAATTCCAACATGGATGCTGATTTATATGGG |
| | TATAAATGGGCTCGCGATAATGTCGGGCAATCAGGTGCGACAATCTATCGCTT |
| | GTATGGGAAGCCCGATGCGCCAGAGTTGTTTCTGAAACATGGCAAAGGTAGC |
| | GTTGCCAATGATGTTACAGATGAGATGGTCAGACTAAACTGGCTGACGGAAT |
| | TTATGCCTCTTCCGACCATCAAGCATTTTATCCGTACTCCTGATGATGCATGGT |
| | TACTCACCACTGCGATCCCCGGAAAAACAGCATTCCAGGTATTAGAAGAATA |
| | TCCTGATTCAGGTGAAAATATTGTTGATGCGCTGGCAGTGTTCCTGCGCCGGT |
| | TGCATTCGATTCCTGTTTGTAATTGTCCTTTTAACAGCGATCGCGTATTTCGTC |
| | TCGCTCAGGCGCAATCACGAATGAATAACGGTTTGGTTGATGCGAGTGATTTT |
| | GATGACGAGCGTAATGGCTGGCCTGTTGAACAAGTCTGGAAAGAAATGCATA |
| | AACTTTTGCCATTCTCACCGGATTCAGTCGTCACTCATGGTGATTTCTCACTTG |
| | ATAACCTTATTTTTGACGAGGGGAAATTAATAGGTTGTATTGATGTTGGACGA |
| | GTCGGAATCGCAGACCGATACCAGGATCTTGCCATCCTATGGAACTGCCTCG |
| | GTGAGTTTTCTCCTTCATTACAGAAACGGCTTTTTCAAAAATATGGTATTGAT |
| | AATCCTGATATGAATAAATTGCAGTTTCATTTGATGCTCGATGAGTTTTTCTA |
| | ATCTCATGACCAAAATCCCTTAACGTGAGTTTTCGTTCCACTGAGCGTCAGAC |
| | CCCGTAGAAAAGATCAAAGGATCTTCTTGAGATCCTTTTTTTCTGCGCGTAAT |
| | CTGCTGCTTGCAAACAAAAAAACCACCGCTACCAGCGGTGGTTTGTTTGCCGG |
| | ATCAAGAGCTACCAACTCTTTTTCCGAAGGTAACTGGCTTCAGCAGAGCGCA |
| | GATACCAAATACTGTCCTTCTAGTGTAGCCGTAGTTAGGCCACCACTTCAAGA |
| | ACTCTGTAGCACCGCCTACATACCTCGCTCTGCTAATCCTGTTACCAGTGGCT |
| | GCTGCCAGTGGCGATAAGTCGTGTCTTACCGGGTTGGACTCAAGACGATAGTT |
| | ACCGGATAAGGCGCAGCGGTCGGGCTGAACGGGGGGTTCGTGCACACAGCCC |
| | AGCTTGGAGCGAACGACCTACACCGAACTGAGATACCTACAGCGTGAGCTAT |
| | GAGAAAGCGCCACGCTTCCCGAAGGGAGAAAGGCGGACAGGTATCCGGTAA |
| | GCGGCAGGGTCGGAACAGGAGAGCGCACGAGGGAGCTTCCAGGGGGAAACG |
| | CCTGGTATCTTTATAGTCCTGTCGGGTTTCGCCACCTCTGACTTGAGCGTCGAT |
| | TTTTGTGATGCTCGTCAGGGGGGCGGAGCCTATGGAAAAACGCCAGCAACGC |
| | GGCCTTTTTACGGTTCCTGGCCTTTTGCTGGCCTTTTGCTCACATGT |
| 26 | Catatttatggggtatatgtgaatatttattacatgcatagaaggtataatgatcatgtcaggatatttgaggtatccacatttgggatt |
| | gtttaaagattaaatgaaatagtgttaaaagtatttaatatgcccttcaacaaatgatgaggaaatcttagaatctgctcagactccttc |
| | agtttacatattaggaaactgaggcacagaaaggagcagagacttgctcaagtccacccaaagcagtagagcattgtggttaaat |
| | gcaggacttcagtcagactgtctgggttcaaatcctggttccacttggacatgggtttccttacataaatcacttcacctctctgagcc |
| | tcagttttctcatatgcaaagtgaggataataataataccttccttacatggttactgatatgagtattaaatgtgccagctcatgtgcct |
| | ggcgtataggaggtgctttataaaccttagctgttaccactcatggcattgccaaatgtgggacgggtctcctgactctctggtgtg |
| | agattgatggaatccacactttccagttcccttttctacctcctgggtatcttctcatatggttgtaagttccttggaggaagggaatgt |
| | ggcttgctctctccaccacgctgagcatataagaggtgctgaatgagcgcttttattcactcctctcatccccagccctcaccagct |
| | gggagttgttgtaggtgtcaattttctgcctctttccaacaccctgtgaggtgactgagcattgtcttccctcccaggcagctcacagt |
| | gt |
| 27 | atggaagatacaaaggagtctaacgtgaagacattttgctccaagaatatcctagccatccttggcttctcctctatcatagctgtga |
| | tagctttgcttgctgtggggttgacccagaacaaagcattgccagaaaacgttaagtatgggattgtgctggatgcgggttcttctc |
| | acacaagtttatacatctataagtggccagcagaaaaggagaatgacacaggcgtggtgcatcaagtagaagaatgcagggtta |
| | aaggtcctggaatctcaaaatttgttcagaaagtaaatgaaataggcatttacctgactgattgcatggaaagagctagggaagtg |
| | attccaaggtcccagcaccaagagacacccgtttacctgggagccacggcaggcatgcggttgctcaggatggaaagtgaaga |
| | gttggcagacagggttctggatgtggtggagaggagcctcagcaactacccctttgacttccagggtgccaggatcattactggc |
| | caagaggaaggtgcctatggctggattactatcaactatctgctgggcaaattcagtcagaaaacaaggtggttcagcatagtccc |
| | atatgaaaccaataatcaggaaacctttggagctttggaccttgggggagcctctacacaagtcacttttgtaccccaaaaccaga |
| | ctatcgagtccccagataatgctctgcaatttcgcctctatggcaaggactacaatgtctacacacatagcttcttgtgctatgggaa |
| | ggatcaggcactctggcagaaactggccaaggacattcaggttgcaagtaatgaaattctcagggacccatgctttcatcctggat |
| | ataagaaggtagtgaacgtaagtgacctttacaagaccccctgcaccaagagatttgagatgactcttccattccagcagtttgaa |
| | atccagggtattggaaactatcaacaatgccatcaaagcatcctggagctcttcaacaccagttactgcccttactcccagtgtgcc |
| | ttcaatgggattttcttgccaccactccagggggattttggggcattttcagctttttactttgtgatgaagtttttaaacttgacatcaga |
| | gaaagtctctcaggaaaaggtgactgagatgatgaaaaagttctgtgctcagccttgggaggagataaaaacatcttacgctgga |
| | gtaaaggagaagtacctgagtgaatactgcttttctggtacctacattctctccctccttctgcaaggctatcatttcacagctgattcc |
| | tgggagcacatccatttcattggcaagatccagggcagcgacgccggctggactttgggctacatgctgaacctgaccaacatg |
| | atcccagctgagcaaccattgtccacacctctctcccactccacctatgtcttcctcatggttctattctccctggtccttttcacagtg |
| | gccatcataggcttgcttatctttcacaagccttcatatttctggaaagatatggta |
| 28 | tgaccagatggacctggctggagaagaagagattgagctctactcaggtgggccctcctccctctggtctcttccggtatccccc |
| | acccctcagcttgctgtagagacggcaatcaggggaaattctggtccctgccctcccgtcagcaccacggacagctcccacgtc |
| | tgtgggacgctctctgcagatggggatgatctcccagccctgccccgcctctccctcgttccccaccagccctctttccagaaattt |
| | ccttcttcatccaagggacttttcctcccagaacccgacacagacaccatcaactgcgaccagttcagcaggctgttgtgtgacat |
| | ggaaggtgatgaagagaccagggaggcttatgccaatatcggtgaggaagcacctgagcccagaaaaggacaatcaagggc |
| | aagagttctttgctgccacttgtcaatatcacccattcatcatgagccacgtcagtcccctcccacagaaatcattgcaagggggat |
| | gcggagcaatggctggaggaacggagactccagggaagagaggggagatggaggccagtgggggaaataggccccttca |
| | ctaatgaccaccaagaaaacaaaatctcatgtttacatcctccacctccatttctatacgcatttctgcttcttgctcttctgtccatcctt |
| | tctacaaagcccataccatacacccctttcccttttcctcccagctccttagccaagctactctagtatttgtaataactagcatttactg |
| | gatactcatagtatgctcattgctgtcc |
| 29 | CCTGCAGGCAGCTGCGCGCTCGCTCGCTCACTGAGGCCGCCCGGGCGTCGGG |
| | CGACCTTTGGTCGCCCGGCCTCAGTGAGCGAGCGAGCGCGCAGAGAGGGAGT |
| | GGCCAACTCCATCACTAGGGGTTCCTGCGGCCGCACGCGTCatatttatggggtatatgtga |
| | atatttattacatgcatagaaggtataatgatcatgtcaggatatttgaggtatccacatttgggattgtttaaagattaaatgaaatagt |
| | gttaaaagtatttaatatgcccttcaacaaatgatgaggaaatcttagaatctgctcagactccttcagtttacatattaggaaactga |
| | ggcacagaaaggagcagagacttgctcaagtccacccaaagcagtagagcattgtggttaaatgcaggacttcagtcagactgt |
| | ctgggttcaaatcctggttccacttggacatgggtttccttacataaatcacttcacctctctgagcctcagttttctcatatgcaaagt |
| | gaggataataataataccttccttacatggttactgatatgagtattaaatgtgccagctcatgtgcctggcgtataggaggtgcttta |
| | taaaccttagctgttaccactcatggcattgccaaatgtgggacgggtctcctgactctctggtgtgagattgatggaatccacactt |
| | tccagttcccttttctacctcctgggtatcttctcatatggttgtaagttccttggaggaagggaatgtggcttgctctctccaccacgc |
| | tgagcatataagaggtgctgaatgagcgcttttattcactcctctcatccccagccctcaccagctgggagttgttgtaggtgtcaat |
| | tttctgcctctttccaacaccctgtgaggtgactgagcattgtcttccctcccaggcagctcacagtgtaagcttgtggacgatatcg |
| | aattcgcacgacattgattattgactagtTATTAATAGTAATCAATTACGGGGTCATTAGTTCAT |
| | AGCCCATATATGGAGTTCCGCGTTACATAACTTACGGTAAATGGCCCGCCTGG |
| | CTGACCGCCCAACGACCCCCGCCCATTGACGTCAATAATGACGTATGTTCCCA |
| | TAGTAACGCCAATAGGGACTTTCCATTGACGTCAATGGGTGGACTATTTACGG |
| | TAAACTGCCCACTTGGCAGTACATCAAGTGTATCATATGCCAAGTACGCCCCC |
| | TATTGACGTCAATGACGGTAAATGGCCCGCCTGGCATTATGCCCAGTACATGA |
| | CCTTATGGGACTTTCCTACTTGGCAGTACATCTACGTATTAGTCATCGCTATTA |
| | CCATGGGTCGAGGTGAGCCCCACGTTCTGCTTCACTCTCCCCATCTCCCCCCC |
| | CTCCCCACCCCCAATTTTGTATTTATTTATTTTTTAATTATTTTGTGCAGCGAT |
| | GGGGGCGGGGGGGGGGGGGGCGCGCGCCAGGCGGGGCGGGGCGGGGCGAG |
| | GGGCGGGGCGGGGCGAGGCGGAGAGGTGCGGCGGCAGCCAATCAGAGCGGC |
| | GCGCTCCGAAAGTTTCCTTTTATGGCGAGGCGGCGGCGGCGGCGGCCCTATA |
| | AAAAGCGAAGCGCGCGGCGGGCGGGAGTCGCTGCGTTGCCTTCGCCCCGTGC |
| | CCCGCTCCGCGCCGCCTCGCGCCGCCCGCCCCGGCTCTGACTGACCGCGTTAC |
| | TCCCACAGGTGAGCGGGCGGGACGGCCCTTCTCCTCCGGGCTGTAATTAGCG |
| | CTTGGTTTAATGACGGCTCGTTTCTTTTCTGTGGCTGCGTGAAAGCCTTAAAG |
| | GGCTCCGGGAGGGCCCTTTGTGCGGGGGGGAGCGGCTCGGGGGGTGCGTGCG |
| | TGTGTGTGTGCGTGGGGAGCGCCGCGTGCGGCCCGCGCTGCCCGGCGGCTGT |
| | GAGCGCTGCGGGCGCGGCGCGGGGCTTTGTGCGCTCCGCGTGTGCGCGAGGG |
| | GAGCGCGGCCGGGGGCGGTGCCCCGCGGTGCGGGGGGGCTGCGAGGGGAAC |
| | AAAGGCTGCGTGCGGGGTGTGTGCGTGGGGGGGTGAGCAGGGGGTGTGGGC |
| | GCGGCGGTCGGGCTGTAACCCCCCCCTGCACCCCCCTCCCCGAGTTGCTGAGC |
| | ACGGCCCGGCTTCGGGTGCGGGGCTCCGTGCGGGGCGTGGCGCGGGGCTCGC |
| | CGTGCCGGGCGGGGGGTGGCGGCAGGTGGGGGTGCCGGGCGGGGCGGGGCC |
| | GCCTCGGGCCGGGGAGGGCTCGGGGGAGGGGCGCGGCGGCCCCGGAGCGCC |
| | GGCGGCTGTCGAGGCGCGGCGAGCCGCAGCCATTGCCTTTTATGGTAATCGT |
| | GCGAGAGGGCGCAGGGACTTCCTTTGTCCCAAATCTGGCGGAGCCGAAATCT |
| | GGGAGGCGCCGCCGCACCCCCTCTAGCGGGCGCGGGCGAAGCGGTGCGGCGC |
| | CGGCAGGAAGGAAATGGGCGGGGAGGGCCTTCGTGCGTCGCCGCGCCGCCGT |
| | CCCCTTCTCCATCTCCAGCCTCGGGGCTGCCGCAGGGGGACGGCTGCCTTCGG |
| | GGGGGACGGGGCAGGGCGGGGTTCGGCTTCTGGCGTGTGACCGGCGGCTCTA |
| | GAGCCTCTGCTAACCATGTTCATGCCTTCTTCTTTTTCCTACAGgggggatccgtttatct |
| | gcagaattcgcccttgacgtcgccaccatggaagatacaaaggagtctaacgtgaagacattttgctccaagaatatcctagccat |
| | ccttggcttctcctctatcatagctgtgatagctttgcttgctgtggggttgacccagaacaaagcattgccagaaaacgttaagtat |
| | gggattgtgctggatgcgggttcttctcacacaagtttatacatctataagtggccagcagaaaaggagaatgacacaggcgtgg |
| | tgcatcaagtagaagaatgcagggttaaaggtcctggaatctcaaaatttgttcagaaagtaaatgaaataggcatttacctgactg |
| | attgcatggaaagagctagggaagtgattccaaggtcccagcaccaagagacacccgtttacctgggagccacggcaggcatg |
| | cggttgctcaggatggaaagtgaagagttggcagacagggttctggatgtggtggagaggagcctcagcaactacccctttgac |
| | ttccagggtgccaggatcattactggccaagaggaaggtgcctatggctggattactatcaactatctgctgggcaaattcagtca |
| | gaaaacaaggtggttcagcatagtcccatatgaaaccaataatcaggaaacctttggagctttggaccttgggggagcctctaca |
| | caagtcacttttgtaccccaaaaccagactatcgagtccccagataatgctctgcaatttcgcctctatggcaaggactacaatgtct |
| | acacacatagcttcttgtgctatgggaaggatcaggcactctggcagaaactggccaaggacattcaggttgcaagtaatgaaatt |
| | ctcagggacccatgctttcatcctggatataagaaggtagtgaacgtaagtgacctttacaagaccccctgcaccaagagatttga |
| | gatgactcttccattccagcagtttgaaatccagggtattggaaactatcaacaatgccatcaaagcatcctggagctcttcaacac |
| | cagttactgcccttactcccagtgtgccttcaatgggattttcttgccaccactccagggggattttggggcattttcagctttttacttt |
| | gtgatgaagtttttaaacttgacatcagagaaagtctctcaggaaaaggtgactgagatgatgaaaaagttctgtgctcagccttgg |
| | gaggagataaaaacatcttacgctggagtaaaggagaagtacctgagtgaatactgcttttctggtacctacattctctccctccttc |
| | tgcaaggctatcatttcacagctgattcctgggagcacatccatttcattggcaagatccagggcagcgacgccggctggactttg |
| | ggctacatgctgaacctgaccaacatgatcccagctgagcaaccattgtccacacctctctcccactccacctatgtcttcctcatg |
| | gttctattctccctggtccttttcacagtggccatcataggcttgcttatctttcacaagccttcatatttctggaaagatatggtataatg |
| | atagCCGCTGATCAGCCTCGACTGTGCCTTCTAGTTGCCAGCCATCTGTTGTTTG |
| | CCCCTCCCCCGTGCCTTCCTTGACCCTGGAAGGTGCCACTCCCACTGTCCTTTC |
| | CTAATAAAATGAGGAAATTGCATCGCATTGTCTGAGTAGGTGTCATTCTATTC |
| | TGGGGGGTGGGGTGGGGCAGGACAGCAAGGGGGAGGATTGGGAAGACAATA |
| | GCAGGCATGCTGGGGATGCGGTGGGCTCTATGGGTCGACtgaccagatggacctggctgg |
| | agaagaagagattgagctctactcaggtgggccctcctccctctggtctcttccggtatcccccacccctcagcttgctgtagaga |
| | cggcaatcaggggaaattctggtccctgccctcccgtcagcaccacggacagctcccacgtctgtgggacgctctctgcagatg |
| | gggatgatctcccagccctgccccgcctctccctcgttccccaccagccctctttccagaaatttccttcttcatccaagggacttttc |
| | ctcccagaacccgacacagacaccatcaactgcgaccagttcagcaggctgttgtgtgacatggaaggtgatgaagagaccag |
| | ggaggcttatgccaatatcggtgaggaagcacctgagcccagaaaaggacaatcaagggcaagagttctttgctgccacttgtc |
| | aatatcacccattcatcatgagccacgtcagtcccctcccacagaaatcattgcaagggggatgcggagcaatggctggaggaa |
| | cggagactccagggaagagaggggagatggaggccagtgggggaaataggccccttcactaatgaccaccaagaaaacaa |
| | aatctcatgtttacatcctccacctccatttctatacgcatttctgcttcttgctcttctgtccatcctttctacaaagcccataccatacac |
| | ccctttcccttttcctcccagctccttagccaagctactctagtatttgtaataactagcatttactggatactcatagtatgctcattgct |
| | gtccGGTAACCACGTGCGGACCGAGGCTGCAGCGTCGTCCTCCCTAGGAACCCC |
| | TAGTGATGGAGTTGGCCACTCCCTCTCTGCGCGCTCGCTCGCTCACTGAGGCC |
| | GGGCGACCAAAGGTCGCCCGACGCCCGGGCTTTGCCCGGGCGGCCTCAGTGA |
| | GCGAGCGAGCGCGCAGCTGCCTGCAGGGGCGCCTGATGCGGTATTTTCTCCTT |
| | ACGCATCTGTGCGGTATTTCACACCGCATACGTCAAAGCAACCATAGTACGC |
| | GCCCTGTAGCGGCGCATTAAGCGCGGCGGGTGTGGTGGTTACGCGCAGCGTG |
| | ACCGCTACACTTGCCAGCGCCCTAGCGCCCGCTCCTTTCGCTTTCTTCCCTTCC |
| | TTTCTCGCCACGTTCGCCGGCTTTCCCCGTCAAGCTCTAAATCGGGGGCTCCC |
| | TTTAGGGTTCCGATTTAGTGCTTTACGGCACCTCGACCCCAAAAAACTTGATT |
| | TGGGTGATGGTTCACGTAGTGGGCCATCGCCCTGATAGACGGTTTTTCGCCCT |
| | TTGACGTTGGAGTCCACGTTCTTTAATAGTGGACTCTTGTTCCAAACTGGAAC |
| | AACACTCAACCCTATCTCGGGCTATTCTTTTGATTTATAAGGGATTTTGCCGAT |
| | TTCGGCCTATTGGTTAAAAAATGAGCTGATTTAACAAAAATTTAACGCGAATT |
| | TTAACAAAATATTAACGTTTACAATTTTATGGTGCACTCTCAGTACAATCTGC |
| | TCTGATGCCGCATAGTTAAGCCAGCCCCGACACCCGCCAACACCCGCTGACG |
| | CGCCCTGACGGGCTTGTCTGCTCCCGGCATCCGCTTACAGACAAGCTGTGACC |
| | GTCTCCGGGAGCTGCATGTGTCAGAGGTTTTCACCGTCATCACCGAAACGCGC |
| | GAGACGAAAGGGCCTCGTGATACGCCTATTTTTATAGGTTAATGTCATGAACA |
| | ATAAAACTGTCTGCTTACATAAACAGTAATACAAGGGGTGTTATGAGCCATA |
| | TTCAACGGGAAACGTCGAGGCCGCGATTAAATTCCAACATGGATGCTGATTT |
| | ATATGGGTATAAATGGGCTCGCGATAATGTCGGGCAATCAGGTGCGACAATC |
| | TATCGCTTGTATGGGAAGCCCGATGCGCCAGAGTTGTTTCTGAAACATGGCAA |
| | AGGTAGCGTTGCCAATGATGTTACAGATGAGATGGTCAGACTAAACTGGCTG |
| | ACGGAATTTATGCCTCTTCCGACCATCAAGCATTTTATCCGTACTCCTGATGA |
| | TGCATGGTTACTCACCACTGCGATCCCCGGAAAAACAGCATTCCAGGTATTAG |
| | AAGAATATCCTGATTCAGGTGAAAATATTGTTGATGCGCTGGCAGTGTTCCTG |
| | CGCCGGTTGCATTCGATTCCTGTTTGTAATTGTCCTTTTAACAGCGATCGCGTA |
| | TTTCGTCTCGCTCAGGCGCAATCACGAATGAATAACGGTTTGGTTGATGCGAG |
| | TGATTTTGATGACGAGCGTAATGGCTGGCCTGTTGAACAAGTCTGGAAAGAA |
| | ATGCATAAACTTTTGCCATTCTCACCGGATTCAGTCGTCACTCATGGTGATTTC |
| | TCACTTGATAACCTTATTTTTGACGAGGGGAAATTAATAGGTTGTATTGATGT |
| | TGGACGAGTCGGAATCGCAGACCGATACCAGGATCTTGCCATCCTATGGAAC |
| | TGCCTCGGTGAGTTTTCTCCTTCATTACAGAAACGGCTTTTTCAAAAATATGG |
| | TATTGATAATCCTGATATGAATAAATTGCAGTTTCATTTGATGCTCGATGAGT |
| | TTTTCTAATCTCATGACCAAAATCCCTTAACGTGAGTTTTCGTTCCACTGAGCG |
| | TCAGACCCCGTAGAAAAGATCAAAGGATCTTCTTGAGATCCTTTTTTTCTGCG |
| | CGTAATCTGCTGCTTGCAAACAAAAAAACCACCGCTACCAGCGGTGGTTTGTT |
| | TGCCGGATCAAGAGCTACCAACTCTTTTTCCGAAGGTAACTGGCTTCAGCAGA |
| | GCGCAGATACCAAATACTGTCCTTCTAGTGTAGCCGTAGTTAGGCCACCACTT |
| | CAAGAACTCTGTAGCACCGCCTACATACCTCGCTCTGCTAATCCTGTTACCAG |
| | TGGCTGCTGCCAGTGGCGATAAGTCGTGTCTTACCGGGTTGGACTCAAGACG |
| | ATAGTTACCGGATAAGGCGCAGCGGTCGGGCTGAACGGGGGGTTCGTGCACA |
| | CAGCCCAGCTTGGAGCGAACGACCTACACCGAACTGAGATACCTACAGCGTG |
| | AGCTATGAGAAAGCGCCACGCTTCCCGAAGGGAGAAAGGCGGACAGGTATC |
| | CGGTAAGCGGCAGGGTCGGAACAGGAGAGCGCACGAGGGAGCTTCCAGGGG |
| | GAAACGCCTGGTATCTTTATAGTCCTGTCGGGTTTCGCCACCTCTGACTTGAG |
| | CGTCGATTTTTGTGATGCTCGTCAGGGGGGCGGAGCCTATGGAAAAACGCCA |
| | GCAACGCGGCCTTTTTACGGTTCCTGGCCTTTTGCTGGCCTTTTGCTCACATGT |
| 30 | CCTGCAGGCAGCTGCGCGCTCGCTCGCTCACTGAGGCCGCCCGGGCGTCGGG |
| | CGACCTTTGGTCGCCCGGCCTCAGTGAGCGAGCGAGCGCGCAGAGAGGGAGT |
| | GGCCAACTCCATCACTAGGGGTTCCTGCGGCCGCACGCGTgttctagggtggaaactaag |
| | agaatgatgtacctagagggcgctggaagctctaaagccctagcagttactgcttttactattagtggtcgtttttttctcccccccgc |
| | cccccgacaaatcaacagaacaaagaaaattacctaaacagcaaggacatagggaggaacttcttggcacagaactttccaaac |
| | actttttcctgaagggatacaagaagcaagaaaggtactctttcactaggaccttctctgagctgtcctcaggatgcttttgggacta |
| | tttttcttacccagagaatggagaaaccctgcagggaattcccaagctgtagttataaacagaagttctccttctgctaggtagcatt |
| | caaagatcttaatcttctgggtttccgttttctcgaatgaaaaatgcaggtccgagcagttaactggctggggcaccattagcaagtc |
| | acttagcatctctggggccagtctgcaaagcgagggggcagccttaatgtgcctccagcctgaagtcctagaatgagcgcccgg |
| | tgtcccaagctggggcgcgcaccccagatcggagggcgccgatgtacagacagcaaactcacccagtctagtgcatgccttctt |
| | aaacatcacgagactctaagaaaaggaaactgaaaacgggaaagtccctctctctaacctggcactgcgtcgctggcttggaga |
| | caggtgacggtccctgcgggccttgtcctgattggctgggcacgcgtttaatataagtggaggcgtcgcgctgggggcattcct |
| | gaagctaagcttgtggacgatatcgaattcgcacgacattgattattgactagtTATTAATAGTAATCAATTAC |
| | GGGGTCATTAGTTCATAGCCCATATATGGAGTTCCGCGTTACATAACTTACGG |
| | TAAATGGCCCGCCTGGCTGACCGCCCAACGACCCCCGCCCATTGACGTCAAT |
| | AATGACGTATGTTCCCATAGTAACGCCAATAGGGACTTTCCATTGACGTCAAT |
| | GGGTGGACTATTTACGGTAAACTGCCCACTTGGCAGTACATCAAGTGTATCAT |
| | ATGCCAAGTACGCCCCCTATTGACGTCAATGACGGTAAATGGCCCGCCTGGC |
| | ATTATGCCCAGTACATGACCTTATGGGACTTTCCTACTTGGCAGTACATCTAC |
| | GTATTAGTCATCGCTATTACCATGGGTCGAGGTGAGCCCCACGTTCTGCTTCA |
| | CTCTCCCCATCTCCCCCCCCTCCCCACCCCCAATTTTGTATTTATTTATTTTTTA |
| | ATTATTTTGTGCAGCGATGGGGGCGGGGGGGGGGGGGGCGCGCGCCAGGCG |
| | GGGCGGGGCGGGGCGAGGGGCGGGGCGGGGCGAGGCGGAGAGGTGCGGCG |
| | GCAGCCAATCAGAGCGGCGCGCTCCGAAAGTTTCCTTTTATGGCGAGGCGGC |
| | GGCGGCGGCGGCCCTATAAAAAGCGAAGCGCGCGGCGGGCGGGAGTCGCTG |
| | CGTTGCCTTCGCCCCGTGCCCCGCTCCGCGCCGCCTCGCGCCGCCCGCCCCGG |
| | CTCTGACTGACCGCGTTACTCCCACAGGTGAGCGGGCGGGACGGCCCTTCTCC |
| | TCCGGGCTGTAATTAGCGCTTGGTTTAATGACGGCTCGTTTCTTTTCTGTGGCT |
| | GCGTGAAAGCCTTAAAGGGCTCCGGGAGGGCCCTTTGTGCGGGGGGGAGCGG |
| | CTCGGGGGGTGCGTGCGTGTGTGTGTGCGTGGGGAGCGCCGCGTGCGGCCCG |
| | CGCTGCCCGGCGGCTGTGAGCGCTGCGGGCGCGGCGCGGGGCTTTGTGCGCT |
| | CCGCGTGTGCGCGAGGGGAGCGCGGCCGGGGGCGGTGCCCCGCGGTGCGGG |
| | GGGGCTGCGAGGGGAACAAAGGCTGCGTGCGGGGTGTGTGCGTGGGGGGGT |
| | GAGCAGGGGGTGTGGGCGCGGCGGTCGGGCTGTAACCCCCCCCTGCACCCCC |
| | CTCCCCGAGTTGCTGAGCACGGCCCGGCTTCGGGTGCGGGGCTCCGTGCGGG |
| | GCGTGGCGCGGGGCTCGCCGTGCCGGGCGGGGGGTGGCGGCAGGTGGGGGT |
| | GCCGGGCGGGGCGGGGCCGCCTCGGGCCGGGGAGGGCTCGGGGGAGGGGCG |
| | CGGCGGCCCCGGAGCGCCGGCGGCTGTCGAGGCGCGGCGAGCCGCAGCCATT |
| | GCCTTTTATGGTAATCGTGCGAGAGGGCGCAGGGACTTCCTTTGTCCCAAATC |
| | TGGCGGAGCCGAAATCTGGGAGGCGCCGCCGCACCCCCTCTAGCGGGCGCGG |
| | GCGAAGCGGTGCGGCGCCGGCAGGAAGGAAATGGGCGGGGAGGGCCTTCGT |
| | GCGTCGCCGCGCCGCCGTCCCCTTCTCCATCTCCAGCCTCGGGGCTGCCGCAG |
| | GGGGACGGCTGCCTTCGGGGGGGACGGGGCAGGGCGGGGTTCGGCTTCTGGC |
| | GTGTGACCGGCGGCTCTAGAGCCTCTGCTAACCATGTTCATGCCTTCTTCTTTT |
| | TCCTACAGgggggatccgtttatctgcagaattcgcccttgacgtcgccaccatggaagatacaaaggagtctaacgtga |
| | agacattttgctccaagaatatcctagccatccttggcttctcctctatcatagctgtgatagctttgcttgctgtggggttgacccaga |
| | acaaagcattgccagaaaacgttaagtatgggattgtgctggatgcgggttcttctcacacaagtttatacatctataagtggccag |
| | cagaaaaggagaatgacacaggcgtggtgcatcaagtagaagaatgcagggttaaaggtcctggaatctcaaaatttgttcaga |
| | aagtaaatgaaataggcatttacctgactgattgcatggaaagagctagggaagtgattccaaggtcccagcaccaagagacac |
| | ccgtttacctgggagccacggcaggcatgcggttgctcaggatggaaagtgaagagttggcagacagggttctggatgtggtg |
| | gagaggagcctcagcaactacccctttgacttccagggtgccaggatcattactggccaagaggaaggtgcctatggctggatt |
| | actatcaactatctgctgggcaaattcagtcagaaaacaaggtggttcagcatagtcccatatgaaaccaataatcaggaaaccttt |
| | ggagctttggaccttgggggagcctctacacaagtcacttttgtaccccaaaaccagactatcgagtccccagataatgctctgca |
| | atttcgcctctatggcaaggactacaatgtctacacacatagcttcttgtgctatgggaaggatcaggcactctggcagaaactgg |
| | ccaaggacattcaggttgcaagtaatgaaattctcagggacccatgctttcatcctggatataagaaggtagtgaacgtaagtgac |
| | ctttacaagaccccctgcaccaagagatttgagatgactcttccattccagcagtttgaaatccagggtattggaaactatcaacaat |
| | gccatcaaagcatcctggagctcttcaacaccagttactgcccttactcccagtgtgccttcaatgggattttcttgccaccactcca |
| | gggggattttggggcattttcagctttttactttgtgatgaagtttttaaacttgacatcagagaaagtctctcaggaaaaggtgactg |
| | agatgatgaaaaagttctgtgctcagccttgggaggagataaaaacatcttacgctggagtaaaggagaagtacctgagtgaata |
| | ctgcttttctggtacctacattctctccctccttctgcaaggctatcatttcacagctgattcctgggagcacatccatttcattggcaag |
| | atccagggcagcgacgccggctggactttgggctacatgctgaacctgaccaacatgatcccagctgagcaaccattgtccaca |
| | cctctctcccactccacctatgtcttcctcatggttctattctccctggtccttttcacagtggccatcataggcttgcttatctttcacaa |
| | gccttcatatttctggaaagatatggtaGGAAGCGGAGCTACTAACTTCAGCCTGCTGAAGCAG |
| | GCTGGAGACGTGGAGGAGAACCCTGGACCTatgaggatatttgctgtctttatattcatgacctactgg |
| | catttgctgaacgcatttactgtcacggttcccaaggacctatatgtggtagagtatggtagcaatatgacaattgaatgcaaattcc |
| | cagtagaaaaacaattagacctggctgcactaattgtctattgggaaatggaggataagaacattattcaatttgtgcatggagagg |
| | aagacctgaaggttcagcatagtagctacagacagagggcccggctgttgaaggaccagctctccctgggaaatgctgcacttc |
| | agatcacagatgtgaaattgcaggatgcaggggtgtaccgctgcatgatcagctatggtggtgccgactacaagcgaattactgt |
| | gaaagtcaatgccccatacaacaaaatcaaccaaagaattttggttgtggatccagtcacctctgaacatgaactgacatgtcagg |
| | ctgagggctaccccaaggccgaagtcatctggacaagcagtgaccatcaagtcctgagtggtaagaccaccaccaccaattcc |
| | aagagagaggagaaacttttcaatgtgaccagcacactgagaatcaacacaacaactaatgagattttctactgcacttttaggag |
| | attagatcctgaggaaaaccatacagctgaattggtcatcccagaactacctctggcacatcctccaaatgaaaggactcacttgg |
| | taattctgggagccatcttattatgccttggtgtagcactgacattcatcttccgtttaagaaaagggagaatgatggatgtgaaaaa |
| | atgtggcatccaagatacaaactcaaagaagcaaagtgatacacatttggaggagacgtaaCCGCTGATCAGCCT |
| | CGACTGTGCCTTCTAGTTGCCAGCCATCTGTTGTTTGCCCCTCCCCCGTGCCTT |
| | CCTTGACCCTGGAAGGTGCCACTCCCACTGTCCTTTCCTAATAAAATGAGGAA |
| | ATTGCATCGCATTGTCTGAGTAGGTGTCATTCTATTCTGGGGGGTGGGGTGGG |
| | GCAGGACAGCAAGGGGGAGGATTGGGAAGACAATAGCAGGCATGCTGGGGA |
| | TGCGGTGGGCTCTATGGGTCGACccagcgtgagtctctcctaccctcccgctctggtccttcctctcccgctc |
| | tgcaccctctgtggccctcgctgtgctctctcgctccgtgacttcccttctccaagttctccttggtggcccgccgtggggctagtcc |
| | agggctggatctcggggaagcggcggggtggcctgggagtggggaagggggtgcgcacccgggacgcgcgctacttgccc |
| | ctttcggcggggagcaggggagacctttggcctacggcgacgggagggtcgggacaaagtttagggcgtcgataagcgtcag |
| | agcgccgaggttgggggagggtttctcttccgctctttcgcggggcctctggctcccccagcgcagctggagtgggggacggg |
| | taggctcgtcccaaaggcgcggcgctgaggtttgtgaacgcgtggaggggcgcttggggtctgggggaggcgtcgcccgggt |
| | aagcctgtctgctgcggctctgcttcccttagactggagagctgtggacttcgtctaggcgcccgctaagttcgcatgtcctagca |
| | cctctgggtctatgtggggccacaccgtggggaggaaacagcacgcgacgtttgtagaatgcttggctgtgatacaaagcggttt |
| | cgaataattaacttatttgttcccatcacatgtcacttttaaaaaattataagaactacccgttattgacatctttctgtgtgccaaggact |
| | ttatgtgctttgcgtcatttaattttgaaaacagttatcttccgccatagataactactatggttatcttctGGTAACCACGTG |
| | CGGACCGAGGCTGCAGCGTCGTCCTCCCTAGGAACCCCTAGTGATGGAGTTG |
| | GCCACTCCCTCTCTGCGCGCTCGCTCGCTCACTGAGGCCGGGCGACCAAAGGT |
| | CGCCCGACGCCCGGGCTTTGCCCGGGCGGCCTCAGTGAGCGAGCGAGCGCGC |
| | AGCTGCCTGCAGGGGCGCCTGATGCGGTATTTTCTCCTTACGCATCTGTGCGG |
| | TATTTCACACCGCATACGTCAAAGCAACCATAGTACGCGCCCTGTAGCGGCG |
| | CATTAAGCGCGGCGGGTGTGGTGGTTACGCGCAGCGTGACCGCTACACTTGC |
| | CAGCGCCCTAGCGCCCGCTCCTTTCGCTTTCTTCCCTTCCTTTCTCGCCACGTT |
| | CGCCGGCTTTCCCCGTCAAGCTCTAAATCGGGGGCTCCCTTTAGGGTTCCGAT |
| | TTAGTGCTTTACGGCACCTCGACCCCAAAAAACTTGATTTGGGTGATGGTTCA |
| | CGTAGTGGGCCATCGCCCTGATAGACGGTTTTTCGCCCTTTGACGTTGGAGTC |
| | CACGTTCTTTAATAGTGGACTCTTGTTCCAAACTGGAACAACACTCAACCCTA |
| | TCTCGGGCTATTCTTTTGATTTATAAGGGATTTTGCCGATTTCGGCCTATTGGT |
| | TAAAAAATGAGCTGATTTAACAAAAATTTAACGCGAATTTTAACAAAATATT |
| | AACGTTTACAATTTTATGGTGCACTCTCAGTACAATCTGCTCTGATGCCGCAT |
| | AGTTAAGCCAGCCCCGACACCCGCCAACACCCGCTGACGCGCCCTGACGGGC |
| | TTGTCTGCTCCCGGCATCCGCTTACAGACAAGCTGTGACCGTCTCCGGGAGCT |
| | GCATGTGTCAGAGGTTTTCACCGTCATCACCGAAACGCGCGAGACGAAAGGG |
| | CCTCGTGATACGCCTATTTTTATAGGTTAATGTCATGAACAATAAAACTGTCT |
| | GCTTACATAAACAGTAATACAAGGGGTGTTATGAGCCATATTCAACGGGAAA |
| | CGTCGAGGCCGCGATTAAATTCCAACATGGATGCTGATTTATATGGGTATAAA |
| | TGGGCTCGCGATAATGTCGGGCAATCAGGTGCGACAATCTATCGCTTGTATGG |
| | GAAGCCCGATGCGCCAGAGTTGTTTCTGAAACATGGCAAAGGTAGCGTTGCC |
| | AATGATGTTACAGATGAGATGGTCAGACTAAACTGGCTGACGGAATTTATGC |
| | CTCTTCCGACCATCAAGCATTTTATCCGTACTCCTGATGATGCATGGTTACTCA |
| | CCACTGCGATCCCCGGAAAAACAGCATTCCAGGTATTAGAAGAATATCCTGA |
| | TTCAGGTGAAAATATTGTTGATGCGCTGGCAGTGTTCCTGCGCCGGTTGCATT |
| | CGATTCCTGTTTGTAATTGTCCTTTTAACAGCGATCGCGTATTTCGTCTCGCTC |
| | AGGCGCAATCACGAATGAATAACGGTTTGGTTGATGCGAGTGATTTTGATGA |
| | CGAGCGTAATGGCTGGCCTGTTGAACAAGTCTGGAAAGAAATGCATAAACTT |
| | TTGCCATTCTCACCGGATTCAGTCGTCACTCATGGTGATTTCTCACTTGATAAC |
| | CTTATTTTTGACGAGGGGAAATTAATAGGTTGTATTGATGTTGGACGAGTCGG |
| | AATCGCAGACCGATACCAGGATCTTGCCATCCTATGGAACTGCCTCGGTGAGT |
| | TTTCTCCTTCATTACAGAAACGGCTTTTTCAAAAATATGGTATTGATAATCCTG |
| | ATATGAATAAATTGCAGTTTCATTTGATGCTCGATGAGTTTTTCTAATCTCATG |
| | ACCAAAATCCCTTAACGTGAGTTTTCGTTCCACTGAGCGTCAGACCCCGTAGA |
| | AAAGATCAAAGGATCTTCTTGAGATCCTTTTTTTCTGCGCGTAATCTGCTGCTT |
| | GCAAACAAAAAAACCACCGCTACCAGCGGTGGTTTGTTTGCCGGATCAAGAG |
| | CTACCAACTCTTTTTCCGAAGGTAACTGGCTTCAGCAGAGCGCAGATACCAAA |
| | TACTGTCCTTCTAGTGTAGCCGTAGTTAGGCCACCACTTCAAGAACTCTGTAG |
| | CACCGCCTACATACCTCGCTCTGCTAATCCTGTTACCAGTGGCTGCTGCCAGT |
| | GGCGATAAGTCGTGTCTTACCGGGTTGGACTCAAGACGATAGTTACCGGATA |
| | AGGCGCAGCGGTCGGGCTGAACGGGGGGTTCGTGCACACAGCCCAGCTTGGA |
| | GCGAACGACCTACACCGAACTGAGATACCTACAGCGTGAGCTATGAGAAAGC |
| | GCCACGCTTCCCGAAGGGAGAAAGGCGGACAGGTATCCGGTAAGCGGCAGG |
| | GTCGGAACAGGAGAGCGCACGAGGGAGCTTCCAGGGGGAAACGCCTGGTAT |
| | CTTTATAGTCCTGTCGGGTTTCGCCACCTCTGACTTGAGCGTCGATTTTTGTGA |
| | TGCTCGTCAGGGGGGCGGAGCCTATGGAAAAACGCCAGCAACGCGGCCTTTT |
| | TACGGTTCCTGGCCTTTTGCTGGCCTTTTGCTCACATGT |
| 31 | CCTGCAGGCAGCTGCGCGCTCGCTCGCTCACTGAGGCCGCCCGGGCGTCGGG |
| | CGACCTTTGGTCGCCCGGCCTCAGTGAGCGAGCGAGCGCGCAGAGAGGGAGT |
| | GGCCAACTCCATCACTAGGGGTTCCTGCGGCCGCACGCGTgttctagggtggaaactaag |
| | agaatgatgtacctagagggcgctggaagctctaaagccctagcagttactgcttttactattagtggtcgtttttttctcccccccgc |
| | cccccgacaaatcaacagaacaaagaaaattacctaaacagcaaggacatagggaggaacttcttggcacagaactttccaaac |
| | actttttcctgaagggatacaagaagcaagaaaggtactctttcactaggaccttctctgagctgtcctcaggatgcttttgggacta |
| | tttttcttacccagagaatggagaaaccctgcagggaattcccaagctgtagttataaacagaagttctccttctgctaggtagcatt |
| | caaagatcttaatcttctgggtttccgttttctcgaatgaaaaatgcaggtccgagcagttaactggctggggcaccattagcaagtc |
| | acttagcatctctggggccagtctgcaaagcgagggggcagccttaatgtgcctccagcctgaagtcctagaatgagcgcccgg |
| | tgtcccaagctggggcgcgcaccccagatcggagggcgccgatgtacagacagcaaactcacccagtctagtgcatgccttctt |
| | aaacatcacgagactctaagaaaaggaaactgaaaacgggaaagtccctctctctaacctggcactgcgtcgctggcttggaga |
| | caggtgacggtccctgcgggccttgtcctgattggctgggcacgcgtttaatataagtggaggcgtcgcgctggcgggcattcct |
| | gaagctaagcttgtggacgatatcgaattcgcacgacattgattattgactagtTATTAATAGTAATCAATTAC |
| | GGGGTCATTAGTTCATAGCCCATATATGGAGTTCCGCGTTACATAACTTACGG |
| | TAAATGGCCCGCCTGGCTGACCGCCCAACGACCCCCGCCCATTGACGTCAAT |
| | AATGACGTATGTTCCCATAGTAACGCCAATAGGGACTTTCCATTGACGTCAAT |
| | GGGTGGACTATTTACGGTAAACTGCCCACTTGGCAGTACATCAAGTGTATCAT |
| | ATGCCAAGTACGCCCCCTATTGACGTCAATGACGGTAAATGGCCCGCCTGGC |
| | ATTATGCCCAGTACATGACCTTATGGGACTTTCCTACTTGGCAGTACATCTAC |
| | GTATTAGTCATCGCTATTACCATGGGTCGAGGTGAGCCCCACGTTCTGCTTCA |
| | CTCTCCCCATCTCCCCCCCCTCCCCACCCCCAATTTTGTATTTATTTATTTTTTA |
| | ATTATTTTGTGCAGCGATGGGGGCGGGGGGGGGGGGGGCGCGCGCCAGGCG |
| | GGGCGGGGCGGGGCGAGGGGCGGGGCGGGGCGAGGCGGAGAGGTGCGGCG |
| | GCAGCCAATCAGAGCGGCGCGCTCCGAAAGTTTCCTTTTATGGCGAGGCGGC |
| | GGCGGCGGCGGCCCTATAAAAAGCGAAGCGCGCGGCGGGCGGGAGTCGCTG |
| | CGTTGCCTTCGCCCCGTGCCCCGCTCCGCGCCGCCTCGCGCCGCCCGCCCCGG |
| | CTCTGACTGACCGCGTTACTCCCACAGGTGAGCGGGCGGGACGGCCCTTCTCC |
| | TCCGGGCTGTAATTAGCGCTTGGTTTAATGACGGCTCGTTTCTTTTCTGTGGCT |
| | GCGTGAAAGCCTTAAAGGGCTCCGGGAGGGCCCTTTGTGCGGGGGGGAGCGG |
| | CTCGGGGGGTGCGTGCGTGTGTGTGTGCGTGGGGAGCGCCGCGTGCGGCCCG |
| | CGCTGCCCGGCGGCTGTGAGCGCTGCGGGCGCGGCGCGGGGCTTTGTGCGCT |
| | CCGCGTGTGCGCGAGGGGAGCGCGGCCGGGGGCGGTGCCCCGCGGTGCGGG |
| | GGGGCTGCGAGGGGAACAAAGGCTGCGTGCGGGGTGTGTGCGTGGGGGGGT |
| | GAGCAGGGGGTGTGGGCGCGGCGGTCGGGCTGTAACCCCCCCCTGCACCCCC |
| | CTCCCCGAGTTGCTGAGCACGGCCCGGCTTCGGGTGCGGGGCTCCGTGCGGG |
| | GCGTGGCGCGGGGCTCGCCGTGCCGGGCGGGGGGTGGCGGCAGGTGGGGGT |
| | GCCGGGCGGGGCGGGGCCGCCTCGGGCCGGGGAGGGCTCGGGGGAGGGGCG |
| | CGGCGGCCCCGGAGCGCCGGCGGCTGTCGAGGCGCGGCGAGCCGCAGCCATT |
| | GCCTTTTATGGTAATCGTGCGAGAGGGCGCAGGGACTTCCTTTGTCCCAAATC |
| | TGGCGGAGCCGAAATCTGGGAGGCGCCGCCGCACCCCCTCTAGCGGGCGCGG |
| | GCGAAGCGGTGCGGCGCCGGCAGGAAGGAAATGGGCGGGGAGGGCCTTCGT |
| | GCGTCGCCGCGCCGCCGTCCCCTTCTCCATCTCCAGCCTCGGGGCTGCCGCAG |
| | GGGGACGGCTGCCTTCGGGGGGGACGGGGCAGGGCGGGGTTCGGCTTCTGGC |
| | GTGTGACCGGCGGCTCTAGAGCCTCTGCTAACCATGTTCATGCCTTCTTCTTTT |
| | TCCTACAGgggggatccgtttatctgcagaattcgcccttgacgtcgccaccatggctgaacaagtccttcctcaggctttg |
| | tatttgagcaatatgcggaaagctgtgaagatacgggagagaactccagaagacatttttaaacctactaatgggatcattcatcat |
| | tttaaaaccatgcaccgatacacactggaaatgttcagaacttgccagttttgtcctcagtttcgggagatcatccacaaagccctca |
| | tcgacagaaacatccaggccaccctggaaagccagaagaaactcaactggtgtcgagaagtccggaagcttgtggcgctgaa |
| | aacgaacggtgacggcaattgcctcatgcatgccacttctcagtacatgtggggcgttcaggacacagacttggtactgaggaa |
| | ggcgctgttcagcacgctcaaggaaacagacacacgcaactttaaattccgctggcaactggagtctctcaaatctcaggaatttg |
| | ttgaaacggggctttgctatgatactcggaactggaatgatgaatgggacaatcttatcaaaatggcttccacagacacacccatg |
| | gcccgaagtggacttcagtacaactcactggaagaaatacacatatttgtcctttgcaacatcctcagaaggccaatcattgtcattt |
| | cagacaaaatgctaagaagtttggaatcaggttccaatttcgcccctttgaaagtgggtggaatttacttgcctctccactggcctgc |
| | ccaggaatgctacagataccccattgttctcggctatgacagccatcattttgtacccttggtgaccctgaaggacagtgggcctg |
| | aaatccgagctgttccacttgttaacagagaccggggaagatttgaagacttaaaagttcactttttgacagatcctgaaaatgaga |
| | tgaaggagaagctcttaaaagagtacttaatggtgatagaaatccccgtccaaggctgggaccatggcacaactcatctcatcaat |
| | gccgcaaagttggatgaagctaacttaccaaaagaaatcaatctggtagatgattactttgaacttgttcagcatgagtacaagaaa |
| | tggcaggaaaacagcgagcaggggaggagagaggggcacgcccagaatcccatggaaccttccgtgccccagctttctctca |
| | tggatgtaaaatgtgaaacgcccaactgccccttcttcatgtctgtgaacacccagcctttatgccatgagtgctcagagaggcgg |
| | caaaagaatcaaaacaaactcccaaagctgaactccaagccgggccctgaggggctccctggcatggcgctcggggcctctc |
| | ggggagaagcctatgagcccttggcgtggaaccctgaggagtccactggggggcctcattcggccccaccgacagcaccca |
| | gcccttttctgttcagtgagaccactgccatgaagtgcaggagccccggctgccccttcacactgaatgtgcagcacaacggattt |
| | tgtgaacgttgccacaacgcccggcaacttcacgccagccacgccccagaccacacaaggcacttggatcccgggaagtgcc |
| | aagcctgcctccaggatgttaccaggacatttaatgggatctgcagtacttgcttcaaaaggactacagcagaggcctcctccag |
| | cctcagcaccagcctccctccttcctgtcaccagcgttccaagtcagatccctcgcggctcgtccggagcccctccccgcattctt |
| | gccacagagctggaaacgacgcccctgctggctgcctgtctcaagctgcacggactcctggggacaggacggggacgagca |
| | agtgcagaaaagccggctgcgtgtattttgggactccagaaaacaagggcttttgcacactgtgtttcatcgagtacagagaaaa |
| | caaacattttgctgctgcctcagggaaagtcagtcccacagcgtccaggttccagaacaccattccgtgcctggggagggaatg |
| | cggcacccttggaagcaccatgtttgaaggatactgccagaagtgtttcattgaagctcagaatcagagatttcatgaggccaaaa |
| | ggacagaagagcaactgagatcgagccagcgcagagatgtgcctcgaaccacacaaagcacctcaaggcccaagtgcgccc |
| | gggcctcctgcaagaacatcctggcctgccgcagcgaggagctctgcatggagtgtcagcatcccaaccagaggatgggccc |
| | tggggcccaccggggtgagcctgcccccgaagacccccccaagcagcgttgccgggcccccgcctgtgatcattttggcaat |
| | gccaagtgcaacggctactgcaacgaatgctttcagttcaagcagatgtatggcGGAAGCGGAGCTACTAACT |
| | TCAGCCTGCTGAAGCAGGCTGGAGACGTGGAGGAGAACCCTGGACCTatgaggat |
| | atttgctgtctttatattcatgacctactggcatttgctgaacgcatttactgtcacggttcccaaggacctatatgtggtagagtatggt |
| | agcaatatgacaattgaatgcaaattcccagtagaaaaacaattagacctggctgcactaattgtctattgggaaatggaggataa |
| | gaacattattcaatttgtgcatggagaggaagacctgaaggttcagcatagtagctacagacagagggcccggctgttgaagga |
| | ccagctctccctgggaaatgctgcacttcagatcacagatgtgaaattgcaggatgcaggggtgtaccgctgcatgatcagctat |
| | ggtggtgccgactacaagcgaattactgtgaaagtcaatgccccatacaacaaaatcaaccaaagaattttggttgtggatccagt |
| | cacctctgaacatgaacttacatgtcaggctgagggctaccccaaggccgaagtcatctggacaagcagtgaccatcaagtcct |
| | gagtggtaagaccaccaccaccaattccaagagagaggagaaacttttcaatgtgaccagcacactgagaatcaacacaacaa |
| | ctaatgagattttctactgcacttttaggagattagatcctgaggaaaaccatacagctgaattggtcatcccagaactacctctggc |
| | acatcctccaaatgaaaggactcacttggtaattctgggagccatcttattatgccttggtgtagcactgacattcatcttccgtttaag |
| | aaaagggagaatgatggatgtgaaaaaatgtggcatccaagatacaaactcaaagaagcaaagtgatacacatttggaggaga |
| | cgtaaCCGCTGATCAGCCTCGACTGTGCCTTCTAGTTGCCAGCCATCTGTTGTTT |
| | GCCCCTCCCCCGTGCCTTCCTTGACCCTGGAAGGTGCCACTCCCACTGTCCTTT |
| | CCTAATAAAATGAGGAAATTGCATCGCATTGTCTGAGTAGGTGTCATTCTATT |
| | CTGGGGGGTGGGGTGGGGCAGGACAGCAAGGGGGAGGATTGGGAAGACAAT |
| | AGCAGGCATGCTGGGGATGCGGTGGGCTCTATGGGTCGACccagcgtgagtctctcctac |
| | cctcccgctctggtccttcctctcccgctctgcaccctctgtggccctcgctgtgctctctcgctccgtgacttcccttctccaagttct |
| | ccttggtggcccgccgtggggctagtccagggctggatctcggggaagcggcggggtggcctgggagtggggaagggggt |
| | gcgcacccgggacgcgcgctacttgcccctttcggcggggagcaggggagacctttggcctacggcgacgggagggtcggg |
| | acaaagtttagggcgtcgataagcgtcagagcgccgaggttgggggagggtttctcttccgctctttcgcggggcctctggctcc |
| | cccagcgcagctggagtgggggacgggtaggctcgtcccaaaggcgcggcgctgaggtttgtgaacgcgtggaggggcgct |
| | tggggtctgggggaggcgtcgcccgggtaagcctgtctgctgcggctctgcttcccttagactggagagctgtggacttcgtcta |
| | ggcgcccgctaagttcgcatgtcctagcacctctgggtctatgtggggccacaccgtggggaggaaacagcacgcgacgtttgt |
| | agaatgcttggctgtgatacaaagcggtttcgaataattaacttatttgttcccatcacatgtcacttttaaaaaattataagaactacc |
| | cgttattgacatctttctgtgtgccaaggactttatgtgctttgcgtcatttaattttgaaaacagttatcttccgccatagataactactat |
| | ggttatcttctGGTAACCACGTGCGGACCGAGGCTGCAGCGTCGTCCTCCCTAGGAA |
| | CCCCTAGTGATGGAGTTGGCCACTCCCTCTCTGCGCGCTCGCTCGCTCACTGA |
| | GGCCGGGCGACCAAAGGTCGCCCGACGCCCGGGCTTTGCCCGGGCGGCCTCA |
| | GTGAGCGAGCGAGCGCGCAGCTGCCTGCAGGGGCGCCTGATGCGGTATTTTC |
| | TCCTTACGCATCTGTGCGGTATTTCACACCGCATACGTCAAAGCAACCATAGT |
| | ACGCGCCCTGTAGCGGCGCATTAAGCGCGGCGGGTGTGGTGGTTACGCGCAG |
| | CGTGACCGCTACACTTGCCAGCGCCCTAGCGCCCGCTCCTTTCGCTTTCTTCCC |
| | TTCCTTTCTCGCCACGTTCGCCGGCTTTCCCCGTCAAGCTCTAAATCGGGGGCT |
| | CCCTTTAGGGTTCCGATTTAGTGCTTTACGGCACCTCGACCCCAAAAAACTTG |
| | ATTTGGGTGATGGTTCACGTAGTGGGCCATCGCCCTGATAGACGGTTTTTCGC |
| | CCTTTGACGTTGGAGTCCACGTTCTTTAATAGTGGACTCTTGTTCCAAACTGG |
| | AACAACACTCAACCCTATCTCGGGCTATTCTTTTGATTTATAAGGGATTTTGC |
| | CGATTTCGGCCTATTGGTTAAAAAATGAGCTGATTTAACAAAAATTTAACGCG |
| | AATTTTAACAAAATATTAACGTTTACAATTTTATGGTGCACTCTCAGTACAAT |
| | CTGCTCTGATGCCGCATAGTTAAGCCAGCCCCGACACCCGCCAACACCCGCTG |
| | ACGCGCCCTGACGGGCTTGTCTGCTCCCGGCATCCGCTTACAGACAAGCTGTG |
| | ACCGTCTCCGGGAGCTGCATGTGTCAGAGGTTTTCACCGTCATCACCGAAACG |
| | CGCGAGACGAAAGGGCCTCGTGATACGCCTATTTTTATAGGTTAATGTCATGA |
| | ACAATAAAACTGTCTGCTTACATAAACAGTAATACAAGGGGTGTTATGAGCC |
| | ATATTCAACGGGAAACGTCGAGGCCGCGATTAAATTCCAACATGGATGCTGA |
| | TTTATATGGGTATAAATGGGCTCGCGATAATGTCGGGCAATCAGGTGCGACA |
| | ATCTATCGCTTGTATGGGAAGCCCGATGCGCCAGAGTTGTTTCTGAAACATGG |
| | CAAAGGTAGCGTTGCCAATGATGTTACAGATGAGATGGTCAGACTAAACTGG |
| | CTGACGGAATTTATGCCTCTTCCGACCATCAAGCATTTTATCCGTACTCCTGAT |
| | GATGCATGGTTACTCACCACTGCGATCCCCGGAAAAACAGCATTCCAGGTATT |
| | AGAAGAATATCCTGATTCAGGTGAAAATATTGTTGATGCGCTGGCAGTGTTCC |
| | TGCGCCGGTTGCATTCGATTCCTGTTTGTAATTGTCCTTTTAACAGCGATCGCG |
| | TATTTCGTCTCGCTCAGGCGCAATCACGAATGAATAACGGTTTGGTTGATGCG |
| | AGTGATTTTGATGACGAGCGTAATGGCTGGCCTGTTGAACAAGTCTGGAAAG |
| | AAATGCATAAACTTTTGCCATTCTCACCGGATTCAGTCGTCACTCATGGTGAT |
| | TTCTCACTTGATAACCTTATTTTTGACGAGGGGAAATTAATAGGTTGTATTGA |
| | TGTTGGACGAGTCGGAATCGCAGACCGATACCAGGATCTTGCCATCCTATGG |
| | AACTGCCTCGGTGAGTTTTCTCCTTCATTACAGAAACGGCTTTTTCAAAAATA |
| | TGGTATTGATAATCCTGATATGAATAAATTGCAGTTTCATTTGATGCTCGATG |
| | AGTTTTTCTAATCTCATGACCAAAATCCCTTAACGTGAGTTTTCGTTCCACTGA |
| | GCGTCAGACCCCGTAGAAAAGATCAAAGGATCTTCTTGAGATCCTTTTTTTCT |
| | GCGCGTAATCTGCTGCTTGCAAACAAAAAAACCACCGCTACCAGCGGTGGTT |
| | TGTTTGCCGGATCAAGAGCTACCAACTCTTTTTCCGAAGGTAACTGGCTTCAG |
| | CAGAGCGCAGATACCAAATACTGTCCTTCTAGTGTAGCCGTAGTTAGGCCACC |
| | ACTTCAAGAACTCTGTAGCACCGCCTACATACCTCGCTCTGCTAATCCTGTTA |
| | CCAGTGGCTGCTGCCAGTGGCGATAAGTCGTGTCTTACCGGGTTGGACTCAAG |
| | ACGATAGTTACCGGATAAGGCGCAGCGGTCGGGCTGAACGGGGGGTTCGTGC |
| | ACACAGCCCAGCTTGGAGCGAACGACCTACACCGAACTGAGATACCTACAGC |
| | GTGAGCTATGAGAAAGCGCCACGCTTCCCGAAGGGAGAAAGGCGGACAGGT |
| | ATCCGGTAAGCGGCAGGGTCGGAACAGGAGAGCGCACGAGGGAGCTTCCAG |
| | GGGGAAACGCCTGGTATCTTTATAGTCCTGTCGGGTTTCGCCACCTCTGACTT |
| | GAGCGTCGATTTTTGTGATGCTCGTCAGGGGGGCGGAGCCTATGGAAAAACG |
| | CCAGCAACGCGGCCTTTTTACGGTTCCTGGCCTTTTGCTGGCCTTTTGCTCACA |
| | TGT |
| 42 | accgctctcagaccagaaacgtccacacccgccctccgatggcctgtcgccctggctaggttttagggtcagtgggatcctcctt |
| | ccactggacccgggagaagacgctcaacagccccctccttcccctccttcctctccttcctctccttcccccctccctgcgccgct |
| | ccagagcgcaacaaccattttcccagccaggagcacaccgtgtccacgcgccacagcgatctcactgattggtcgggctcctg |
| | gtaaacaaggaccgggcagccaatgggagggatgtgcacgagggcagcacgagcctccgggccagcgctcgcgtggctctt |
| | ctggcccgggctactatatagagacgtttccgcctcctgcttgaaactaacccctctttttctccaaaggagtgcttgtggagatcgg |
| | atcttttctccagcaattgggggaaagaaggctttttctctgaattagcttagtgtaaccagcggcgtatattttttaggcgccttttcgaaaa |
| | cctagtagttaatattcatttgtttaaatcttattttatttttaagctcaaactgcttaagaataccttaattccttaaagtgaaataatttt |
| | ttgcaaaggggtttcctcgatttggagctttttttttcttccaccgtcatttctaactcttaaaaccaactcagttccatcatggtgatgttc |
| | aagaagatcaagtcttttgaggtggtctttaacgaccctgaaaaggtgtacggcagtggcgagaaggtggctggccgggtgata |
| | gtggaggtgtgtg |
| 43 | atgtctcgctccgttgccttagctgtgctcgcgctactctctctttctggattagaggctgtcatggcgccccgaaccctcttcctgG |
| | GTGGAGGCGGTTCAGGCGGAGGTGGCTCTGGCGGTGGCGGATCGatccagcgtactc |
| | caaagattcaggtttactcacgtcatccagcagagaatggaaagtcaaatttcctgaattgctatgtgtctgggtttcatccatccga |
| | cattgaagttgacttactgaagaatggagagagaattgaaaaagtggagcattcagacttgtctttcagcaaggactggtctttctat |
| | ctcttgtactacactgaattcacccccactgaaaaagatgagtatgcctgccgtgtgaaccatgtgactttgtcacagcccaagata |
| | gttaagtgggatcgagacatgGGTGGTGGTGGTTCTGGTGGTGGTGGTTCTGGCGGCGGC |
| | GGCTCCGGTGGTGGTGGATCCggctcccactccttgaagtatttccacacttccgtgtcccggcccggccgcg |
| | gggagccccgcttcatctctgtgggctacgtggacgacacccagttcgtgcgcttcgacaacgacgccgcgagtccgaggatg |
| | gtgccgcgggcgccgtggatggagcaggaggggtcagagtattgggaccgggagacacggagcgccagggacaccgcac |
| | agattttccgagtgaatctgcggacgctgcgcggctactacaatcagagcgaggccgggtctcacaccctgcagtggatgcatg |
| | gctgcgagctggggcccgacgggcgcttcctccgcgggtatgaacagttcgcctacgacggcaaggattatctcaccctgaat |
| | gaggacctgcgctcctggaccgcggtggacacggcggctcagatctccgagcaaaagtcaaatgatgcctctgaggcggagc |
| | accagagagcctacctggaagacacatgcgtggagtggctccacaaatacctggagaaggggaaggagacgctgcttcacct |
| | ggagcccccaaagacacacgtgactcaccaccccatctctgaccatgaggccaccctgaggtgctgggccctgggcttctacc |
| | ctgcggagatcacactgacctggcagcaggatggggagggccatacccaggacacggagctcgtggagaccaggcctgca |
| | ggggatggaaccttccagaagtgggcagctgtggtggtgccttctggagaggagcagagatacacgtgccatgtgcagcatga |
| | ggggctacccgagcccgtcaccctgagatggaagccggcttcccagcccaccatccccatcgtgggcatcattgctggcctgg |
| | ttctccttggatctgtggtctctggagctgtggttgctgctgtgatatggaggaagaagagctcaggtggaaaaggagggagcta |
| | ctctaaggctgagtggagcgacagtgcccaggggtctgagtctcacagcttg |
| 44 | cagggatcccagcagtgcaaacagacttcggagtacctgcgctatgaagacacgcttcttctggaagaccagccaacaggtaa |
| | gcggcccaattcattgttggagggtgaaagctgattagagaagagaattgaatacacaaaacctgtacgaaatgttttaagttgctc |
| | agtttgagtggtttgaattacgtgttgttgcttccttttttctgttttaatttgcagacattctcctccccccccaaaaaaaagggtgatttg |
| | tacaattttttatggtgctgtgtcctaaaggggatcctgaggggcgttgcctcgggtagttaaagtcttatgtgtgcataagttgcttat |
| | tctttgtctacttcctatttgagatgttagtagagaactgtcctgggtgaatctttcagtattgcagggcttggcaacttgctgcccgac |
| | aaaatacatcagaatttctctttaagaacaatatgggatggattaaaaaatatatatatgggatgaaattgggggtacttcaatacctt |
| | gcatgccacccaagcattccttatcacacagatgcattttaagtgtaacagcaagcctaatggctactcgattttctttcccttcaggt |
| | gagaatgagatggtgatcatgagacctggaaacaaatatgagtacaagttcggctttgagcttcctcaggggtaaatatcagctaa |
| | atgcatctttgaacttttctgtctaaaatatcttgccctcctttgatcacttactgttcttggagagcgttttaaaattttcattttcttgac |
| 45 | CCTGCAGGCAGCTGCGCGCTCGCTCGCTCACTGAGGCCGCCCGGGCGTCGGG |
| | CGACCTTTGGTCGCCCGGCCTCAGTGAGCGAGCGAGCGCGCAGAGAGGGAGT |
| | GGCCAACTCCATCACTAGGGGTTCCTGCGGCCGCACGCGTaccgctctcagaccagaaac |
| | gtccacacccgccctccgatggcctgtcgccctggctaggttttagggtcagtgggatcctccttccactggacccgggagaag |
| | acgctcaacagccccctccttcccctccttcctctccttcctctccttcccccctccctgcgccgctccagagcgcaacaaccatttt |
| | cccagccaggagcacaccgtgtccacgcgccacagcgatctcactgattggtcgggctcctggtaaacaaggaccgggcagc |
| | caatgggagggatgtgcacgagggcagcacgagcctccgggccagcgctcgcgtggctcttctggcccgggctactatatag |
| | agacgtttccgcctcctgcttgaaactaacccctctttttctccaaaggagtgcttgtggagatcggatcttttctccagcaattgggg |
| | gaaagaaggctttttctctgaattagcttagtgtaaccagcggcgtatattttttaggcgccttttcgaaaacctagtagttaatattcat |
| | ttgtttaaatcttattttatttttaagctcaaactgcttaagaataccttaattccttaaagtgaaataattttttgcaaaggggtttcctcgat |
| | ttggagctttttttttcttccaccgtcatttctaactcttaaaaccaactcagttccatcatggtgatgttcaagaagatcaagtcttttgag |
| | gtggtctttaacgaccctgaaaaggtgtacggcagtggcgagaaggtggctggccgggtgatagtggaggtgtgtgaagcttgt |
| | ggacgatatcgaattcgcacgacattgattattgactagtTATTAATAGTAATCAATTACGGGGTCATT |
| | AGTTCATAGCCCATATATGGAGTTCCGCGTTACATAACTTACGGTAAATGGCC |
| | CGCCTGGCTGACCGCCCAACGACCCCCGCCCATTGACGTCAATAATGACGTAT |
| | GTTCCCATAGTAACGCCAATAGGGACTTTCCATTGACGTCAATGGGTGGACTA |
| | TTTACGGTAAACTGCCCACTTGGCAGTACATCAAGTGTATCATATGCCAAGTA |
| | CGCCCCCTATTGACGTCAATGACGGTAAATGGCCCGCCTGGCATTATGCCCAG |
| | TACATGACCTTATGGGACTTTCCTACTTGGCAGTACATCTACGTATTAGTCAT |
| | CGCTATTACCATGGGTCGAGGTGAGCCCCACGTTCTGCTTCACTCTCCCCATC |
| | TCCCCCCCCTCCCCACCCCCAATTTTGTATTTATTTATTTTTTAATTATTTTGTG |
| | CAGCGATGGGGGCGGGGGGGGGGGGGGCGCGCGCCAGGCGGGGCGGGGCGG |
| | GGCGAGGGGCGGGGCGGGGCGAGGCGGAGAGGTGCGGCGGCAGCCAATCAG |
| | AGCGGCGCGCTCCGAAAGTTTCCTTTTATGGCGAGGCGGCGGCGGCGGCGGC |
| | CCTATAAAAAGCGAAGCGCGCGGCGGGCGGGAGTCGCTGCGTTGCCTTCGCC |
| | CCGTGCCCCGCTCCGCGCCGCCTCGCGCCGCCCGCCCCGGCTCTGACTGACCG |
| | CGTTACTCCCACAGGTGAGCGGGCGGGACGGCCCTTCTCCTCCGGGCTGTAAT |
| | TAGCGCTTGGTTTAATGACGGCTCGTTTCTTTTCTGTGGCTGCGTGAAAGCCTT |
| | AAAGGGCTCCGGGAGGGCCCTTTGTGCGGGGGGGAGCGGCTCGGGGGGTGC |
| | GTGCGTGTGTGTGTGCGTGGGGAGCGCCGCGTGCGGCCCGCGCTGCCCGGCG |
| | GCTGTGAGCGCTGCGGGCGCGGCGCGGGGCTTTGTGCGCTCCGCGTGTGCGC |
| | GAGGGGAGCGCGGCCGGGGGCGGTGCCCCGCGGTGCGGGGGGGCTGCGAGG |
| | GGAACAAAGGCTGCGTGCGGGGTGTGTGCGTGGGGGGGTGAGCAGGGGGTG |
| | TGGGCGCGGCGGTCGGGCTGTAACCCCCCCCTGCACCCCCCTCCCCGAGTTGC |
| | TGAGCACGGCCCGGCTTCGGGTGCGGGGCTCCGTGCGGGGCGTGGCGCGGGG |
| | CTCGCCGTGCCGGGCGGGGGGTGGCGGCAGGTGGGGGTGCCGGGCGGGGCG |
| | GGGCCGCCTCGGGCCGGGGAGGGCTCGGGGGAGGGGCGCGGCGGCCCCGGA |
| | GCGCCGGCGGCTGTCGAGGCGCGGCGAGCCGCAGCCATTGCCTTTTATGGTA |
| | ATCGTGCGAGAGGGCGCAGGGACTTCCTTTGTCCCAAATCTGGCGGAGCCGA |
| | AATCTGGGAGGCGCCGCCGCACCCCCTCTAGCGGGCGCGGGCGAAGCGGTGC |
| | GGCGCCGGCAGGAAGGAAATGGGCGGGGAGGGCCTTCGTGCGTCGCCGCGC |
| | CGCCGTCCCCTTCTCCATCTCCAGCCTCGGGGCTGCCGCAGGGGGACGGCTGC |
| | CTTCGGGGGGGACGGGGCAGGGCGGGGTTCGGCTTCTGGCGTGTGACCGGCG |
| | GCTCTAGAGCCTCTGCTAACCATGTTCATGCCTTCTTCTTTTTCCTACAGggggga |
| | tccgtttatctgcagaattcgcccttgacgtcgccaccatgaggaggatgtgggccacgcaggggctggcggtggcgctggctc |
| | tgagcgtgctgccgggcagccgggcgctgcggccgggcgactgcgaagtttgtatttcttatctgggaagattttaccaggacct |
| | caaagacagagatgtcacattctcaccagccactattgaaaacgaacttataaagttctgccgggaagcaagaggcaaagagaa |
| | tcggttgtgctactatatcggggccacagatgatgcagccaccaaaatcatcaatgaggtatcaaagcctctggcccaccacatc |
| | cctgtggagaagatctgtgagaagcttaagaagaaggacagccagatatgtgagcttaagtatgacaagcagatcgacctgagc |
| | acagtggacctgaagaagctccgagttaaagagctgaagaagattctggatgactggggggagacatgcaaaggctgtgcag |
| | aaaagtctgactacatccggaagataaatgaactgatgcctaaatatgcccccaaggcagccagtgcacggaccgatttgGG |
| | AAGCGGAGCTACTAACTTCAGCCTGCTGAAGCAGGCTGGAGACGTGGAGGAG |
| | AACCCTGGACCTatgtctcgctccgttgccttagctgtgctcgcgctactctctctttctggattagaggctgtcatggcg |
| | ccccgaaccctcttcctgGGTGGAGGCGGTTCAGGCGGAGGTGGCTCTGGCGGTGGCGG |
| | ATCGatccagcgtactccaaagattcaggtttactcacgtcatccagcagagaatggaaagtcaaatttcctgaattgctatgtg |
| | tctgggtttcatccatccgacattgaagttgacttactgaagaatggagagagaattgaaaaagtggagcattcagacttgtctttca |
| | gcaaggactggtctttctatctcttgtactacactgaattcacccccactgaaaaagatgagtatgcctgccgtgtgaaccatgtgac |
| | tttgtcacagcccaagatagttaagtgggatcgagacatgGGTGGTGGTGGTTCTGGTGGTGGTGGTT |
| | CTGGCGGCGGCGGCTCCGGTGGTGGTGGATCCggctcccactccttgaagtatttccacacttccgt |
| | gtcccggcccggccgcggggagccccgcttcatctctgtgggctacgtggacgacacccagttcgtgcgcttcgacaacgacg |
| | ccgcgagtccgaggatggtgccgcgggcgccgtggatggagcaggaggggtcagagtattgggaccgggagacacggag |
| | cgccagggacaccgcacagattttccgagtgaatctgcggacgctgcgcggctactacaatcagagcgaggccgggtctcaca |
| | ccctgcagtggatgcatggctgcgagctggggcccgacgggcgcttcctccgcgggtatgaacagttcgcctacgacggcaa |
| | ggattatctcaccctgaatgaggacctgcgctcctggaccgcggtggacacggcggctcagatctccgagcaaaagtcaaatg |
| | atgcctctgaggcggagcaccagagagcctacctggaagacacatgcgtggagtggctccacaaatacctggagaaggggaa |
| | ggagacgctgcttcacctggagcccccaaagacacacgtgactcaccaccccatctctgaccatgaggccaccctgaggtgct |
| | gggccctgggcttctaccctgcggagatcacactgacctggcagcaggatggggagggccatacccaggacacggagctcgt |
| | ggagaccaggcctgcaggggatggaaccttccagaagtgggcagctgtggtggtgccttctggagaggagcagagatacacg |
| | tgccatgtgcagcatgaggggctacccgagcccgtcaccctgagatggaagccggcttcccagcccaccatccccatcgtggg |
| | catcattgctggcctggttctccttggatctgtggtctctggagctgtggttgctgctgtgatatggaggaagaagagctcaggtgg |
| | aaaaggagggagctactctaaggctgagtggagcgacagtgcccaggggtctgagtctcacagcttgtaatgatagCCGCT |
| | GATCAGCCTCGACTGTGCCTTCTAGTTGCCAGCCATCTGTTGTTTGCCCCTCCC |
| | CCGTGCCTTCCTTGACCCTGGAAGGTGCCACTCCCACTGTCCTTTCCTAATAA |
| | AATGAGGAAATTGCATCGCATTGTCTGAGTAGGTGTCATTCTATTCTGGGGGG |
| | TGGGGTGGGGCAGGACAGCAAGGGGGAGGATTGGGAAGACAATAGCAGGCA |
| | TGCTGGGGATGCGGTGGGCTCTATGGGTCGACcagggatcccagcagtgcaaacagacttcgga |
| | gtacctgcgctatgaagacacgcttcttctggaagaccagccaacaggtaagcggcccaattcattgttggagggtgaaagctga |
| | ttagagaagagaattgaatacacaaaacctgtacgaaatgttttaagttgctcagtttgagtggtttgaattacgtgttgttgcttccttt |
| | tttctgttttaatttgcagacattctcctccccccccaaaaaaaagggtgatttgtacaattttttatggtgctgtgtcctaaaggggatc |
| | ctgaggggcgttgcctcgggtagttaaagtcttatgtgtgcataagttgcttattctttgtctacttcctatttgagatgttagtagagaa |
| | ctgtcctgggtgaatctttcagtattgcagggcttggcaacttgctgcccgacaaaatacatcagaatttctctttaagaacaatatgg |
| | gatggattaaaaaatatatatatgggatgaaattgggggtacttcaataccttgcatgccacccaagcattccttatcacacagatgc |
| | attttaagtgtaacagcaagcctaatggctactcgattttctttcccttcaggtgagaatgagatggtgatcatgagacctggaaaca |
| | aatatgagtacaagttcggctttgagcttcctcaggggtaaatatcagctaaatgcatctttgaacttttctgtctaaaatatcttgccct |
| | cctttgatcacttactgttcttggagagcgttttaaaattttcattttcttgacGGTAACCACGTGCGGACCGAGG |
| | CTGCAGCGTCGTCCTCCCTAGGAACCCCTAGTGATGGAGTTGGCCACTCCCTC |
| | TCTGCGCGCTCGCTCGCTCACTGAGGCCGGGCGACCAAAGGTCGCCCGACGC |
| | CCGGGCTTTGCCCGGGCGGCCTCAGTGAGCGAGCGAGCGCGCAGCTGCCTGC |
| | AGGGGCGCCTGATGCGGTATTTTCTCCTTACGCATCTGTGCGGTATTTCACAC |
| | CGCATACGTCAAAGCAACCATAGTACGCGCCCTGTAGCGGCGCATTAAGCGC |
| | GGCGGGTGTGGTGGTTACGCGCAGCGTGACCGCTACACTTGCCAGCGCCCTA |
| | GCGCCCGCTCCTTTCGCTTTCTTCCCTTCCTTTCTCGCCACGTTCGCCGGCTTTC |
| | CCCGTCAAGCTCTAAATCGGGGGCTCCCTTTAGGGTTCCGATTTAGTGCTTTA |
| | CGGCACCTCGACCCCAAAAAACTTGATTTGGGTGATGGTTCACGTAGTGGGC |
| | CATCGCCCTGATAGACGGTTTTTCGCCCTTTGACGTTGGAGTCCACGTTCTTTA |
| | ATAGTGGACTCTTGTTCCAAACTGGAACAACACTCAACCCTATCTCGGGCTAT |
| | TCTTTTGATTTATAAGGGATTTTGCCGATTTCGGCCTATTGGTTAAAAAATGA |
| | GCTGATTTAACAAAAATTTAACGCGAATTTTAACAAAATATTAACGTTTACAA |
| | TTTTATGGTGCACTCTCAGTACAATCTGCTCTGATGCCGCATAGTTAAGCCAG |
| | CCCCGACACCCGCCAACACCCGCTGACGCGCCCTGACGGGCTTGTCTGCTCCC |
| | GGCATCCGCTTACAGACAAGCTGTGACCGTCTCCGGGAGCTGCATGTGTCAG |
| | AGGTTTTCACCGTCATCACCGAAACGCGCGAGACGAAAGGGCCTCGTGATAC |
| | GCCTATTTTTATAGGTTAATGTCATGAACAATAAAACTGTCTGCTTACATAAA |
| | CAGTAATACAAGGGGTGTTATGAGCCATATTCAACGGGAAACGTCGAGGCCG |
| | CGATTAAATTCCAACATGGATGCTGATTTATATGGGTATAAATGGGCTCGCGA |
| | TAATGTCGGGCAATCAGGTGCGACAATCTATCGCTTGTATGGGAAGCCCGAT |
| | GCGCCAGAGTTGTTTCTGAAACATGGCAAAGGTAGCGTTGCCAATGATGTTA |
| | CAGATGAGATGGTCAGACTAAACTGGCTGACGGAATTTATGCCTCTTCCGACC |
| | ATCAAGCATTTTATCCGTACTCCTGATGATGCATGGTTACTCACCACTGCGAT |
| | CCCCGGAAAAACAGCATTCCAGGTATTAGAAGAATATCCTGATTCAGGTGAA |
| | AATATTGTTGATGCGCTGGCAGTGTTCCTGCGCCGGTTGCATTCGATTCCTGTT |
| | TGTAATTGTCCTTTTAACAGCGATCGCGTATTTCGTCTCGCTCAGGCGCAATC |
| | ACGAATGAATAACGGTTTGGTTGATGCGAGTGATTTTGATGACGAGCGTAAT |
| | GGCTGGCCTGTTGAACAAGTCTGGAAAGAAATGCATAAACTTTTGCCATTCTC |
| | ACCGGATTCAGTCGTCACTCATGGTGATTTCTCACTTGATAACCTTATTTTTGA |
| | CGAGGGGAAATTAATAGGTTGTATTGATGTTGGACGAGTCGGAATCGCAGAC |
| | CGATACCAGGATCTTGCCATCCTATGGAACTGCCTCGGTGAGTTTTCTCCTTC |
| | ATTACAGAAACGGCTTTTTCAAAAATATGGTATTGATAATCCTGATATGAATA |
| | AATTGCAGTTTCATTTGATGCTCGATGAGTTTTTCTAATCTCATGACCAAAATC |
| | CCTTAACGTGAGTTTTCGTTCCACTGAGCGTCAGACCCCGTAGAAAAGATCAA |
| | AGGATCTTCTTGAGATCCTTTTTTTCTGCGCGTAATCTGCTGCTTGCAAACAAA |
| | AAAACCACCGCTACCAGCGGTGGTTTGTTTGCCGGATCAAGAGCTACCAACT |
| | CTTTTTCCGAAGGTAACTGGCTTCAGCAGAGCGCAGATACCAAATACTGTCCT |
| | TCTAGTGTAGCCGTAGTTAGGCCACCACTTCAAGAACTCTGTAGCACCGCCTA |
| | CATACCTCGCTCTGCTAATCCTGTTACCAGTGGCTGCTGCCAGTGGCGATAAG |
| | TCGTGTCTTACCGGGTTGGACTCAAGACGATAGTTACCGGATAAGGCGCAGC |
| | GGTCGGGCTGAACGGGGGGTTCGTGCACACAGCCCAGCTTGGAGCGAACGAC |
| | CTACACCGAACTGAGATACCTACAGCGTGAGCTATGAGAAAGCGCCACGCTT |
| | CCCGAAGGGAGAAAGGCGGACAGGTATCCGGTAAGCGGCAGGGTCGGAACA |
| | GGAGAGCGCACGAGGGAGCTTCCAGGGGGAAACGCCTGGTATCTTTATAGTC |
| | CTGTCGGGTTTCGCCACCTCTGACTTGAGCGTCGATTTTTGTGATGCTCGTCAG |
| | GGGGGCGGAGCCTATGGAAAAACGCCAGCAACGCGGCCTTTTTACGGTTCCT |
| | GGCCTTTTGCTGGCCTTTTGCTCACATGT |
| 46 | atgtgtccccgagccgcgcgggcgcccgcgacgctactcctcgccctgggcgcggtgctgtggcctgcggctggcgcctggg |
| | agcttacgattttgcacaccaacgacgtgcacagccggctggagcagaccagcgaggactccagcaagtgcgtcaacgccag |
| | ccgctgcatgggtggcgtggctcggctcttcaccaaggttcagcagatccgccgcgccgaacccaacgtgctgctgctggacg |
| | ccggcgaccagtaccagggcactatctggttcaccgtgtacaagggcgccgaggtggcgcacttcatgaacgccctgcgctac |
| | gatgccatggcactgggaaatcatgaatttgataatggtgtggaaggactgatcgagccactcctcaaagaggccaaatttccaat |
| | tctgagtgcaaacattaaagcaaaggggccactagcatctcaaatatcaggactttatttgccatataaagttcttcctgttggtgatg |
| | aagttgtgggaatcgttggatacacttccaaagaaaccccttttctctcaaatccagggacaaatttagtgtttgaagatgaaatcac |
| | tgcattacaacctgaagtagataagttaaaaactctaaatgtgaacaaaattattgcactgggacattcgggttttgaaatggataaa |
| | ctcatcgctcagaaagtgaggggtgtggacgtcgtggtgggaggacactccaacacatttctttacacaggcaatccaccttcca |
| | aagaggtgcctgctgggaagtacccattcatagtcacttctgatgatgggcggaaggttcctgtagtccaggcctatgcttttggca |
| | aatacctaggctatctgaagatcgagtttgatgaaagaggaaacgtcatctcttcccatggaaatcccattcttctaaacagcagca |
| | ttcctgaagatccaagcataaaagcagacattaacaaatggaggataaaattggataattattctacccaggaattagggaaaaca |
| | attgtctatctggatggctcctctcaatcatgccgctttagagaatgcaacatgggcaacctgatttgtgatgcaatgattaacaaca |
| | acctgagacacacggatgaaatgttctggaaccacgtatccatgtgcattttaaatggaggtggtatccggtcgcccattgatgaa |
| | cgcaacaatggcacaattacctgggagaacctggctgctgtattgccctttggaggcacatttgacctagtccagttaaaaggttc |
| | caccctgaagaaggcctttgagcatagcgtgcaccgctacggccagtccactggagagttcctgcaggtgggcggaatccatgt |
| | ggtgtatgatctttcccgaaaacctggagacagagtagtcaaattagatgttctttgcaccaagtgtcgagtgcccagttatgaccct |
| | ctcaaaatggacgaggtatataaggtgatcctcccaaacttcctggccaatggtggagatgggttccagatgataaaagatgaatt |
| | attaagacatgactctggtgaccaagatatcaacgtggtttctacatatatctccaaaatgaaagtaatttatccagcagttgaaggtc |
| | ggatcaagttttccacaggaagtcactgccatggaagcttttctttaatatttctttcactttgggcagtgatctttgttttataccaa |
| 47 | CCTGCAGGCAGCTGCGCGCTCGCTCGCTCACTGAGGCCGCCCGGGCGTCGGG |
| | CGACCTTTGGTCGCCCGGCCTCAGTGAGCGAGCGAGCGCGCAGAGAGGGAGT |
| | GGCCAACTCCATCACTAGGGGTTCCTGCGGCCGCACGCGTgttctagggtggaaactaag |
| | agaatgatgtacctagagggcgctggaagctctaaagccctagcagttactgcttttactattagtggtcgtttttttctcccccccgc |
| | cccccgacaaatcaacagaacaaagaaaattacctaaacagcaaggacatagggaggaacttcttggcacagaactttccaaac |
| | actttttcctgaagggatacaagaagcaagaaaggtactctttcactaggaccttctctgagctgtcctcaggatgcttttgggacta |
| | tttttcttacccagagaatggagaaaccctgcagggaattcccaagctgtagttataaacagaagttctccttctgctaggtagcatt |
| | caaagatcttaatcttctgggtttccgttttctcgaatgaaaaatgcaggtccgagcagttaactggctggggcaccattagcaagtc |
| | acttagcatctctggggccagtctgcaaagcgagggggcagccttaatgtgcctccagcctgaagtcctagaatgagcgcccgg |
| | tgtcccaagctggggcgcgcaccccagatcggagggcgccgatgtacagacagcaaactcacccagtctagtgcatgccttctt |
| | aaacatcacgagactctaagaaaaggaaactgaaaacgggaaagtccctctctctaacctggcactgcgtcgctggcttggaga |
| | caggtgacggtccctgcgggccttgtcctgattggctgggcacgcgtttaatataagtggaggcgtcgcgctggcgggcattcct |
| | gaagctaagcttgtggacgatatcgaattcgcacgacattgattattgactagtTATTAATAGTAATCAATTAC |
| | GGGGTCATTAGTTCATAGCCCATATATGGAGTTCCGCGTTACATAACTTACGG |
| | TAAATGGCCCGCCTGGCTGACCGCCCAACGACCCCCGCCCATTGACGTCAAT |
| | AATGACGTATGTTCCCATAGTAACGCCAATAGGGACTTTCCATTGACGTCAAT |
| | GGGTGGACTATTTACGGTAAACTGCCCACTTGGCAGTACATCAAGTGTATCAT |
| | ATGCCAAGTACGCCCCCTATTGACGTCAATGACGGTAAATGGCCCGCCTGGC |
| | ATTATGCCCAGTACATGACCTTATGGGACTTTCCTACTTGGCAGTACATCTAC |
| | GTATTAGTCATCGCTATTACCATGGGTCGAGGTGAGCCCCACGTTCTGCTTCA |
| | CTCTCCCCATCTCCCCCCCCTCCCCACCCCCAATTTTGTATTTATTTATTTTTTA |
| | ATTATTTTGTGCAGCGATGGGGGCGGGGGGGGGGGGGGCGCGCGCCAGGCG |
| | GGGCGGGGCGGGGCGAGGGGCGGGGCGGGGCGAGGCGGAGAGGTGCGGCG |
| | GCAGCCAATCAGAGCGGCGCGCTCCGAAAGTTTCCTTTTATGGCGAGGCGGC |
| | GGCGGCGGCGGCCCTATAAAAAGCGAAGCGCGCGGCGGGCGGGAGTCGCTG |
| | CGTTGCCTTCGCCCCGTGCCCCGCTCCGCGCCGCCTCGCGCCGCCCGCCCCGG |
| | CTCTGACTGACCGCGTTACTCCCACAGGTGAGCGGGCGGGACGGCCCTTCTCC |
| | TCCGGGCTGTAATTAGCGCTTGGTTTAATGACGGCTCGTTTCTTTTCTGTGGCT |
| | GCGTGAAAGCCTTAAAGGGCTCCGGGAGGGCCCTTTGTGCGGGGGGGAGCGG |
| | CTCGGGGGGTGCGTGCGTGTGTGTGTGCGTGGGGAGCGCCGCGTGCGGCCCG |
| | CGCTGCCCGGCGGCTGTGAGCGCTGCGGGCGCGGCGCGGGGCTTTGTGCGCT |
| | CCGCGTGTGCGCGAGGGGAGCGCGGCCGGGGGCGGTGCCCCGCGGTGCGGG |
| | GGGGCTGCGAGGGGAACAAAGGCTGCGTGCGGGGTGTGTGCGTGGGGGGGT |
| | GAGCAGGGGGTGTGGGCGCGGCGGTCGGGCTGTAACCCCCCCCTGCACCCCC |
| | CTCCCCGAGTTGCTGAGCACGGCCCGGCTTCGGGTGCGGGGCTCCGTGCGGG |
| | GCGTGGCGCGGGGCTCGCCGTGCCGGGCGGGGGGTGGCGGCAGGTGGGGGT |
| | GCCGGGGGGGGGGGGCCGCCTCGGGCCGGGGAGGGCTCGGGGGAGGGGCG |
| | CGGCGGCCCCGGAGCGCCGGCGGCTGTCGAGGCGCGGCGAGCCGCAGCCATT |
| | GCCTTTTATGGTAATCGTGCGAGAGGGCGCAGGGACTTCCTTTGTCCCAAATC |
| | TGGCGGAGCCGAAATCTGGGAGGCGCCGCCGCACCCCCTCTAGCGGGCGCGG |
| | GCGAAGCGGTGCGGCGCCGGCAGGAAGGAAATGGGCGGGGAGGGCCTTCGT |
| | GCGTCGCCGCGCCGCCGTCCCCTTCTCCATCTCCAGCCTCGGGGCTGCCGCAG |
| | GGGGACGGCTGCCTTCGGGGGGGACGGGGCAGGGCGGGGTTCGGCTTCTGGC |
| | GTGTGACCGGCGGCTCTAGAGCCTCTGCTAACCATGTTCATGCCTTCTTCTTTT |
| | TCCTACAGgggggatccgtttatctgcagaattcgcccttgacgtcgccaccatggaagatacaaaggagtctaacgtga |
| | agacattttgctccaagaatatcctagccatccttggcttctcctctatcatagctgtgatagctttgcttgctgtggggttgacccaga |
| | acaaagcattgccagaaaacgttaagtatgggattgtgctggatgcgggttcttctcacacaagtttatacatctataagtggccag |
| | cagaaaaggagaatgacacaggcgtggtgcatcaagtagaagaatgcagggttaaaggtcctggaatctcaaaatttgttcaga |
| | aagtaaatgaaataggcatttacctgactgattgcatggaaagagctagggaagtgattccaaggtcccagcaccaagagacac |
| | ccgtttacctgggagccacggcaggcatgcggttgctcaggatggaaagtgaagagttggcagacagggttctggatgtggtg |
| | gagaggagcctcagcaactacccctttgacttccagggtgccaggatcattactggccaagaggaaggtgcctatggctggatt |
| | actatcaactatctgctgggcaaattcagtcagaaaacaaggtggttcagcatagtcccatatgaaaccaataatcaggaaaccttt |
| | ggagctttggaccttgggggagcctctacacaagtcacttttgtaccccaaaaccagactatcgagtccccagataatgctctgca |
| | atttcgcctctatggcaaggactacaatgtctacacacatagcttcttgtgctatgggaaggatcaggcactctggcagaaactgg |
| | ccaaggacattcaggttgcaagtaatgaaattctcagggacccatgctttcatcctggatataagaaggtagtgaacgtaagtgac |
| | ctttacaagaccccctgcaccaagagatttgagatgactcttccattccagcagtttgaaatccagggtattggaaactatcaacaat |
| | gccatcaaagcatcctggagctcttcaacaccagttactgcccttactcccagtgtgccttcaatgggattttcttgccaccactcca |
| | gggggattttggggcattttcagctttttactttgtgatgaagtttttaaacttgacatcagagaaagtctctcaggaaaaggtgactg |
| | agatgatgaaaaagttctgtgctcagccttgggaggagataaaaacatcttacgctggagtaaaggagaagtacctgagtgaata |
| | ctgcttttctggtacctacattctctccctccttctgcaaggctatcatttcacagctgattcctgggagcacatccatttcattggcaag |
| | atccagggcagcgacgccggctggactttgggctacatgctgaacctgaccaacatgatcccagctgagcaaccattgtccaca |
| | cctctctcccactccacctatgtcttcctcatggttctattctccctggtccttttcacagtggccatcataggcttgcttatctttcacaa |
| | gccttcatatttctggaaagatatggtaGGAAGCGGAGCTACTAACTTCAGCCTGCTGAAGCAG |
| | GCTGGAGACGTGGAGGAGAACCCTGGACCTatgtgtccccgagccgcgcgggcgcccgcgacg |
| | ctactcctcgccctgggcgcggtgctgtggcctgcggctggcgcctgggagcttacgattttgcacaccaacgacgtgcacagc |
| | cggctggagcagaccagcgaggactccagcaagtgcgtcaacgccagccgctgcatgggtggcgtggctcggctcttcacca |
| | aggttcagcagatccgccgcgccgaacccaacgtgctgctgctggacgccggcgaccagtaccagggcactatctggttcacc |
| | gtgtacaagggcgccgaggtggcgcacttcatgaacgccctgcgctacgatgccatggcactgggaaatcatgaatttgataat |
| | ggtgtggaaggactgatcgagccactcctcaaagaggccaaatttccaattctgagtgcaaacattaaagcaaaggggccacta |
| | gcatctcaaatatcaggactttatttgccatataaagttcttcctgttggtgatgaagttgtgggaatcgttggatacacttccaaagaa |
| | accccttttctctcaaatccagggacaaatttagtgtttgaagatgaaatcactgcattacaacctgaagtagataagttaaaaactct |
| | aaatgtgaacaaaattattgcactgggacattcgggttttgaaatggataaactcatcgctcagaaagtgaggggtgtggacgtcg |
| | tggtgggaggacactccaacacatttctttacacaggcaatccaccttccaaagaggtgcctgctgggaagtacccattcatagtc |
| | acttctgatgatgggcggaaggttcctgtagtccaggcctatgcttttggcaaatacctaggctatctgaagatcgagtttgatgaaa |
| | gaggaaacgtcatctcttcccatggaaatcccattcttctaaacagcagcattcctgaagatccaagcataaaagcagacattaac |
| | aaatggaggataaaattggataattattctacccaggaattagggaaaacaattgtctatctggatggctcctctcaatcatgccgct |
| | ttagagaatgcaacatgggcaacctgatttgtgatgcaatgattaacaacaacctgagacacacggatgaaatgttctggaaccac |
| | gtatccatgtgcattttaaatggaggtggtatccggtcgcccattgatgaacgcaacaatggcacaattacctgggagaacctggc |
| | tgctgtattgccctttggaggcacatttgacctagtccagttaaaaggttccaccctgaagaaggcctttgagcatagcgtgcaccg |
| | ctacggccagtccactggagagttcctgcaggtgggcggaatccatgtggtgtatgatctttcccgaaaacctggagacagagta |
| | gtcaaattagatgttctttgcaccaagtgtcgagtgcccagttatgaccctctcaaaatggacgaggtatataaggtgatcctccca |
| | aacttcctggccaatggtggagatgggttccagatgataaaagatgaattattaagacatgactctggtgaccaagatatcaacgt |
| | ggtttctacatatatctccaaaatgaaagtaatttatccagcagttgaaggtcggatcaagttttccacaggaagtcactgccatgga |
| | agcttttctttaatatttctttcactttgggcagtgatctttgttttataccaaGGAAGCGGAGCTACTAACTTCAG |
| | CCTGCTGAAGCAGGCTGGAGACGTGGAGGAGAACCCTGGACCTatgaggatatttgct |
| | gtctttatattcatgacctactggcatttgctgaacgcatttactgtcacggttcccaaggacctatatgtggtagagtatggtagcaat |
| | atgacaattgaatgcaaattcccagtagaaaaacaattagacctggctgcactaattgtctattgggaaatggaggataagaacatt |
| | attcaatttgtgcatggagaggaagacctgaaggttcagcatagtagctacagacagagggcccggctgttgaaggaccagctc |
| | tccctgggaaatgctgcacttcagatcacagatgtgaaattgcaggatgcaggggtgtaccgctgcatgatcagctatggtggtg |
| | ccgactacaagcgaattactgtgaaagtcaatgccccatacaacaaaatcaaccaaagaattttggttgtggatccagtcacctct |
| | gaacatgaactgacatgtcaggctgagggctaccccaaggccgaagtcatctggacaagcagtgaccatcaagtcctgagtgg |
| | taagaccaccaccaccaattccaagagagaggagaaacttttcaatgtgaccagcacactgagaatcaacacaacaactaatga |
| | gattttctactgcacttttaggagattagatcctgaggaaaaccatacagctgaattggtcatcccagaactacctctggcacatcct |
| | ccaaatgaaaggactcacttggtaattctgggagccatcttattatgccttggtgtagcactgacattcatcttccgtttaagaaaagg |
| | gagaatgatggatgtgaaaaaatgtggcatccaagatacaaactcaaagaagcaaagtgatacacatttggaggagacgtaaC |
| | CGCTGATCAGCCTCGACTGTGCCTTCTAGTTGCCAGCCATCTGTTGTTTGCCCC |
| | TCCCCCGTGCCTTCCTTGACCCTGGAAGGTGCCACTCCCACTGTCCTTTCCTAA |
| | TAAAATGAGGAAATTGCATCGCATTGTCTGAGTAGGTGTCATTCTATTCTGGG |
| | GGGTGGGGTGGGGCAGGACAGCAAGGGGGAGGATTGGGAAGACAATAGCAG |
| | GCATGCTGGGGATGCGGTGGGCTCTATGGGTCGACccagcgtgagtctctcctaccctcccgct |
| | ctggtccttcctctcccgctctgcaccctctgtggccctcgctgtgctctctcgctccgtgacttcccttctccaagttctccttggtgg |
| | cccgccgtggggctagtccagggctggatctcggggaagcggcggggtggcctgggagtggggaagggggtgcgcaccc |
| | gggacgcgcgctacttgcccctttcggggggagcaggggagacctttggcctacggcgacgggagggtcgggacaaagttt |
| | agggcgtcgataagcgtcagagcgccgaggttgggggagggtttctcttccgctctttcgcggggcctctggctcccccagcgc |
| | agctggagtgggggacgggtaggctcgtcccaaaggcgcggcgctgaggtttgtgaacgcgtggaggggcgcttggggtct |
| | gggggaggcgtcgcccgggtaagcctgtctgctgcggctctgcttcccttagactggagagctgtggacttcgtctaggcgccc |
| | gctaagttcgcatgtcctagcacctctgggtctatgtggggccacaccgtggggaggaaacagcacgcgacgtttgtagaatgct |
| | tggctgtgatacaaagcggtttcgaataattaacttatttgttcccatcacatgtcacttttaaaaaattataagaactacccgttattga |
| | catctttctgtgtgccaaggactttatgtgctttgcgtcatttaattttgaaaacagttatcttccgccatagataactactatggttatctt |
| | ctGGTAACCACGTGCGGACCGAGGCTGCAGCGTCGTCCTCCCTAGGAACCCCT |
| | AGTGATGGAGTTGGCCACTCCCTCTCTGCGCGCTCGCTCGCTCACTGAGGCCG |
| | GGCGACCAAAGGTCGCCCGACGCCCGGGCTTTGCCCGGGCGGCCTCAGTGAG |
| | CGAGCGAGCGCGCAGCTGCCTGCAGGGGCGCCTGATGCGGTATTTTCTCCTTA |
| | CGCATCTGTGCGGTATTTCACACCGCATACGTCAAAGCAACCATAGTACGCGC |
| | CCTGTAGCGGCGCATTAAGCGCGGCGGGTGTGGTGGTTACGCGCAGCGTGAC |
| | CGCTACACTTGCCAGCGCCCTAGCGCCCGCTCCTTTCGCTTTCTTCCCTTCCTT |
| | TCTCGCCACGTTCGCCGGCTTTCCCCGTCAAGCTCTAAATCGGGGGCTCCCTT |
| | TAGGGTTCCGATTTAGTGCTTTACGGCACCTCGACCCCAAAAAACTTGATTTG |
| | GGTGATGGTTCACGTAGTGGGCCATCGCCCTGATAGACGGTTTTTCGCCCTTT |
| | GACGTTGGAGTCCACGTTCTTTAATAGTGGACTCTTGTTCCAAACTGGAACAA |
| | CACTCAACCCTATCTCGGGCTATTCTTTTGATTTATAAGGGATTTTGCCGATTT |
| | CGGCCTATTGGTTAAAAAATGAGCTGATTTAACAAAAATTTAACGCGAATTTT |
| | AACAAAATATTAACGTTTACAATTTTATGGTGCACTCTCAGTACAATCTGCTC |
| | TGATGCCGCATAGTTAAGCCAGCCCCGACACCCGCCAACACCCGCTGACGCG |
| | CCCTGACGGGCTTGTCTGCTCCCGGCATCCGCTTACAGACAAGCTGTGACCGT |
| | CTCCGGGAGCTGCATGTGTCAGAGGTTTTCACCGTCATCACCGAAACGCGCG |
| | AGACGAAAGGGCCTCGTGATACGCCTATTTTTATAGGTTAATGTCATGAACAA |
| | TAAAACTGTCTGCTTACATAAACAGTAATACAAGGGGTGTTATGAGCCATATT |
| | CAACGGGAAACGTCGAGGCCGCGATTAAATTCCAACATGGATGCTGATTTAT |
| | ATGGGTATAAATGGGCTCGCGATAATGTCGGGCAATCAGGTGCGACAATCTA |
| | TCGCTTGTATGGGAAGCCCGATGCGCCAGAGTTGTTTCTGAAACATGGCAAA |
| | GGTAGCGTTGCCAATGATGTTACAGATGAGATGGTCAGACTAAACTGGCTGA |
| | CGGAATTTATGCCTCTTCCGACCATCAAGCATTTTATCCGTACTCCTGATGAT |
| | GCATGGTTACTCACCACTGCGATCCCCGGAAAAACAGCATTCCAGGTATTAG |
| | AAGAATATCCTGATTCAGGTGAAAATATTGTTGATGCGCTGGCAGTGTTCCTG |
| | CGCCGGTTGCATTCGATTCCTGTTTGTAATTGTCCTTTTAACAGCGATCGCGTA |
| | TTTCGTCTCGCTCAGGCGCAATCACGAATGAATAACGGTTTGGTTGATGCGAG |
| | TGATTTTGATGACGAGCGTAATGGCTGGCCTGTTGAACAAGTCTGGAAAGAA |
| | ATGCATAAACTTTTGCCATTCTCACCGGATTCAGTCGTCACTCATGGTGATTTC |
| | TCACTTGATAACCTTATTTTTGACGAGGGGAAATTAATAGGTTGTATTGATGT |
| | TGGACGAGTCGGAATCGCAGACCGATACCAGGATCTTGCCATCCTATGGAAC |
| | TGCCTCGGTGAGTTTTCTCCTTCATTACAGAAACGGCTTTTTCAAAAATATGG |
| | TATTGATAATCCTGATATGAATAAATTGCAGTTTCATTTGATGCTCGATGAGT |
| | TTTTCTAATCTCATGACCAAAATCCCTTAACGTGAGTTTTCGTTCCACTGAGCG |
| | TCAGACCCCGTAGAAAAGATCAAAGGATCTTCTTGAGATCCTTTTTTTCTGCG |
| | CGTAATCTGCTGCTTGCAAACAAAAAAACCACCGCTACCAGCGGTGGTTTGTT |
| | TGCCGGATCAAGAGCTACCAACTCTTTTTCCGAAGGTAACTGGCTTCAGCAGA |
| | GCGCAGATACCAAATACTGTCCTTCTAGTGTAGCCGTAGTTAGGCCACCACTT |
| | CAAGAACTCTGTAGCACCGCCTACATACCTCGCTCTGCTAATCCTGTTACCAG |
| | TGGCTGCTGCCAGTGGCGATAAGTCGTGTCTTACCGGGTTGGACTCAAGACG |
| | ATAGTTACCGGATAAGGCGCAGCGGTCGGGCTGAACGGGGGGTTCGTGCACA |
| | CAGCCCAGCTTGGAGCGAACGACCTACACCGAACTGAGATACCTACAGCGTG |
| | AGCTATGAGAAAGCGCCACGCTTCCCGAAGGGAGAAAGGCGGACAGGTATC |
| | CGGTAAGCGGCAGGGTCGGAACAGGAGAGCGCACGAGGGAGCTTCCAGGGG |
| | GAAACGCCTGGTATCTTTATAGTCCTGTCGGGTTTCGCCACCTCTGACTTGAG |
| | CGTCGATTTTTGTGATGCTCGTCAGGGGGGCGGAGCCTATGGAAAAACGCCA |
| | GCAACGCGGCCTTTTTACGGTTCCTGGCCTTTTGCTGGCCTTTTGCTCACATGT |
| 52 | atgaggaggatgtgggccacgcaggggctggcggtggcgctggctctgagcgtgctgccgggcagccgggcgctgcggcc |
| | gggcgactgcgaagtttgtatttcttatctgggaagattttaccaggacctcaaagacagagatgtcacattctcaccagccactatt |
| | gaaaacgaacttataaagttctgccgggaagcaagaggcaaagagaatcggttgtgctactatatcggggccacagatgatgca |
| | gccaccaaaatcatcaatgaggtatcaaagcctctggcccaccacatccctgtggagaagatctgtgagaagcttaagaagaag |
| | gacagccagatatgtgagcttaagtatgacaagcagatcgacctgagcacagtggacctgaagaagctccgagttaaagagct |
| | gaagaagattctggatgactggggggagacatgcaaaggctgtgcagaaaagtctgactacatccggaagataaatgaactgat |
| | gcctaaatatgcccccaaggcagccagtgcacggaccgatttgGGAAGCGGAGCTACTAACTTCAGCC |
| | TGCTGAAGCAGGCTGGAGACGTGGAGGAGAACCCTGGACCTatggctgaacaagtcctt |
| | cctcaggctttgtatttgagcaatatgcggaaagctgtgaagatacgggagagaactccagaagacatttttaaacctactaatgg |
| | gatcattcatcattttaaaaccatgcaccgatacacactggaaatgttcagaacttgccagttttgtcctcagtttcgggagatcatcc |
| | acaaagccctcatcgacagaaacatccaggccaccctggaaagccagaagaaactcaactggtgtcgagaagtccggaagctt |
| | gtggcgctgaaaacgaacggtgacggcaattgcctcatgcatgccacttctcagtacatgtggggcgttcaggacacagacttg |
| | gtactgaggaaggcgctgttcagcacgctcaaggaaacagacacacgcaactttaaattccgctggcaactggagtctctcaaat |
| | ctcaggaatttgttgaaacggggctttgctatgatactcggaactggaatgatgaatgggacaatcttatcaaaatggcttccacag |
| | acacacccatggcccgaagtggacttcagtacaactcactggaagaaatacacatatttgtcctttgcaacatcctcagaaggcca |
| | atcattgtcatttcagacaaaatgctaagaagtttggaatcaggttccaatttcgcccctttgaaagtgggtggaatttacttgcctctc |
| | cactggcctgcccaggaatgctacagataccccattgttctcggctatgacagccatcattttgtacccttggtgaccctgaaggac |
| | agtgggcctgaaatccgagctgttccacttgttaacagagaccggggaagatttgaagacttaaaagttcactttttgacagatcct |
| | gaaaatgagatgaaggagaagctcttaaaagagtacttaatggtgatagaaatccccgtccaaggctgggaccatggcacaact |
| | catctcatcaatgccgcaaagttggatgaagctaacttaccaaaagaaatcaatctggtagatgattactttgaacttgttcagcatg |
| | agtacaagaaatggcaggaaaacagcgagcaggggaggagagaggggcacgcccagaatcccatggaaccttccgtgccc |
| | cagctttctctcatggatgtaaaatgtgaaacgcccaactgccccttcttcatgtctgtgaacacccagcctttatgccatgagtgctc |
| | agagaggcggcaaaagaatcaaaacaaactcccaaagctgaactccaagccgggccctgaggggctccctggcatggcgct |
| | cggggcctctcggggagaagcctatgagcccttggcgtggaaccctgaggagtccactggggggcctcattcggccccaccg |
| | acagcacccagcccttttctgttcagtgagaccactgccatgaagtgcaggagccccggctgccccttcacactgaatgtgcagc |
| | acaacggattttgtgaacgttgccacaacgcccggcaacttcacgccagccacgccccagaccacacaaggcacttggatccc |
| | gggaagtgccaagcctgcctccaggatgttaccaggacatttaatgggatctgcagtacttgcttcaaaaggactacagcagagg |
| | cctcctccagcctcagcaccagcctccctccttcctgtcaccagcgttccaagtcagatccctcgcggctcgtccggagcccctc |
| | cccgcattcttgccacagagctggaaacgacgcccctgctggctgcctgtctcaagctgcacggactcctggggacaggacgg |
| | ggacgagcaagtgcagaaaagccggctgcgtgtattttgggactccagaaaacaagggcttttgcacactgtgtttcatcgagta |
| | cagagaaaacaaacattttgctgctgcctcagggaaagtcagtcccacagcgtccaggttccagaacaccattccgtgcctggg |
| | gagggaatgcggcacccttggaagcaccatgtttgaaggatactgccagaagtgtttcattgaagctcagaatcagagatttcatg |
| | aggccaaaaggacagaagagcaactgagatcgagccagcgcagagatgtgcctcgaaccacacaaagcacctcaaggccc |
| | aagtgcgcccgggcctcctgcaagaacatcctggcctgccgcagcgaggagctctgcatggagtgtcagcatcccaaccaga |
| | ggatgggccctggggcccaccggggtgagcctgcccccgaagacccccccaagcagcgttgccgggcccccgcctgtgatc |
| | attttggcaatgccaagtgcaacggctactgcaacgaatgctttcagttcaagcagatgtatggcGGAAGCGGAGCTA |
| | CTAACTTCAGCCTGCTGAAGCAGGCTGGAGACGTGGAGGAGAACCCTGGACC |
| | Tatgaggatatttgctgtctttatattcatgacctactggcatttgctgaacgcatttactgtcacggttcccaaggacctatatgtggt |
| | agagtatggtagcaatatgacaattgaatgcaaattcccagtagaaaaacaattagacctggctgcactaattgtctattgggaaat |
| | ggaggataagaacattattcaatttgtgcatggagaggaagacctgaaggttcagcatagtagctacagacagagggcccggct |
| | gttgaaggaccagctctccctgggaaatgctgcacttcagatcacagatgtgaaattgcaggatgcaggggtgtaccgctgcatg |
| | atcagctatggtggtgccgactacaagcgaattactgtgaaagtcaatgccccatacaacaaaatcaaccaaagaattttggttgt |
| | ggatccagtcacctctgaacatgaactgacatgtcaggctgagggctaccccaaggccgaagtcatctggacaagcagtgacc |
| | atcaagtcctgagtggtaagaccaccaccaccaattccaagagagaggagaaacttttcaatgtgaccagcacactgagaatca |
| | acacaacaactaatgagattttctactgcacttttaggagattagatcctgaggaaaaccatacagctgaattggtcatcccagaact |
| | acctctggcacatcctccaaatgaaaggactcacttggtaattctgggagccatcttattatgccttggtgtagcactgacattcatct |
| | tccgtttaagaaaagggagaatgatggatgtgaaaaaatgtggcatccaagatacaaactcaaagaagcaaagtgatacacattt |
| | ggaggagacgtaa |
| 53 | atggaagatacaaaggagtctaacgtgaagacattttgctccaagaatatcctagccatccttggcttctcctctatcatagctgtga |
| | tagctttgcttgctgtggggttgacccagaacaaagcattgccagaaaacgttaagtatgggattgtgctggatgcgggttcttctc |
| | acacaagtttatacatctataagtggccagcagaaaaggagaatgacacaggcgtggtgcatcaagtagaagaatgcagggtta |
| | aaggtcctggaatctcaaaatttgttcagaaagtaaatgaaataggcatttacctgactgattgcatggaaagagctagggaagtg |
| | attccaaggtcccagcaccaagagacacccgtttacctgggagccacggcaggcatgcggttgctcaggatggaaagtgaaga |
| | gttggcagacagggttctggatgtggtggagaggagcctcagcaactacccctttgacttccagggtgccaggatcattactggc |
| | caagaggaaggtgcctatggctggattactatcaactatctgctgggcaaattcagtcagaaaacaaggtggttcagcatagtccc |
| | atatgaaaccaataatcaggaaacctttggagctttggaccttgggggagcctctacacaagtcacttttgtaccccaaaaccaga |
| | ctatcgagtccccagataatgctctgcaatttcgcctctatggcaaggactacaatgtctacacacatagcttcttgtgctatgggaa |
| | ggatcaggcactctggcagaaactggccaaggacattcaggttgcaagtaatgaaattctcagggacccatgctttcatcctggat |
| | ataagaaggtagtgaacgtaagtgacctttacaagaccccctgcaccaagagatttgagatgactcttccattccagcagtttgaa |
| | atccagggtattggaaactatcaacaatgccatcaaagcatcctggagctcttcaacaccagttactgcccttactcccagtgtgcc |
| | ttcaatgggattttcttgccaccactccagggggattttggggcattttcagctttttactttgtgatgaagtttttaaacttgacatcaga |
| | gaaagtctctcaggaaaaggtgactgagatgatgaaaaagttctgtgctcagccttgggaggagataaaaacatcttacgctgga |
| | gtaaaggagaagtacctgagtgaatactgcttttctggtacctacattctctccctccttctgcaaggctatcatttcacagctgattcc |
| | tgggagcacatccatttcattggcaagatccagggcagcgacgccggctggactttgggctacatgctgaacctgaccaacatg |
| | atcccagctgagcaaccattgtccacacctctctcccactccacctatgtcttcctcatggttctattctccctggtccttttcacagtg |
| | gccatcataggcttgcttatctttcacaagccttcatatttctggaaagatatggtaGGAAGCGGAGCTACTAACT |
| | TCAGCCTGCTGAAGCAGGCTGGAGACGTGGAGGAGAACCCTGGACCTatgaggat |
| | atttgctgtctttatattcatgacctactggcatttgctgaacgcatttactgtcacggttcccaaggacctatatgtggtagagtatggt |
| | agcaatatgacaattgaatgcaaattcccagtagaaaaacaattagacctggctgcactaattgtctattgggaaatggaggataa |
| | gaacattattcaatttgtgcatggagaggaagacctgaaggttcagcatagtagctacagacagagggcccggctgttgaagga |
| | ccagctctccctgggaaatgctgcacttcagatcacagatgtgaaattgcaggatgcaggggtgtaccgctgcatgatcagctat |
| | ggtggtgccgactacaagcgaattactgtgaaagtcaatgccccatacaacaaaatcaaccaaagaattttggttgtggatccagt |
| | cacctctgaacatgaactgacatgtcaggctgagggctaccccaaggccgaagtcatctggacaagcagtgaccatcaagtcct |
| | gagtggtaagaccaccaccaccaattccaagagagaggagaaacttttcaatgtgaccagcacactgagaatcaacacaacaa |
| | ctaatgagattttctactgcacttttaggagattagatcctgaggaaaaccatacagctgaattggtcatcccagaactacctctggc |
| | acatcctccaaatgaaaggactcacttggtaattctgggagccatcttattatgccttggtgtagcactgacattcatcttccgtttaag |
| | aaaagggagaatgatggatgtgaaaaaatgtggcatccaagatacaaactcaaagaagcaaagtgatacacatttggaggaga |
| | cgtaa |
| 54 | atggctgaacaagtccttcctcaggctttgtatttgagcaatatgcggaaagctgtgaagatacgggagagaactccagaagaca |
| | tttttaaacctactaatgggatcattcatcattttaaaaccatgcaccgatacacactggaaatgttcagaacttgccagttttgtcctca |
| | gtttcgggagatcatccacaaagccctcatcgacagaaacatccaggccaccctggaaagccagaagaaactcaactggtgtc |
| | gagaagtccggaagcttgtggcgctgaaaacgaacggtgacggcaattgcctcatgcatgccacttctcagtacatgtggggcg |
| | ttcaggacacagacttggtactgaggaaggcgctgttcagcacgctcaaggaaacagacacacgcaactttaaattccgctggc |
| | aactggagtctctcaaatctcaggaatttgttgaaacggggctttgctatgatactcggaactggaatgatgaatgggacaatcttat |
| | caaaatggcttccacagacacacccatggcccgaagtggacttcagtacaactcactggaagaaatacacatatttgtcctttgca |
| | acatcctcagaaggccaatcattgtcatttcagacaaaatgctaagaagtttggaatcaggttccaatttcgcccctttgaaagtggg |
| | tggaatttacttgcctctccactggcctgcccaggaatgctacagataccccattgttctcggctatgacagccatcattttgtaccct |
| | tggtgaccctgaaggacagtgggcctgaaatccgagctgttccacttgttaacagagaccggggaagatttgaagacttaaaagt |
| | tcactttttgacagatcctgaaaatgagatgaaggagaagctcttaaaagagtacttaatggtgatagaaatccccgtccaaggctg |
| | ggaccatggcacaactcatctcatcaatgccgcaaagttggatgaagctaacttaccaaaagaaatcaatctggtagatgattactt |
| | tgaacttgttcagcatgagtacaagaaatggcaggaaaacagcgagcaggggaggagagaggggcacgcccagaatcccat |
| | ggaaccttccgtgccccagctttctctcatggatgtaaaatgtgaaacgcccaactgccccttcttcatgtctgtgaacacccagcc |
| | tttatgccatgagtgctcagagaggcggcaaaagaatcaaaacaaactcccaaagctgaactccaagccgggccctgaggggc |
| | tccctggcatggcgctcggggcctctcggggagaagcctatgagcccttggcgtggaaccctgaggagtccactggggggcc |
| | tcattcggccccaccgacagcacccagcccttttctgttcagtgagaccactgccatgaagtgcaggagccccggctgccccttc |
| | acactgaatgtgcagcacaacggattttgtgaacgttgccacaacgcccggcaacttcacgccagccacgccccagaccacac |
| | aaggcacttggatcccgggaagtgccaagcctgcctccaggatgttaccaggacatttaatgggatctgcagtacttgcttcaaaa |
| | ggactacagcagaggcctcctccagcctcagcaccagcctccctccttcctgtcaccagcgttccaagtcagatccctcgcggct |
| | cgtccggagcccctccccgcattcttgccacagagctggaaacgacgcccctgctggctgcctgtctcaagctgcacggactcc |
| | tggggacaggacggggacgagcaagtgcagaaaagccggctgcgtgtattttgggactccagaaaacaagggcttttgcaca |
| | ctgtgtttcatcgagtacagagaaaacaaacattttgctgctgcctcagggaaagtcagtcccacagcgtccaggttccagaacac |
| | cattccgtgcctggggagggaatgcggcacccttggaagcaccatgtttgaaggatactgccagaagtgtttcattgaagctcag |
| | aatcagagatttcatgaggccaaaaggacagaagagcaactgagatcgagccagcgcagagatgtgcctcgaaccacacaaa |
| | gcacctcaaggcccaagtgcgcccgggcctcctgcaagaacatcctggcctgccgcagcgaggagctctgcatggagtgtca |
| | gcatcccaaccagaggatgggccctggggcccaccggggtgagcctgcccccgaagacccccccaagcagcgttgccggg |
| | cccccgcctgtgatcattttggcaatgccaagtgcaacggctactgcaacgaatgctttcagttcaagcagatgtatggcGGA |
| | AGCGGAGCTACTAACTTCAGCCTGCTGAAGCAGGCTGGAGACGTGGAGGAGA |
| | ACCCTGGACCTatgaggatatttgctgtctttatattcatgacctactggcatttgctgaacgcatttactgtcacggttccc |
| | aaggacctatatgtggtagagtatggtagcaatatgacaattgaatgcaaattcccagtagaaaaacaattagacctggctgcact |
| | aattgtctattgggaaatggaggataagaacattattcaatttgtgcatggagaggaagacctgaaggttcagcatagtagctaca |
| | gacagagggcccggctgttgaaggaccagctctccctgggaaatgctgcacttcagatcacagatgtgaaattgcaggatgcag |
| | gggtgtaccgctgcatgatcagctatggtggtgccgactacaagcgaattactgtgaaagtcaatgccccatacaacaaaatcaa |
| | ccaaagaattttggttgtggatccagtcacctctgaacatgaacttacatgtcaggctgagggctaccccaaggccgaagtcatct |
| | ggacaagcagtgaccatcaagtcctgagtggtaagaccaccaccaccaattccaagagagaggagaaacttttcaatgtgacca |
| | gcacactgagaatcaacacaacaactaatgagattttctactgcacttttaggagattagatcctgaggaaaaccatacagctgaat |
| | tggtcatcccagaactacctctggcacatcctccaaatgaaaggactcacttggtaattctgggagccatcttattatgccttggtgt |
| | agcactgacattcatcttccgtttaagaaaagggagaatgatggatgtgaaaaaatgtggcatccaagatacaaactcaaagaag |
| | caaagtgatacacatttggaggagacgtaa |
| 55 | atgaggaggatgtgggccacgcaggggctggcggtggcgctggctctgagcgtgctgccgggcagccgggcgctgcggcc |
| | gggcgactgcgaagtttgtatttcttatctgggaagattttaccaggacctcaaagacagagatgtcacattctcaccagccactatt |
| | gaaaacgaacttataaagttctgccgggaagcaagaggcaaagagaatcggttgtgctactatatcggggccacagatgatgca |
| | gccaccaaaatcatcaatgaggtatcaaagcctctggcccaccacatccctgtggagaagatctgtgagaagcttaagaagaag |
| | gacagccagatatgtgagcttaagtatgacaagcagatcgacctgagcacagtggacctgaagaagctccgagttaaagagct |
| | gaagaagattctggatgactggggggagacatgcaaaggctgtgcagaaaagtctgactacatccggaagataaatgaactgat |
| | gcctaaatatgcccccaaggcagccagtgcacggaccgatttgGGAAGCGGAGCTACTAACTTCAGCC |
| | TGCTGAAGCAGGCTGGAGACGTGGAGGAGAACCCTGGACCTatgtctcgctccgttgcct |
| | tagctgtgctcgcgctactctctctttctggattagaggctgtcatggcgccccgaaccctcttcctgGGTGGAGGCGGT |
| | TCAGGCGGAGGTGGCTCTGGCGGTGGCGGATCGatccagcgtactccaaagattcaggtttactc |
| | acgtcatccagcagagaatggaaagtcaaatttcctgaattgctatgtgtctgggtttcatccatccgacattgaagttgacttactga |
| | agaatggagagagaattgaaaaagtggagcattcagacttgtctttcagcaaggactggtctttctatctcttgtactacactgaatt |
| | cacccccactgaaaaagatgagtatgcctgccgtgtgaaccatgtgactttgtcacagcccaagatagttaagtgggatcgagac |
| | atgGGTGGTGGTGGTTCTGGTGGTGGTGGTTCTGGCGGCGGCGGCTCCGGTGGT |
| | GGTGGATCCggctcccactccttgaagtatttccacacttccgtgtcccggcccggccgcggggagccccgcttcatctc |
| | tgtgggctacgtggacgacacccagttcgtgcgcttcgacaacgacgccgcgagtccgaggatggtgccgcgggcgccgtgg |
| | atggagcaggaggggtcagagtattgggaccgggagacacggagcgccagggacaccgcacagattttccgagtgaatctg |
| | cggacgctgcgcggctactacaatcagagcgaggccgggtctcacaccctgcagtggatgcatggctgcgagctggggcccg |
| | acgggcgcttcctccgcgggtatgaacagttcgcctacgacggcaaggattatctcaccctgaatgaggacctgcgctcctgga |
| | ccgcggtggacacggcggctcagatctccgagcaaaagtcaaatgatgcctctgaggcggagcaccagagagcctacctgga |
| | agacacatgcgtggagtggctccacaaatacctggagaaggggaaggagacgctgcttcacctggagcccccaaagacacac |
| | gtgactcaccaccccatctctgaccatgaggccaccctgaggtgctgggccctgggcttctaccctgcggagatcacactgacct |
| | ggcagcaggatggggagggccatacccaggacacggagctcgtggagaccaggcctgcaggggatggaaccttccagaag |
| | tgggcagctgtggtggtgccttctggagaggagcagagatacacgtgccatgtgcagcatgaggggctacccgagcccgtcac |
| | cctgagatggaagccggcttcccagcccaccatccccatcgtgggcatcattgctggcctggttctccttggatctgtggtctctg |
| | gagctgtggttgctgctgtgatatggaggaagaagagctcaggtggaaaaggagggagctactctaaggctgagtggagcgac |
| | agtgcccaggggtctgagtctcacagcttg |
| 56 | atggaagatacaaaggagtctaacgtgaagacattttgctccaagaatatcctagccatccttggcttctcctctatcatagctgtga |
| | tagctttgcttgctgtggggttgacccagaacaaagcattgccagaaaacgttaagtatgggattgtgctggatgcgggttcttctc |
| | acacaagtttatacatctataagtggccagcagaaaaggagaatgacacaggcgtggtgcatcaagtagaagaatgcagggtta |
| | aaggtcctggaatctcaaaatttgttcagaaagtaaatgaaataggcatttacctgactgattgcatggaaagagctagggaagtg |
| | attccaaggtcccagcaccaagagacacccgtttacctgggagccacggcaggcatgcggttgctcaggatggaaagtgaaga |
| | gttggcagacagggttctggatgtggtggagaggagcctcagcaactacccctttgacttccagggtgccaggatcattactggc |
| | caagaggaaggtgcctatggctggattactatcaactatctgctgggcaaattcagtcagaaaacaaggtggttcagcatagtccc |
| | atatgaaaccaataatcaggaaacctttggagctttggaccttgggggagcctctacacaagtcacttttgtaccccaaaaccaga |
| | ctatcgagtccccagataatgctctgcaatttcgcctctatggcaaggactacaatgtctacacacatagcttcttgtgctatgggaa |
| | ggatcaggcactctggcagaaactggccaaggacattcaggttgcaagtaatgaaattctcagggacccatgctttcatcctggat |
| | ataagaaggtagtgaacgtaagtgacctttacaagaccccctgcaccaagagatttgagatgactcttccattccagcagtttgaa |
| | atccagggtattggaaactatcaacaatgccatcaaagcatcctggagctcttcaacaccagttactgcccttactcccagtgtgcc |
| | ttcaatgggattttcttgccaccactccagggggattttggggcattttcagctttttactttgtgatgaagtttttaaacttgacatcaga |
| | gaaagtctctcaggaaaaggtgactgagatgatgaaaaagttctgtgctcagccttgggaggagataaaaacatcttacgctgga |
| | gtaaaggagaagtacctgagtgaatactgcttttctggtacctacattctctccctccttctgcaaggctatcatttcacagctgattcc |
| | tgggagcacatccatttcattggcaagatccagggcagcgacgccggctggactttgggctacatgctgaacctgaccaacatg |
| | atcccagctgagcaaccattgtccacacctctctcccactccacctatgtcttcctcatggttctattctccctggtccttttcacagtg |
| | gccatcataggcttgcttatctttcacaagccttcatatttctggaaagatatggtaGGAAGCGGAGCTACTAACT |
| | TCAGCCTGCTGAAGCAGGCTGGAGACGTGGAGGAGAACCCTGGACCTatgtgtccc |
| | cgagccgcgcgggcgcccgcgacgctactcctcgccctgggcgcggtgctgtggcctgcggctggcgcctgggagcttacg |
| | attttgcacaccaacgacgtgcacagccggctggagcagaccagcgaggactccagcaagtgcgtcaacgccagccgctgca |
| | tgggtggcgtggctcggctcttcaccaaggttcagcagatccgccgcgccgaacccaacgtgctgctgctggacgccggcgac |
| | cagtaccagggcactatctggttcaccgtgtacaagggcgccgaggtggcgcacttcatgaacgccctgcgctacgatgccatg |
| | gcactgggaaatcatgaatttgataatggtgtggaaggactgatcgagccactcctcaaagaggccaaatttccaattctgagtgc |
| | aaacattaaagcaaaggggccactagcatctcaaatatcaggactttatttgccatataaagttcttcctgttggtgatgaagttgtgg |
| | gaatcgttggatacacttccaaagaaaccccttttctctcaaatccagggacaaatttagtgtttgaagatgaaatcactgcattaca |
| | acctgaagtagataagttaaaaactctaaatgtgaacaaaattattgcactgggacattcgggttttgaaatggataaactcatcgct |
| | cagaaagtgaggggtgtggacgtcgtggtgggaggacactccaacacatttctttacacaggcaatccaccttccaaagaggtg |
| | cctgctgggaagtacccattcatagtcacttctgatgatgggcggaaggttcctgtagtccaggcctatgcttttggcaaataccta |
| | ggctatctgaagatcgagtttgatgaaagaggaaacgtcatctcttcccatggaaatcccattcttctaaacagcagcattcctgaa |
| | gatccaagcataaaagcagacattaacaaatggaggataaaattggataattattctacccaggaattagggaaaacaattgtctat |
| | ctggatggctcctctcaatcatgccgctttagagaatgcaacatgggcaacctgatttgtgatgcaatgattaacaacaacctgaga |
| | cacacggatgaaatgttctggaaccacgtatccatgtgcattttaaatggaggtggtatccggtcgcccattgatgaacgcaacaa |
| | tggcacaattacctgggagaacctggctgctgtattgccctttggaggcacatttgacctagtccagttaaaaggttccaccctgaa |
| | gaaggcctttgagcatagcgtgcaccgctacggccagtccactggagagttcctgcaggtgggcggaatccatgtggtgtatga |
| | tctttcccgaaaacctggagacagagtagtcaaattagatgttctttgcaccaagtgtcgagtgcccagttatgaccctctcaaaatg |
| | gacgaggtatataaggtgatcctcccaaacttcctggccaatggtggagatgggttccagatgataaaagatgaattattaagaca |
| | tgactctggtgaccaagatatcaacgtggtttctacatatatctccaaaatgaaagtaatttatccagcagttgaaggtcggatcaag |
| | ttttccacaggaagtcactgccatggaagcttttctttaatatttctttcactttgggcagtgatctttgttttataccaaGGAAGCG |
| | GAGCTACTAACTTCAGCCTGCTGAAGCAGGCTGGAGACGTGGAGGAGAACCC |
| | TGGACCTatgaggatatttgctgtctttatattcatgacctactggcatttgctgaacgcatttactgtcacggttcccaaggac |
| | ctatatgtggtagagtatggtagcaatatgacaattgaatgcaaattcccagtagaaaaacaattagacctggctgcactaattgtct |
| | attgggaaatggaggataagaacattattcaatttgtgcatggagaggaagacctgaaggttcagcatagtagctacagacagag |
| | ggcccggctgttgaaggaccagctctccctgggaaatgctgcacttcagatcacagatgtgaaattgcaggatgcaggggtgta |
| | ccgctgcatgatcagctatggtggtgccgactacaagcgaattactgtgaaagtcaatgccccatacaacaaaatcaaccaaaga |
| | attttggttgtggatccagtcacctctgaacatgaactgacatgtcaggctgagggctaccccaaggccgaagtcatctggacaa |
| | gcagtgaccatcaagtcctgagtggtaagaccaccaccaccaattccaagagagaggagaaacttttcaatgtgaccagcacac |
| | tgagaatcaacacaacaactaatgagattttctactgcacttttaggagattagatcctgaggaaaaccatacagctgaattggtcat |
| | cccagaactacctctggcacatcctccaaatgaaaggactcacttggtaattctgggagccatcttattatgccttggtgtagcactg |
| | acattcatcttccgtttaagaaaagggagaatgatggatgtgaaaaaatgtggcatccaagatacaaactcaaagaagcaaagtg |
| | atacacatttggaggagacgtaa |
| 57 (89†) | GTTCATGCGCAAGAGGATCG |
| 58 | atggaagatacaaaggagtctaacgtgaagacattttgctccaagaatatcctagccatccttggcttctcctctatcatagctgtga |
| | tagctttgcttgctgtggggttgacccagaacaaagcattgccagaaaacgttaagtatgggattgtgctggatgcgggttcttctc |
| | acacaagtttatacatctataagtggccagcagaaaaggagaatgacacaggcgtggtgcatcaagtagaagaatgcagggtta |
| | aaggtcctggaatctcaaaatttgttcagaaagtaaatgaaataggcatttacctgactgattgcatggaaagagctagggaagtg |
| | attccaaggtcccagcaccaagagacacccgtttacctgggagccacggcaggcatgcggttgctcaggatggaaagtgaaga |
| | gttggcagacagggttctggatgtggtggagaggagcctcagcaactacccctttgacttccagggtgccaggatcattactggc |
| | caagaggaaggtgcctatggctggattactatcaactatctgctgggcaaattcagtcagaaaacaaggtggttcagcatagtccc |
| | atatgaaaccaataatcaggaaacctttggagctttggaccttgggggagcctctacacaagtcacttttgtaccccaaaaccaga |
| | ctatcgagtccccagataatgctctgcaatttcgcctctatggcaaggactacaatgtctacacacatagcttcttgtgctatgggaa |
| | ggatcaggcactctggcagaaactggccaaggacattcaggttgcaagtaatgaaattctcagggacccatgctttcatcctggat |
| | ataagaaggtagtgaacgtaagtgacctttacaagaccccctgcaccaagagatttgagatgactcttccattccagcagtttgaa |
| | atccagggtattggaaactatcaacaatgccatcaaagcatcctggagctcttcaacaccagttactgcccttactcccagtgtgcc |
| | ttcaatgggattttcttgccaccactccagggggattttggggcattttcagctttttactttgtgatgaagtttttaaacttgacatcaga |
| | gaaagtctctcaggaaaaggtgactgagatgatgaaaaagttctgtgctcagccttgggaggagataaaaacatcttacgctgga |
| | gtaaaggagaagtacctgagtgaatactgcttttctggtacctacattctctccctccttctgcaaggctatcatttcacagctgattcc |
| | tgggagcacatccatttcattggcaagatccagggcagcgacgccggctggactttgggctacatgctgaacctgaccaacatg |
| | atcccagctgagcaaccattgtccacacctctctcccactccacctatgtcttcctcatggttctattctccctggtccttttcacagtg |
| | gccatcataggcttgcttatctttcacaagccttcatatttctggaaagatatggtaGGAAGCGGAGCTACTAACT |
| | TCAGCCTGCTGAAGCAGGCTGGAGACGTGGAGGAGAACCCTGGACCTatgtgtccc |
| | cgagccgcgcgggcgcccgcgacgctactcctcgccctgggcgcggtgctgtggcctgcggctggcgcctgggagcttacg |
| | attttgcacaccaacgacgtgcacagccggctggagcagaccagcgaggactccagcaagtgcgtcaacgccagccgctgca |
| | tgggtggcgtggctcggctcttcaccaaggttcagcagatccgccgcgccgaacccaacgtgctgctgctggacgccggcgac |
| | cagtaccagggcactatctggttcaccgtgtacaagggcgccgaggtggcgcacttcatgaacgccctgcgctacgatgccatg |
| | gcactgggaaatcatgaatttgataatggtgtggaaggactgatcgagccactcctcaaagaggccaaatttccaattctgagtgc |
| | aaacattaaagcaaaggggccactagcatctcaaatatcaggactttatttgccatataaagttcttcctgttggtgatgaagttgtgg |
| | gaatcgttggatacacttccaaagaaaccccttttctctcaaatccagggacaaatttagtgtttgaagatgaaatcactgcattaca |
| | acctgaagtagataagttaaaaactctaaatgtgaacaaaattattgcactgggacattcgggttttgaaatggataaactcatcgct |
| | cagaaagtgaggggtgtggacgtcgtggtgggaggacactccaacacatttctttacacaggcaatccaccttccaaagaggtg |
| | cctgctgggaagtacccattcatagtcacttctgatgatgggcggaaggttcctgtagtccaggcctatgcttttggcaaataccta |
| | ggctatctgaagatcgagtttgatgaaagaggaaacgtcatctcttcccatggaaatcccattcttctaaacagcagcattcctgaa |
| | gatccaagcataaaagcagacattaacaaatggaggataaaattggataattattctacccaggaattagggaaaacaattgtctat |
| | ctggatggctcctctcaatcatgccgctttagagaatgcaacatgggcaacctgatttgtgatgcaatgattaacaacaacctgaga |
| | cacacggatgaaatgttctggaaccacgtatccatgtgcattttaaatggaggtggtatccggtcgcccattgatgaacgcaacaa |
| | tggcacaattacctgggagaacctggctgctgtattgccctttggaggcacatttgacctagtccagttaaaaggttccaccctgaa |
| | gaaggcctttgagcatagcgtgcaccgctacggccagtccactggagagttcctgcaggtgggcggaatccatgtggtgtatga |
| | tctttcccgaaaacctggagacagagtagtcaaattagatgttctttgcaccaagtgtcgagtgcccagttatgaccctctcaaaatg |
| | gacgaggtatataaggtgatcctcccaaacttcctggccaatggtggagatgggttccagatgataaaagatgaattattaagaca |
| | tgactctggtgaccaagatatcaacgtggtttctacatatatctccaaaatgaaagtaatttatccagcagttgaaggtcggatcaag |
| | ttttccacaggaagtcactgccatggaagcttttctttaatatttctttcactttgggcagtgatctttgttttataccaa |
| 59 | AGGATACGTTTTTCTGTTGGGC |
| 60 | GGAGAACGGGAAAAGAGCGA |

†Spacer sequence is the RNA version of the target sequence upstream of the PAM site.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method of producing insulin in a mammalian subject, comprising:
a) administering to a mammalian subject at least one immune attenuating drug;
b) implanting a perforated cell delivery device comprising a genetically modified cell, or population thereof, into the mammalian subject; and
c) maturing the genetically modified cell, or population thereof, in the perforated cell delivery device in the mammalian subject such that the mature cell, or population thereof, produces insulin secreting cells, thereby producing insulin in the mammalian subject.

2. The method of claim 1, wherein
inflammation associated with foreign body response of the perforated cell delivery device loaded with the genetically modified cell, or population thereof in the mammalian subject is reduced compared to administering a comparative perforated cell delivery device loaded with the genetically modified cell, or population thereof, without the at least one immune attenuating drug.

3. (canceled)

4. (canceled)

5. (canceled)

6. The method of claim 1, wherein the genetically modified cell is an allogeneic pancreatic endoderm cell.

7. The method of claim 1, wherein the genetically modified cell comprises (a) a nucleic acid comprising a nucleotide sequence encoding programmed death-ligand 1 (PD-L1) inserted within a gene encoding beta-2 microglobulin (B2M) and (b) a nucleic acid comprising a nucleotide sequence encoding HLA class I histocompatibility antigen, alpha chain E (HLA-E) inserted within a gene encoding thioredoxin interacting protein (TXNIP), wherein the genetically modified cell expresses PD-L1 and HLA-E and has reduced or eliminated expression of B2M and TXNIP.

8. The method of claim 7, wherein the nucleic acid of (a) further comprises a nucleotide sequence encoding TNFAIP3 and/or wherein the nucleic acid of (b) further comprises a nucleotide sequence encoding MANF.

9. The method of claim 8, wherein the nucleic acid of (a) comprises the nucleotide sequence encoding TNFAIP3 linked to a nucleotide sequence encoding a P2A peptide linked to the nucleotide sequence encoding PD-L1 such that the nucleic acid of (a) comprises a TNFAIP3-P2A-PD-L1 polynucleotide sequence, wherein the TNFAIP3-P2A-PD-L1 polynucleotide sequence comprises the sequence of SEQ ID NO: 54.

10. (canceled)

11. (canceled)

12. (canceled)

13. The method of claim 8, wherein the nucleic acid of (b) comprises the nucleotide sequence encoding MANF linked to a nucleotide sequence encoding a P2A peptide linked to the nucleotide sequence encoding HLA-E such that the nucleic acid of (b) comprises a MANF-P2A-HLA-E polynucleotide sequence, wherein the MANF-P2A-HLA-E polynucleotide sequence comprises the sequence of SEQ ID NO: 55.

14.-22. (canceled)

23. The method of claim 1, wherein the at least one immune attenuating drug comprises a JAK1 or JAK2 inhibitor, a TNFα or TNFβ blocker, an ILIR blocker, a calcineurin inhibitor, an anti-thymocyte globulin or any combination thereof.

24. (canceled)

25. The method of claim 23, wherein the at least one immune attenuating drug comprises the calcineurin inhibitor, the anti-thymocyte globulin, or both.

26. The method of claim 25, wherein the calcineurin inhibitor is tacrolimus or cyclosporine A.

27. The method of claim 26, wherein the at least one immune attenuating drug comprises tacrolimus, the anti-thymocyte globulin, or both.

28.-36. (canceled)

37. The method of claim 1, wherein each of the at least one immune attenuating drug is administered by oral administration, intravenous administration, subcutaneous administration, or any combination thereof.

38. (canceled)

39. The method of claim 1, wherein each of the at least one immune attenuating drug is administered to the subject in a cycle of at least one week.

40. (canceled)

41. (canceled)

42. The method of claim 39, wherein the cycle begins prior to, after, or concurrently with the implanting of b).

43. The method of claim 42, wherein the cycle;
begins about 24 hours, about 12 hours, about 6 hours, about 5 hours, about 4 hours, about 3 hours, about 2 hours, about 1 hour or less, prior to the implanting of b); or
begins about 0.5 hour, about 1 hour, about 2 hours, about 3 hours, about 4 hours, about 5 hours, about 6 hours, about 12 hours, about 24 hours or more after the implanting of b).

44.-48. (canceled)

49. The method of claim 1, wherein b) comprises implanting more than one perforated cell delivery device into the mammalian subject.

50. The method of claim 49, wherein about 1.0×10⁷ to about 2.0×10⁷ genetically modified cells per kilogram are administered to the subject.

51. The method of claim 1, wherein the mammalian subject is human.

52. The method of claim 1, wherein C-peptide level is increased in the serum of the subject following the implanting of b), wherein the increase is relative to (i) the C-peptide level of the subject prior to the implanting of b); (ii) the C-peptide level in one or more untreated subjects; and/or (iii) a reference C-peptide level.

53.-59. (canceled)

60. A kit, comprising a perforated cell delivery device, a genetically modified cell, or population thereof, and at least one immune attenuating drug.

61.-90. (canceled)
